(19) United States
(12) Patent Application Publication
(10) Pub. No.: US 2022/0282268 A1
(43) Pub. Date: Sep. 8, 2022

(54) PYRENOID-LIKE STRUCTURES

(71) Applicant: THE UNIVERSITY COURT OF THE UNIVERSITY OF EDINBURGH, Edinburgh (GB)

(72) Inventors: Alistair James MCCORMICK, Edinburgh Lothian (GB); Nicola Jane ATKINSON, Edinburgh Lothian (GB)

(21) Appl. No.: 17/632,057

(22) PCT Filed: Jul. 31, 2020
(86) PCT No.: PCT/GB2020/051853

§ 371 Date: Feb. 1, 2022

(30) Foreign Application Priority Data
Aug. 2, 2019 (GB) 1911068.3

(51) Int. Cl. C12N 15/82; C12N 9/88

(52) U.S. Cl. CPC C12N 15/8257; C12N 9/88; C12Y 401/01039

(57) ABSTRACT

Aspects of the present disclosure relate to genetically altered plants having a modified Rubisco and further having a modified Essential Pyrenoid Component 1 (EPYC1) for formation of an aggregate of modified Rubisco and EPYC1 polypeptides. Other aspects of the present disclosure relate to methods of making such plants as well as cultivating these genetically altered plants.

### CROSS-REFERENCE TO RELATED APPLICATION

This application claims the benefit of U.K. Application No. 1911068.3, filed Aug. 2, 2019, which is hereby incorporated by reference in its entirety.

### SUBMISSION OF SEQUENCE LISTING AS ASCII TEXT FILE

The content of the following submission on ASCII text file is incorporated herein by reference in its entirety: a computer readable form (CRF) of the Sequence Listing (file name: 794542000841SEQLIST.TXT, date recorded: Jul. 15, 2020, size: 175 KB).

### TECHNICAL FIELD

The present disclosure relates to genetically altered plants. In particular, the present disclosure relates to genetically altered plants with a modified Rubisco and a modified Essential Pyrenoid Component 1 (EPYC1) for formation of an aggregate of modified Rubisco and EPYC1 polypeptides.

### BACKGROUND

Several photosynthetic organisms, including cyanobacteria, algae and a group of land plants called hornworts, have evolved biophysical CO₂-concentrating mechanisms (CCMs) that actively increase the CO₂ concentration around ribulose 1,5-biphosphate carboxylase oxygenase (Rubisco). The CCM improves Rubisco efficiency, because Rubisco has a relatively low affinity for CO₂ and a slow turnover rate. The algal CCM is composed of inorganic carbon (Ci) transporters at the plasma membrane and chloroplast envelope, which work together to deliver above ambient concentrations of CO₂ to Rubisco within the pyrenoid, a liquid-like organelle in the chloroplast.

The most common form of CO₂ assimilation in higher plants, including staple crops such as rice, wheat, and soybean, is C₃ photosynthesis. In C₃ photosynthesis, CO₂ delivery to chloroplasts occurs by passive diffusion, which limits photosynthetic efficiencies. Moreover, it has been estimated that the competitive side reaction with O₂ catalyzed by Rubisco (photorespiration) can result in a loss of productivity of up to 50% in C₃ plants (South, et al., JIPB (2018) 60: 1217-1230). Transferring the algal CCM mechanism into higher plants would address many of the inefficiencies of C₃ photosynthesis without requiring extensive morphological or genetic changes. In fact, key components of the algal CCM have been shown to localize correctly in higher plants (Atkinson, et al., Plant Biotech. J. (2016) 14: 1302-1315).

In order for CO₂ to be effectively concentrated in a CCM, Rubisco must be aggregated. The pyrenoid in the green alga Chlamydomonas reinhardtii contains Essential Pyrenoid Component 1 (EPYC1), which is a Rubisco linker protein that acts to aggregate Rubisco in the pyrenoid (Mackinder, et al., PNAS (2016) 113: 5958-5963). Rubisco and EPYC1 from C. reinhardtii have been shown to be necessary and sufficient to induce the liquid-liquid phase separation characteristic of pyrenoids (Wunder, et al., Nat. Commun. (2018) 9: 5076). The Rubisco small subunit (SSU, encoded by the rbcS nuclear gene family) of C. reinhardtii can complement severely SSU-deficient A. thaliana mutants (Atkinson, et al., New Phyt. (2017) 214: 655-667). Plants expressing the C. reinhardtii SSU can assemble hybrid Rubisco containing higher plant Rubisco large subunits (LSUs) and C. reinhardtii Rubisco SSUs, and this hybrid Rubisco has only slightly impaired Rubisco function compared to endogenous A. thaliana Rubisco. Further, plants with hybrid Rubisco have comparable plant growth to wild type plants. Moreover, plants with hybrid Rubisco have similar overall Rubisco levels as severely SSU-deficient A. thaliana mutants complemented with A. thaliana SSUs. In contrast, the replacement of tobacco Rubisco with cyanobacterial Rubisco produced poorer growing transplastomic plants, even when grown at greatly elevated CO₂ concentrations, due to the low affinity of cyanobacterial Rubisco for CO₂ and its low level of expression (Lin, et al., Nature (2014) 513: 547-550; Occhialini, et al., Plant J. (2016) 85: 148-160; Long, et al., Nat. Commun. (2018) 9: 3570).

Despite the success in engineering plants to have hybrid Rubisco, attempts to aggregate Rubisco in higher plants have been unsuccessful. Unlike previously tested algal CCM components, C. reinhardtii EPYC1 was unable to localize to the chloroplast when expressed in higher plants. Further, when EPYC1 was expressed in plants with hybrid Rubisco, aggregate was not observed. The addition of a higher plant chloroplast-targeting peptide to EPYC1 resulted in correctly localized EPYC1, however even when EPYC1 was localized to the chloroplast Rubisco aggregate was not observed.

### BRIEF SUMMARY

Surprisingly, it was found that the removal of the endogenous EPYC1 leader sequence and the replacement of this leader sequence with a better-processed heterologous leader sequence resulted in observable EPYC1 aggregate in higher plants. Increased expression of EPYC1 due to additional modifications, such as the use of a double terminator, further improved EPYC1 aggregates. In addition, it was also surprisingly found that the C. reinhardtii Rubisco SSU α-helices, and optionally the β-sheets and βA-βB loop, were necessary and sufficient for observing EPYC1 aggregate in higher plants. The surprising new modified EPYC1, as well as the necessary C. reinhardtii Rubisco SSU structural motifs, identified by the inventors serves as the basis for many of the aspects and their various embodiments of the present disclosure.

An aspect of the disclosure includes a genetically altered higher plant or part thereof including a modified Rubisco for formation of an aggregate of modified Rubisco and Essential Pyrenoid Component 1 (EPYC1) polypeptides. An additional embodiment of this aspect includes the modified Rubisco being an algal Rubisco small subunit (SSU) polypeptide or a modified higher plant Rubisco SSU polypeptide wherein at least part of the higher plant Rubisco SSU polypeptide is replaced with at least part of an algal Rubisco SSU polypeptide. In a further embodiment of this aspect, which may be combined with any of the preceding embodiments, the genetically altered higher plant or part thereof further includes the EPYC1 polypeptides and the aggregate. Yet another embodiment of this aspect, which may be combined with any of the preceding embodiments, includes the aggregate being detectable by confocal microscopy, transmission electron microscopy (TEM), cryo-electron microscopy (cryo-EM), or a liquid-liquid phase separation assay. Still another embodiment of this aspect, which may be combined with any of the preceding embodiments that has a modified higher plant Rubisco, includes the modified higher plant Rubisco polypeptide including an endogenous Rubisco SSU polypeptide. In yet another embodiment of this aspect, which may be combined with any of the preceding embodiments that has a modified higher plant Rubisco, the modified higher plant Rubisco SSU polypeptide was modified by substituting one or more higher plant Rubisco SSU α-helices with one or more algal Rubisco SSU α-helices; substituting one or more higher plant Rubisco SSU β-strands with one or more algal Rubisco SSU β-strands; and/or substituting a higher plant Rubisco SSU βA-βB loop with an algal Rubisco SSU βA-βB loop. An additional embodiment of this aspect includes the higher plant Rubisco SSU polypeptide being modified by substituting two higher plant Rubisco SSU α-helices with two algal Rubisco SSU α-helices. A further embodiment of this aspect includes the two higher plant Rubisco SSU α-helices corresponding to amino acids 23-35 and amino acids 80-93 in SEQ ID NO: 1 and the two algal Rubisco SSU α-helices corresponding to amino acids 23-35 and amino acids 86-99 in SEQ ID NO: 2. Yet another embodiment of this aspect that can be combined with any of the preceding embodiments that has two higher plant Rubisco SSU α-helices being substituted with two algal Rubisco SSU α-helices, the higher plant Rubisco SSU polypeptide being further modified by substituting four higher plant Rubisco SSU β-strands with four algal Rubisco SSU β-strands, and by substituting a higher plant Rubisco SSU βA-βB loop with an algal Rubisco SSU βA-βB loop. An additional embodiment of this aspect includes the four higher plant Rubisco SSU β-strands corresponding to amino acids 39-45, amino acids 68-70, amino acids 98-105, and amino acids 110-118 in SEQ ID NO: 1, the four algal Rubisco SSU β-strands corresponding to amino acids 39-45, amino acids 74-76, amino acids 104-111, and amino acids 116-124 in SEQ ID NO: 2, the higher plant Rubisco SSU βA-βB loop corresponding to amino acids 46-67 in SEQ ID NO: 1, and the algal Rubisco SSU βA-βB loop corresponding to amino acids 46-73 in SEQ ID NO: 2.

Still another embodiment of this aspect, which may be combined with any of the preceding embodiments that has a modified higher plant Rubisco, includes the higher plant Rubisco SSU polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, or SEQ ID NO: 156. Yet another embodiment of this aspect, which may be combined with any of the preceding embodiments that has a modified higher plant Rubisco, includes the algal Rubisco SSU polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 2, SEQ ID NO: 30, SEQ ID NO: 157, SEQ ID NO: 158, SEQ ID NO: 159, SEQ ID NO: 160, SEQ ID NO: 161, SEQ ID NO: 162, SEQ ID NO: 163, or SEQ ID NO: 164. In an additional embodiment of this aspect, the algal Rubisco SSU polypeptide is SEQ ID NO: 2, SEQ ID NO: 30, SEQ ID NO: 157, SEQ ID NO: 158, SEQ ID NO: 159, SEQ ID NO: 160, SEQ ID NO: 161, SEQ ID NO: 162, SEQ ID NO: 163, or SEQ ID NO: 164. A further embodiment of this aspect, which may be combined with any of the preceding embodiments that has a modified higher plant Rubisco, includes the modified higher plant Rubisco SSU polypeptide having increased affinity for the EPYC1 polypeptide as compared to the higher plant Rubisco SSU polypeptide without the modification.

An additional aspect of the disclosure includes a genetically altered higher plant or part thereof including EPYC1 polypeptides for formation of an aggregate of modified Rubiscos and the EPYC1 polypeptides. A further embodiment of any of the preceding aspects includes the EPYC1 polypeptides being algal EPYC1 polypeptides. An additional embodiment of this aspect includes the algal EPYC1 polypeptides having an amino acid sequence having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 34, SEQ ID NO: 35, SEQ ID NO: 165, SEQ ID NO: 166, or SEQ ID NO: 167. In yet another embodiment of this aspect, the algal EPYC1 polypeptide is SEQ ID NO: 34, SEQ ID NO: 35, SEQ ID NO: 165, SEQ ID NO: 166, or SEQ ID NO: 167. Still another embodiment of any of the preceding aspects includes the EPYC1 polypeptides being modified EPYC1 polypeptides. A further embodiment of this aspect includes the modified EPYC1 polypeptides including one or more, two or more, four or more, or eight tandem copies of a first algal EPYC1 repeat region. An additional embodiment of this aspect includes the modified EPYC1 polypeptides including four tandem copies or eight tandem copies of the first algal EPYC1 repeat region. Yet another embodiment of this aspect, which may be combined with any of the preceding embodiments including modified EPYC1 polypeptides including tandem copies of a first algal EPYC1 repeat region, includes the first algal EPYC1 repeat region being a polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 36. A further embodiment of this aspect includes the first algal EPYC1 repeat region being SEQ ID NO: 36. Still another embodiment of this aspect, which may be combined with any of the preceding embodiments including modified EPYC1, includes the modified EPYC1 polypeptides being expressed without the native EPYC1 leader sequence and/or including a C-terminal cap. Yet another embodiment of this aspect includes the native EPYC1 leader sequence including a polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 42, and the C-terminal cap including a polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 41. A further embodiment of this aspect includes the C-terminal cap being SEQ ID NO: 41. Still another embodiment of this aspect, which may be combined with any of the preceding embodiments including modified EPYC1, includes the modified EPYC1 polypeptide having increased affinity for Rubisco SSU polypeptide as compared to the corresponding unmodified EPYC1 polypeptide.

In yet another embodiment of this aspect, which may be combined with any of the preceding embodiments, the aggregate is localized to a chloroplast stroma of at least one chloroplast of a plant cell. A further embodiment of this aspect includes the plant cell being a leaf mesophyll cell. In still another embodiment of this aspect, which may be combined with any of the preceding embodiments, the plant is selected from the group of cowpea, soybean, cassava, rice, soy, wheat, or other C3 crop plants.

A further aspect of the disclosure includes a genetically altered higher plant or part thereof including a first nucleic acid sequence encoding an EPYC1 polypeptide and a second nucleic acid sequence encoding a modified Rubisco. An additional embodiment of this aspect includes the first nucleic acid sequence being operably linked to a first promoter. A further embodiment of this aspect includes the first promoter being selected from the group of a constitutive promoter, an inducible promoter, a leaf specific promoter, or a mesophyll cell specific promoter. Yet another embodiment of this aspect includes the first promoter being a constitutive promoter selected from the group of a CaMV35S promoter, a derivative of the CaMV35S promoter, a CsVMV promoter, a derivative of the CsVMV promoter, a maize ubiquitin promoter, a trefoil promoter, a vein mosaic cassava virus promoter, and an A. thaliana UBQ10 promoter. Still another embodiment of this aspect, which may be combined with any of the preceding embodiments, includes the first nucleic acid sequence being operably linked to a third nucleic acid sequence encoding a chloroplastic transit peptide functional in the higher plant cell, and the first nucleic acid sequence not including the native EPYC1 leader sequence and not being operably linked to the native EPYC1 leader sequence. An additional embodiment of this aspect includes the chloroplastic transit peptide being a polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 63. Yet another embodiment of this aspect includes the chloroplastic transit peptide being SEQ ID NO: 63. In a further embodiment of this aspect that can be combined with any of the preceding embodiments that has a native EPYC1 leader sequence, the native EPYC1 leader sequence corresponds to nucleotides 60-137 of SEQ ID NO: 65. In still another embodiment of this aspect that can be combined with any of the preceding embodiments, the first nucleic acid sequence is operably linked to one or two terminators. A further embodiment of this aspect includes the one two terminators being selected from the group of a HSP terminator, a NOS terminator, an OCS terminator, an intronless extensin terminator, a 35S terminator, a pinII terminator, a rbcS terminator, an actin terminator, or any combination thereof.

Still another embodiment of this aspect, which may be combined with any of the preceding embodiments, includes the second nucleic acid sequence being operably linked to a second promoter. In a further embodiment of this aspect, the second promoter is selected from the group of a constitutive promoter, an inducible promoter, a leaf specific promoter, or a mesophyll cell specific promoter. In an additional embodiment of this aspect, the second promoter is a constitutive promoter selected from the group of a CaMV35S promoter, a derivative of the CaMV35S promoter, a CsVMV promoter, a derivative of the CsVMV promoter, a maize ubiquitin promoter, a trefoil promoter, a vein mosaic cassava virus promoter, or an A. thaliana UBQ10 promoter. In yet another embodiment of this aspect that can be combined with any of the preceding embodiments that has a second nucleic acid sequence being operably linked to a second promoter, the second nucleic acid sequence encodes an algal Rubisco SSU polypeptide. In an additional embodiment of this aspect, the second nucleic acid sequence is operably linked to a fourth nucleic acid sequence encoding a chloroplastic transit peptide functional in the higher plant cell and the second nucleic acid sequence does not encode the native algal SSU leader sequence and is not operably linked to a nucleic acid sequence encoding the native algal SSU leader sequence. In a further embodiment of this aspect, the chloroplastic transit peptide is a polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 64. In yet another embodiment of this aspect, the chloroplastic transit peptide is SEQ ID NO: 64. In still another embodiment of this aspect that can be combined with any of the preceding embodiments that has a native algal SSU leader sequence, the native algal SSU leader sequence corresponds to amino acids 1 to 45 of SEQ ID NO: 32. In a further embodiment of this aspect that can be combined with any of the preceding embodiments that has a second nucleic acid sequence being operably linked to a second promoter, the second nucleic acid sequence is operably linked to a terminator. In an additional embodiment of this aspect, the terminator is selected from the group of a HSP terminator, a NOS terminator, an OCS terminator, an intronless extensin terminator, a 35S terminator, a pinII terminator, a rbcS terminator, or an actin terminator. In yet another embodiment of this aspect that can be combined with any of the preceding embodiments that has a second nucleic acid sequence being operably linked to a second promoter, the second nucleic acid sequence encodes a modified higher plant Rubisco SSU polypeptide wherein at least part of the higher plant Rubisco SSU polypeptide is replaced with at least part of an algal Rubisco SSU polypeptide. A further embodiment of this aspect, which can be combined with any of the preceding embodiments, includes the EPYC1 polypeptide being the EPYC1 polypeptide of any one of the preceding embodiments. An additional embodiment of this aspect includes the Rubisco SSU polypeptide being the Rubisco SSU polypeptide of any one of the preceding embodiments.

Yet another embodiment of this aspect, which may be combined with any of the preceding embodiments, includes at least one cell of the plant or part thereof including an aggregate of the Rubisco polypeptide and the EPYC1 polypeptide. A further embodiment of this aspect includes the aggregate being localized to a chloroplast stroma of at least one chloroplast of at least one plant cell. An additional embodiment of this aspect includes the plant cell being a leaf mesophyll cell. In still another embodiment of this aspect, which may be combined with any of the preceding embodiments that has a plant or part thereof including an aggregate of the Rubisco polypeptide and the EPYC1 polypeptide, the aggregate is detectable by confocal microscopy, transmission electron microscopy (TEM), cryo-electron microscopy (cryo-EM), or a liquid-liquid phase separation assay. In yet another embodiment of this aspect, which may be combined with any of the preceding embodiments, the plant is selected from the group of cowpea, soybean, cassava, rice, wheat, or other C3 crop plants. A further embodiment of this aspect that can be combined with any of the preceding embodiments includes a genetically altered higher plant cell produced from the plant or plant part of any one of the preceding embodiments.

Another aspect of the disclosure includes methods of producing the genetically altered higher plant of any of the preceding embodiments including a) introducing a first nucleic acid sequence encoding an EPYC1 polypeptide into a plant cell, tissue, or other explant; b) regenerating the plant cell, tissue, or other explant into a genetically altered plantlet; and c) growing the genetically altered plantlet into a genetically altered plant with the first nucleic acid encoding the EPYC1 polypeptide. An additional embodiment of this aspect further includes introducing a second nucleic acid sequence encoding a modified Rubisco SSU polypeptide into a plant cell, tissue, or other explant prior to step (a) or concurrently with step (a), wherein the genetically altered plant of step (c) further includes the second nucleic acid encoding the modified Rubisco SSU polypeptide. An additional embodiment of this aspect further includes identifying successful introduction of the first nucleic acid sequence and, optionally, the second nucleic acid sequence by screening or selecting the plant cell, tissue, or other explant prior to step (b); screening or selecting plantlets between step (b) and (c); or screening or selecting plants after step (c). In yet another embodiment of this aspect, which may be combined with any of the preceding embodiments, transformation is done using a transformation method selected from the group of particle bombardment (i.e., biolistics, gene gun), Agrobacterium-mediated transformation, Rhizobium-mediated transformation, or protoplast transfection or transformation.

Still another embodiment of this aspect that can be combined with any of the preceding embodiments includes the first nucleic acid sequence being introduced with a first vector, and the second nucleic acid sequence being introduced with a second vector. In a further embodiment of this aspect, the first nucleic acid sequence is operably linked to a first promoter. In an additional embodiment of this aspect, the first promoter is selected from the group of a constitutive promoter, an inducible promoter, a leaf specific promoter, or a mesophyll cell specific promoter. In yet another embodiment of this aspect, the first promoter is a constitutive promoter selected from the group of a CaMV35S promoter, a derivative of the CaMV35S promoter, a CsVMV promoter, a derivative of the CsVMV promoter, a maize ubiquitin promoter, a trefoil promoter, a vein mosaic cassava virus promoter, or an A. thaliana UBQ10 promoter. In still another embodiment of this aspect that can be combined with any of the preceding embodiments, the first nucleic acid sequence is operably linked to a third nucleic acid sequence encoding a chloroplastic transit peptide functional in the higher plant cell and the first nucleic acid sequence does not include the native EPYC1 leader sequence and is not operably linked to the native EPYC1 leader sequence. In yet another embodiment of this aspect, the chloroplastic transit peptide is a polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 63. In still another embodiment of this aspect, the endogenous chloroplastic transit peptide is SEQ ID NO: 63. Yet another embodiment of this aspect that can be combined with any of the preceding embodiments that has a native EPYC1 leader sequence includes the native EPYC1 leader sequence corresponding to nucleotides 60 to 137 of SEQ ID NO: 65. In a further embodiment of this aspect that can be combined with any of the preceding embodiments, the first nucleic acid sequence is operably linked to one or two terminators. In an additional embodiment of this aspect, the one or two terminators are selected from the group of a HSP terminator, a NOS terminator, an OCS terminator, an intronless extensin terminator, a 35S terminator, a pinII terminator, an rbcS terminator, an actin terminator, or any combination thereof.

An additional embodiment of this aspect that can be combined with any of the preceding embodiments includes the second nucleic acid sequence being operably linked to a second promoter. A further embodiment of this aspect includes the second promoter being selected from the group consisting of a constitutive promoter, an inducible promoter, a leaf specific promoter, and a mesophyll cell specific promoter. Yet another embodiment of this aspect includes the second promoter being a constitutive promoter selected from the group consisting of a CaMV35S promoter, a derivative of the CaMV35S promoter, a CsVMV promoter, a derivative of the CsVMV promoter, a maize ubiquitin promoter, a trefoil promoter, a vein mosaic cassava virus promoter, or an A. thaliana UBQ10 promoter. Still another embodiment of this aspect that can be combined with any of the preceding embodiments that has the second nucleic acid sequence being operably linked to a second promoter includes the second nucleic acid sequence encoding an algal SSU polypeptide. An additional embodiment of this aspect includes the second nucleic acid sequence being operably linked to a fourth nucleic acid sequence encoding a chloroplastic transit peptide functional in the higher plant cell and the second nucleic acid sequence not encoding the native SSU leader sequence and not being operably linked to a nucleic acid sequence encoding the native SSU leader sequence. A further embodiment of this aspect includes the chloroplastic transit peptide being a polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 64. Yet another embodiment of this aspect includes the chloroplastic transit peptide being SEQ ID NO: 64. An additional embodiment of this aspect, which can be combined with any of the preceding embodiments that has a native SSU leader sequence, includes the native SSU leader sequence corresponding to amino acids 1 to 45 of SEQ ID NO: 32. Still another embodiment of this aspect that can be combined with any of the preceding embodiments that has the second nucleic acid sequence being operably linked to a second promoter includes the second nucleic acid sequence being operably linked to a terminator. A further embodiment of this aspect includes the terminator being selected from the group of a HSP terminator, a NOS terminator, an OCS terminator, an intronless extensin terminator, a 35S terminator, a pinII terminator, an rbcS terminator, or an actin terminator. In a further embodiment of this aspect that can be combined with any of the preceding embodiments that has the second nucleic acid sequence being operably linked to a second promoter, the second nucleic acid sequence encodes a modified higher plant Rubisco SSU polypeptide wherein at least part of the higher plant Rubisco SSU polypeptide is replaced with at least part of an algal Rubisco SSU polypeptide.

In an additional embodiment of this aspect that can be combined with any of the preceding embodiments that has a second vector, the second vector includes one or more gene editing components that target a nuclear genome sequence operably linked to a nucleic acid encoding an endogenous Rubisco SSU polypeptide. A further embodiment of this aspect includes one or more gene editing components being selected from the group of a ribonucleoprotein complex that targets the nuclear genome sequence; a vector comprising a TALEN protein encoding sequence, wherein the TALEN protein targets the nuclear genome sequence; a vector comprising a ZFN protein encoding sequence, wherein the ZFN protein targets the nuclear genome sequence; an oligonucleotide donor (ODN), wherein the ODN targets the nuclear genome sequence; or a vector comprising a CRISPR/Cas enzyme encoding sequence and a targeting sequence, wherein the targeting sequence targets the nuclear genome sequence. Yet another embodiment of this aspect that can be combined with any of the preceding embodiments that has gene editing includes the result of gene editing being at least part of the higher plant Rubisco SSU polypeptide being replaced with at least part of an algal Rubisco SSU polypeptide. A further embodiment of this aspect, which can be combined with any of the preceding embodiments, includes the EPYC1 polypeptide being the EPYC1 polypeptide of any one of the preceding embodiments. An additional embodiment of this aspect includes the Rubisco SSU polypeptide being the Rubisco SSU polypeptide of any one of the preceding embodiments.

Yet another embodiment of this aspect that can be combined with any of the preceding embodiments that has a first nucleic acid sequence being operably linked to a third nucleic acid sequence encoding a chloroplastic transit peptide functional in the higher plant cell and the first nucleic acid sequence not comprising the native EPYC1 leader sequence and not being operably linked to the native EPYC1 leader sequence includes and that has the first nucleic acid sequence being operably linked to one or two terminators includes the first vector including a first copy of the first nucleic acid sequence wherein the first nucleic acid sequence does not include the native EPYC1 leader sequence and is not operably linked to the native EPYC1 leader sequence, wherein the first nucleic acid sequence is operably linked to the third nucleic acid sequence encoding a chloroplastic transit peptide functional in the higher plant cell, wherein the first nucleic acid sequence is operably linked to the first promoter, and wherein the first nucleic acid sequence is operably linked to one terminator; and wherein the first vector further includes a second copy of the first nucleic acid sequence wherein the first nucleic acid sequence does not include the native EPYC1 leader sequence and is not operably linked to the native EPYC1 leader sequence, wherein the first nucleic acid sequence is operably linked to the third nucleic acid sequence encoding a chloroplastic transit peptide functional in the higher plant cell, wherein the first nucleic acid sequence is operably linked to a third promoter, and wherein the first nucleic acid sequence is operably linked to two terminators. A further embodiment of this aspect includes the first promoter being selected from the group of a constitutive promoter, an inducible promoter, a leaf specific promoter, or a mesophyll cell specific promoter; wherein the third promoter is selected from the group of a constitutive promoter, an inducible promoter, a leaf specific promoter, or a mesophyll cell specific promoter; and wherein the first and third promoters are not the same. Yet another embodiment of this aspect includes the chloroplastic transit peptide being a polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 63. Still another embodiment of this aspect includes the native EPYC1 leader sequence corresponding to nucleotides 60 to 137 of SEQ ID NO: 65. An additional embodiment of this aspect includes the terminators being selected from the group of a HSP terminator, a NOS terminator, an OCS terminator, an intronless extensin terminator, a 35S terminator, a pinII terminator, a rbcS terminator, an actin terminator, or any combination thereof. A further embodiment of this aspect that can be combined with any of the preceding embodiments includes a plant or plant part produced by the method of any one of the preceding embodiments.

A further aspect of the disclosure includes methods of cultivating the genetically altered plant of any of the preceding embodiments that has a genetically altered plant, including the steps of: a) planting a genetically altered seedling, a genetically altered plantlet, a genetically altered cutting, a genetically altered tuber, a genetically altered root, or a genetically altered seed in soil to produce the genetically altered plant or grafting the genetically altered seedling, the genetically altered plantlet, or the genetically altered cutting to a root stock or a second plant grown in soil to produce the genetically altered plant; b) cultivating the plant to produce harvestable seed, harvestable leaves, harvestable roots, harvestable cuttings, harvestable wood, harvestable fruit, harvestable kernels, harvestable tubers, and/or harvestable grain; and harvesting the harvestable seed, harvestable leaves, harvestable roots, harvestable cuttings, harvestable wood, harvestable fruit, harvestable kernels, harvestable tubers, and/or harvestable grain; and c) harvesting the harvestable seed, harvestable leaves, harvestable roots, harvestable cuttings, harvestable wood, harvestable fruit, harvestable kernels, harvestable tubers, and/or harvestable grain.

### ENUMERATED EMBODIMENTS

1. A genetically altered higher plant or part thereof, comprising a modified Rubisco for formation of an aggregate of Essential Pyrenoid Component 1 (EPYC1) polypeptides and modified Rubiscos, wherein the modified Rubisco comprises an algal Rubisco small subunit (SSU) polypeptide or a modified higher plant Rubisco SSU polypeptide wherein at least part of the higher plant Rubisco SSU polypeptide is replaced with at least part of an algal Rubisco SSU polypeptide.
2. The plant or part thereof of embodiment 1, further comprising the EPYC1 polypeptides and the aggregate.
3. The plant or part thereof of embodiment 1, wherein the modified Rubisco comprising the algal Rubisco SSU polypeptide has increased affinity for the EPYC1 polypeptides as compared to unmodified Rubisco.
4. The plant or part thereof of embodiment 1, wherein the modified higher plant Rubisco SSU polypeptide was modified by substituting one or more higher plant Rubisco SSU α-helices with one or more algal Rubisco SSU α-helices; substituting one or more higher plant Rubisco SSU β-strands with one or more algal Rubisco SSU β-strands; and/or substituting a higher plant Rubisco SSU βA-βB loop with an algal Rubisco SSU βA-βB loop.
5. The plant or part thereof of embodiment 1, wherein the modified higher plant Rubisco SSU polypeptide has increased affinity for the EPYC1 polypeptides as compared to the higher plant Rubisco SSU polypeptide without the modification.
6. A genetically altered higher plant or part thereof, comprising EPYC1 polypeptides for formation of an aggregate of the EPYC1 polypeptides and modified Rubiscos.
7. The plant or part thereof of embodiment 6, wherein the EPYC1 polypeptides are algal EPYC1 polypeptides or modified EPYC1 polypeptides comprising one or more, two or more, four or more, or eight tandem copies of a first algal EPYC1 repeat region.
8. The plant or part thereof of embodiment 7, wherein the algal EPYC1 polypeptides are truncated mature EPYC1 polypeptides.
9. The plant or part thereof of embodiment 8, wherein the truncated mature EPYC1 polypeptides have increased affinity for the modified Rubiscos as compared to the non-truncated EPYC1 polypeptides.
10. The plant or part thereof of embodiment 7, wherein the modified EPYC1 polypeptides are expressed without the native EPYC1 leader sequence and/or comprise a C-terminal cap.
11. The plant or part thereof of embodiment 10, wherein the modified EPYC1 polypeptides have increased affinity for the modified Rubiscos as compared to the corresponding unmodified EPYC1 polypeptide.
12. The plant or part thereof of embodiment 6, wherein the aggregate is localized to a chloroplast stroma of at least one chloroplast of a plant cell, and wherein the plant cell is a leaf mesophyll cell.
13. A genetically altered higher plant or part thereof, comprising a first nucleic acid sequence encoding an EPYC1 polypeptide and a second nucleic acid sequence encoding a modified Rubisco polypeptide.
14. The plant or part thereof of embodiment 13, wherein the first nucleic acid sequence is operably linked to a third nucleic acid sequence encoding a chloroplastic transit peptide functional in the higher plant cell, and wherein the first nucleic acid sequence does not comprise the native EPYC1 leader sequence and is not operably linked to the native EPYC1 leader sequence, and wherein the second nucleic acid sequence is operably linked to a fourth nucleic acid sequence encoding a chloroplastic transit peptide functional in the higher plant cell and wherein the second nucleic acid sequence does not encode the native algal SSU leader sequence and is not operably linked to a nucleic acid sequence encoding the native algal SSU leader sequence.
15. The plant or part thereof of embodiment 13, wherein the EPYC1 polypeptide is a truncated mature EPYC1 polypeptide or a modified EPYC1 polypeptide comprising one or more, two or more, four or more, or eight tandem copies of a first algal EPYC1 repeat region.
16. The plant or part thereof of embodiment 13, wherein the modified Rubisco polypeptide comprises an algal Rubisco small subunit (SSU) polypeptide or a modified higher plant Rubisco SSU polypeptide wherein at least part of the higher plant Rubisco SSU polypeptide is replaced with at least part of an algal Rubisco SSU polypeptide.
17. The plant or part thereof of embodiment 13, wherein the plant or part thereof further comprises an aggregate of the modified Rubisco polypeptides and the EPYC1 polypeptides.
18. A method of producing the genetically altered higher plant of embodiment 1, comprising:
a) introducing a first nucleic acid sequence encoding an EPYC1 polypeptide into a plant cell, tissue, or other explant;
b) regenerating the plant cell, tissue, or other explant into a genetically altered plantlet; and
c) growing the genetically altered plantlet into a genetically altered plant with the first nucleic acid encoding the EPYC1 polypeptide.
   19. The method of embodiment 18, further comprising introducing a second nucleic acid sequence encoding a modified Rubisco SSU polypeptide into a plant cell, tissue, or other explant prior to step (a) or concurrently with step (a), wherein the genetically altered plant of step (c) further comprises the second nucleic acid encoding the modified Rubisco SSU polypeptide.
   20. The method of embodiment 18, wherein the first nucleic acid sequence is introduced with a first vector, and wherein the first vector comprises a first copy of the first nucleic acid sequence wherein the first nucleic acid sequence does not comprise the native EPYC1 leader sequence and is not operably linked to the native EPYC1 leader sequence, wherein the first nucleic acid sequence is operably linked to the third nucleic acid sequence encoding a chloroplastic transit peptide functional in the higher plant cell, wherein the first nucleic acid sequence is operably linked to the first promoter, and wherein the first nucleic acid sequence is operably linked to one terminator; and wherein the first vector further comprises a second copy of the first nucleic acid sequence wherein the first nucleic acid sequence does not comprise the native EPYC1 leader sequence and is not operably linked to the native EPYC1 leader sequence, wherein the first nucleic acid sequence is operably linked to the third nucleic acid sequence encoding a chloroplastic transit peptide functional in the higher plant cell, wherein the first nucleic acid sequence is operably linked to a third promoter, and wherein the first nucleic acid sequence is operably linked to two terminators.
   21. A genetically altered higher plant or part thereof, comprising a modified Rubisco for formation of an aggregate of modified Rubisco and Essential Pyrenoid Component 1 (EPYC1) polypeptides.
   22. The plant or part thereof of embodiment 21, wherein the modified Rubisco comprises an algal Rubisco small subunit (SSU) polypeptide or a modified higher plant Rubisco SSU polypeptide wherein at least part of the higher plant Rubisco SSU polypeptide is replaced with at least part of an algal Rubisco SSU polypeptide.
   23. The plant or part thereof of embodiment 21 or embodiment 22, further comprising the EPYC1 polypeptides and the aggregate.
   24. The plant or part thereof of any one of embodiments 21-23, wherein the aggregate is detectable by confocal microscopy, transmission electron microscopy (TEM), cryo-electron microscopy (cryo-EM), or a liquid-liquid phase separation assay.
   25. The plant or part thereof of any one of embodiments 22-24, wherein the modified higher plant Rubisco polypeptide comprises an endogenous Rubisco SSU polypeptide.
   26. The plant or part thereof of any one of embodiments 22-25, wherein the modified higher plant Rubisco SSU polypeptide was modified by substituting one or more higher plant Rubisco SSU α-helices with one or more algal Rubisco SSU α-helices; substituting one or more higher plant Rubisco SSU β-strands with one or more algal Rubisco SSU β-strands; and/or substituting a higher plant Rubisco SSU βA-βB loop with an algal Rubisco SSU βA-βB loop.
   27. The plant or part thereof of embodiment 26, wherein the higher plant Rubisco SSU polypeptide is modified by substituting two higher plant Rubisco SSU α-helices with two algal Rubisco SSU α-helices.
   28. The plant or part thereof of embodiment 27, wherein the two higher plant Rubisco SSU α-helices correspond to amino acids 23-35 and amino acids 80-93 in SEQ ID NO: 1 and the two algal Rubisco SSU α-helices correspond to amino acids 23-35 and amino acids 86-99 in SEQ ID NO: 2.
   29. The plant or part thereof of embodiment 27 or embodiment 28, wherein the higher plant Rubisco SSU polypeptide is further modified by substituting four higher plant Rubisco SSU β-strands with four algal Rubisco SSU β-strands, and by substituting a higher plant Rubisco SSU βA-βB loop with an algal Rubisco SSU βA-βB loop.
   30. The plant or part thereof of embodiment 29, wherein the four higher plant Rubisco SSU β-strands correspond to amino acids 39-45, amino acids 68-70, amino acids 98-105, and amino acids 110-118 in SEQ ID NO: 1, the four algal Rubisco SSU β-strands correspond to amino acids 39-45, amino acids 74-76, amino acids 104-111, and amino acids 116-124 in SEQ ID NO: 2, the higher plant Rubisco SSU βA-βB loop corresponds to amino acids 46-67 in SEQ ID NO: 1, and the algal Rubisco SSU βA-βB loop corresponds to amino acids 46-73 in SEQ ID NO: 2.
   31. The plant or part thereof of any one of embodiments 22-30, wherein the higher plant Rubisco SSU polypeptide had at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, or SEQ ID NO: 156.
   32. The plant or part thereof of any one of embodiments 22-31, wherein the algal Rubisco SSU polypeptide has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 2, SEQ ID NO: 30, SEQ ID NO: 157, SEQ ID NO: 158, SEQ ID NO: 159, SEQ ID NO: 160, SEQ ID NO: 161, SEQ ID NO: 162, SEQ ID NO: 163, or SEQ ID NO: 164.
   33. The plant or part thereof of embodiment 32, wherein the algal Rubisco SSU polypeptide is SEQ ID NO: 2, SEQ ID NO: 30, SEQ ID NO: 157, SEQ ID NO: 158, SEQ ID NO: 159, SEQ ID NO: 160, SEQ ID NO: 161, SEQ ID NO: 162, SEQ ID NO: 163, or SEQ ID NO: 164.
   34. The plant or part thereof of any one of embodiments 22-31, wherein the modified higher plant Rubisco SSU polypeptide has increased affinity for the EPYC1 polypeptide as compared to the higher plant Rubisco SSU polypeptide without the modification.
   35. A genetically altered higher plant or part thereof, comprising EPYC1 polypeptides for formation of an aggregate of modified Rubiscos and the EPYC1 polypeptides.
   36. The plant or part thereof of any one of embodiments 21-35, wherein the EPYC1 polypeptides are algal EPYC1 polypeptides.
   37. The plant or part thereof of embodiment 35 or embodiment 36, wherein the algal EPYC1 polypeptides comprise an amino acid sequence having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 34, SEQ ID NO: 35, SEQ ID NO: 165, SEQ ID NO: 166, or SEQ ID NO: 167.
   38. The plant or part thereof of embodiment 37, wherein the algal EPYC1 polypeptide is SEQ ID NO: 34, SEQ ID NO: 35, SEQ ID NO: 165, SEQ ID NO: 166, or SEQ ID NO: 167.
   39. The plant or part thereof of any one of embodiments 21-37, wherein the EPYC1 polypeptides are modified EPYC1 polypeptides.
   40. The plant or part thereof of embodiment 39, wherein the modified EPYC1 polypeptides comprise one or more, two or more, four or more, or eight tandem copies of a first algal EPYC1 repeat region.
   41. The plant or part thereof of embodiment 40, wherein the modified EPYC1 polypeptides comprise four tandem copies or eight tandem copies of the first algal EPYC1 repeat region.
   42. The plant or part thereof of embodiment 40 or embodiment 41, wherein the first algal EPYC1 repeat region is a polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 36.
   43. The plant or part thereof of embodiment 42, wherein the first algal EPYC1 repeat region is SEQ ID NO: 36.
   44. The plant or part thereof of any one of embodiments 39-43, wherein the modified EPYC1 polypeptides are expressed without the native EPYC1 leader sequence and/or comprise a C-terminal cap.
   45. The plant or part thereof of embodiment 44, wherein the native EPYC1 leader sequence comprises a polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 42, and wherein the C-terminal cap comprises a polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 41.
   46. The plant or part thereof of embodiment 45, wherein the C-terminal cap is SEQ ID NO: 41.
   47. The plant or part thereof of any one of embodiments 39-46, wherein the modified EPYC1 polypeptide has increased affinity for the Rubisco SSU polypeptide as compared to the corresponding unmodified EPYC1 polypeptide.
   48. The plant or part thereof of any one of embodiments 21-47, wherein the aggregate is localized to a chloroplast stroma of at least one chloroplast of a plant cell.
   49. The plant of embodiment 48, wherein the plant cell is a leaf mesophyll cell.
   50. The plant of any one of embodiments 21-49, wherein the plant is selected from the group consisting of cowpea, soybean, cassava, rice, soy, wheat, and other C3 crop plants.
   51. A genetically altered higher plant or part thereof, comprising a first nucleic acid sequence encoding an EPYC1 polypeptide and a second nucleic acid sequence encoding a modified Rubisco.
   52. The plant or part thereof of embodiment 51, wherein the first nucleic acid sequence is operably linked to a first promoter.
   53. The plant or part thereof of embodiment 52, wherein the first promoter is selected from the group consisting of a constitutive promoter, an inducible promoter, a leaf specific promoter, and a mesophyll cell specific promoter.
   54. The plant or part thereof of embodiment 53, wherein the first promoter is a constitutive promoter selected from the group consisting of a CaMV35S promoter, a derivative of the CaMV35S promoter, a CsVMV promoter, a derivative of the CsVMV promoter, a maize ubiquitin promoter, a trefoil promoter, a vein mosaic cassava virus promoter, and an A. thaliana UBQ10 promoter.
   55. The plant or part thereof of any one of embodiments 51-54, wherein the first nucleic acid sequence is operably linked to a third nucleic acid sequence encoding a chloroplastic transit peptide functional in the higher plant cell, and wherein the first nucleic acid sequence does not comprise the native EPYC1 leader sequence and is not operably linked to the native EPYC1 leader sequence.
   56. The plant or part thereof of embodiment 55, wherein the chloroplastic transit peptide is a polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 63.
   57. The plant or part thereof of embodiment 56, wherein the chloroplastic transit peptide is SEQ ID NO: 63.
   58. The plant or part thereof of any one of embodiments 55-57, wherein the native EPYC1 leader sequence corresponds to nucleotides 60 to 137 of SEQ ID NO: 65.
   59. The plant or part thereof of any one of embodiments 51-58, wherein the first nucleic acid sequence is operably linked to one or two terminators.
   60. The plant or part thereof of embodiment 59, wherein the one two terminators are selected from the group consisting of a HSP terminator, a NOS terminator, an OCS terminator, an intronless extensin terminator, a 35S terminator, a pinII terminator, a rbcS terminator, an actin terminator, and any combination thereof.
   61. The plant or part thereof of any one of embodiments 51-60, wherein the second nucleic acid sequence is operably linked to a second promoter.
   62. The plant or part thereof of embodiment 61, wherein the second promoter is selected from the group consisting of a constitutive promoter, an inducible promoter, a leaf specific promoter, and a mesophyll cell specific promoter.
   63. The plant or part thereof of embodiment 62, wherein the second promoter is a constitutive promoter selected from the group consisting of a CaMV35S promoter, a derivative of the CaMV35S promoter, a CsVMV promoter, a derivative of the CsVMV promoter, a maize ubiquitin promoter, a trefoil promoter, a vein mosaic cassava virus promoter, and an A. thaliana UBQ10 promoter.
   64. The plant or part thereof of any one of embodiments 61-63, wherein the second nucleic acid sequence encodes an algal Rubisco SSU polypeptide.
   65. The plant or part thereof of embodiment 64, wherein the second nucleic acid sequence is operably linked to a fourth nucleic acid sequence encoding a chloroplastic transit peptide functional in the higher plant cell and wherein the second nucleic acid sequence does not encode the native algal SSU leader sequence and is not operably linked to a nucleic acid sequence encoding the native algal SSU leader sequence.
   66. The plant or part thereof of embodiment 65, wherein the chloroplastic transit peptide is a polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 64.
   67. The plant or part thereof of embodiment 66, wherein the chloroplastic transit peptide is SEQ ID NO: 64.
   68. The plant or part thereof of any one of embodiments 65-67, wherein the native SSU leader sequence corresponds to amino acids 1 to 45 of SEQ ID NO: 32.
   69. The plant or part thereof of any one of embodiments 61-68, wherein the second nucleic acid sequence is operably linked to a terminator.
   70. The plant or part thereof of embodiment 69, wherein the terminator is selected from the group consisting of a HSP terminator, a NOS terminator, an OCS terminator, an intronless extensin terminator, a 35S terminator, a pinII terminator, a rbcS terminator, and an actin terminator.
   71. The plant or part thereof of any one of embodiments 61-63, wherein the second nucleic acid sequence encodes a modified higher plant Rubisco SSU polypeptide wherein at least part of the higher plant Rubisco SSU polypeptide is replaced with at least part of an algal Rubisco SSU polypeptide.
   72. The plant or part thereof of any one of embodiments 51-71, wherein the EPYC1 polypeptide is the EPYC1 polypeptide of any one of embodiments 36-47.
   73. The plant or part thereof of any one of embodiments 51-72, wherein the Rubisco SSU polypeptide is the Rubisco SSU polypeptide of any one of embodiments 25-34.
   74. The plant or part thereof of any one of embodiments 51-73, wherein at least one cell of the plant or part thereof comprises an aggregate of the Rubisco polypeptide and the EPYC1 polypeptide.
   75. The plant or part thereof of embodiment 74, wherein the aggregate is localized to a chloroplast stroma of at least one chloroplast of at least one plant cell.
   76. The plant of embodiment 75, wherein the plant cell is a leaf mesophyll cell.
   77. The plant of any one of embodiments 74-76, wherein the aggregate is detectable by confocal microscopy, transmission electron microscopy (TEM), cryo-electron microscopy (cryo-EM), or a liquid-liquid phase separation assay.
   78. The plant of any one of embodiments 71-77, wherein the plant is selected from the group consisting of cowpea, soybean, cassava, rice, wheat, and other C3 crop plants.
   79. A genetically altered higher plant cell obtainable from the plant or plant part of any one of embodiments 21-78.
   80. A method of producing the genetically altered higher plant of any one of embodiments 21-79, comprising:
d) introducing a first nucleic acid sequence encoding an EPYC1 polypeptide into a plant cell, tissue, or other explant;
e) regenerating the plant cell, tissue, or other explant into a genetically altered plantlet; and
f) growing the genetically altered plantlet into a genetically altered plant with the first nucleic acid encoding the EPYC1 polypeptide.
   81. The method of embodiment 80, further comprising introducing a second nucleic acid sequence encoding a modified Rubisco SSU polypeptide into a plant cell, tissue, or other explant prior to step (a) or concurrently with step (a), wherein the genetically altered plant of step (c) further comprises the second nucleic acid encoding the modified Rubisco SSU polypeptide.
   82. The method of embodiment 80 or embodiment 81, further comprising identifying successful introduction of the first nucleic acid sequence and, optionally, the second nucleic acid sequence by screening or selecting the plant cell, tissue, or other explant prior to step (b); screening or selecting plantlets between step (b) and (c); or screening or selecting plants after step (c).
   83. The method of any one of embodiments 80-82, wherein transformation is done using a transformation method selected from the group consisting of particle bombardment (i.e., biolistics, gene gun), Agrobacterium-mediated transformation, Rhizobium-mediated transformation, and protoplast transfection or transformation.
   84. The method of any one of embodiments 81-83, wherein the first nucleic acid sequence is introduced with a first vector, and wherein the second nucleic acid sequence is introduced with a second vector.
   85. The method of embodiment 84, wherein the first nucleic acid sequence is operably linked to a first promoter.
   86. The method of embodiment 85, wherein the first promoter is selected from the group consisting of a constitutive promoter, an inducible promoter, a leaf specific promoter, and a mesophyll cell specific promoter.
   87. The method of embodiment 86, wherein the first promoter is a constitutive promoter selected from the group consisting of a CaMV35S promoter, a derivative of the CaMV35S promoter, a CsVMV promoter, a derivative of the CsVMV promoter, a maize ubiquitin promoter, a trefoil promoter, a vein mosaic cassava virus promoter, and an A. thaliana UBQ10 promoter.
   88. The method of any one of embodiments 80-87, wherein the first nucleic acid sequence is operably linked to a third nucleic acid sequence encoding a chloroplastic transit peptide functional in the higher plant cell and wherein the first nucleic acid sequence does not comprise the native EPYC1 leader sequence and is not operably linked to the native EPYC1 leader sequence.
   89. The method of embodiment 88, wherein the chloroplastic transit peptide is a polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 63.
   90. The method of embodiment 89, wherein the endogenous chloroplastic transit peptide is SEQ ID NO: 63.
   91. The method of any one of embodiments 88-90, wherein the native EPYC1 leader sequence corresponds to nucleotides 60 to 137 of SEQ ID NO: 65.
   92. The method of any one of embodiments 80-91, wherein the first nucleic acid sequence is operably linked to one or two terminators.
   93. The method of embodiment 92, wherein the one or two terminators are selected from the group consisting of a HSP terminator, a NOS terminator, an OCS terminator, an intronless extensin terminator, a 35S terminator, a pinII terminator, a rbcS terminator, an actin terminator, and any combination thereof.
   94. The method of any one of embodiments 81-93, wherein the second nucleic acid sequence is operably linked to a second promoter.
   95. The method of embodiment 94, wherein the second promoter is selected from the group consisting of a constitutive promoter, an inducible promoter, a leaf specific promoter, and a mesophyll cell specific promoter.
   96. The method of embodiment 95, wherein the second promoter is a constitutive promoter selected from the group consisting of a CaMV35S promoter, a derivative of the CaMV35S promoter, a CsVMV promoter, a derivative of the CsVMV promoter, a maize ubiquitin promoter, a trefoil promoter, a vein mosaic cassava virus promoter, and an A. thaliana UBQ10 promoter.
   97. The method of any one of embodiments 94-96, wherein the second nucleic acid sequence encodes an algal SSU polypeptide.
   98. The method of embodiment 97, wherein the second nucleic acid sequence is operably linked to a fourth nucleic acid sequence encoding a chloroplastic transit peptide functional in the higher plant cell and wherein the second nucleic acid sequence does not encode the native algal SSU leader sequence and is not operably linked to a nucleic acid sequence encoding the native algal SSU leader sequence.
   99. The method of embodiment 98, wherein the chloroplastic transit peptide is a polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 64.
   100. The method of embodiment 99, wherein the chloroplastic transit peptide is SEQ ID NO: 64.
   101. The method of any one of embodiments 98-100, wherein the native algal SSU leader sequence corresponds amino acids 1 to 45 of SEQ ID NO: 32.
   102. The method of any one of embodiments 94-101, wherein the second nucleic acid sequence is operably linked to a terminator.
   103. The method of embodiment 102, wherein the terminator is selected from the group consisting of a HSP terminator, a NOS terminator, an OCS terminator, an intronless extensin terminator, a 35S terminator, a pinII terminator, a rbcS terminator, and an actin terminator.
   104. The method of any one of embodiments 94-96, wherein the second nucleic acid sequence encodes a modified higher plant Rubisco SSU polypeptide wherein at least part of the higher plant Rubisco SSU polypeptide is replaced with at least part of an algal Rubisco SSU polypeptide.
   105. The method of embodiment 104, wherein the second vector comprises one or more gene editing components that target a nuclear genome sequence operably linked to a nucleic acid encoding an endogenous Rubisco SSU polypeptide.
   106. The method of embodiment 105, wherein one or more gene editing components are selected from the group consisting of a ribonucleoprotein complex that targets the nuclear genome sequence; a vector comprising a TALEN protein encoding sequence, wherein the TALEN protein targets the nuclear genome sequence; a vector comprising a ZFN protein encoding sequence, wherein the ZFN protein targets the nuclear genome sequence; an oligonucleotide donor (ODN), wherein the ODN targets the nuclear genome sequence; and a vector comprising a CRISPR/Cas enzyme encoding sequence and a targeting sequence, wherein the targeting sequence targets the nuclear genome sequence.
   107. The method of embodiment 105 or embodiment 106, wherein the result of gene editing is that at least part of the higher plant Rubisco SSU polypeptide is replaced with at least part of an algal Rubisco SSU polypeptide.
   108. The method of any one of embodiments 80-107, wherein the EPYC1 polypeptide is the EPYC1 polypeptide of any one of embodiments 36-47.
   109. The method of any one of embodiments 81-108, wherein the Rubisco SSU polypeptide is the Rubisco SSU polypeptide of any one of embodiments 25-34.
   110. The method of embodiment 88 or embodiment 92, wherein the first vector comprises a first copy of the first nucleic acid sequence wherein the first nucleic acid sequence does not comprise the native EPYC1 leader sequence and is not operably linked to the native EPYC1 leader sequence, wherein the first nucleic acid sequence is operably linked to the third nucleic acid sequence encoding a chloroplastic transit peptide functional in the higher plant cell, wherein the first nucleic acid sequence is operably linked to the first promoter, and wherein the first nucleic acid sequence is operably linked to one terminator; and wherein the first vector further comprises a second copy of the first nucleic acid sequence wherein the first nucleic acid sequence does not comprise the native EPYC1 leader sequence and is not operably linked to the native EPYC1 leader sequence, wherein the first nucleic acid sequence is operably linked to the third nucleic acid sequence encoding a chloroplastic transit peptide functional in the higher plant cell, wherein the first nucleic acid sequence is operably linked to a third promoter, and wherein the first nucleic acid sequence is operably linked to two terminators.
   111. The method of embodiment 110, wherein the first promoter is selected from the group consisting of a constitutive promoter, an inducible promoter, a leaf specific promoter, and a mesophyll cell specific promoter; wherein the third promoter is selected from the group consisting of a constitutive promoter, an inducible promoter, a leaf specific promoter, and a mesophyll cell specific promoter; and wherein the first and third promoters are not the same.
   112. The method of embodiment 111, wherein the chloroplastic transit peptide is a polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO: 63.
   113. The method of embodiment 112, wherein the native EPYC1 leader sequence corresponds to nucleotides 60 to 137 of SEQ ID NO: 65.
   114. The method of embodiment 113, wherein the terminators are selected from the group consisting of a HSP terminator, a NOS terminator, an OCS terminator, an intronless extensin terminator, a 35S terminator, a pinII terminator, a rbcS terminator, an actin terminator, and any combination thereof 115. A plant or plant part produced by the method of any one of embodiments 80-114.
   116. A method of cultivating the genetically altered plant of any one of embodiments 21-79 and 115, comprising the steps of:
a) planting a genetically altered seedling, a genetically altered plantlet, a genetically altered cutting, a genetically altered tuber, a genetically altered root, or a genetically altered seed in soil to produce the genetically altered plant or grafting the genetically altered seedling, the genetically altered plantlet, or the genetically altered cutting to a root stock or a second plant grown in soil to produce the genetically altered plant;
b) cultivating the plant to produce harvestable seed, harvestable leaves, harvestable roots, harvestable cuttings, harvestable wood, harvestable fruit, harvestable kernels, harvestable tubers, and/or harvestable grain; and
c) harvesting the harvestable seed, harvestable leaves, harvestable roots, harvestable cuttings, harvestable wood, harvestable fruit, harvestable kernels, harvestable tubers, and/or harvestable grain.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D show the structures of Essential Pyrenoid Component 1 (EPYC1) and the Rubisco small subunit (SSU). FIG. 1A shows a schematic of EPYC1 where the four repeat regions are shown in light gray (first repeat region), gray (second repeat region), dark gray (third repeat region), and darkest gray (fourth repeat region), the predicted α-helix in each repeat region is shown in black, and the N- and C-termini are shown in white. FIG. 1B shows the sequence of EPYC1 (SEQ ID NO: 34), with the four repeat regions aligned (highlighted in light gray (SEQ ID NO: 36), gray (SEQ ID NO: 69), dark gray (SEQ ID NO: 70), and darker gray (SEQ ID NO: 71), and the predicted α-helix (SEQ ID NO: 169, SEQ ID NO: 170) in each repeat region shown in bold and underlined. The N-terminus (SEQ ID NO: 68) and C-terminus (SEQ ID NO: 41) are shown in gray, and the predicted cleavage site of the chloroplastic transit peptide between 26 (V) and 27 (A) is indicated by a black arrowhead. FIG. 1C shows the predicted model of the Rubisco SSU 1A from Arabidopsis thaliana (1A_{At}) with four β-sheets (shown in light gray and labelled), two α-helical regions (shown in dark gray and labelled), and one βA-βB loop (shown at the top in gray and labelled). FIG. 1D shows an amino acid alignment of the mature A. thaliana SSU 1A (1A_{At}; SEQ ID NO: 1) and the mature Chlamydomonas reinhardtii SSU 1 (S1_{Cr}; SEQ ID NO: 2), with the α-helices highlighted in dark gray, the β-sheets highlighted in light gray, and the βA-βB loop highlighted in gray. The four amino acids that differ between the two C. reinhardtii SSUs (S1_{Cr} and S2_{Cr}) are shown in bold (S1_{Cr}, shown, has T, A, T, and F, at those positions, while S2_{Cr}, not shown, has S, S, S, and W at those positions, respectively).

FIGS. 2A-2C show results of yeast two-hybrid (Y2H) experiments to measure interaction between EPYC1 and different SSUs. FIG. 2A shows Y2H interactions on yeast synthetic minimal media (SD media) lacking leucine (L) and tryptophan (W) (SD-L-W) and yeast synthetic minimal media (SD media) lacking L, W and histidine (H) (SD-L-W-H), where interaction strength is demonstrated by growth on increasing concentrations of the inhibitor 3-Amino-1,2,4-triazole (3-AT; growth at 10 mM 3-AT=strong interaction) (EPYC1=C. reinhardtii EPYC1; S1_{Cr}=C. reinhardtii SSU 1; S2_{Cr}=C. reinhardtii SSU 2; 1A_{At}=A. thaliana SSU 1A; and 1A_{At}MOD=modified 1A_{At} carrying the two α-helical regions from C. reinhardtii). FIG. 2B shows Y2H controls, including positive controls (BD + and AD +), negative controls (BD − and AD −), expression of genes of interest in different vectors, and tests of self-interaction (LSU_{Cr}=C. reinhardtii Rubisco large subunit). FIG. 2C shows additional Y2H controls (AtCP12=A. thaliana CP12-2 (gene ID: AT3G62410); CAH3=C. reinhardtii carbonic anhydrase 3 (gene ID: Cre09.g415700.t1.2); LCIB=C. reinhardtii low-Co₂ inducible protein B (gene ID: Cre10.g452800.t1.2); LCIC=C. reinhardtii low-Co₂ inducible protein C (gene ID: Cre06.g307500.t1.1); and LSU_{At}=A. thaliana Rubisco large subunit). For FIGS. 2A-2C, BD=binding domain (i.e., the listed gene is expressed in the pGBKT7 vector), AD=activation domain (i.e., the listed gene is expressed in the pGADT7 vector), and OD=cell density at which yeast cells were plated, measured by optical density at 600 nm (OD₆₀₀).

FIGS. 3A-3C show native and modified A. thaliana and C. reinhardtii SSUs as well as their interactions with EPYC1. FIG. 3A shows an alignment of the peptide sequences of the mature SSUs from A. thaliana 1A_{At} (At1g67090); SEQ ID NO: 1) and from C. reinhardtii (S1_{Cr} (Cre02.g120100.t1.2; SEQ ID NO: 30); and S2_{Cr} (Cre02.g120150.t1.2; SEQ ID NO: 2)). FIG. 3B shows the peptide sequences 1A_{At} (At1g67090; SEQ ID NO: 1), S1_{Cr} (Cre02.g120100.t1.2; SEQ ID NO: 30) and S2_{Cr} (Cre02.g120150.t1.2; SEQ ID NO: 2) with residues that differ between S1_{Cr} and S2_{Cr} shown in bold. Modified versions of 1A_{At} (1A_{At}Mod ((3-sheet)=A. thaliana β-sheets replaced with C. reinhardtii β-sheets (SEQ ID NO: 23); 1A_{At}Mod (loop)=A. thaliana βA-βB loop replaced with C. reinhardtii βA-βB loop (SEQ ID NO: 24; 1A_{At}Mod ((3-sheet and loop)=A. thaliana β-sheets and βA-βB loop replaced with C. reinhardtii β-sheets and βA-βB loop (SEQ ID NO: 25); 1A_{At}Mod (A. thaliana α-helices)=α-helices replaced with C. reinhardtii α-helices (SEQ ID NO: 26); 1A_{At}Mod (α-helices and (3-sheet)=A. thaliana α-helices and β-sheets replaced with C. reinhardtii α-helices and β-sheets (SEQ ID NO: 27); 1A_{At}Mod (α-helices, β-sheet and loop)=A. thaliana α-helices, β-sheets, and βA-βB loop replaced with C. reinhardtii α-helices, β-sheets, and βA-βB loop (SEQ ID NO: 28); 1A_{At}Mod with 1A_{At}-TP used for plant transformation (Atkinson et al., 2017)=1A_{At}Mod (α-helices) with A. thaliana Rubisco small subunit 1A transit peptide (1A_{At}-TP; underlined) (SEQ ID NO: 33)) and S2_{Cr} (S2_{Cr} with 1A_{At}-TP used for plant transformation (Atkinson et al., 2017)=S2_{Cr} with 1A_{At}-TP (underlined) (SEQ ID NO: 22)) are also shown. In FIGS. 3A-3B, A. thaliana α-helices are highlighted in lightest gray (SEQ ID NO: 3, SEQ ID NO: 4), C. reinhardtii α-helices are highlighted in dark gray (SEQ ID NO: 10, SEQ ID NO: 12), A. thaliana β-sheets are highlighted in light gray (SEQ ID NO: 5, SEQ ID NO: 6, SEQ ID NO: 7, SEQ ID NO: 8), C. reinhardtii β-sheets are highlighted in gray (SEQ ID NO: 11, SEQ ID NO: 6, SEQ ID NO: 13, SEQ ID NO: 14) (except for the β-sheet with residues TMW (SEQ ID NO: 6), which is the same in A. thaliana and C. reinhardtii), the A. thaliana βA-βB loop is highlighted in light gray (SEQ ID NO: 9), and the C. reinhardtii βA-βB loop is highlighted in darkest gray (SEQ ID NO: 15). FIG. 3C shows the results of Y2H experiments using differing concentrations of 3-AT to measure interaction strength between EPYC1 and modified versions of 1A_{At} (1A_{At}MOD), in which different 1A_{At} components (α-helices, β-sheets, and the βA-βB loop) have been replaced with those from S1_{Cr} as indicated (peptide sequences of 1A_{At}MOD versions are shown in FIG. 3B). Interaction strength is indicated by a heat map (key on right side; the higher the concentration of 3-AT at which growth was observed, the stronger the interaction). Two biological replicates were done, and experiments were repeated at least twice each. Appropriate controls were included to ensure exclusion of false positives/negatives.

FIGS. 4A-4K show native and modified versions of C. reinhardtii EPYC1 and their interactions with S1_{Cr}. The four repeat regions of EPYC1 are highlighted lightest gray (first repeat region), gray (second repeat region), dark gray (third repeat region), and darkest gray (fourth repeat region). FIGS. 4A-4B show the peptide sequence of full-length native EPYC1 (Cre10.g436550.t1.2; (SEQ ID NO: 34)) as well as modified EPYC1 with different truncations from the N-terminus. In FIG. 4A, N-ter=N-terminus (SEQ ID NO: 68); N-ter+1rep=N-terminus plus first repeat region (SEQ ID NO: 43); N-ter+2reps=N-terminus, first repeat region, and second repeat region (SEQ ID NO: 44); N-ter+3reps=N-terminus, first repeat region, second repeat region, and third repeat region (SEQ ID NO: 45); and N-ter+4reps=N-terminus, first repeat region, second repeat region, third repeat region, and fourth repeat region (SEQ ID NO: 46). In FIG. 4B, 4reps+C-ter/mEPYC1=first repeat region, second repeat region, third repeat region, fourth repeat region, and C-terminus (SEQ ID NO: 47); 3reps+C-ter=second repeat region, third repeat region, fourth repeat region, and C-terminus (SEQ ID NO: 48); 2reps+C-ter=third repeat region, fourth repeat region, and C-terminus (SEQ ID NO: 49); 1rep+C-ter=fourth repeat region and C-terminus (SEQ ID NO: 50); and C-ter=C-terminus (SEQ ID NO: 41). FIGS. 4C-4D show the alignment of the native EPYC1 protein and the variant EPYC1 proteins with different truncations from the N-terminus (peptide sequences shown in FIGS. 4A-4B). FIG. 4C shows the alignment of the N-terminal portion of the native and truncated EPYC1 proteins. FIG. 4D shows the alignment of the C terminal portion of the native and truncated EPYC1 proteins. FIGS. 4E-4F show the peptide sequences of full-length native EPYC1 (Cre10.g436550.t1.2; SEQ ID NO: 34) as well as modified EPYC1 where repeat regions were substituted with different combinations of the first repeat region with point mutations (shown in bold) in the alpha helix (EPYC1-α1), the second repeat region with point mutations (shown in bold) in the alpha helix (EPYC1-α2), the third repeat region with point mutations (shown in bold) in the alpha helix (EPYC1-α3), and the fourth repeat region with point mutations (shown in bold) in the alpha helix (EPYC1-α4) In FIG. 4E, EPYC1 (Cre10.g436550.t1.2)=full-length native EPYC1 (SEQ ID NO: 34); EPYC1-α1=full-length EPYC1 with the first repeat region replaced with EPYC1-α1 (SEQ ID NO: 51); EPYC1-α1,2=full-length EPYC1 with the first repeat region replaced with EPYC1-α1 and the second repeat region replaced with EPYC1-α2 (SEQ ID NO: 52); and EPYC1-α1,2,3=full-length EPYC1 with the first repeat region replaced with EPYC1-α1, the second repeat region replaced with EPYC1-α2, and the third repeat region replaced with EPYC1-α3 (SEQ ID NO: 53). In FIG. 4F, EPYC1-α1,2,3,4=full-length EPYC1 with the first repeat region replaced with EPYC1-α1, the second repeat region replaced with EPYC1-α2, the third repeat region replaced with EPYC1-α3, and the fourth repeat region replaced with EPYC1-α4 (SEQ ID NO: 54); EPYC1-α3,4=full-length EPYC1 with the third repeat region replaced with EPYC1-α3 and the fourth repeat region replaced with EPYC1-α4 (SEQ ID NO: 55); and EPYC1-α4=full-length EPYC1 with the fourth repeat region replaced with EPYC1-α4 (SEQ ID NO: 56). FIGS. 4G-4H show the alignment of the native EPYC1 protein and the variant EPYC1 proteins with repeat region substitutions with alpha helix point mutation repeat regions (peptide sequences shown in FIGS. 4E-4F). FIG. 4G shows the alignment of the N-terminal portion of the native and truncated EPYC1 proteins. FIG. 4H shows the alignment of the C terminal portion of the native and truncated EPYC1 proteins. FIG. 4I shows an immunoblot of native EPYC1 and N-terminus truncated modified versions of EPYC1 in yeast. FIG. 4J shows interaction strengths, as measured by Y2H experiments, between S1_{Cr} and modified versions of EPYC1 (peptide sequences of the modified versions of EPYC1 tested in this panel are shown in FIGS. 4A-4B). FIG. 4K shows interaction strengths, as measured by Y2H experiments, between S1_{Cr} and additional modified versions of EPYC1 (peptide sequences of the modified versions of EPYC1 tested in this panel are shown in FIGS. 4E-4F). For FIGS. 4J-4K, interaction strength is indicated by a heat map (key on right side; the higher the concentration of 3-AT at which growth was observed, the stronger the interaction), and the four repeat regions of EPYC1 are shown from left to right in block diagrams (N-terminus in white, first repeat region in lightest gray, second repeat region in gray, third repeat region in gray, fourth repeat region in black, and C-terminus in white) with region substitutions with alpha helix point mutation repeat regions indicated by black or dark gray vertical bars within the blocks. Two biological replicates were done, and experiments were repeated at least twice each.

FIGS. 5A-5F show EPYC1 modifications made to increase the interaction strength with SSUs and results from experiments to test the EPYC1 modifications. FIG. 5A shows the peptide sequences of 1, 2, 4, or 8 tandem repeats of the first repeat region (synthetic EPYC1 1 rep (SEQ ID NO: 36), synthetic EPYC1 2 reps (SEQ ID NO: 37), synthetic EPYC1 4 reps (SEQ ID NO: 38), and synthetic EPYC1 8 reps (SEQ ID NO: 39)), the peptide sequences of the first repeat region with an additional alpha-helix inserted (shown in bold and underlined) (synthetic EPYC1 2 α-helices 1 rep (SEQ ID NO: 57)), four copies of the first repeat region, each with an additional alpha-helix inserted (shown in bold and underlined) (synthetic EPYC1 2 α-helices 4 reps (SEQ ID NO: 58)), and three versions of the first repeat region each containing a point mutation (shown in bold and larger font) in the alpha-helix of the first repeat (synthetic EPYC1 modified α-helix 1 rep (SEQ ID NO: 59), synthetic EPYC1 α-helix knockout A (SEQ ID NO: 60), and synthetic EPYC1 α-helix knockout B (SEQ ID NO: 61), respectively). FIGS. 5B-5D show the alignment of the native EPYC1 protein and the synthetic EPYC1 proteins with different numbers of tandem repeats (peptide sequences shown in FIG. 5A). FIG. 5B shows the alignment of the N-terminal portion of the native and synthetic EPYC1 proteins. FIG. 5C shows the alignment of the central portion of the native and synthetic EPYC1 proteins. FIG. 5D shows the alignment of the C terminal portion of the native and truncated EPYC1 proteins. FIG. 5E shows interaction strengths, as measured by Y2H experiments, between S1_{Cr} and synthetic variants of EPYC1 based on the first repeat regions (lightest gray) and the predicted α-helix (indicated by vertical bars filled with darkest gray for the α-helix, lightest gray for the modified α-helix, lighter gray for α-helix knockout A, or light gray for α-helix knockout B) (peptide sequences of the synthetic variants of EPYC1 tested in this panel are shown in FIG. 5A). Interaction strength is indicated by a heat map (key on right side; the higher the concentration of 3-AT at which growth was observed, the stronger the interaction). FIG. 5F shows the predicted coiled coil domain probability for the first repeat region of EPYC1 and for synthetic variants of the first repeat region of EPYC1 using the PCOILS bioinformatic tool. Matching color-coded amino acid sequences are shown beneath the graph, with residues that differ from the wild-type sequence shown in bold and underlined. At top is the EPYC1 1 rep (wildtype) sequence (SEQ ID NO: 36); second from top is the α-helix knockout B sequence (SEQ ID NO: 60); third from top is the α-helix knockout A sequence (SEQ ID NO: 61); fourth from top is the modified α-helix sequence (SEQ ID NO: 59); and at bottom is the 2 α-helices sequence (SEQ ID NO: 57). The inlaid graph shows the coiled coil domain probability for full-length EPYC1.

FIGS. 6A-6C show immunoprecipitation and intact protein mass spectrometry of mature EPYC1 from C. reinhardtii. FIG. 6A shows a coomassie-stained SDS-PAGE gel containing C. reinhardtii cell lysate (input), the contents of the wash during the immunoprecipitation process (wash) and the eluted immunoprecipitated EPYC1 (IP). FIG. 6B shows the electrospray ionization (ESI) charge state distribution of EPYC1. FIG. 6C shows the deconvoluted neutral molecular mass, in Daltons (Da), of EPYC1.

FIGS. 7A-7C show a map of the binary vector used to express EPYC1 in higher plants, as well as assay results showing EPYC1 expression in higher plants. FIG. 7A shows a map of the binary vector carrying 1A_{At}-TP::EPYC1 (SEQ ID NO: 67) used for plant transformation, with the A. thaliana Rubisco small subunit 1A transit peptide (1A_{At}-TP) in gray, EPYC1 in light gray, the 35S constitutive promoter (35S) and octopine synthase terminator (ocs) both shown in gray, the origin of replication from the plasmid pVS1 that permits replication of low-copy plasmids in Agrobacterium tumefaciens (oriV) shown in lightest gray, the expression cassette for aminoglycoside adenylyltransferase conferring resistance to spectinomycin (SmR) shown in darkest gray, high-copy-number ColE1/pMB1/pBR322/pUC origin of replication (ori) shown in lightest gray, trans-acting replication protein that binds to and activates oriV (trfA) shown in darkest gray, pFAST-R selection cassette (monomeric tagRFP from E. quadricolor fused to the coding sequence of oleosin1 (OLE1, A. thaliana) (Shimada, et al., Plant J. (2010) 61: 519-528-667) showing the olesin1 promoter (Olesin pro) in white, the olesin1 5′ UTR (Olesin 5′ UTR) in gray, a modified olesin1 gene (Olesin) in darkest gray with a dotted darkest gray line, the fluorescent tag (TagRFP) in darkest gray, the olesin1 terminator (Olesin term) in white, the right border sequence required for integration of the T-DNA into the plant cell genome (RB T-DNA repeat) in gray, and the left border sequence required for integration of the T-DNA into the plant cell genome (LB T-DNA repeat) in gray. FIG. 7B shows transient expression in N. benthamiana of the following constructs: EPYC1 fused with the green fluorescent protein (GFP) without the 1A_{At} chloroplastic transit peptide (EPYC1::GFP, top row), EPYC1 fused with GFP with the 1A_{At} chloroplastic transit peptide (1A_{At}-TP::EPYC1::GFP, middle row), and the A. thaliana 1A small subunit of Rubisco fused with GFP (RbcS1A::GFP, bottom row). FIG. 7C shows stable expression in A. thaliana of the following constructs: EPYC1 fused with GFP without the 1A_{At} chloroplastic transit peptide (EPYC1::GFP, top row), and EPYC1 fused with GFP with the 1A_{At} chloroplastic transit peptide (1A_{At}-TP::EPYC1::GFP, bottom row). For FIGS. 7B-7C, the GFP channel is shown in the left column, the chlorophyll autofluorescence channel is shown in the middle column, an overlay of GFP and chlorophyll is shown in the right column with overlapping signals in white, and the scale bars represent 10 μm.

FIGS. 8A-8E show protein expression and growth data from higher plants expressing EPYC1. FIG. 8A shows immunoblots against 1A_{At}-TP::EPYC1 from protein extracted from A. thaliana plant lines expressing 1A_{At}-TP::EPYC1 in the following three backgrounds: wild-type (EPYC1, top row), Rubisco small subunit mutant 1a3b mutant complemented with S2_{Cr} (S2_{Cr}_EPYC1, middle row), and 1a3b complemented with 1A_{At}MOD (1A_{At}MOD_EPYC1, bottom row). The immunoblots display the relative EPYC1 expression levels in three independently transformed homozygous T3 lines (Line 1, Line 2, Line 3) per background, compared to their corresponding segregants (Seg 1, Seg 2, Seg 3) lacking EPYC1. FIG. 8B shows fresh and dry weights of plants harvested at 31 days from plants of the lines in FIG. 8A. Data from three independently transformed homozygous T3 lines (indicated by “_1”, “_2”, “_3”) per background (EPYC1, S2_{Cr}_EPYC1, 1A_{At}MOD_EPYC1) are shown with white bars, while data from corresponding segregants lacking EPYC1 for each line are shown with black bars. Values are the means±standard error of measurements made on 12 rosettes, and asterisks indicate a significant difference between transformed lines and segregants (P<0.05) as determined by Student's paired sample t-tests. FIG. 8C shows rosette growth of the nine transformed lines described in FIGS. 8A-8B. Rosette growth is measured by area in mm², values are the means±standard error of measurements made on 16 rosettes, and data from three independently transformed homozygous T3 lines per background (EPYC1, S2_{Cr}_EPYC1, 1A_{At}MOD_EPYC1) are shown with black circles, while data from corresponding segregants lacking EPYC1 for each line are shown with white circles. FIG. 8D shows an immunoblot comparing the banding patterns of EPYC1 extracted from different expression systems. Lane 1: Protein from A. thaliana stable expression line EPYC1_1 extracted in sample loading buffer with 200 mM DTT. Lane 2: Protein from EPYC1_1 line extracted with an immunoprecipitation (IP) extraction buffer including protease inhibitors. Lane 3: Protein from C. reinhardtii (strain CC-1690m) extracted with the IP extraction buffer. Lane 4: Protein from yeast expressing EPYC1::GAL4 binding domain extracted in yeast lysis buffer. The blot was probed with the anti-EPYC1 antibody from Mackinder, et al., PNAS (2016) 113: 5958-5963. FIG. 8E shows immunoblots illustrating the ratiometric comparison of the abundances of EPYC1 (top) to the Rubisco large subunit (LSU; bottom) in C. reinhardtii (left) and A. thaliana line S2_{Cr}_EPYC1 (right). The quantities of soluble protein loaded per lane are displayed above each blot in μg, and three independent biological replicates were assayed.

FIGS. 9A-9E show results of methods characterizing interactions between EPYC1 and Rubisco in higher plants. FIG. 9A shows the results of co-immunoprecipitation of Rubisco with EPYC1 from four different transgenic A. thaliana lines, performed using Protein-A coated beads that had been cross-linked to an anti-EPYC1 antibody. The top row shows data from the Rubisco small subunit mutant 1a3b mutant complemented with S2_{Cr} and expressing EPYC1 fused with the 1A_{At}TP. The second row shows data from the 1a3b mutant complemented with 1A_{At}MOD and expressing EPYC1 fused with the 1A_{At}TP. The third row shows data from wild-type (WT) plants expressing EPYC1 fused with the 1A_{At}TP. The bottom row shows data from 1a3b complemented with S2_{Cr} without EPYC1. The blots on the left (EPYC1 IP) show the results when probed with an anti-EPYC1 antibody (from Mackinder, et al., PNAS (2016) 113: 5958-5963), while the blots on the right (Co-IP) show the results when probed with an antibody against the Rubisco large subunit (LSU). Lanes (columns) from left to right display results from the input (Input), flow-through (F-T), 4th wash (Wash), and boiling elute (Elute). Negative controls (Neg.) differed: Neg. (*) was a control where the anti-EPYC1 antibody on the Protein-A beads was replaced with anti-HA antibody and the IP was continued as before, Neg. (**) was a control where the anti-EPYC1 antibody on the Protein-A beads was replaced with no antibody and the IP was continued as before (for both, only the eluted sample is shown). Triple asterisks (***) indicate a non-specific band observed with the anti-EPYC1 antibody in all samples including the control line not expressing EPYC1 (S2_{Cr}). FIG. 9B shows bimolecular fluorescence complementation assays in three N. benthamiana lines transiently expressing proteins fused at the C-terminus to either YFP^{N} or YFP^{C}. The top row displays data from a plant expressing the C. reinhardtii Rubisco small subunit 2 (S2_{Cr}) fused to YFP^{N} (S2_{Cr}::YFP^{N}) and EPYC1 fused to YFP^{C} (EPYC1::YFP^{C}). The middle row displays data from a plant expressing EPYC1 fused to YFP^{N} (EPYC1::YFP^{N}) and S2_{Cr} fused to YFP^{C} (S2_{Cr}::YFP^{C}). The bottom row displays data from a plant expressing modified 1A_{At} carrying the two α-helical regions from C. reinhardtii (1A_{At}MOD) fused to YFP^{N} (1A_{At}MOD::YFP^{N}) and EPYC1 fused to YFP^{C} (EPYC1::YFP^{C}). FIG. 9C shows bimolecular fluorescence complementation assays in three additional N. benthamiana lines transiently expressing proteins fused at the C-terminus to either YFP^{N} or YFP^{C}. The top row displays data from a plant expressing EPYC1 fused to YFP^{N} (EPCY1::YFP^{N}) and 1A_{At}MOD fused to YFP^{C} (1A_{At}MOD::YFP^{C}). The middle row displays data from a plant expressing the A. thaliana SSU 1A (1A_{At}) fused to YFP^{N} (1A_{At}::YFP^{N}) and EPYC1 fused to YFP^{C} (EPYC1::YFP^{C}). The bottom row displays data from a plant expressing EPYC1 fused to YFP^{N} (EPYC1::YFP^{N}) and 1A_{At} fused to YFP^{C} (1A_{At}::YFP^{C}). FIG. 9D shows negative control bimolecular fluorescence complementation assays in three N. benthamiana lines transiently expressing proteins fused at the C-terminus to either YFP^{N} or YFP^{C}. The top row displays data from a plant expressing AtCP12 fused to YFP^{N} (AtCP12::YFP^{N}) and EPYC1 fused to YFP^{C} (EPYC1::YFP^{C}). The middle row displays data from a plant expressing EPYC1 fused to YFP^{N} (EPYC1::YFP^{N}) and AtCP12 fused to YFP^{C} (AtCP12::YFP^{C}). The bottom row displays data from a plant expressing AtCP12 fused to YFP^{N} (AtCP12::YFP^{N}) and 1A_{At} fused to YFP^{C} (1A_{At}::YFP^{C}). FIG. 9E shows additional negative control bimolecular fluorescence complementation assays in two additional N. benthamiana lines. The top row displays data from a plant transiently expressing 1A_{At} fused to YFP^{N} (1A_{At}::YFP^{N}) and AtCP12 fused to YFP^{C} (AtCP12::YFP^{C}). The bottom row displays data from a non-transformed plant. In FIGS. 9B-9D, the signals in the left column are reconstituted YFP fluorescence, the signals in the middle column are chlorophyll autofluorescence, an overlay of the YFP and chlorophyll channels is in the right column, with overlapping signals shown in white, and the scale bars represent 10 μm.

FIGS. 10A-10E show in vitro phase separation data for Rubisco and EPYC1 mixtures. FIG. 10A shows images of tubes containing 15 μM Rubisco (extracted from C. reinhardtii (Cr), from A. thaliana wild-type plants (At), from A. thaliana S2_{Cr} plants (S2c), or no Rubisco (-)) and 10 μM EPYC1 (in four tubes on right; no EPYC1 was added three tubes on left) at about 3 minutes after mixing at room temperature. FIG. 10B shows differential interference contrast (DIC) and epifluorescence (GFP) microscopy images of in vitro samples containing different concentrations and ratios of EPYC1 and Rubisco, as indicated. Fluorescence in samples containing EPYC1 is due to the inclusion of EPYC1::GFP (final EPYC1 concentration includes 0.25 μM of EPYC1::GFP). In the two leftmost columns, the Rubisco was purified from C. reinhardtii; in the two middle columns, the Rubisco was purified from A. thaliana S2_{Cr} plants (S2_{Cr}); and in the two rightmost columns, the Rubisco was purified from wild-type A. thaliana plants (Arabidopsis). The scale bar represents 15 μm. FIG. 10C shows time-course microscopy images of droplet fusion in an in vitro sample containing 15 μM of isolated S2_{Cr} Rubisco and 10 μM of EPYC1. The top row displays the differential interference contrast (DIC) channel, and the bottom row displays the epifluorescence (GFP) channel. The elapsed time in seconds (s), relative to the first image, of each image in the series is displayed at the top. The scale bar represents 5 μm. FIG. 10D shows droplet sedimentation analysis by SDS-PAGE for samples containing 40 μM of Rubisco (Rubisco was extracted from C. reinhardtii (Cr), A. thaliana S2_{Cr} plants (S2_{Cr}), or wild-type A. thaliana plants (At); sample without Rubisco indicated by -) and different μM concentrations of EPYC1 as indicated (0 μM, 3.75 μM, or 10 μM). FIG. 10E shows additional droplet sedimentation analysis droplet sedimentation analysis by SDS-PAGE for samples containing 15 μM of Rubisco (Rubisco was extracted from C. reinhardtii (Cr), A. thaliana S2_{Cr} plants (S2_{Cr}), or wild-type A. thaliana plants (At)) and different μM concentrations of EPYC1 as indicated (3.75 μM or 10 μM). For FIGS. 10D-10E, the samples were droplets of demixed Rubisco and EPYC1 that were harvested by centrifugation, and both the supernatant fraction (bulk solution; S) and the resuspended pellet fraction (droplet; P) were run on the gel (M represents the marker lane, with the size key displayed in kDa along the left; locations of the bands corresponding to the Rubisco large subunit (LSU), EPYC1, and the Rubisco small subunit (SSU) are indicated along the right).

FIGS. 11A-11B show localization data of Rubisco in higher plant chloroplasts. FIG. 11A shows transmission electron microscopy images of immunogold labeling of Rubisco in chloroplasts of A. thaliana S2_{Cr} lines expressing EPYC1 (scale bars are 0.5 μm). FIG. 11B shows transmission electron microscopy images of immunogold labeling of Rubisco in chloroplasts of A. thaliana 1a3b mutant plants complemented with S2_{Cr} without EPYC1 (scale bars are 0.5 μm).

FIGS. 12A-12L show TobiEPYC1 gene expression cassettes, a map of the binary vector used to express TobiEPYC1 in higher plants, and fluorescent microscopy images of plants and protoplasts expressing TobiEPYC1. FIG. 12A shows six different gene expression cassettes for variants of native and synthetic EPYC1 with a truncated version of the EPYC1 N-terminus (TobiEPYC1 variants). Each cassette contains the following, from left to right: the 35S promotor (35s pro; gray); a 57-residue chloroplast signal peptide from A. thaliana Rubisco SSU 1A (SP1A; black); a truncated version of the EPYC1 N-terminus (unlabelled; lightest gray); EPYC1 repeat regions (first repeat region in lightest gray; second repeat region in gray; third repeat region in gray; and fourth repeat region in black), with the predicted α-helix in each repeat region (black); the EPYC1 C-terminus (unlabelled; lightest gray); and double terminators HSP (dark gray) and nos (gray). Cassettes 2, 4, and 6 also contain a C-terminal green fluorescent protein tag (GFP; light gray), before the terminators. FIG. 12B shows the arrangement of the TobiEPYC1 gene expression cassettes in the vector, which face away from each other. The first cassette (clockwise) is driven by the cassava vein mosaic virus promoter (CsVMV pro), the heat shock protein (A. thaliana) terminator (HSP term) and nopaline synthase (A. tumefaciens) terminator (Nos term). The second cassette (anti-clockwise) is driven by the 35S promoter (35S prom) and only a single terminator—the octopine synthase terminator (OCS term). FIG. 12C shows a map of the binary vector carrying TobiEPYC1::GFP (cassette 2 from FIG. 12A; arrangement of cassette 2 in the vector in FIG. 12B) used for plant transformation (SEQ ID NO: 168), with the A. thaliana Rubisco small subunit 1A transit peptide (1A_{At}-TP) in gray, TobiEPYC1 in light gray, the 35S constitutive promoter (35S pro) and the CsVMV constitutive promoter (CsVMV pro) both shown in white, the 6×HA tag shown in gray, eGFP shown in light gray, codon optimized turbo GFP (tGFP) shown in darkest gray with a dotted dark gray line, the HSP terminator (HSP term) shown in gray, the Nos terminator (Nos term) shown in white, the OCS terminator (OCS term) shown in white, the origin of replication from the plasmid pVS1 that permits replication of low-copy plasmids in A. tumefaciens (oriV) shown in lightest gray, high-copy-number ColE1/pMB1/pBR322/pUC origin of replication (ori) shown in lightest gray, the expression cassette for aminoglycoside phosphotransferase conferring resistance to kanamycin (KanR) shown in lightest gray, stability protein from the Pseudomonas plasmid pVS1 (pVS1 StaA) shown in darkest gray, replication protein from the plasmid pVS1 (pVS1 RepA) shown in darkest gray, pFAST-R selection cassette (monomeric tagRFP from E. quadricolor fused to the coding sequence of oleosin1 (OLE1, A. thaliana) (Shimada, et al., Plant J. (2010) 61: 519-528-667) showing the olesin1 promoter (Olesin pro) in white, the olesin1 5′ UTR (Olesin 5′ UTR) in gray, a modified olesin1 gene (Olesin) in darkest gray with a dotted dark gray line, the fluorescent tag (TagRFP) in darkest gray, the olesin1 terminator (Olesin term) in white, the right border sequence required for integration of the T-DNA into the plant cell genome (RB T-DNA repeat) in lightest gray, and the left border sequence required for integration of the T-DNA into the plant cell genome (LB T-DNA repeat) in lightest gray. FIG. 12D shows fluorescence microscopy images of transient expression of TobiEPYC1::GFP in N. benthamiana (GFP channel on the left, imaged at a gain of 25 and 2% laser; chlorophyll autofluorescence channel in the middle; overlay of the GFP and chlorophyll channels on the right, with overlapping regions shown in white). FIG. 12E shows a fluorescence microscopy images of transient expression of TobiEPYC1::GFP in N. benthamiana (GFP channel, imaged at a gain of 10 and 1% laser). FIG. 12F shows fluorescence microscopy images of stable expression of TobiEPYC1::GFP in A. thaliana S2_{cr} lines (GFP channel on the left; chlorophyll autofluorescence channel in the middle; overlay of the GFP and chlorophyll channels on the right, with overlapping regions shown in white). FIG. 12G shows fluorescence microscopy images of protoplasts from A. thaliana S2_{Cr} lines stably expressing TobiEPYC1::GFP (GFP channel on the left; chlorophyll autofluorescence channel second from left; bright field image second from right; overlay of the GFP, chlorophyll, and bright field images on the right, with regions of overlapping fluorescence shown in white). FIG. 12H shows fluorescence microscopy images of another set of protoplasts from A. thaliana S2_{Cr} lines stably expressing TobiEPYC1::GFP (GFP channel on the left; chlorophyll autofluorescence channel in the middle; overlay of the GFP and chlorophyll channels on the right). FIG. 12I shows fluorescence microscopy images of another set of protoplasts from A. thaliana S2_{Cr} lines stably expressing TobiEPYC1::GFP with arrows indicating the region of the TobiEPYC1 aggregate (GFP channel on the left; chlorophyll autofluorescence channel in the middle; overlay of the GFP and chlorophyll channels on the right). FIG. 12J shows fluorescence microscopy images of another set of protoplasts from A. thaliana S2_{Cr} lines stably expressing TobiEPYC1::GFP (GFP channel on the left; chlorophyll autofluorescence channel second from left; bright field image second from right; overlay of the GFP, chlorophyll, and bright field images on the right). FIG. 12K shows chloroplasts from recently-popped protoplasts from A. thaliana plants stably expressing TobiEPYC1::GFP with dashed arrows indicating EPYC1 aggregates outside of chloroplasts (GFP channel on the left; chlorophyll autofluorescence channel second from left; bright field image second from right; overlay of the GFP, chlorophyll, and bright field images on the right). FIG. 12L shows fluorescence microscopy images of protoplasts from wild type A. thaliana stably expressing TobiEPYC1::GFP (GFP channel on the left; chlorophyll autofluorescence channel in the middle; overlay of GFP and chlorophyll channels on the right, with regions of overlapping fluorescence shown in white). For FIGS. 12D-12L, the scale bar is 10 μm, and the images are representative images.

FIGS. 13A-13E show results from fluorescence recovery after photobleaching (FRAP) experiments. FIG. 13A shows images from a fluorescence recovery after photobleaching (FRAP) time course in two samples (shown across the top and across the bottom, respectively) of TobiEPYC1::GFP aggregates in A. thaliana S2_{Cr} tissue (scale bar=5 μm). The images on the far left show the aggregate before the bleaching event (Pre-bleach), and the white circle overlaid on the pre-bleach image marks the area that was targeted for bleaching. The images on the right show the aggregate at various time points after the bleaching event, with the time elapsed post-bleach displayed in seconds (0.6 seconds, 2.6 seconds, 7.4 seconds, 9 seconds, 16 seconds, and 24 seconds). FIG. 13B shows an exemplary image from the imaging time course (time point 0.6 seconds in FIG. 13A) with overlays indicating the circular regions of interest (ROI) from which the signal was analyzed (bleached region circled above; non-bleached region circled below; scale bar=2.5 μm). FIG. 13C shows FRAP curves for the ROI indicated in FIG. 13B. The raw fluorescence signal intensities from the ROI during the time course (data correspond to the top dataset in FIG. 13A) are displayed, with the time of the bleach event marked by a black vertical line. Data from the bleached ROI are plotted in gray. Data from the non-bleached ROI are plotted in dark gray. FIG. 13D shows FRAP curves for the ROI indicated in FIG. 13B after normalization to the non-bleached signal at each time point (data correspond to the top dataset in FIG. 13A). Data are shaded as in FIG. 13C. FIG. 13E shows Western blots using α-EPYC1 to probe protein extracts from A. thaliana S2_{Cr} plants stably expressing TobiEPYC1. Each of the three leftmost lanes contains protein extract from a different plant (TobiEPYC1 1, TobiEPYC1 2, and TobiEPYC1 3) expressing the TobiEPYC1 gene expression cassette (shown in FIG. 12A), the lane fourth from the left and the lane on the right contain protein extracts from A. thaliana S2Cr lines not expressing TobiEPYC1, and the second from the right lane contains protein extract from a plant expressing the 4 reps TobiEPYC1 gene expression cassette (shown in FIG. 12A) (protein weights in kDa are overlaid in white; gray arrows on the right indicate the positions of bands that correspond to EPYC1; the black arrow indicates a non-specific band).

FIGS. 14A-14C show amino acid alignments of C. reinhardtii RbcS1 with Rubisco SSUs from algal species Volvox carteri and Gonium pectorale. FIGS. 14A-14B show the alignment of C. reinhardtii S1_{Cr} (SEQ ID NO: 30) with Rubisco SSUs from V. carteri (SEQ ID NO: 157, SEQ ID NO: 158, SEQ ID NO: 159, SEQ ID NO: 160, SEQ ID NO: 161, SEQ ID NO: 162, SEQ ID NO: 163)). FIG. 14A shows the alignment of the N-terminal portion of C. reinhardtii RbcS1 and the V. carteri Rubisco SSUs. FIG. 14B shows the alignment of the C terminal portion of C. reinhardtii RbcS1 and the V. carteri Rubisco SSUs. FIG. 14C shows the alignment of C. reinhardtii S1_{Cr} (SEQ ID NO: 30) with the G. pectorale SSU (SEQ ID NO: 164) For FIGS. 14A-14C, alignment of the α-helices is shown in bold.

FIG. 15 shows an amino acid alignment of C. reinhardtii EPYC1 (SEQ ID NO: 34) with EPYC1 homologs from algal species V. carteri (SEQ ID NO: 166), G. pectorale (SEQ ID NO: 167), and Tetrabaena socialis (SEQ ID NO: 168), with the alignment of the α-helices shown in bold.

FIG. 16 shows a schematic representation of the binary vector for dual GFP expression (EPYC1-dGFP). This vector encodes two constructs in opposite directions: EPYC1 fused at the C-terminus to turboGFP (tGFP; left side), and EPYC1 fused at the C-terminus to enhanced GFP (eGFP; right side). In both constructs, EPYC1 is truncated at amino acid residue 27 (indicated by the small triangles pointing down) and fused at the N-terminus to the chloroplastic A. thaliana Rubisco small subunit 1A transit peptide (RbcS1A TP). EPYC1-tGFP expression is driven by the cauliflower mosaic virus 35S promoter (35S prom; leftward-pointing triangle). EPYC1-eGFP is driven by the cassava vein mosaic virus promotor (CsVMV prom; rightward-pointing triangle). For the eGFP expression cassette, a dual terminator system comprising the heat shock protein terminator (HSP ter) and the nopaline synthase terminator (nos ter) was used to increase expression. For the tGFP expression cassette, a single octopine synthase terminator (ocs ter) was used.

FIG. 17 shows immunoblots depicting EPYC1 protein levels in A. thaliana transgenic plants and controls. The top two immunoblots were made with anti-EPYC1 antibodies. The bottom two immunoblots are loading controls made with anti-actin antibodies. Each column contains soluble protein extract from a different plant. The eight columns on the left are all from transgenic plants in the A. thaliana 1a3b Rubisco mutant background complemented with an SSU from C. reinhardtii (S2_{Cr}). The two columns on the right are from transgenic plants in a wild-type background (WT). In the S2_{Cr} background, extract from three different T2 transgenic plants expressing EPYC1-dGFP are shown in the columns labeled Ep1, Ep2, and Ep3, respectively. Extract from the azygous segregants of those plants are shown in the columns labeled Az1, Az2, and Az3, respectively. Extract from S2_{Cr} plants transformed with only EPYC1::eGFP or only EPYC1::tGFP are shown in the columns labeled eGFP and tGFP, respectively. The columns labeled EpWT and EpAz show extracts from a T2 EPYC1-dGFP WT transformant and azygous segregant, respectively. The positions of bands matching the weights of EPYC1::eGFP (63.9 kDa), EPYC1::tGFP (55.4 kDa), and actin are marked along the right side.

FIGS. 18A-18L show condensate formation in transgenic A. thaliana chloroplasts expressing EPYC1. FIG. 18A shows confocal microscopy images of expression of EPYC1-dGFP in A. thaliana plants of three different backgrounds: wild-type (WT; top row), the 1a3b Rubisco mutant complemented with a C. reinhardtii Rubisco small subunit (S2_{Cr}; middle row), and the 1a3b Rubisco mutant complemented with a native A. thaliana Rubisco small subunit that was modified to contain the two C. reinhardtii small subunit α-helices necessary for pyrenoid formation (1A_{At}MOD; bottom row). The images in the left column show the GFP channel. The images in the right column show an overlay of the GFP channel with chlorophyll autofluorescence. The scale bars represent 10 μm. FIG. 18B shows transmission electron microscopy images of chloroplasts from 21-day-old S2_{Cr} plants that have not been transformed with EPYC1-dGFP (left) and 21-day-old S2_{Cr} transgenic lines that are expressing EPYC1-dGFP (right). The scale bars represent 0.5 μm. The arrow points to the condensate in the stroma of the EPYC1-expressing chloroplast on the right. FIG. 18C shows two channels of a confocal microscopy image of A. thaliana S2_{Cr} chloroplasts expressing EPYC1-dGFP. The image on the left shows chlorophyll autofluorescence. The image on the right shows an overlay of the GFP channel with chlorophyll autofluorescence. The arrow points to a dark spot in the chlorophyll autofluorescence of one chloroplast, indicating that chlorophyll autofluorescence is reduced at the site of EPYC1-dGFP accumulation. The scale bar represents 5 μm. FIG. 18D shows a z-projection of a super-resolution structured illumination microscopy (SIM) image of EPYC1-dGFP condensates inside chloroplasts of A. thaliana S2_{Cr} chloroplasts expressing EPYC1-dGFP. The image is an overlay of the GFP and chlorophyll autofluorescence channels. Arrows indicate round regions of high GFP signal. The scale bar represents 2 μm. FIG. 18E shows a three-dimensional projection of the same chloroplasts shown in FIG. 18D that has been rotated to display the depth (z) dimension. The image is an overlay of the GFP and chlorophyll autofluorescence channels. Dashed arrows indicate the relative x, y, and z axes of the image volume. Solid arrows indicate round regions of high GFP signal. The scale bar represents 1 μm. FIG. 18F shows an exemplary comparison of the condensate size in a SIM image of a chloroplast of an A. thaliana S2_{Cr} plant expressing EPYC1-dGFP (left) with that of a pyrenoid in a transmission electron microscopy (TEM) image of a C. reinhardtii cell (right). The scale bar in the TEM image represents 0.5 μm. 2 μm labelled bars span the width of the GFP-expressing region in the A. thaliana chloroplast (left) and the C. reinhardtii pyrenoid (right), respectively. FIGS. 18G-18H show confocal fluorescence microscopy images of transgenic A. thaliana S2_{Cr} leaf tissue expressing EPYC1-dGFP. The left panels show the GFP channel. The middle panels show chlorophyll autofluorescence. The right panels show an overlay of the GFP and chlorophyll channels. FIG. 18G shows a maximum projection of a z-stack of a single cell, in which condensates can be seen in every chloroplast. The scale bar represents 5 μm. FIG. 18H shows images of transgenic A. thaliana S2_{Cr} lines Ep1-3 with different expression levels of EPYC1-dGFP (as shown in FIG. 17). The scale bars represent 10 μm. FIG. 18I shows representative confocal fluorescence microscopy images of condensates in transgenic A. thaliana S2_{Cr} plants expressing a single EPYC1 expression cassette of EPYC1 fused at the C-terminus to either tGFP (EPYC1::tGFP; top row) or eGFP (EPYC1::eGFP; bottom row). The left images show the GFP channel. The middle images show chlorophyll autofluorescence. The right images show the overlay of the GFP and chlorophyll channels. The scale bars represent 10 μm. FIGS. 18J-18L show scatterplots of data derived from confocal images of C. reinhardtii pyrenoids (n=55) and chloroplasts of the three EPYC1-dGFP-expressing transgenic A. thaliana S2_{Cr} transgenic lines (Ep1-3; n=42). FIG. 18J shows the diameter of the pyrenoids (for C. reinhardtii cells) or condensates (for transgenic A. thaliana) in μm, with the mean diameter represented by wide horizontal lines and the standard error of the mean (SEM) represented by error bars. FIG. 18K shows the volume of the condensates in μm plotted against the estimated volume in μm of their respective chloroplasts, with data from each of Ep1-3 plotted in a different shade. FIG. 18L shows a plot of the estimated percent of chloroplast volume occupied by the condensate for transgenic A. thaliana S2_{Cr} transgenic lines Ep1-3 (n=27 chloroplasts for each line). The wide horizontal bars represent the mean value for each line, and the error bars represent SEM.

FIGS. 19A-19C show in planta fluorescence microscopy analyses of the liquid-liquid phase separation properties of the EPYC1-dGFP condensates in A. thaliana chloroplasts. FIG. 19A shows GFP fluorescence intensity distribution plots across cross-sections of 28 WT (left), 17 S2_{Cr} (middle), and 22 1A_{At}MOD chloroplasts expressing EPYC1-dGFP. Each plot line represents data from a different chloroplast. Normalized GFP fluorescence is shown on the y-axis. Normalized relative distance across the chloroplast is shown on the x-axes. FIGS. 19B-19C show fluorescence recovery after photobleaching (FRAP) assays in S2_{Cr} transgenic A. thaliana line expressing EPYC1-dGFP. FIG. 19B shows still images from the GFP channel in representative FRAP time-courses on condensates in live (top) and fixed (bottom) leaf tissue. The left-most images show the GFP distribution before the bleaching event. The images on the right show the GFP distribution over time after the bleaching event. The elapsed time since the bleaching event is shown above the images in seconds. The scale bar represents 1 μm. FIG. 19C shows a plot of the fluorescence recovery of condensates in 13-16 chloroplasts. The y-axis shows the GFP signal in the bleached area relative to the non-bleached area, in which the signal from the non-bleached area has been defined as 1 (dashed horizontal line). The x-axis shows the elapsed time in seconds, with the time of the bleach event marked by an arrow. The data shown in light gray are from condensates in live tissue, while the data shown in dark gray are from fixed tissue. The solid lines represent the mean for each data set, and the shaded region represents the standard error of the mean.

FIGS. 20A-20F show immunological and fractionation data on protein localization in condensates. FIG. 20A shows anti-EPYC1 (top row), anti-Rubisco large subunit (LSU; second row), anti-Rubisco small subunit (SSU, third row), and anti-C. reinhardtii Rubisco small subunit 2 (CrRbcS2; bottom row) immunoblots against whole leaf tissue (input), the supernatant following condensate extraction and centrifugation (supernatant) and the insoluble pellet (pellet). The anti-SSU and anti-LSU antibodies are polyclonal Rubisco antibodies with greater specificities for higher plant Rubisco than for C. reinhardtii Rubisco. The columns contain samples from wild-type A. thaliana plants not expressing EPYC1 (WT), A. thaliana 1a3b Rubisco mutant plants complemented with the C. reinhardtii Rubisco small subunit and not expressing EPYC1 (S2_{Cr}), and S2_{Cr} plants expressing EPYC1-dGFP (S2_{Cr} EPYC1). For the WT sample, only the input is shown. Arrows indicate bands matching the expected molecular weights of the C. reinhardtii Rubisco small subunit 2 (CrRbcS2; 15.5 kD); the A. thaliana Rubisco small subunits 1B, 2B, and 3B (AtRbcS1B, AtRbcS2B and AtRbcS3B, respectively; 14.8 kD); and the A. thaliana Rubisco small subunit 1A (AtRbcS1A; 14.7 kD). FIG. 20B shows a coomassie-stained SDS-PAGE gel showing the composition of the pelleted condensate. Columns are labeled as in FIG. 20A. Arrows indicate bands matching the expected molecular weights of the EPYC1-GFP fusion protein (EPYC1::GFP) with the two arrows next to the EPYC1::GFP label showing the two tagged versions of EPYC1, EPYC1:eGFP and EPY1:tGFP; the Rubisco large subunit (LSU; 55 kD); the C. reinhardtii Rubisco small subunit 2 (CrRbcS2; 15.5 kD); and the A. thaliana Rubisco small subunits (AtRbcS). FIG. 20C shows fluorescence microscopy images of GFP signal from condensates from pellets from S2_{Cr} plants that have been transformed with EPYC1-GFP (S2_{Cr} EPYC1 pellet, top image) and that have not been transformed with EPYC1-GFP (S2_{Cr} pellet, bottom image). The scale bar represents 50 μm. FIG. 20D shows representative immunogold electron microscope (EM) images of chloroplasts of an S2Cr A. thaliana plant expressing EPYC1-dGFP probed with polyclonal anti-Rubisco (left) or anti-CrRbcS2 (right). Immunogold-labeled sections in the right image are circled. The scale bar represents 0.5 μm. FIG. 20E shows scatterplots of the proportion of immunogold particles that were inside the condensate compared to the remainder of the chloroplast in immunogold EM images of S2Cr A. thaliana plant expressing EPYC1-dGFP. Data are from 37-39 chloroplasts when probed with either the polyclonal anti-Rubisco antibody (Rubisco antibody) or the anti-C. reinhardtii Rubisco small subunit 2 antibody (CrRbcS2 antibody). The lines superimposed on the scatterplots represent the mean and SEM. FIG. 20F shows a representative TEM image of chloroplasts with condensates in a cross-section of a mesophyll cell from a transgenic A. thaliana S2_{Cr} plant expressing EPYC1-dGFP. The section was probed by immunogold labelling (small black dots indicated by arrows at one chloroplast) with anti-Rubisco antibodies. The scale bar represents 1 μm.

FIGS. 21A-21K show the impact of EPYC1-mediated condensation of Rubisco on growth and photosynthesis in transgenic A. thaliana plants. FIG. 21A shows fresh weight in milligrams (FW(mg)) of transgenic A. thaliana plants expressing EPYC1-dGFP WT (black bars) and of the respective azygous segregants of each line white bars) grown in 200 μmol photons m⁻² s⁻¹ light. FIG. 21B shows dry weight in milligrams (DW(mg)) of transgenic A. thaliana plants expressing EPYC1-dGFP WT (black bars) and of the respective azygous segregants of each line (white bars) grown in 200 μmol photons m⁻² s⁻¹ light. FIG. 21C shows fresh weight in milligrams (FW(mg)) of transgenic A. thaliana plants expressing EPYC1-dGFP WT (black bars) and of the respective azygous segregants of each line white bars) grown in 900 μmol photons m⁻² s⁻¹ light. FIG. 21D shows dry weight in milligrams (DW(mg)) of transgenic A. thaliana plants expressing EPYC1-dGFP WT (black bars) and of the respective azygous segregants of each line (white bars) grown in 900 μmol photons m⁻² s⁻¹ light. In FIGS. 21A-21D, displayed data are from three T2 EPYC1-dGFP S2_{Cr} transgenic lines (EP1, EP2, and EP3, respectively) and an EPYC1-dGFP WT transformant (EpWT) and their respective azygous segregants. Plants were measured after 32 days of growth. The bars represent the mean and the error bars represent the SEM for >12 individual plants for each line. Asterisks indicate a significant difference (P<0.05) in growth between the S2_{Cr} background and the WT background as determined by ANOVA; transgenic/control lines in the same background (i.e., S2_{Cr} or WT) had no significant differences in growth. FIGS. 21E-21G show plots of rosette area (in mm²) over time (in days post germination) for the same eight S2_{Cr} transgenic transformants and azygous segregants whose weights are displayed in FIGS. 21A-21D. Transgenic lines are labeled as in FIGS. 21A-21D. The azygous segregants of transgenic lines EP1-3 are labeled Az1-3, respectively. The azygous segregant of EpWT is labeled AzWT. The x-axis displays days post germination. Data points represent the mean of >12 individual plants for each line. Error bars represent the SEM. FIGS. 21E-21F show data from plants grown under 200 μmol photons m⁻² s⁻¹ light. FIG. 21E shows an overlay of the same data plotted in FIG. 21F. FIG. 21G shows data from plants grown under 900 μmol photons m's⁻¹ light. FIG. 21H shows a plot of net CO₂ assimilation (A) in μmol CO₂ m⁻² s⁻¹ for the same eight A. thaliana lines described in FIGS. 21A-G. The x-axis displays the intercellular CO₂ concentration (G) under saturating light (1500 μmol photons s⁻¹). Plant lines are labeled as in FIG. 21C. Data points and error bars show the mean and SEM, respectively, of measurements made on individual leaves from ten or more individual rosettes. FIGS. 21I-21K show photosynthetic parameters derived from gas exchange data from the same eight A. thaliana lines included in FIGS. 21A-21D. Plant lines are labeled as in FIGS. 21A-21B. The plots display the mean and SEM of measurements made on 15 to 24 whole rosettes. Asterisks indicate a significant difference (P<0.05) as determined by ANOVA. FIG. 21I shows a plot of the net CO₂ assimilation rate (A_{Rubisco}) in terms of μmol CO₂ per second, at ambient extracellular concentrations of CO₂, normalized to μmol of Rubisco sites. FIG. 21J shows a plot of the maximum rate of Rubisco carboxylation (V_{cmax}) in terms of μmol CO₂ m⁻² s⁻¹. FIG. 21K shows a plot of the maximum electron transport rate (Jₘₐₓ) in terms of μmol electrons (e⁻) m⁻²s⁻¹.

### DETAILED DESCRIPTION

The following description sets forth exemplary methods, parameters, and the like. It should be recognized, however, that such description is not intended as a limitation on the scope of the present disclosure but is instead provided as a description of exemplary embodiments.

### Genetically Altered Plants

An aspect of the disclosure includes a genetically altered higher plant or part thereof including a modified Rubisco for formation of an aggregate of modified Rubisco and Essential Pyrenoid Component 1 (EPYC1) polypeptides. An aggregate of modified Rubisco and EPYC1 may also be referred to as a condensate of modified Rubisco and EPYC1. An additional embodiment of this aspect includes the modified Rubisco being an algal Rubisco small subunit (SSU) polypeptide or a modified higher plant Rubisco SSU polypeptide wherein at least part of the higher plant Rubisco SSU polypeptide is replaced with at least part of an algal Rubisco SSU polypeptide. In a further embodiment of this aspect, which may be combined with any of the preceding embodiments, the genetically altered higher plant or part thereof further includes the EPYC1 polypeptides and the aggregate. Yet another embodiment of this aspect, which may be combined with any of the preceding embodiments, includes the aggregate being detectable by confocal microscopy, transmission electron microscopy (TEM), cryo-electron microscopy (cryo-EM), a liquid-liquid phase separation assay, or a phase separation assay. Yet another embodiment of this aspect includes the aggregate being detectable by assaying chlorophyll autofluorescence and observing a displacement of chlorophyll autofluorescence when the aggregate is present. A preferred embodiment, which may be combined with any of the preceding embodiments, includes the aggregate being detectable by confocal microscopy in vivo. A further embodiment of this aspect includes the aggregate undergoing internal mixing. An additional embodiment of this aspect includes the aggregate displacing chloroplast thylakoid membranes. Still another embodiment of this aspect, which may be combined with any of the preceding embodiments that has a modified higher plant Rubisco, includes the modified higher plant Rubisco polypeptide including an endogenous Rubisco SSU polypeptide. In yet another embodiment of this aspect, which may be combined with any of the preceding embodiments that has a modified higher plant Rubisco, the modified higher plant Rubisco SSU polypeptide was modified by substituting one or more higher plant Rubisco SSU α-helices with one or more algal Rubisco SSU α-helices; substituting one or more higher plant Rubisco SSU β-strands with one or more algal Rubisco SSU β-strands; and/or substituting a higher plant Rubisco SSU βA-βB loop with an algal Rubisco SSU βA-βB loop. An additional embodiment of this aspect includes the higher plant Rubisco SSU polypeptide being modified by substituting two higher plant Rubisco SSU α-helices with two algal Rubisco SSU α-helices. A further embodiment of this aspect includes the two higher plant Rubisco SSU α-helices corresponding to amino acids 23-35 and amino acids 80-93 in SEQ ID NO: 1 and the two algal Rubisco SSU α-helices corresponding to amino acids 23-35 and amino acids 86-99 in SEQ ID NO: 2. Yet another embodiment of this aspect that can be combined with any of the preceding embodiments that has two higher plant Rubisco SSU α-helices being substituted with two algal Rubisco SSU α-helices, the higher plant Rubisco SSU polypeptide being further modified by substituting four higher plant Rubisco SSU β-strands with four algal Rubisco SSU β-strands, and by substituting a higher plant Rubisco SSU βA-βB loop with an algal Rubisco SSU βA-βB loop. An additional embodiment of this aspect includes the four higher plant Rubisco SSU β-strands corresponding to amino acids 39-45, amino acids 68-70, amino acids 98-105, and amino acids 110-118 in SEQ ID NO: 1, the four algal Rubisco SSU β-strands corresponding to amino acids 39-45, amino acids 74-76, amino acids 104-111, and amino acids 116-124 in SEQ ID NO: 2, the higher plant Rubisco SSU βA-βB loop corresponding to amino acids 46-67 in SEQ ID NO: 1, and the algal Rubisco SSU βA-βB loop corresponding to amino acids 46-73 in SEQ ID NO: 2.

Still another embodiment of this aspect, which may be combined with any of the preceding embodiments that has a modified higher plant Rubisco, includes the higher plant Rubisco SSU polypeptide having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, or SEQ ID NO: 156. Yet another embodiment of this aspect, which may be combined with any of the preceding embodiments that has a modified higher plant Rubisco, includes the algal Rubisco SSU polypeptide having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 2, SEQ ID NO: 30, SEQ ID NO: 157, SEQ ID NO: 158, SEQ ID NO: 159, SEQ ID NO: 160, SEQ ID NO: 161, SEQ ID NO: 162, SEQ ID NO: 163, or SEQ ID NO: 164. In an additional embodiment of this aspect, the algal Rubisco SSU polypeptide is SEQ ID NO: 2, SEQ ID NO: 30, SEQ ID NO: 157, SEQ ID NO: 158, SEQ ID NO: 159, SEQ ID NO: 160, SEQ ID NO: 161, SEQ ID NO: 162, SEQ ID NO: 163, or SEQ ID NO: 164. A further embodiment of this aspect, which may be combined with any of the preceding embodiments that has a modified higher plant Rubisco, includes the modified higher plant Rubisco SSU polypeptide having increased or altered affinity for the EPYC1 polypeptide as compared to the higher plant Rubisco SSU polypeptide without the modification.

An additional aspect of the disclosure includes a genetically altered higher plant or part thereof including EPYC1 polypeptides for formation of an aggregate of modified Rubiscos and the EPYC1 polypeptides. An aggregate of modified Rubisco and EPYC1 may also be referred to as a condensate of modified Rubisco and EPYC1. A further embodiment of any of the preceding aspects includes the EPYC1 polypeptides being algal EPYC1 polypeptides. An additional embodiment of this aspect includes the algal EPYC1 polypeptides having an amino acid sequence having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 34, SEQ ID NO: 35, SEQ ID NO: 165, SEQ ID NO: 166, or SEQ ID NO: 167. In yet another embodiment of this aspect, the algal EPYC1 polypeptide is SEQ ID NO: 34, SEQ ID NO: 35, SEQ ID NO: 165, SEQ ID NO: 166, or SEQ ID NO: 167. An additional embodiment of this aspect includes EPYC1 being the mature or truncated form of EPYC1 corresponding to SEQ ID NO: 35. A further embodiment of this aspect includes the full-length form of EPYC1 corresponding to SEQ ID NO: 34 being truncated between residues 26 (V) and 27 (A) to form the mature native form of EPYC1 corresponding to SEQ ID NO: 35. Still another embodiment of any of the preceding aspects includes the EPYC1 polypeptides being modified EPYC1 polypeptides. A further embodiment of this aspect includes the modified EPYC1 polypeptides including one or more, two or more, four or more, or eight tandem copies of a first algal EPYC1 repeat region. An additional embodiment of this aspect includes the modified EPYC1 polypeptides including four tandem copies or eight tandem copies of the first algal EPYC1 repeat region. Yet another embodiment of this aspect, which may be combined with any of the preceding embodiments including modified EPYC1 polypeptides including tandem copies of a first algal EPYC1 repeat region, includes the first algal EPYC1 repeat region being a polypeptide having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 36. A further embodiment of this aspect includes the first algal EPYC1 repeat region being SEQ ID NO: 36. Still another embodiment of this aspect, which may be combined with any of the preceding embodiments including modified EPYC1, includes the modified EPYC1 polypeptides being expressed without the native EPYC1 leader sequence and/or including a C-terminal cap. Yet another embodiment of this aspect includes the native EPYC1 leader sequence including a polypeptide having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 42, and the C-terminal cap including a polypeptide having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 41. A further embodiment of this aspect includes the C-terminal cap being SEQ ID NO: 41. Still another embodiment of this aspect, which may be combined with any of the preceding embodiments including modified EPYC1, includes the modified EPYC1 polypeptide having increased affinity for Rubisco SSU polypeptide as compared to the corresponding unmodified EPYC1 polypeptide.

In yet another embodiment of this aspect, which may be combined with any of the preceding embodiments, the aggregate is localized to a chloroplast stroma of at least one chloroplast of a plant cell. The aggregate may also be referred to as the condensate. A further embodiment of this aspect includes the plant cell being a leaf mesophyll cell. In still another embodiment of this aspect, which may be combined with any of the preceding embodiments, the plant is selected from the group of cowpea (e.g., black-eyed pea, catjang, yardlong bean, Vigna unguiculata), soy (e.g., soybean, soya bean, Glycine max, Glycine soja), cassava (e.g., manioc, yucca, Manihot esculenta), rice (e.g., indica rice, japonica rice, aromatic rice, glutinous rice, Oryza sativa, Oryza glaberrima), wheat (e.g., common wheat, spelt, durum, einkorn, emmer, kamut, Triticum aestivum, Triticum spelta, Triticum durum, Triticum urartu, Triticum monococcum, Triticum turanicum, Triticum spp.), barley (e.g., Hordeum vulgare), rye (i.e., Secale cereale), oat (i.e., Avena sativa), tomato (e.g., Solanum lycopersicum), potato (e.g., russet potatoes, yellow potatoes, red potatoes, Solanum tuberosum), canola (e.g., Brassica rapa, Brassica napus, Brassica juncea), or other C3 crop plants. In some embodiments, the plant is tobacco (i.e., Nicotiana tabacum, Nicotiana edwardsonii, Nicotiana plumbagnifolia, Nicotiana longijlora, Nicotiana benthamiana) or Arabidopsis (i.e., rockcress, thale cress, Arabidopsis thaliana).

A further aspect of the disclosure includes a genetically altered higher plant or part thereof including a first nucleic acid sequence encoding an EPYC1 polypeptide and a second nucleic acid sequence encoding a modified Rubisco. An additional embodiment of this aspect includes EPYC1 being the mature or truncated form of EPYC1 corresponding to SEQ ID NO: 35. A further embodiment of this aspect includes the full-length form of EPYC1 corresponding to SEQ ID NO: 34 being truncated between residues 26 (V) and 27 (A) to form the mature native form of EPYC1 corresponding to SEQ ID NO: 35. Yet another embodiment of this aspect includes the first nucleic acid sequence being introduced with a binary vector comprising two separate expression cassettes, wherein each expression cassette comprises the first nucleic acid sequence. An additional embodiment of this aspect includes the first nucleic acid sequence being operably linked to a first promoter. A further embodiment of this aspect includes the first promoter being selected from the group of a constitutive promoter, an inducible promoter, a leaf specific promoter, or a mesophyll cell specific promoter. Yet another embodiment of this aspect includes the first promoter being a constitutive promoter selected from the group of a CaMV35S promoter, a derivative of the CaMV35S promoter, a CsVMV promoter, a derivative of the CsVMV promoter, a maize ubiquitin promoter, a trefoil promoter, a vein mosaic cassava virus promoter, and an A. thaliana UBQ10 promoter. Still another embodiment of this aspect, which may be combined with any of the preceding embodiments, includes the first nucleic acid sequence being operably linked to a third nucleic acid sequence encoding a chloroplastic transit peptide functional in the higher plant cell, and the first nucleic acid sequence not including the native EPYC1 leader sequence and not being operably linked to the native EPYC1 leader sequence. An additional embodiment of this aspect includes the chloroplastic transit peptide being a polypeptide having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 63. Yet another embodiment of this aspect includes the chloroplastic transit peptide being SEQ ID NO: 63. In a further embodiment of this aspect that can be combined with any of the preceding embodiments that has a native EPYC1 leader sequence, the native EPYC1 leader sequence corresponds to nucleotides 60-137 of SEQ ID NO: 65. In still another embodiment of this aspect that can be combined with any of the preceding embodiments, the first nucleic acid sequence is operably linked to one or two terminators. A further embodiment of this aspect includes the one two terminators being selected from the group of a HSP terminator, a NOS terminator, an OCS terminator, an intronless extensin terminator, a 35S terminator, a pinII terminator, a rbcS terminator, an actin terminator, or any combination thereof.

Still another embodiment of this aspect, which may be combined with any of the preceding embodiments, includes the second nucleic acid sequence being operably linked to a second promoter. In a further embodiment of this aspect, the second promoter is selected from the group of a constitutive promoter, an inducible promoter, a leaf specific promoter, or a mesophyll cell specific promoter. In an additional embodiment of this aspect, the second promoter is a constitutive promoter selected from the group of a CaMV35S promoter, a derivative of the CaMV35S promoter, a CsVMV promoter, a derivative of the CsVMV promoter, a maize ubiquitin promoter, a trefoil promoter, a vein mosaic cassava virus promoter, or an A. thaliana UBQ10 promoter. In yet another embodiment of this aspect that can be combined with any of the preceding embodiments that has a second nucleic acid sequence being operably linked to a second promoter, the second nucleic acid sequence encodes an algal Rubisco SSU polypeptide. In an additional embodiment of this aspect, the second nucleic acid sequence is operably linked to a fourth nucleic acid sequence encoding a chloroplastic transit peptide functional in the higher plant cell and the second nucleic acid sequence does not encode the native algal SSU leader sequence and is not operably linked to a nucleic acid sequence encoding the native algal SSU leader sequence. In a further embodiment of this aspect, the chloroplastic transit peptide is a polypeptide having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 64. In yet another embodiment of this aspect, the chloroplastic transit peptide is SEQ ID NO: 64. In still another embodiment of this aspect that can be combined with any of the preceding embodiments that has a native algal SSU leader sequence, the native algal SSU leader sequence corresponds to amino acids 1 to 45 of SEQ ID NO: 32. In yet another embodiment of this aspect that can be combined with any of the preceding embodiments that has a native algal SSU leader sequence, the native algal SSU leader sequence corresponds to amino acids 1 to 45 of SEQ ID NO: 31. In a further embodiment of this aspect that can be combined with any of the preceding embodiments that has a second nucleic acid sequence being operably linked to a second promoter, the second nucleic acid sequence is operably linked to a terminator. In an additional embodiment of this aspect, the terminator is selected from the group of a HSP terminator, a NOS terminator, an OCS terminator, an intronless extensin terminator, a 35S terminator, a pinII terminator, a rbcS terminator, or an actin terminator. In yet another embodiment of this aspect that can be combined with any of the preceding embodiments that has a second nucleic acid sequence being operably linked to a second promoter, the second nucleic acid sequence encodes a modified higher plant Rubisco SSU polypeptide wherein at least part of the higher plant Rubisco SSU polypeptide is replaced with at least part of an algal Rubisco SSU polypeptide. A further embodiment of this aspect, which can be combined with any of the preceding embodiments, includes the EPYC1 polypeptide being the EPYC1 polypeptide of any one of the preceding embodiments. An additional embodiment of this aspect includes EPYC1 being the mature or truncated form of EPYC1 corresponding to SEQ ID NO: 35. A further embodiment of this aspect includes the full-length form of EPYC1 corresponding to SEQ ID NO: 34 being truncated between residues 26 (V) and 27 (A) to form the mature native form of EPYC1 corresponding to SEQ ID NO: 35. An additional embodiment of this aspect includes the Rubisco SSU polypeptide being the Rubisco SSU polypeptide of any one of the preceding embodiments.

Yet another embodiment of this aspect, which may be combined with any of the preceding embodiments, includes at least one cell of the plant or part thereof including an aggregate of the Rubisco polypeptide and the EPYC1 polypeptide. A further embodiment of this aspect includes the aggregate being localized to a chloroplast stroma of at least one chloroplast of at least one plant cell. An additional embodiment of this aspect includes the plant cell being a leaf mesophyll cell. In still another embodiment of this aspect, which may be combined with any of the preceding embodiments that has a plant or part thereof including an aggregate of the Rubisco polypeptide and the EPYC1 polypeptide, the aggregate is detectable by confocal microscopy, transmission electron microscopy (TEM), cryo-electron microscopy (cryo-EM), or a liquid-liquid phase separation assay. Yet another embodiment of this aspect includes the aggregate being detectable by assaying chlorophyll autofluorescence and observing a displacement of chlorophyll autofluorescence when the aggregate is present. A preferred embodiment, which may be combined with any of the preceding embodiments, includes the aggregate being detectable by confocal microscopy in vivo. A further embodiment of this aspect includes the aggregate undergoing internal mixing. An additional embodiment of this aspect includes the aggregate displacing chloroplast thylakoid membranes. In yet another embodiment of this aspect, which may be combined with any of the preceding embodiments, the plant is selected from the group of cowpea (e.g., black-eyed pea, catjang, yardlong bean, Vigna unguiculata), soy (e.g., soybean, soya bean, Glycine max, Glycine soja), cassava (e.g., manioc, yucca, Manihot esculenta), rice (e.g., indica rice, japonica rice, aromatic rice, glutinous rice, Oryza sativa, Oryza glaberrima), wheat (e.g., common wheat, spelt, durum, einkorn, emmer, kamut, Triticum aestivum, Triticum spelta, Triticum durum, Triticum urartu, Triticum monococcum, Triticum turanicum, Triticum spp.), barley (e.g., Hordeum vulgare), rye (i.e., Secale cereale), oat (i.e., Avena sativa), tomato (e.g., Solanum lycopersicum), potato (e.g., russet potatoes, yellow potatoes, red potatoes, Solanum tuberosum), canola (e.g., Brassica rapa, Brassica napus, Brassica juncea), or other C3 crop plants. In some embodiments, the plant is tobacco (i.e., Nicotiana tabacum, Nicotiana edwardsonii, Nicotiana plumbagnifolia, Nicotiana longijlora, Nicotiana benthamiana) or Arabidopsis (i.e., rockcress, thale cress, Arabidopsis thaliana).

A further embodiment of this aspect that can be combined with any of the preceding embodiments includes a genetically altered higher plant cell produced from the plant or plant part of any one of the preceding embodiments. Yet another embodiment of this aspect that can be combined with any of the preceding embodiments with respect to plant part includes the plant part being a leaf, a stem, a root, a tuber, a flower, a seed, a kernel, a grain, a fruit, a cell, or a portion thereof and the genetically altered plant part including the one or more genetic alterations. A further embodiment of this aspect includes the plant part being a fruit, a tuber, a kernel, or a grain. Still another embodiment of this aspect that can be combined with any of the preceding embodiments with respect to pollen grain or ovules includes a genetically altered pollen grain or a genetically altered ovule of the plant of any one of the preceding embodiments, wherein the genetically altered pollen grain or the genetically altered ovule includes the one or more genetic alterations. A further embodiment of this aspect that can be combined with any of the preceding embodiments includes a genetically altered protoplast produced from the genetically altered plant of any of the preceding embodiments, wherein the genetically altered protoplast includes the one or more genetic alterations. An additional embodiment of this aspect that can be combined with any of the preceding embodiments includes a genetically altered tissue culture produced from protoplasts or cells from the genetically altered plant of any one of the preceding embodiments, wherein the cells or protoplasts are produced from a plant part selected from the group of leaf, leaf mesophyll cell, anther, pistil, stem, petiole, root, root tip, tuber, fruit, seed, kernel, grain, flower, cotyledon, hypocotyl, embryo, or meristematic cell, wherein the genetically altered tissue culture includes the one or more genetic alterations. An additional embodiment of this aspect includes a genetically altered plant regenerated from the genetically altered tissue culture that includes the one or more genetic alterations. Yet another embodiment of this aspect that can be combined with any of the preceding embodiments includes a genetically altered plant seed produced from the genetically altered plant of any one of the preceding embodiments.

### Methods of Producing and Cultivating Genetically Altered Plants

Another aspect of the disclosure includes methods of producing the genetically altered higher plant of any of the preceding embodiments including a) introducing a first nucleic acid sequence encoding an EPYC1 polypeptide into a plant cell, tissue, or other explant; b) regenerating the plant cell, tissue, or other explant into a genetically altered plantlet; and c) growing the genetically altered plantlet into a genetically altered plant with the first nucleic acid encoding the EPYC1 polypeptide. An additional embodiment of this aspect includes EPYC1 being the mature or truncated form of EPYC1 corresponding to SEQ ID NO: 35. A further embodiment of this aspect includes the full-length form of EPYC1 corresponding to SEQ ID NO: 34 being truncated between residues 26 (V) and 27 (A) to form the mature native form of EPYC1 corresponding to SEQ ID NO: 35. An additional embodiment of this aspect further includes introducing a second nucleic acid sequence encoding a modified Rubisco SSU polypeptide into a plant cell, tissue, or other explant prior to step (a) or concurrently with step (a), wherein the genetically altered plant of step (c) further includes the second nucleic acid encoding the modified Rubisco SSU polypeptide. An additional embodiment of this aspect further includes identifying successful introduction of the first nucleic acid sequence and, optionally, the second nucleic acid sequence by screening or selecting the plant cell, tissue, or other explant prior to step (b); screening or selecting plantlets between step (b) and (c); or screening or selecting plants after step (c). In yet another embodiment of this aspect, which may be combined with any of the preceding embodiments, transformation is done using a transformation method selected from the group of particle bombardment (i.e., biolistics, gene gun), Agrobacterium-mediated transformation, Rhizobium-mediated transformation, or protoplast transfection or transformation.

Still another embodiment of this aspect that can be combined with any of the preceding embodiments includes the first nucleic acid sequence being introduced with a first vector, and the second nucleic acid sequence being introduced with a second vector. An additional embodiment of this aspect includes the first nucleic acid sequence being introduced with a binary vector comprising two separate expression cassettes, wherein each expression cassette comprises the first nucleic acid sequence. In a further embodiment of this aspect, the first nucleic acid sequence is operably linked to a first promoter. In an additional embodiment of this aspect, the first promoter is selected from the group of a constitutive promoter, an inducible promoter, a leaf specific promoter, or a mesophyll cell specific promoter. In yet another embodiment of this aspect, the first promoter is a constitutive promoter selected from the group of a CaMV35S promoter, a derivative of the CaMV35S promoter, a CsVMV promoter, a derivative of the CsVMV promoter, a maize ubiquitin promoter, a trefoil promoter, a vein mosaic cassava virus promoter, or an A. thaliana UBQ10 promoter. In still another embodiment of this aspect that can be combined with any of the preceding embodiments, the first nucleic acid sequence is operably linked to a third nucleic acid sequence encoding a chloroplastic transit peptide functional in the higher plant cell and the first nucleic acid sequence does not include the native EPYC1 leader sequence and is not operably linked to the native EPYC1 leader sequence. In yet another embodiment of this aspect, the chloroplastic transit peptide is a polypeptide having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 63. In still another embodiment of this aspect, the endogenous chloroplastic transit peptide is SEQ ID NO: 63. Yet another embodiment of this aspect that can be combined with any of the preceding embodiments that has a native EPYC1 leader sequence includes the native EPYC1 leader sequence corresponding to nucleotides 60 to 137 of SEQ ID NO: 65. In a further embodiment of this aspect that can be combined with any of the preceding embodiments, the first nucleic acid sequence is operably linked to one or two terminators. In an additional embodiment of this aspect, the one or two terminators are selected from the group of a HSP terminator, a NOS terminator, an OCS terminator, an intronless extensin terminator, a 35S terminator, a pinII terminator, a rbcS terminator, an actin terminator, or any combination thereof.

An additional embodiment of this aspect that can be combined with any of the preceding embodiments includes the second nucleic acid sequence being operably linked to a second promoter. A further embodiment of this aspect includes the second promoter being selected from the group consisting of a constitutive promoter, an inducible promoter, a leaf specific promoter, and a mesophyll cell specific promoter. Yet another embodiment of this aspect includes the second promoter being a constitutive promoter selected from the group consisting of a CaMV35S promoter, a derivative of the CaMV35S promoter, a CsVMV promoter, a derivative of the CsVMV promoter, a maize ubiquitin promoter, a trefoil promoter, a vein mosaic cassava virus promoter, or an A. thaliana UBQ10 promoter. Still another embodiment of this aspect that can be combined with any of the preceding embodiments that has the second nucleic acid sequence being operably linked to a second promoter includes the second nucleic acid sequence encoding an algal SSU polypeptide. An additional embodiment of this aspect includes the second nucleic acid sequence being operably linked to a fourth nucleic acid sequence encoding a chloroplastic transit peptide functional in the higher plant cell and the second nucleic acid sequence not encoding the native SSU leader sequence and not being operably linked to a nucleic acid sequence encoding the native SSU leader sequence. A further embodiment of this aspect includes the chloroplastic transit peptide being a polypeptide having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 64. Yet another embodiment of this aspect includes the chloroplastic transit peptide being SEQ ID NO: 64. An additional embodiment of this aspect that can be combined with any of the preceding embodiments, which has a native SSU leader sequence, includes the native SSU leader sequence corresponding to amino acids 1 to 45 of SEQ ID NO: 32. In yet another embodiment of this aspect that can be combined with any of the preceding embodiments that has a native algal SSU leader sequence, the native algal SSU leader sequence corresponds to amino acids 1 to 45 of SEQ ID NO: 31. Still another embodiment of this aspect that can be combined with any of the preceding embodiments that has the second nucleic acid sequence being operably linked to a second promoter includes the second nucleic acid sequence being operably linked to a terminator. A further embodiment of this aspect includes the terminator being selected from the group of a HSP terminator, a NOS terminator, an OCS terminator, an intronless extensin terminator, a 35S terminator, a pinII terminator, a rbcS terminator, or an actin terminator. In a further embodiment of this aspect that can be combined with any of the preceding embodiments that has the second nucleic acid sequence being operably linked to a second promoter, the second nucleic acid sequence encodes a modified higher plant Rubisco SSU polypeptide wherein at least part of the higher plant Rubisco SSU polypeptide is replaced with at least part of an algal Rubisco SSU polypeptide.

In an additional embodiment of this aspect that can be combined with any of the preceding embodiments that has a second vector, the second vector includes one or more gene editing components that target a nuclear genome sequence operably linked to a nucleic acid encoding an endogenous Rubisco SSU polypeptide. A further embodiment of this aspect includes one or more gene editing components being selected from the group of a ribonucleoprotein complex that targets the nuclear genome sequence; a vector comprising a TALEN protein encoding sequence, wherein the TALEN protein targets the nuclear genome sequence; a vector comprising a ZFN protein encoding sequence, wherein the ZFN protein targets the nuclear genome sequence; an oligonucleotide donor (ODN), wherein the ODN targets the nuclear genome sequence; or a vector comprising a CRISPR/Cas enzyme encoding sequence and a targeting sequence, wherein the targeting sequence targets the nuclear genome sequence. Yet another embodiment of this aspect that can be combined with any of the preceding embodiments that has gene editing includes the result of gene editing being at least part of the higher plant Rubisco SSU polypeptide being replaced with at least part of an algal Rubisco SSU polypeptide. A further embodiment of this aspect, which can be combined with any of the preceding embodiments, includes the EPYC1 polypeptide being the EPYC1 polypeptide of any one of the preceding embodiments. An additional embodiment of this aspect includes the Rubisco SSU polypeptide being the Rubisco SSU polypeptide of any one of the preceding embodiments.

Yet another embodiment of this aspect that can be combined with any of the preceding embodiments that has a first nucleic acid sequence being operably linked to a third nucleic acid sequence encoding a chloroplastic transit peptide functional in the higher plant cell and the first nucleic acid sequence not comprising the native EPYC1 leader sequence and not being operably linked to the native EPYC1 leader sequence includes and that has the first nucleic acid sequence being operably linked to one or two terminators includes the first vector including a first copy of the first nucleic acid sequence wherein the first nucleic acid sequence does not include the native EPYC1 leader sequence and is not operably linked to the native EPYC1 leader sequence, wherein the first nucleic acid sequence is operably linked to the third nucleic acid sequence encoding a chloroplastic transit peptide functional in the higher plant cell, wherein the first nucleic acid sequence is operably linked to the first promoter, and wherein the first nucleic acid sequence is operably linked to one terminator; and wherein the first vector further includes a second copy of the first nucleic acid sequence wherein the first nucleic acid sequence does not include the native EPYC1 leader sequence and is not operably linked to the native EPYC1 leader sequence, wherein the first nucleic acid sequence is operably linked to the third nucleic acid sequence encoding a chloroplastic transit peptide functional in the higher plant cell, wherein the first nucleic acid sequence is operably linked to a third promoter, and wherein the first nucleic acid sequence is operably linked to two terminators. A further embodiment of this aspect includes the first promoter being selected from the group of a constitutive promoter, an inducible promoter, a leaf specific promoter, or a mesophyll cell specific promoter; wherein the third promoter is selected from the group of a constitutive promoter, an inducible promoter, a leaf specific promoter, or a mesophyll cell specific promoter; and wherein the first and third promoters are not the same. Yet another embodiment of this aspect includes the chloroplastic transit peptide being a polypeptide having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 63. Still another embodiment of this aspect includes the native EPYC1 leader sequence corresponding to nucleotides 60 to 137 of SEQ ID NO: 65. An additional embodiment of this aspect includes the terminators being selected from the group of a HSP terminator, a NOS terminator, an OCS terminator, an intronless extensin terminator, a 35S terminator, a pinII terminator, a rbcS terminator, an actin terminator, or any combination thereof. A further embodiment of this aspect that can be combined with any of the preceding embodiments includes a plant or plant part produced by the method of any one of the preceding embodiments.

A further aspect of the disclosure includes methods of cultivating the genetically altered plant of any of the preceding embodiments that has a genetically altered plant, including the steps of: a) planting a genetically altered seedling, a genetically altered plantlet, a genetically altered cutting, a genetically altered tuber, a genetically altered root, or a genetically altered seed in soil to produce the genetically altered plant or grafting the genetically altered seedling, the genetically altered plantlet, or the genetically altered cutting to a root stock or a second plant grown in soil to produce the genetically altered plant; b) cultivating the plant to produce harvestable seed, harvestable leaves, harvestable roots, harvestable cuttings, harvestable wood, harvestable fruit, harvestable kernels, harvestable tubers, and/or harvestable grain; and harvesting the harvestable seed, harvestable leaves, harvestable roots, harvestable cuttings, harvestable wood, harvestable fruit, harvestable kernels, harvestable tubers, and/or harvestable grain; and c) harvesting the harvestable seed, harvestable leaves, harvestable roots, harvestable cuttings, harvestable wood, harvestable fruit, harvestable kernels, harvestable tubers, and/or harvestable grain. An additional embodiment of this aspect includes a plant growth rate and/or photosynthetic efficiency of the genetically altered plant of any of the preceding embodiments being comparable to the plant growth rate and/or photosynthetic efficiency of a WT plant. Yet another embodiment of this aspect includes a plant growth rate and/or photosynthetic efficiency of the genetically altered plant of any of the preceding embodiments being improved as compared to the plant growth rate and/or photosynthetic efficiency of a WT plant. Still another embodiment of this aspect includes a yield of the genetically altered plant of any of the preceding embodiments being improved as compared to the yield of a WT plant. A further embodiment of this aspect includes the yield being improved by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

### Molecular Biological Methods to Produce Genetically Altered Plants and Plant Cells

One embodiment of the present invention provides a genetically altered plant or plant cell containing a modified Rubisco and an Essential Pyrenoid Component 1 (EPYC1) for formation of an aggregate or condensate of modified Rubisco and EPYC1 polypeptides. For example, the present disclosure provides plants with a first nucleic acid sequence encoding an EPYC1 polypeptide and a second nucleic acid sequence encoding a modified Rubisco. In addition, the present disclosure provides plants with algal EPYC1 polypeptides, modified EPYC1 polypeptides, algal Rubisco small subunit (SSU) polypeptides, and modified Rubisco SSU polypeptides.

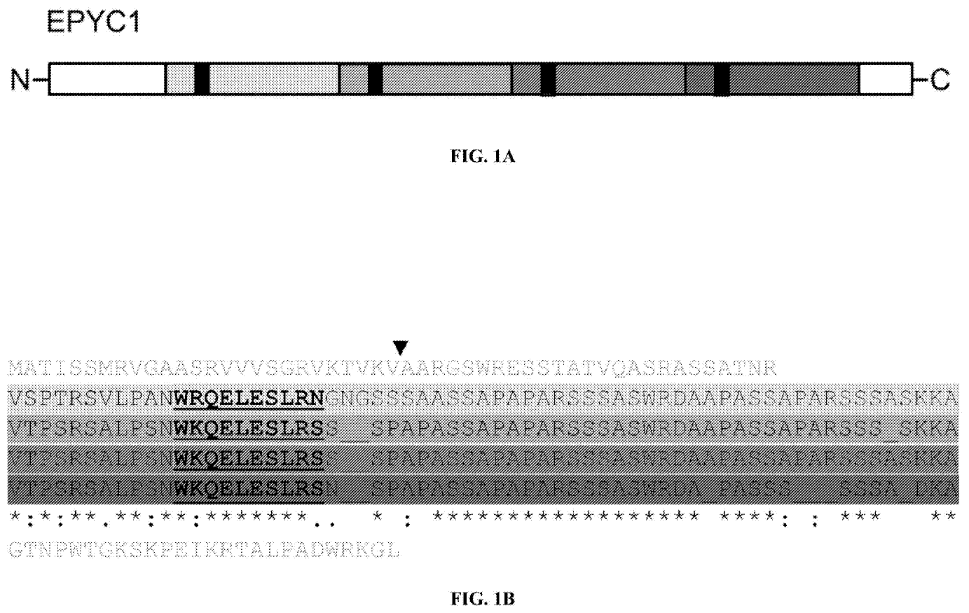

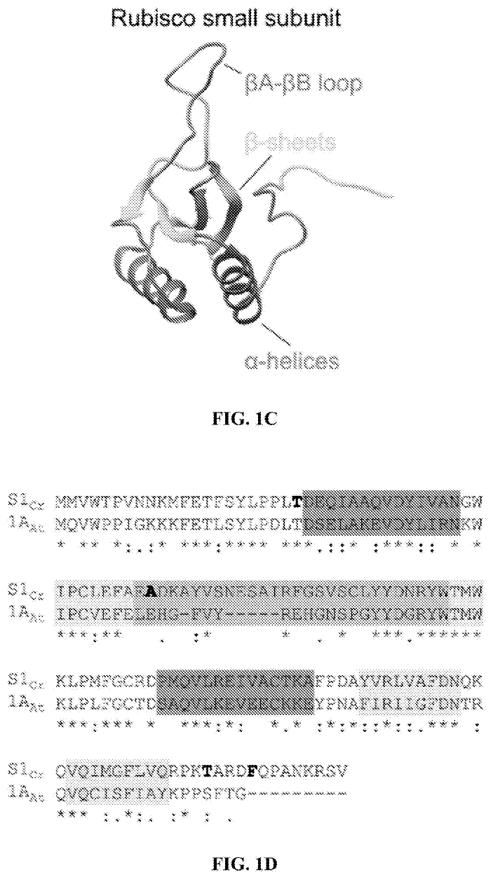

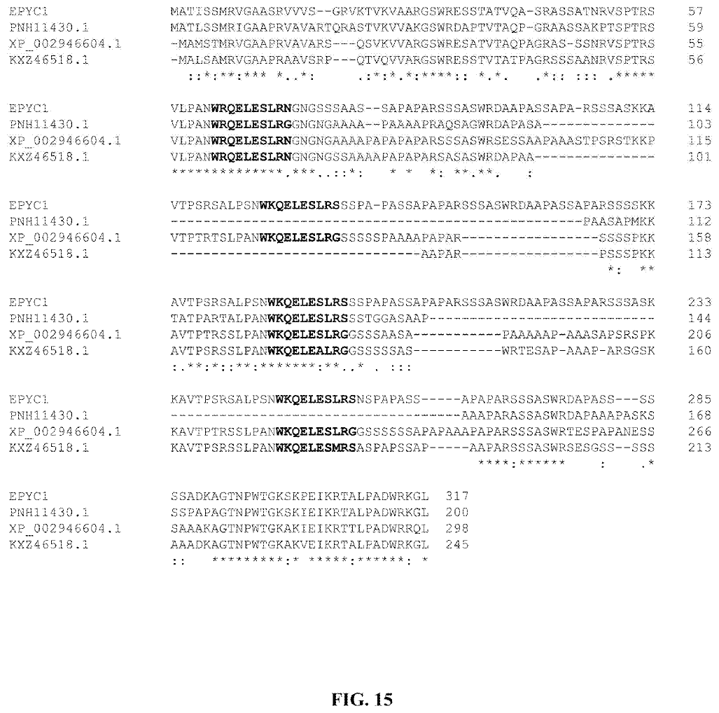

Certain aspects of the present invention relate to the C. reinhardtii protein EPYC1 (C. reinhardtii EPYC1 genomic sequence=SEQ ID NO: 66; C. reinhardtii EPYC1 transcript sequence=SEQ ID NO: 65; C. reinhardtii EPYC1 full length protein=SEQ ID NO: 34; C. reinhardtii mature EPYC1 protein=SEQ ID NO: 35). EPYC1 is a modular protein consisting of four highly similar repeat regions flanked by shorter terminal regions (FIGS. 1A-1B). Each of the four similar repeat regions consists of a predicted disordered domain and a shorter, less disordered domain containing a predicted α-helix. Further aspects of the present invention relate to homologs or orthologs of EPYC1. In some embodiments, a homolog or ortholog of EPYC1 is structurally similar to C. reinhardtii EPYC1. As shown in FIG. 15, three other closely related algal species, namely Volvox carteri, Gonium pectorale, and Tetrabaena socialis, have proteins homologous to C. reinhardtii EPYC1 (SEQ ID NO: 166 (V. carteri); SEQ ID NO: 167 (G. pectorale); SEQ ID NO: 165 (T. socialis)) with the same repeat regions containing predicted α-helices regions as in C. reinhardtii EPYC1.

At the N-terminus of the native C. reinhardtii protein EPYC1, a cleavage site at amino acid 26 in SEQ ID NO: 34 (indicated by a black arrow in FIG. 1B) results in a truncated the N-terminus in the mature EPYC1 protein of SEQ ID NO: 35. Preferably, expression of EPYC1 in higher plants uses a coding sequence such that the EPYC1 protein produced has a truncated N-terminus. An additional embodiment of this aspect includes the truncated N-terminus (i.e., N-terminus of the mature EPYC1 protein) being a polypeptide having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 40. A further embodiment of this aspect includes the truncated N-terminus (i.e., N-terminus of the mature EPYC1 protein) being SEQ ID NO: 40.

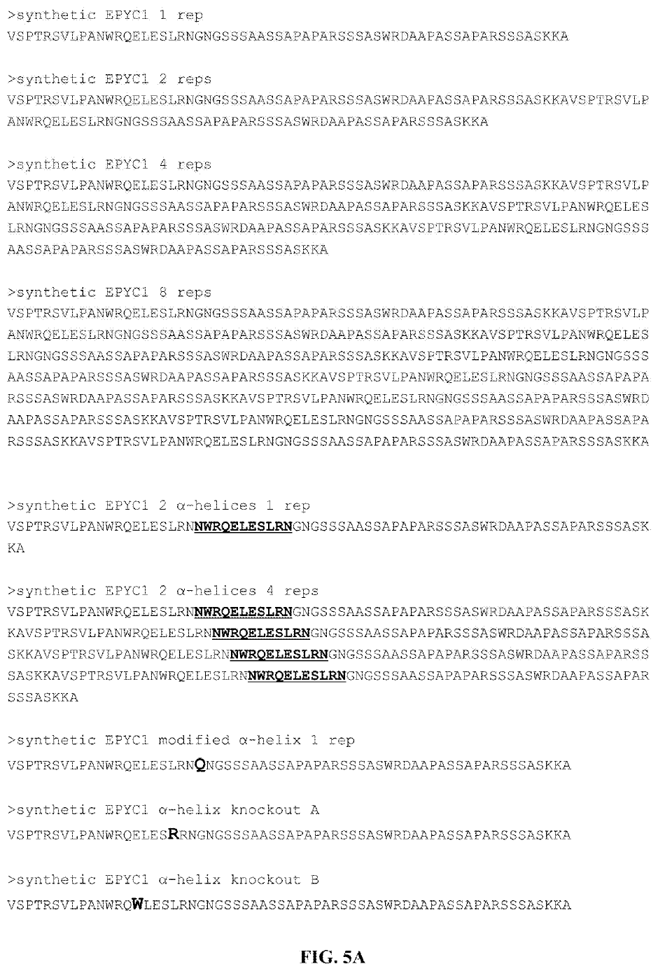

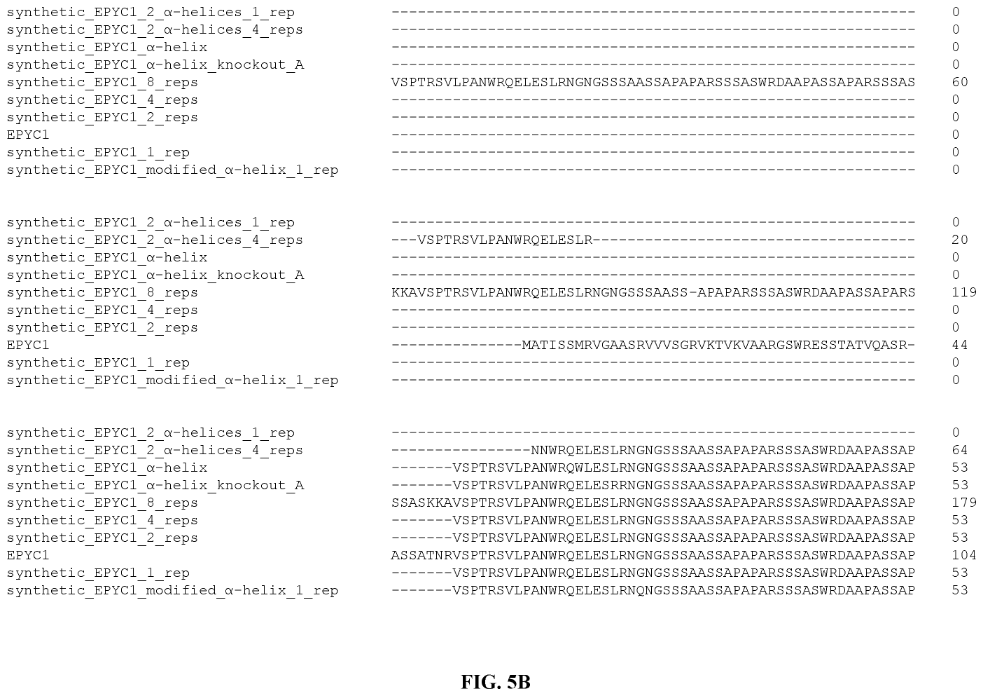

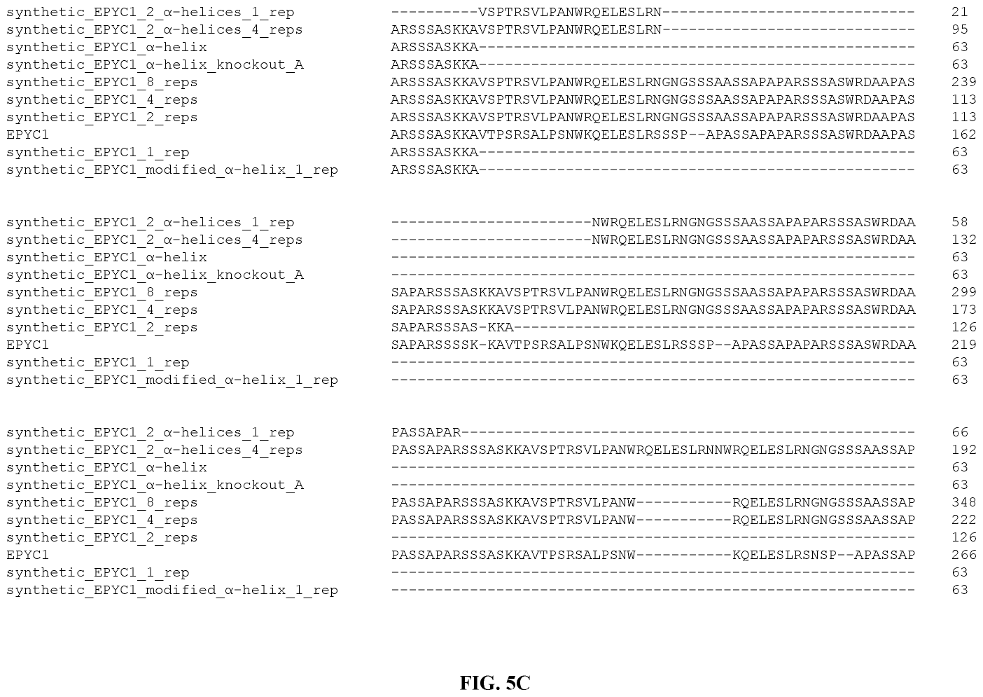

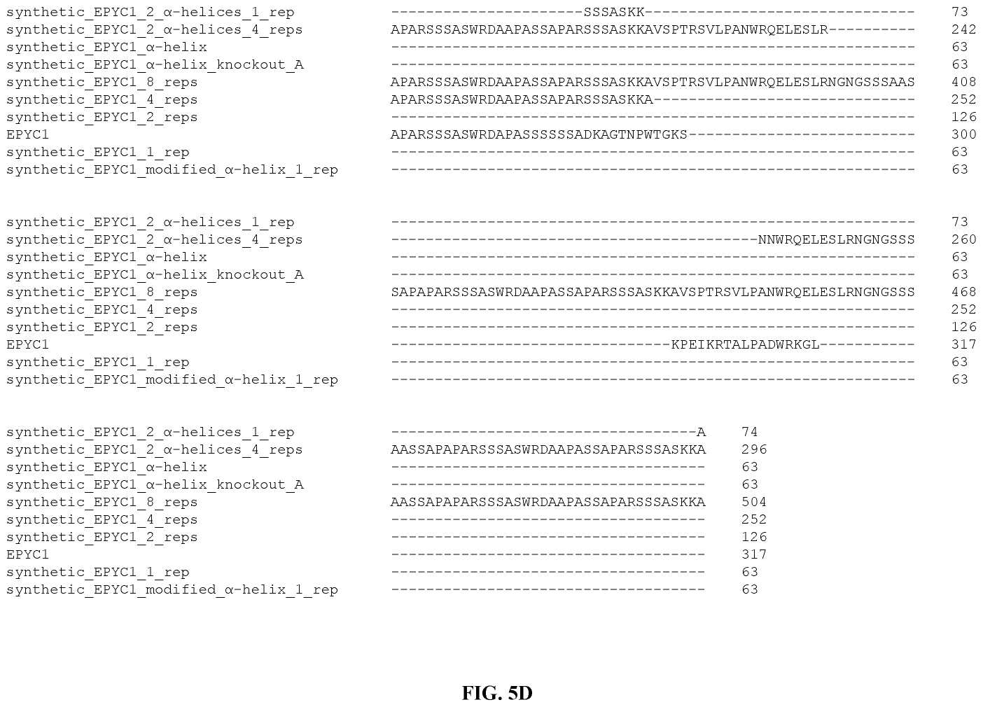

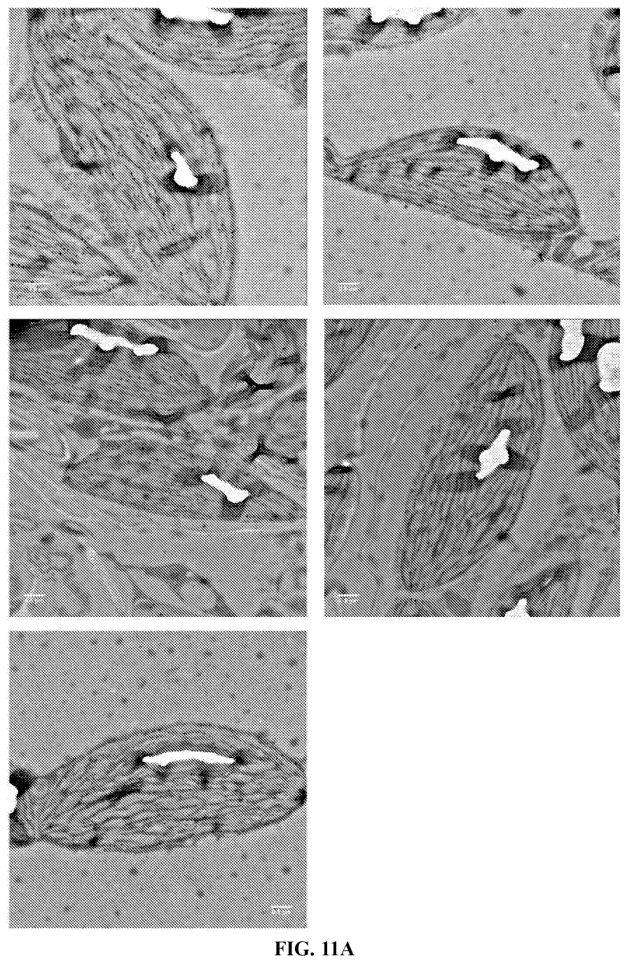

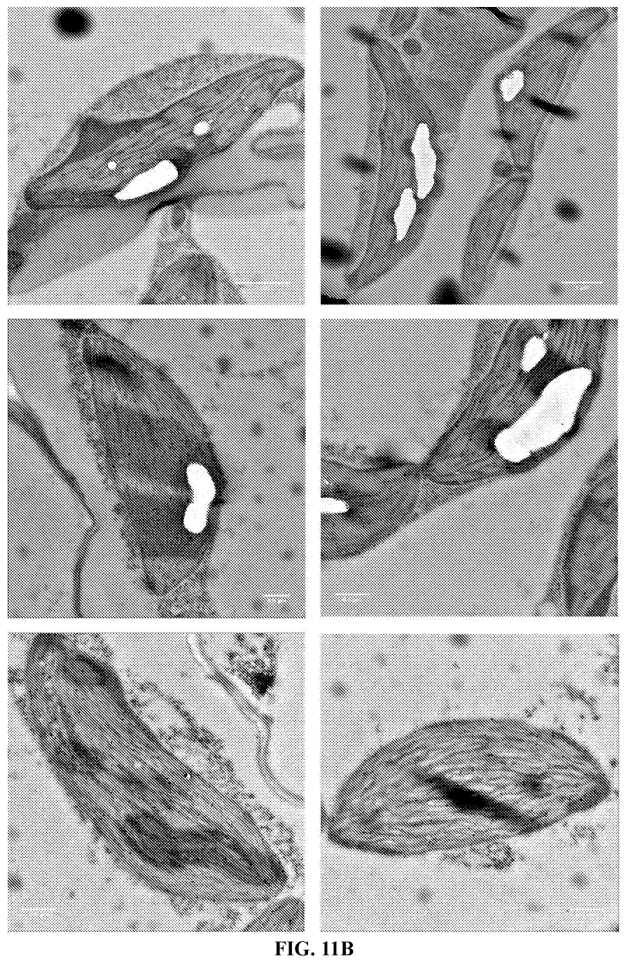

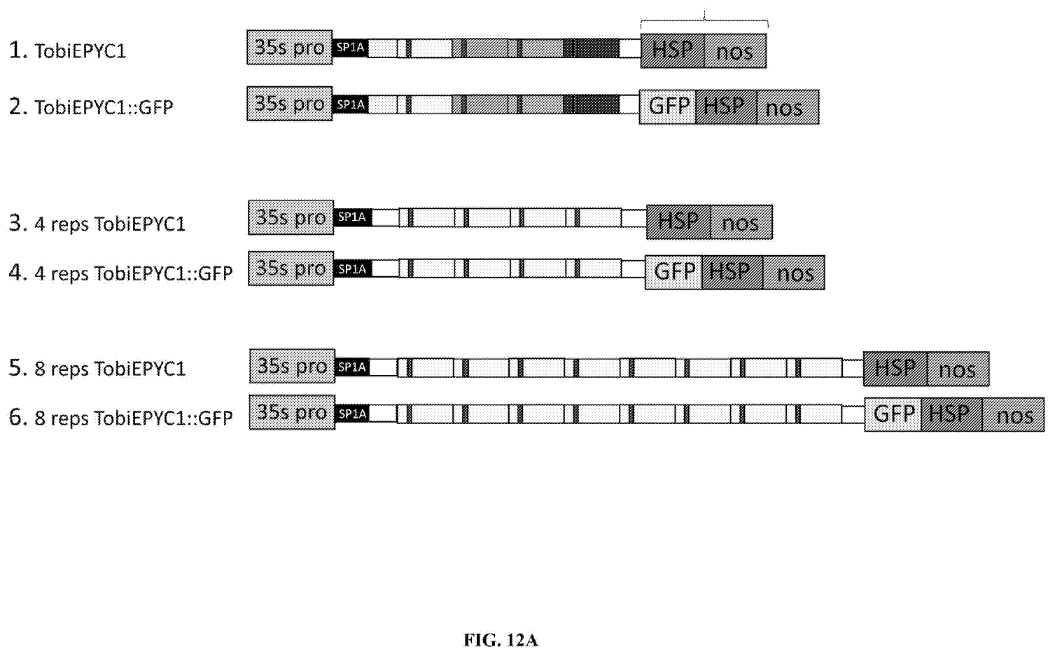

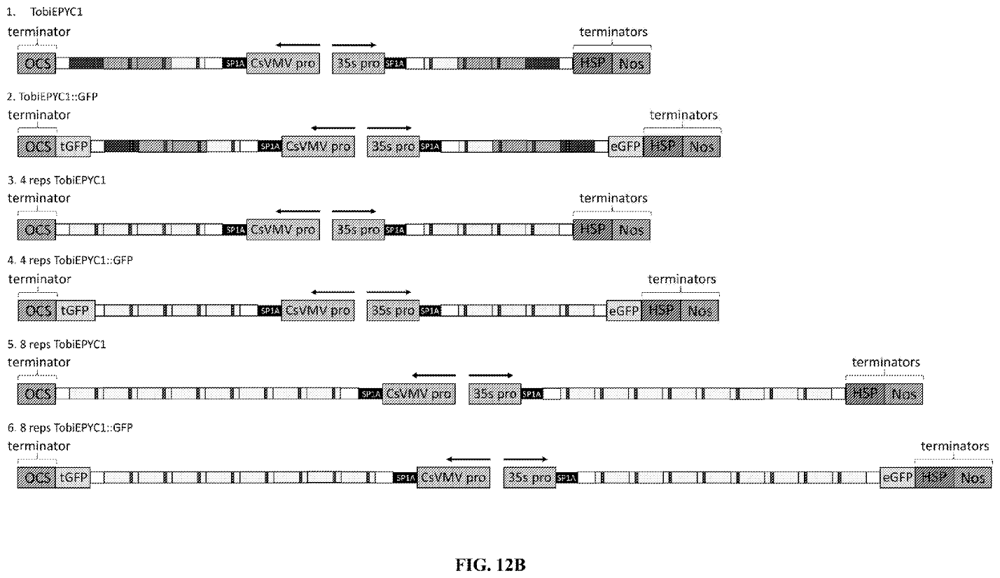

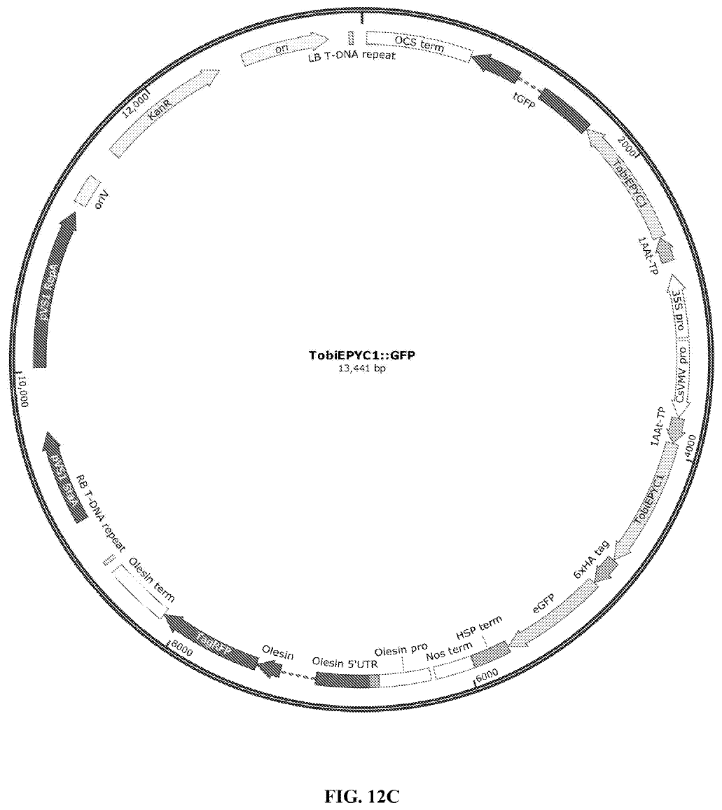

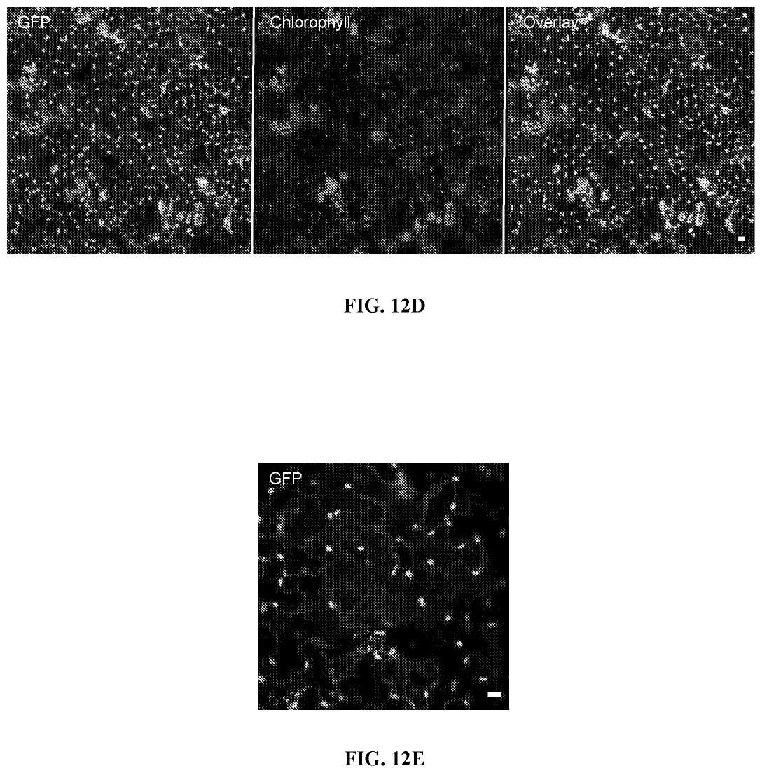

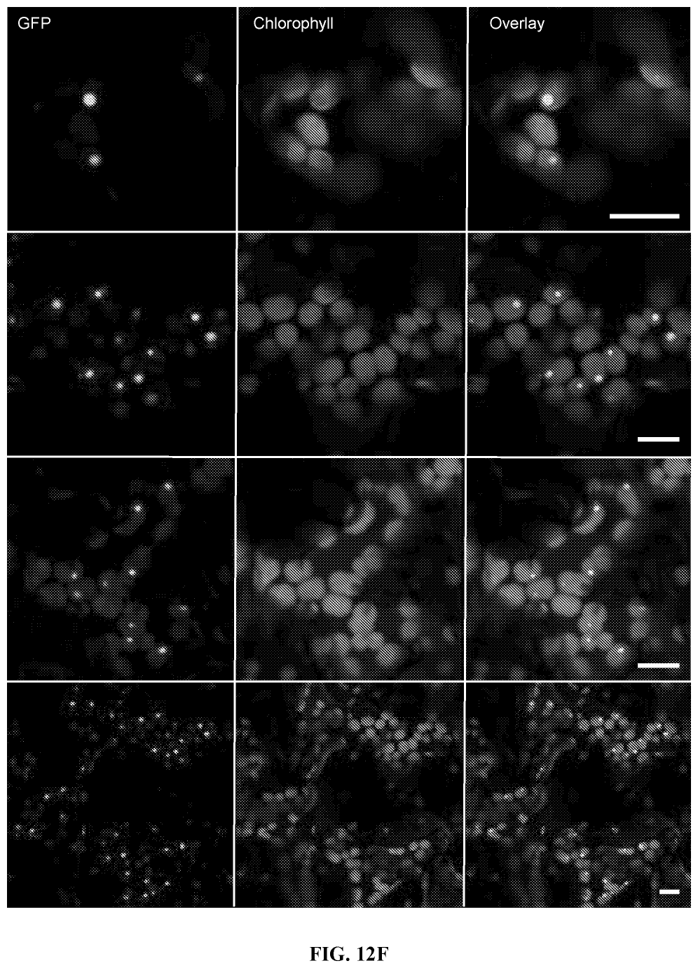

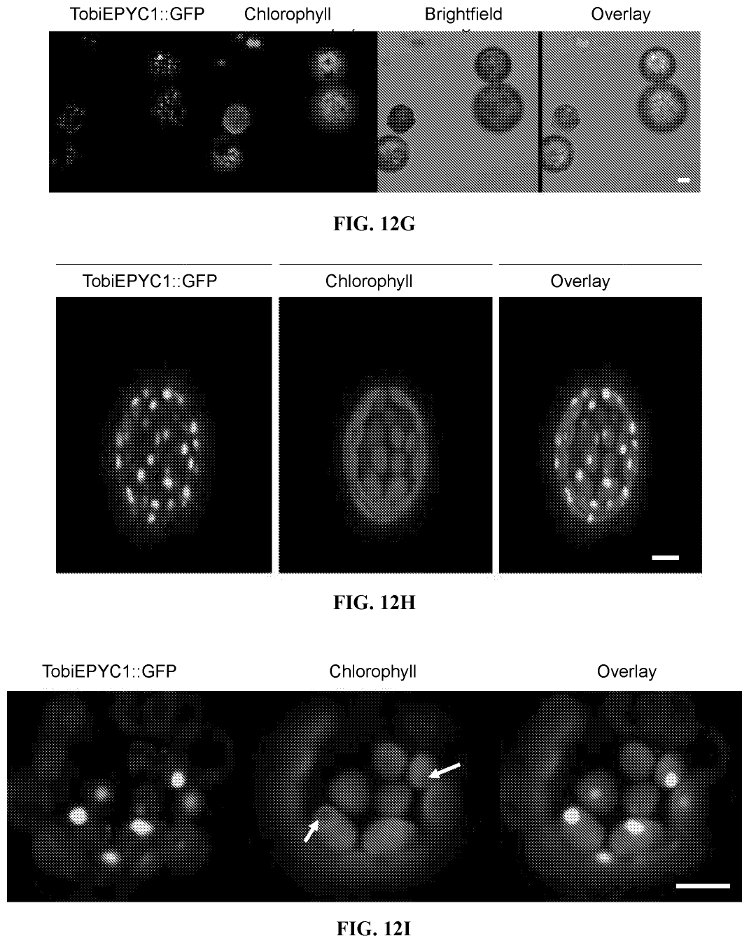

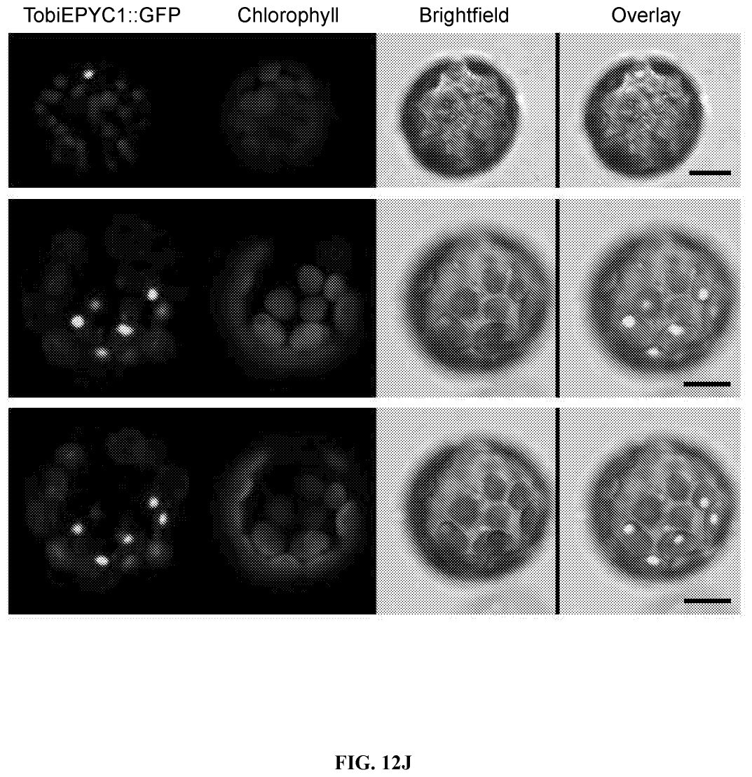

A modified EPYC1 polypeptide of the present invention includes tandem copies of the first EPYC1 repeat domain. A further embodiment of this aspect includes the modified EPYC1 polypeptides including one or more, two or more, four or more, or eight tandem copies of a first algal EPYC1 repeat region. An additional embodiment of this aspect includes the modified EPYC1 polypeptides including four tandem copies or eight tandem copies of the first algal EPYC1 repeat region. Exemplary modified EPYC1 sequences are shown in FIG. 5A. Some embodiments of this aspect include the first algal EPYC1 repeat region being a polypeptide having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 36. A further embodiment of this aspect includes the first algal EPYC1 repeat region being SEQ ID NO: 36. Still another embodiment of this aspect, includes the modified EPYC1 polypeptides being expressed without the native EPYC1 leader sequence and/or including a C-terminal cap. Yet another embodiment of this aspect includes the native EPYC1 leader sequence being a polypeptide having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 42, and the C-terminal cap being a polypeptide having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 41. Still another embodiment of this aspect includes the C-terminal cap being SEQ ID NO: 41. A further embodiment of this aspect includes a truncated N-terminus (i.e., N-terminus of the mature EPYC1 protein) being used in place of the native EPYC1 leader sequence. An additional embodiment of this aspect includes the truncated N-terminus (i.e., N-terminus of the mature EPYC1 protein) being a polypeptide having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 40. A further embodiment of this aspect includes the truncated N-terminus (i.e., N-terminus of the mature EPYC1 protein) being SEQ ID NO: 40. Exemplary gene expression cassettes containing modified EPYC1 sequences without the native EPYC1 leader sequence, with the truncated N-terminus (i.e., N-terminus of the mature EPYC1 protein), and with the C-terminal cap are shown in FIGS. 12A-12B.

For correct targeting of EPYC1 in a higher plant, a higher plant chloroplast targeting sequence is attached to the EPYC1 sequence. In some embodiments, this chloroplast targeting sequence is the 1A_{At} chloroplastic transit peptide. In further embodiments, the chloroplast targeting sequence is the 1B_{At} chloroplastic transit peptide (SEQ ID NO: 18), 2B_{At} chloroplastic transit peptide (SEQ ID NO: 19), or the 3B_{At} chloroplastic transit peptide (SEQ ID NO: 20). In additional embodiments, the chloroplast targeting sequence is obtained from chlorophyll a/b-binding protein, Rubisco activase, ferredoxin, or starch synthase proteins. In additional embodiments, the chloroplast transit sequence is a truncated chloroplast transit sequence (e.g., 55 residues of the 1A_{At} chloroplastic transit peptide). A further embodiment of this aspect includes the chloroplastic transit peptide being a polypeptide having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 64. Yet another embodiment of this aspect includes the chloroplastic transit peptide being SEQ ID NO: 64. Exemplary gene expression cassettes containing the 55 residue 1A_{At} chloroplastic transit peptide attached to EPYC1 sequences (mature EPYC1 and modified EPYC1) are shown in FIGS. 12A-12B. Means known in the art can be used to test chloroplast targeting sequences for their suitability for EPYC1 targeting, and to optimize the length of the chloroplast targeting sequence (e.g., Shen, et al., Sci. Rep. (2017): 46231).

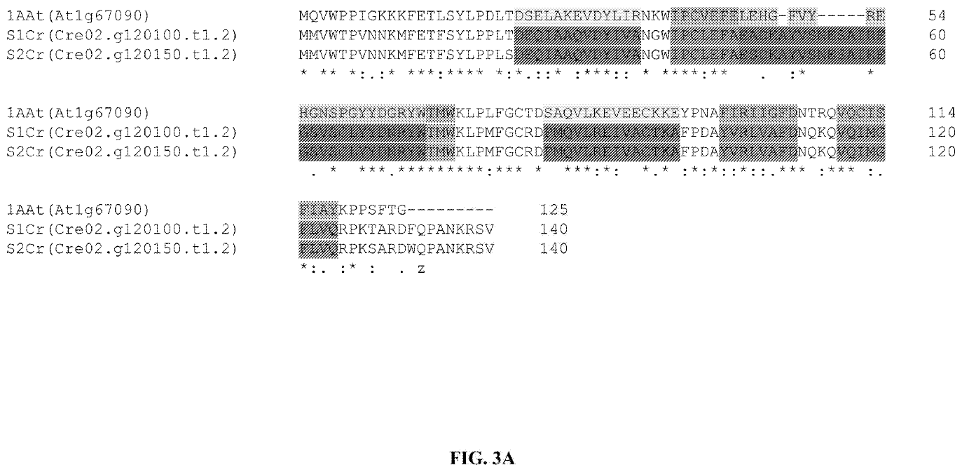

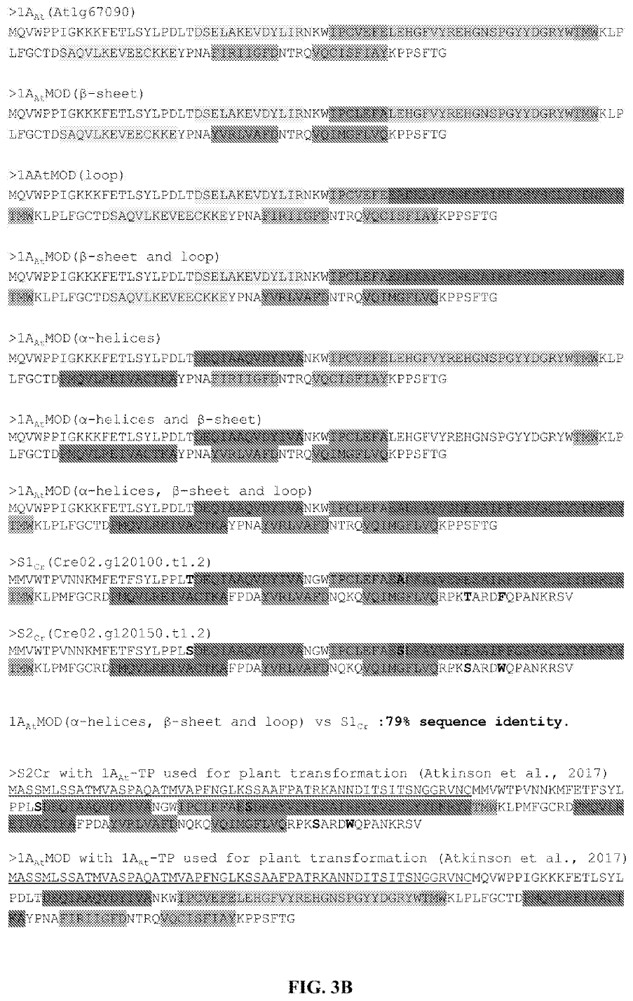

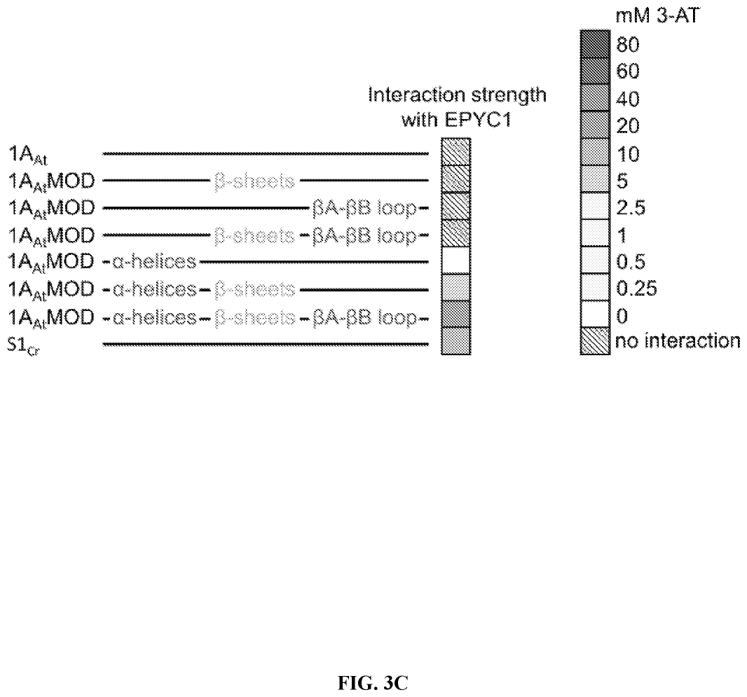

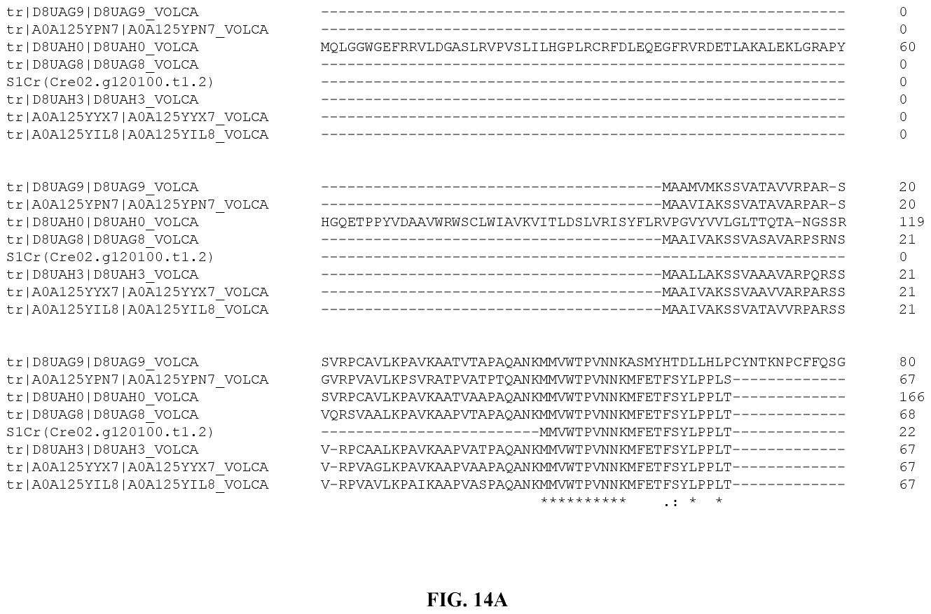

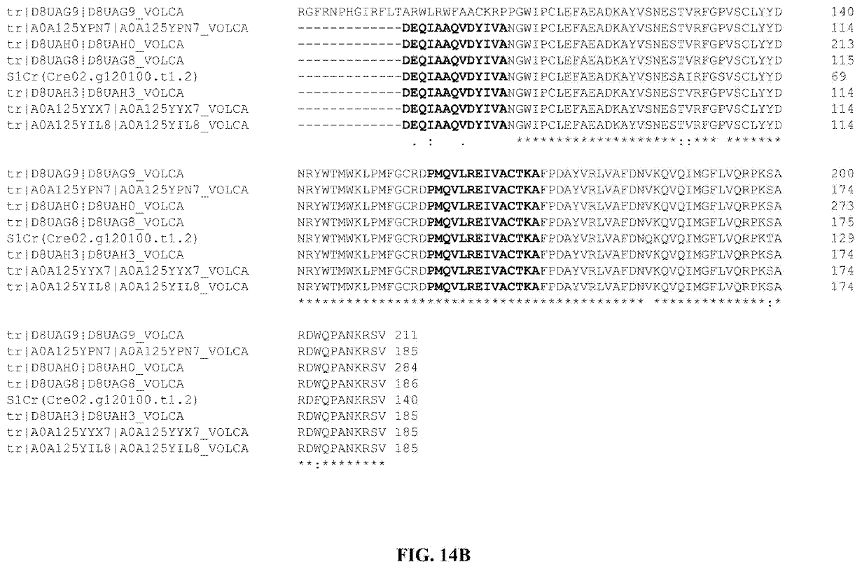

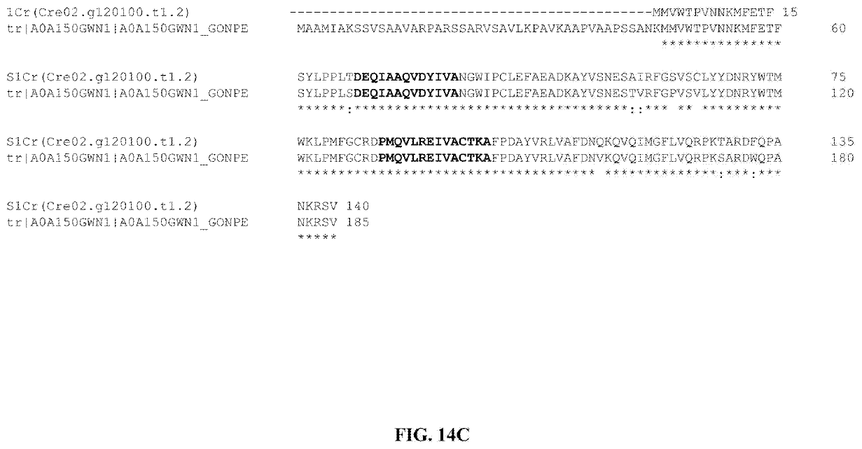

Additional aspects of the present invention relate to an algal Rubisco SSU protein. In some embodiments, the algal Rubisco SSU proteins is a C. reinhardtii Rubisco SSU protein, S1_{Cr} (SEQ ID NO: 30) or S2_{Cr} (SEQ ID NO: 2) (FIGS. 1D and 3D). A further aspect of the present invention relates to algal homologs or orthologs of C. reinhardtii Rubisco SSU. In an additional embodiment of this aspect, the algal Rubisco SSU protein is a V. carteri or a G. pectorale Rubisco SSU proteins (FIGS. 14A-14C; SEQ ID NO: 157, SEQ ID NO: 158, SEQ ID NO: 159, SEQ ID NO: 160, SEQ ID NO: 161; SEQ ID NO: 162; SEQ ID NO: 163, and SEQ ID NO: 164). In another embodiment of this aspect, an algal homolog or ortholog of C. reinhardtii Rubisco SSU has an amino acid sequence that is at least 70%, at least 71%, at least 72%, at least 73%, at least 74%, at least 75%, at least 75%, at least 76%, at least 77%, at least 78%, at least 79%, at least 80%, at least 81%, at least 82%, at least 83%, at least 84%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 30 or SEQ ID NO: 2. A further aspect of the present invention relates to algal Rubisco SSU proteins without algal Rubisco SSU leader sequences. In some embodiments of this aspect, the algal Rubisco SSU leader sequences have amino acid sequence that are at least 70%, at least 71%, at least 72%, at least 73%, at least 74%, at least 75%, at least 75%, at least 76%, at least 77%, at least 78%, at least 79%, at least 80%, at least 81%, at least 82%, at least 83%, at least 84%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 29. In further embodiments of this aspect, the algal Rubisco SSU leader sequence is SEQ ID NO: 29.

A modified Rubisco SSU of the present invention includes a higher plant Rubisco SSU modified by substituting one or more higher plant Rubisco SSU α-helices with one or more algal Rubisco SSU α-helices; substituting one or more higher plant Rubisco SSU β-strands with one or more algal Rubisco SSU β-strands; and/or substituting a higher plant Rubisco SSU βA-βB loop with an algal Rubisco SSU βA-βB loop. In some embodiments, the higher plant Rubisco SSU polypeptide is modified by substituting two higher plant Rubisco SSU α-helices with two algal Rubisco SSU α-helices. In additional embodiments, the higher plant Rubisco SSU polypeptide is further modified by substituting four higher plant Rubisco SSU β-strands with four algal Rubisco SSU β-strands, and by substituting a higher plant Rubisco SSU βA-βB loop with an algal Rubisco SSU βA-βB loop. Higher plant Rubisco SSU polypeptides of the present invention include polypeptides having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, or SEQ ID NO: 156. Algal Rubisco SSU polypeptides of the present invention include polypeptides having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 2, SEQ ID NO: 30, SEQ ID NO: 157, SEQ ID NO: 158, SEQ ID NO: 159, SEQ ID NO: 160, SEQ ID NO: 161, SEQ ID NO: 162, SEQ ID NO: 163, or SEQ ID NO: 164. In an additional embodiment of this aspect, the algal Rubisco SSU polypeptide is SEQ ID NO: 2, SEQ ID NO: 30, SEQ ID NO: 157, SEQ ID NO: 158, SEQ ID NO: 159, SEQ ID NO: 160, SEQ ID NO: 161, SEQ ID NO: 162, SEQ ID NO: 163, or SEQ ID NO: 164. A further embodiment of this aspect includes the two higher plant Rubisco SSU α-helices corresponding to amino acids 23-35 (i.e., SEQ ID NO: 3) and amino acids 80-93 (i.e., SEQ ID NO: 4) in SEQ ID NO: 1 and the two algal Rubisco SSU α-helices corresponding to amino acids 23-35 (i.e., SEQ ID NO: 10) and amino acids 86-99 (i.e., SEQ ID NO: 12) in SEQ ID NO: 2. Yet another embodiment of this aspect that can be combined with any of the preceding embodiments that has two higher plant Rubisco SSU α-helices being substituted with two algal Rubisco SSU α-helices, the higher plant Rubisco SSU polypeptide being further modified by substituting four higher plant Rubisco SSU β-strands with four algal Rubisco SSU β-strands, and by substituting a higher plant Rubisco SSU βA-βB loop with an algal Rubisco SSU βA-βB loop. An additional embodiment of this aspect includes the four higher plant Rubisco SSU β-strands corresponding to amino acids 39-45 (i.e., SEQ ID NO: 5), amino acids 68-70 (i.e., SEQ ID NO: 6), amino acids 98-105 (i.e., SEQ ID NO: 7), and amino acids 110-118 (i.e., SEQ ID NO: 8) in SEQ ID NO: 1, the four algal Rubisco SSU β-strands corresponding to amino acids 39-45 (i.e., SEQ ID NO: 11), amino acids 74-76 (i.e., SEQ ID NO: 6), amino acids 104-111 (i.e., SEQ ID NO: 13), and amino acids 116-124 (i.e., SEQ ID NO: 14) in SEQ ID NO: 2, the higher plant Rubisco SSU βA-βB loop corresponding to amino acids 46-67 (i.e., SEQ ID NO: 9) in SEQ ID NO: 1, and the algal Rubisco SSU βA-βB loop corresponding to amino acids 46-73 (i.e., SEQ ID NO: 15) in SEQ ID NO: 2. In further embodiments, the algal Rubisco SSU βA-βB loop corresponds to SEQ ID NO: 16.

A higher plant chloroplast targeting sequence is attached to the algal Rubisco SSU or the modified Rubisco SSU. In some embodiments, this chloroplast targeting sequence is the 1A_{At} chloroplastic transit peptide. In further embodiments, the chloroplast targeting sequence is the 1B_{At} chloroplastic transit peptide (SEQ ID NO: 18), 2B_{At} chloroplastic transit peptide (SEQ ID NO: 19), or the 3B_{At} chloroplastic transit peptide (SEQ ID NO: 20). In additional embodiments, the chloroplast targeting sequence is obtained from chlorophyll a/b-binding protein, Rubisco activase, ferredoxin, or starch synthase proteins. In additional embodiments, the chloroplast transit sequence is a truncated chloroplast transit sequence (e.g., 57 residues of the 1A_{At} chloroplastic transit peptide). A further embodiment of this aspect includes the chloroplastic transit peptide being a polypeptide having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to SEQ ID NO: 63. Yet another embodiment of this aspect includes the chloroplastic transit peptide being SEQ ID NO: 63. Exemplary sequences containing the 57 residue 1A_{At} chloroplastic transit peptide attached to SSU sequences (S2_{Cr} with 1A_{At}-TP (SEQ ID NO: 22) and 1A_{A1}MOD with 1A_{At}-TP (SEQ ID NO: 33)) are shown in FIG. 3B. Means known in the art can be used to test chloroplast targeting sequences for their suitability for modified Rubisco SSU targeting, and to optimize the length of the chloroplast targeting sequence (e.g., Shen, et al., Sci. Rep. (2017): 46231).

Transformation and generation of genetically altered monocotyledonous and dicotyledonous plant cells is well known in the art. See, e.g., Weising, et al., Ann. Rev. Genet. 22:421-477 (1988); U.S. Pat. No. 5,679,558; Agrobacterium Protocols, ed: Gartland, Humana Press Inc. (1995); and Wang, et al. Acta Hort. 461:401-408 (1998). The choice of method varies with the type of plant to be transformed, the particular application and/or the desired result. The appropriate transformation technique is readily chosen by the skilled practitioner.

Any methodology known in the art to delete, insert or otherwise modify the cellular DNA (e.g., genomic DNA and organelle DNA) can be used in practicing the inventions disclosed herein. For example, a disarmed Ti plasmid, containing a genetic construct for deletion or insertion of a target gene, in Agrobacterium tumefaciens can be used to transform a plant cell, and thereafter, a transformed plant can be regenerated from the transformed plant cell using procedures described in the art, for example, in EP 0116718, EP 0270822, PCT publication WO 84/02913 and published European Patent application (“EP”) 0242246. Ti-plasmid vectors each contain the gene between the border sequences, or at least located to the left of the right border sequence, of the T-DNA of the Ti-plasmid. Of course, other types of vectors can be used to transform the plant cell, using procedures such as direct gene transfer (as described, for example in EP 0233247), pollen mediated transformation (as described, for example in EP 0270356, PCT publication WO 85/01856, and U.S. Pat. No. 4,684,611), plant RNA virus-mediated transformation (as described, for example in EP 0 067 553 and U.S. Pat. No. 4,407,956), liposome-mediated transformation (as described, for example in U.S. Pat. No. 4,536,475), and other methods such as the methods for transforming certain lines of corn (e.g., U.S. Pat. No. 6,140,553; Fromm et al., Bio/Technology (1990) 8, 833-839); Gordon-Kamm et al., The Plant Cell, (1990) 2, 603-618) and rice (Shimamoto et al., Nature, (1989) 338, 274-276; Datta et al., Bio/Technology, (1990) 8, 736-740) and the method for transforming monocots generally (PCT publication WO 92/09696). For cotton transformation, the method described in PCT patent publication WO 00/71733 can be used. For soybean transformation, reference is made to methods known in the art, e.g., Hinchee et al. (Bio/Technology, (1988) 6, 915) and Christou et al. (Trends Biotech, (1990) 8, 145) or the method of WO 00/42207.

Genetically altered plants of the present invention can be used in a conventional plant breeding scheme to produce more genetically altered plants with the same characteristics, or to introduce the genetic alteration(s) in other varieties of the same or related plant species. Seeds, which are obtained from the altered plants, preferably contain the genetic alteration(s) as a stable insert in nuclear DNA or as modifications to an endogenous gene or promoter. Plants comprising the genetic alteration(s) in accordance with the invention include plants comprising, or derived from, root stocks of plants comprising the genetic alteration(s) of the invention, e.g., fruit trees or ornamental plants. Hence, any non-transgenic grafted plant parts inserted on a transformed plant or plant part are included in the invention.

Introduced genetic elements, whether in an expression vector or expression cassette, which result in the expression of an introduced gene, will typically utilize a plant-expressible promoter. A ‘plant-expressible promoter’ as used herein refers to a promoter that ensures expression of the genetic alteration(s) of the invention in a plant cell. Examples of promoters directing constitutive expression in plants are known in the art and include: the strong constitutive 35S promoters (the “35S promoters”) of the cauliflower mosaic virus (CaMV), e.g., of isolates CM 1841 (Gardner et al., Nucleic Acids Res, (1981) 9, 2871-2887), CabbB S (Franck et al., Cell (1980) 21, 285-294; Kay et al., Science, (1987) 236, 4805) and CabbB JI (Hull and Howell, Virology, (1987) 86, 482-493); cassava vein mosaic virus promoter (CsVMV); promoters from the ubiquitin family (e.g., the maize ubiquitin promoter of Christensen et al., Plant Mol Biol, (1992) 18, 675-689, or the A. thaliana UBQ10 promoter of Norris et al. Plant Mol. Biol. (1993) 21, 895-906), the gos2 promoter (de Pater et al., The Plant J (1992) 2, 834-844), the emu promoter (Last et al., Theor Appl Genet, (1990) 81, 581-588), actin promoters such as the promoter described by An et al. (The Plant J, (1996) 10, 107), the rice actin promoter described by Zhang et al. (The Plant Cell, (1991) 3, 1155-1165); promoters of the Cassava vein mosaic virus (WO 97/48819, Verdaguer et al. (Plant Mol Biol, (1998) 37, 1055-1067), the pPLEX series of promoters from Subterranean Clover Stunt Virus (WO 96/06932, particularly the S4 or S7 promoter), an alcohol dehydrogenase promoter, e.g., pAdh1S (GenBank accession numbers X04049, X00581), and the TR1′ promoter and the TR2′ promoter (the “TR1′ promoter” and “TR2′ promoter”, respectively) which drive the expression of the 1′ and 2′ genes, respectively, of the T DNA (Velten et al., EMBO J, (1984) 3, 2723 2730).

Alternatively, a plant-expressible promoter can be a tissue-specific promoter, i.e., a promoter directing a higher level of expression in some cells or tissues of the plant, e.g., in leaf mesophyll cells. In preferred embodiments, leaf mesophyll specific promoters or leaf guard cell specific promoters will be used. Non-limiting examples include the leaf specific Rbcs1A promoter (A. thaliana RuBisCO small subunit 1A (AT1G67090) promoter), GAPA-1 promoter (A. thaliana Glyceraldehyde 3-phosphate dehydrogenase A subunit 1 (AT3G26650) promoter), and FBA2 promoter (A. thaliana Fructose-bisphosphate aldolase 2 317 (AT4G38970) promoter) (Kromdijk et al., Science, 2016). Further non-limiting examples include the leaf mesophyll specific FBPase promoter (Peleget al., Plant J, 2007), the maize or rice rbcS promoter (Nomura et al., Plant Mol Biol, 2000), the leaf guard cell specific A. thaliana KAT1 promoter (Nakamura et al., Plant Phys, 1995), the A. thaliana Myrosinase-Thioglucoside glucohydrolase 1 (TGG1) promoter (Husebye et al., Plant Phys, 2002), the A. thaliana rha1 promoter (Terryn et al., Plant Cell, 1993), the A. thaliana AtCHX20 promoter (Padmanaban et al., Plant Phys, 2007), the A. thaliana HIC (High carbon dioxide) promoter (Gray et al., Nature, 2000), the A. thaliana CYTOCHROME P450 86A2 (CYP86A2) mono-oxygenase promoter (pCYP) (Francia et al., Plant Signal & Behav, 2008; Galbiati et al., The Plant Journal, 2008), the potato ADP-glucose pyrophosphorylase (AGPase) promoter (Muller-Rober et al., The Plant Cell 1994), the grape R2R3 MYB60 transcription factor promoter (Galbiati et al., BMC Plant Bio, 2011), the A. thaliana AtMYB60 promoter (Cominelli et al., Current Bio, 2005; Cominelli et al., BMC Plant Bio, 2011), the A. thaliana At1g22690-promoter (pGC1) (Yang et al., Plant Methods, 2008), and the A. thaliana AtMYB 61 promoter (Liang et al., Curr Biol, 2005). These plant promoters can be combined with enhancer elements, they can be combined with minimal promoter elements, or can comprise repeated elements to ensure the expression profile desired.

In some embodiments, genetic elements to increase expression in plant cells can be utilized. For example, an intron at the 5′ end or 3′ end of an introduced gene, or in the coding sequence of the introduced gene, e.g., the hsp70 intron. Other such genetic elements can include, but are not limited to, promoter enhancer elements, duplicated or triplicated promoter regions, 5′ leader sequences different from another transgene or different from an endogenous (plant host) gene leader sequence, 3′ trailer sequences different from another transgene used in the same plant or different from an endogenous (plant host) trailer sequence.

An introduced gene of the present invention can be inserted in host cell DNA so that the inserted gene part is upstream (i.e., 5′) of suitable 3′ end transcription regulation signals (e.g., transcript formation and polyadenylation signals). This is preferably accomplished by inserting the gene in the plant cell genome (nuclear or chloroplast). Preferred polyadenylation and transcript formation signals include those of the A. tumefaciens nopaline synthase gene (Nos terminator; Depicker et al., J. Molec Appl Gen, (1982) 1, 561-573), the octopine synthase gene (OCS terminator; Gielen et al., EMBO J, (1984) 3:835 845), the A. thaliana heat shock protein terminator (HSP terminator); the SCSV or the Malic enzyme terminators (Schunmann et al., Plant Funct Biol, (2003) 30:453-460), and the T DNA gene 7 (Velten and Schell, Nucleic Acids Res, (1985) 13, 6981 6998), which act as 3′ untranslated DNA sequences in transformed plant cells. In some embodiments, one or more of the introduced genes are stably integrated into the nuclear genome. Stable integration is present when the nucleic acid sequence remains integrated into the nuclear genome and continues to be expressed (e.g., detectable mRNA transcript or protein is produced) throughout subsequent plant generations. Stable integration into and/or editing of the nuclear genome can be accomplished by any known method in the art (e.g., microparticle bombardment, Agrobacterium-mediated transformation, CRISPR/Cas9, electroporation of protoplasts, microinjection, etc.).

The term recombinant or modified nucleic acids refers to polynucleotides which are made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

As used herein, the terms “overexpression” and “upregulation” refer to increased expression (e.g., of mRNA, polypeptides, etc.) relative to expression in a wild type organism (e.g., plant) as a result of genetic modification. In some embodiments, the increase in expression is a slight increase of about 10% more than expression in wild type. In some embodiments, the increase in expression is an increase of 50% or more (e.g., 60%, 70%, 80%, 100%, etc.) relative to expression in wild type. In some embodiments, an endogenous gene is overexpressed. In some embodiments, an exogenous gene is overexpressed by virtue of being expressed. Overexpression of a gene in plants can be achieved through any known method in the art, including but not limited to, the use of constitutive promoters, inducible promoters, high expression promoters, enhancers, transcriptional and/or translational regulatory sequences, codon optimization, modified transcription factors, and/or mutant or modified genes that control expression of the gene to be overexpressed.

Where a recombinant nucleic acid is intended for expression, cloning, or replication of a particular sequence, DNA constructs prepared for introduction into a host cell will typically comprise a replication system (e.g. vector) recognized by the host, including the intended DNA fragment encoding a desired polypeptide, and can also include transcription and translational initiation regulatory sequences operably linked to the polypeptide-encoding segment. Additionally, such constructs can include cellular localization signals (e.g., plasma membrane localization signals). In preferred embodiments, such DNA constructs are introduced into a host cell's genomic DNA, chloroplast DNA or mitochondrial DNA.

In some embodiments, a non-integrated expression system can be used to induce expression of one or more introduced genes. Expression systems (expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides can also be included where appropriate from secreted polypeptides of the same or related species, which allow the protein to cross and/or lodge in cell membranes, cell wall, or be secreted from the cell.

Selectable markers useful in practicing the methodologies of the invention disclosed herein can be positive selectable markers. Typically, positive selection refers to the case in which a genetically altered cell can survive in the presence of a toxic substance only if the recombinant polynucleotide of interest is present within the cell. Negative selectable markers and screenable markers are also well known in the art and are contemplated by the present invention. One of skill in the art will recognize that any relevant markers available can be utilized in practicing the inventions disclosed herein.

Screening and molecular analysis of recombinant strains of the present invention can be performed utilizing nucleic acid hybridization techniques. Hybridization procedures are useful for identifying polynucleotides, such as those modified using the techniques described herein, with sufficient homology to the subject regulatory sequences to be useful as taught herein. The particular hybridization techniques are not essential to the subject invention. As improvements are made in hybridization techniques, they can be readily applied by one of skill in the art. Hybridization probes can be labeled with any appropriate label known to those of skill in the art. Hybridization conditions and washing conditions, for example temperature and salt concentration, can be altered to change the stringency of the detection threshold. See, e.g., Sambrook et al. (1989) vide infra or Ausubel et al. (1995) Current Protocols in Molecular Biology, John Wiley & Sons, NY, N.Y., for further guidance on hybridization conditions.

Additionally, screening and molecular analysis of genetically altered strains, as well as creation of desired isolated nucleic acids can be performed using Polymerase Chain Reaction (PCR). PCR is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. (1985) Science 230:1350-1354). PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Because the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes which can be used are known to those skilled in the art.

Nucleic acids and proteins of the present invention can also encompass homologues of the specifically disclosed sequences. Homology (e.g., sequence identity) can be 50%-100%. In some instances, such homology is greater than 80%, greater than 85%, greater than 90%, or greater than 95%. The degree of homology or identity needed for any intended use of the sequence(s) is readily identified by one of skill in the art. As used herein percent sequence identity of two nucleic acids is determined using an algorithm known in the art, such as that disclosed by Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN, BLASTP, and BLASTX, programs of Altschul et al. (1990) J. Mol. Biol. 215:402-410. BLAST nucleotide searches are performed with the BLASTN program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (BLASTN and BLASTX) are used. See www.ncbi.nih.gov. One of skill in the art can readily determine in a sequence of interest where a position corresponding to amino acid or nucleic acid in a reference sequence occurs by aligning the sequence of interest with the reference sequence using the suitable BLAST program with the default settings (e.g., for BLASTP: Gap opening penalty: 11, Gap extension penalty: 1, Expectation value: 10, Word size: 3, Max scores: 25, Max alignments: 15, and Matrix: blosum62; and for BLASTN: Gap opening penalty: 5, Gap extension penalty:2, Nucleic match: 1, Nucleic mismatch −3, Expectation value: 10, Word size: 11, Max scores: 25, and Max alignments: 15).

Preferred host cells are plant cells. Recombinant host cells, in the present context, are those which have been genetically modified to contain an isolated nucleic molecule, contain one or more deleted or otherwise non-functional genes normally present and functional in the host cell, or contain one or more genes to produce at least one recombinant protein. The nucleic acid(s) encoding the protein(s) of the present invention can be introduced by any means known to the art which is appropriate for the particular type of cell, including without limitation, transformation, lipofection, electroporation or any other methodology known by those skilled in the art.

### Plant Breeding Methods

Plant breeding begins with the analysis of the current germplasm, the definition of problems and weaknesses of the current germplasm, the establishment of program goals, and the definition of specific breeding objectives. The next step is the selection of germplasm that possess the traits to meet the program goals. The selected germplasm is crossed in order to recombine the desired traits and through selection, varieties or parent lines are developed. The goal is to combine in a single variety or hybrid an improved combination of desirable traits from the parental germplasm. These important traits may include higher yield, field performance, improved fruit and agronomic quality, resistance to biological stresses, such as diseases and pests, and tolerance to environmental stresses, such as drought and heat.

Each breeding program should include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.). Promising advanced breeding lines are thoroughly tested and compared to appropriate standards in environments representative of the commercial target area(s) for three years at least. The best lines are candidates for new commercial cultivars; those still deficient in a few traits are used as parents to produce new populations for further selection. These processes, which lead to the final step of marketing and distribution, usually take five to ten years from the time the first cross or selection is made.

The choice of breeding or selection methods depends on the mode of plant reproduction, the heritability of the trait(s) being improved, and the type of cultivar used commercially (e.g., F₁ hybrid cultivar, inbred cultivar, etc.). For highly heritable traits, a choice of superior individual plants evaluated at a single location will be effective, whereas for traits with low heritability, selection should be based on mean values obtained from replicated evaluations of families of related plants. The complexity of inheritance also influences the choice of the breeding method. Backcross breeding is used to transfer one or a few genes for a highly heritable trait into a desirable cultivar (e.g., for breeding disease-resistant cultivars), while recurrent selection techniques are used for quantitatively inherited traits controlled by numerous genes, various recurrent selection techniques are used. Commonly used selection methods include pedigree selection, modified pedigree selection, mass selection, and recurrent selection.

Pedigree selection is generally used for the improvement of self-pollinating crops or inbred lines of cross-pollinating crops. Two parents which possess favorable, complementary traits are crossed to produce an F₁. An F2 population is produced by selfing one or several F₁s or by intercrossing two F₁s (sib mating). Selection of the best individuals is usually begun in the F₂ population; then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding (i.e., recurrent selection) may be used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In addition to phenotypic observations, the genotype of a plant can also be examined. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs—which are also referred to as Microsatellites), and Single Nucleotide Polymorphisms (SNPs).

Molecular markers, or “markers”, can also be used during the breeding process for the selection of qualitative traits. For example, markers closely linked to alleles or markers containing sequences within the actual alleles of interest can be used to select plants that contain the alleles of interest. The use of markers in the selection process is often called genetic marker enhanced selection or marker-assisted selection. Methods of performing marker analysis are generally known to those of skill in the art.

Mutation breeding may also be used to introduce new traits into plant varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development: Theory and Technique, Walter Fehr (1991), Agronomy Books, 1 (https://lib.dr.iastate.edu/agron_books/1).

The production of double haploids can also be used for the development of homozygous lines in a breeding program. Double haploids are produced by the doubling of a set of chromosomes from a heterozygous plant to produce a completely homozygous individual. For example, see Wan, et al., Theor. Appl. Genet., 77:889-892, 1989.

Additional non-limiting examples of breeding methods that may be used include, without limitation, those found in Principles of Plant Breeding, John Wiley and Son, pp. 115-161 (1960); Principles of Cultivar Development: Theory and Technique, Walter Fehr (1991), Agronomy Books, 1 (https://lib.dr.iastate.edu/agron_books/1), which are herewith incorporated by reference.

Having generally described this invention, the same will be better understood by reference to certain specific examples, which are included herein to further illustrate the invention and are not intended to limit the scope of the invention as defined by the claims.

### EXAMPLES

The present disclosure is described in further detail in the following examples which are not in any way intended to limit the scope of the disclosure as claimed. The attached figures are meant to be considered as integral parts of the specification and description of the disclosure. The following examples are offered to illustrate, but not to limit the claimed disclosure.

### Example 1: Rubisco and EPYC1 Interact and can be Engineered to Increase their Interaction Strength

The following example describes the development and engineering of different variants of EPYC1 and different variants of the Rubisco Small Subunit (SSU). The example also describes yeast two-hybrid experiments testing the interactions between EPYC1 variants and Rubisco SSU variants.

### Materials and Methods

Chlamydomonas reinhardtii and Arabidopsis thaliana Rubisco Small Subunits (SSUs) and the C. Reinhardtii Protein Essential Pyrenoid Component 1 (EPYC1)

C. reinhardtii has two similar Rubisco SSU homologs, S1_{Cr} (SEQ ID NO: 30) and S2_{Cr} (SEQ ID NO: 2), which are the same size and have identical α-helices and β-sheets. S1_{Cr} and S2_{Cr} share a 97.1% identity at the protein level, and differ in amino acid sequence by only four residues (indicated in bold in FIG. 1D). One of these four residues is in the βA-βB loop, meaning that this loop has a one residue difference (A47S) between S1_{Cr} and S2_{Cr}. Mature A. thaliana SSU 1A (1A_{At}; SEQ ID NO: 1; structure shown in FIG. 1C) and the C. reinhardtii SSUs are structurally similar, but only have 45.0% identity at the protein level. C. reinhardtii S1_{Cr} and S2_{Cr} (140 amino acids (aa)) are longer overall than 1A_{At} (125 aa), and have a longer βA-βB loop (by 6 aa) and C-terminus (by 9 aa) than 1A_{At}. As shown in FIG. 3A, the α-helices, β-strand, and βA-βB loop regions of the SSUs are substantially different between A. thaliana and C. reinhardtii.

The C. reinhardtii protein EPYC1 is a modular protein consisting of four highly similar repeat regions flanked by shorter terminal regions (FIGS. 1A-1B) (full length EPYC1=SEQ ID NO: 34; mature EPYC1 (i.e., after cleavage site processing)=SEQ ID NO: 35). Each of the four similar repeat regions consists of a predicted disordered domain and a shorter, less disordered domain containing a predicted α-helix. EPYC1 protein aligns in BLAST to proteins in only three other closely related algal species, namely Volvox carteri (VOLCADRAFT_103023, 63.5% identity), Gonium pectorale (GPECTOR_43g955, 42.2% identity), and Tetrabaena socialis (A1O1_04388, 44.9% identity). As shown in FIG. 15, all three homologs also have repeat regions with predicted α-helices regions (as in EPYC1). The Rubisco SSUs of two of these algal species with EPYC1 homologs, V. carteri and G. pectorale, have α-helices that are mostly identical to those of C. reinhardtii S1_{Cr} (see bold text in FIGS. 14A-14C). This strongly indicates that EPYC1 and SSUs interact in a similar way in these species.

### Yeast Two-Hybrid (Y2H)

The yeast two-hybrid plasmid vectors pGBKT7 (binding domain vector) and pGADT7 (activation domain vector) were used to detect interactions between proteins of interest. Genes were amplified using Q5 DNA polymerase (NEB) and the primers listed in Table 1. Both S1_{Cr} and S2_{Cr} were used in initial yeast two-hybrid testing, and then S2_{Cr} was used in later experiments due to being more highly expressed in C. reinhardtii. The coding sequence of EPYC1 was codon optimized for expression in higher plants using an online tool (www.idtdna.com/CodonOpt). All variants of EPYC1 were synthesized as Gblock fragments (IDT), and amplified using the primers listed in Table 1. Amplified genes were then cloned into each vector using the multiple cloning site, thus creating fusions with either the GAL4 DNA binding or activation domain, respectively.

TABLE 1
List of primers used for producing the vectors used
in the yeast two-hybrid assays.

| Primer name | Primer sequence | Vector |
|---|---|---|
| EPYC.1 BD&AD Fw | TTTTGAATTCATGGCTACGATCAGTT | pGBKT7_EPYC1 |
| | CTATGAGAGT (SEQ ID NO: 72) | pGADT7_EPYC1 |
| EPYC.1 BD&AD Rev | ATAGGATCCTCAAAGGCCCTTTCTC | |
| | CAGTCTG (SEQ ID NO: 73) | |
| RbcS1 mature BD&AD | AAAAGAATTCGTGTGGACACCGGTG | pGADT7_S1_{Cr} |
| Fw | AACAACAAG (SEQ ID NO: 74) | pGBKT7_S1_{Cr} |
| RbcS1 BD&AD Rev | ATACCCGGGACGTTTGTTGGCTGGT | |
| | TGGAAATC (SEQ ID NO: 75) | |
| matRbcS2 Fw AD | AAAAGAATTCGTGTGGACACCGGTG | pGADT7_S2_{Cr} |
| | AACAACAAG (SEQ ID NO: 74) | pGBKT7_S2_{Cr} |
| matRbcS2 Rev AD | TATCCCGGGACGTTTGTTGGCTGGTT | |
| | GC (SEQ ID NO: 76) | |
| matRbcS1A (&mod) | AAACCCGGGCATGCAGGTGTGGCCT | pGADT7_1A_{At} |
| Fw AD | CCG (SEQ ID NO: 77) | pGADT7_1AAtMOD |
| matRbcS1A (&mod) | AAAGGATCCTTAACCGGTGAAGCTT | pGADT7_1AAtMOD(β-sheets) |
| Rev AD | GGTGGC (SEQ ID NO: 78) | pGADT7_1AAtMOD(loop) |
| | | pGADT7_1AAtMOD(β- |
| | | sheets+loop) |
| | | pGADT7_1AAtMOD(α- |
| | | helices+β-sheets) |
| | | pGADT7_1AAtMOD(α- |
| | | he1ices+β-sheets+loop) |
| RbcL BD&AD Fw | ATATGAATTCATGGTTCCACAAACA | pGADT7_LSUCr |
| | GAAACTAAAGCA (SEQ ID NO: 79) | pGBKT7_LSUCr |
| RbcL BD&AD Rev | CCCGGATCCTTAAAGTTTGTCAATA | |
| | GTATCAAATTCGA (SEQ ID NO: 80) | |
| Ctr1EPYC.1/LCI5 Rev | TTTGGATCCTCTGTTCGTTGCACTAC | pGBKT7_N-ter EPYC1 |
| BD | TAGCTCTT (SEQ ID NO: 81) | |
| Ctr2EPYC.1/LCI5 Rev | TTTGGATCCGGCCTTCTTTGAAGCTG | pGBKT7_N-ter+1rep EPYC1 |
| BD | AGCTACTT (SEQ ID NO: 82) | |
| Ctr3EPYC.1/LCI5 Rev | AATGGATCCGGCCTTCTTGCTGGAA | pGBKT7_N-ter+2reps EPYC1 |
| BD | GAACTCCTA (SEQ ID NO: 83) | |
| Ctr4EPYC.1/LCI5 Rev | TTTGGATCCTGCTTTTTTGCTCGCCG | pGBKT7_N-ter+3reps EPYC1 |
| BD | ATGAGCTACG (SEQ ID NO: 84) | |
| Ctr5EPYC.1/LCI5 Rev | ATAGGATCCGGCTTTGTCAGCGGAG | pGBKT7_N-ter+4reps EPYC1 |
| BD | GAACTAGATGAC (SEQ ID NO: 85) | |
| Ntr5EPYC.1/LCI5 Fw | TTTTGAATTCGTGAGCCCAACAAGA | pGBKT7_4reps+C-ter EPYC1 |
| | AGCGTTCTC (SEQ ID NO: 86) | |
| Ntr4EPYC.1/LCI5 Fw | TTTTGAATTCGTTACTCCTTCAAGAA | pGBKT7_3reps+C-ter EPYC1 |
| | GTGCCTTGC (SEQ ID NO: 87) | |
| Ntr3EPYC.1/LCI5 Fw | TTTTGAATTCGTCACTCCGTCTCGTT | pGBKT7_2reps+C-ter EPYC1 |
| | CAGCTC (SEQ ID NO: 88) | |
| Ntr2EPYC.1/LCI5 Fw | TTTTGAATTCGTCACCCCTAGTAGAT | pGBKT7_1rep1+C-ter EPYC1 |
| | CGGCC (SEQ ID NO: 89) | |
| Ntr1EPYC.1/LCI5 Fw | AAAAGAATTCGGAACTAATCCTTGG | pGBKT7_C-ter EPYC1 |
| | ACAGGTAAAAGC (SEQ ID NO: 90) | |
| EPYC rep1 A for | ACGTACCGGTCTCCACATCCCGGGG | All pGBKT7_synthEPYC |
| | GTGAGCCCAACAAGAAGCG (SEQ ID | vectors |
| | NO: 91) | |
| EPYC rep1 T rev | ACGTACCGGTCTCCACAAGGATCCG | |
| | GCCTTCTTTGAAGCTGAG (SEQ ID | |
| | NO: 92) | |
| EPYC rep1 B for | ACGTACCGGTCTCCTGTAAGCCCAA | pGBKT7_synthEPYC1 2reps |
| | CAAGAAGCGTTC (SEQ ID NO: 93) | pGBKT7_synthEPYC1 4reps |
| EPYC rep1 B rev | ACGTACCGGTCTCCTACAGCCTTCTT | pGBKT7_synthEPYC1 8reps |
| | TGAAGCTGAG (SEQ ID NO: 94) | |
| EPYC rep1 C for | ACGTACCGGTCTCCGGTTAGCCCAA | pGBKT7_synthEPYC1 4reps |
| | CAAGAAGCGTTC (SEQ ID NO: 95) | pGBKT7_synthEPYC1 2α- |
| EPYC rep1 C rev | ACGTACCGGTCTCCAACCGCCTTCTT | helices 4reps |
| | TGAAGCTGAG (SEQ ID NO: 96) | |
| EPYC rep1 D for | ACGTACCGGTCTCCCGTCAGCCCAA | |
| | CAAGAAGCGTTC (SEQ ID NO: 97) | |
| EPYC rep1 D rev | ACGTACCGGTCTCCGACGGCCTTCT | |
| | TTGAAGCTGAG (SEQ ID NO: 98) | |
| EPYC rep1 A2 for | ACGTACCGGTCTCCACATCCCGGGG | pGBKT7_synthEPYC1 8reps |
| | GTGAG (SEQ ID NO: 99) | |
| EPYC rep1 T2 rev | GCCACTTGGTCTCGACAAGGATCCG | |
| | GCCTTC (SEQ ID NO: 100) | |
| EPYC rep1 E for | CTCTGTGAAGACAGGTCTCGAGTGA | |
| | GCCCAAC (SEQ ID NO: 101) | |
| EPYC rep1 E rev | CTTCGTGAAGGGTCTCACACTGCCT | |
| | TCTTTG (SEQ ID NO: 102) | |
| synthEPYC J for | TTGAATCACTCAGAAATAATTGGAG | pGBKT7_synthEPYC1 2α- |
| | GCAAGAACTTG (SEQ ID NO: 103) | helices lrep |
| synthEPYC J rev | CAAGTTCTTGCCTCCAATTATTTCTG | |
| | AGTGATTCAA (SEQ ID NO: 104) | |
| EPYC rep1 H for | ACGTACCGGTCTCATCAGAACGGCA | pGBKT7_synthEPYC1 |
| | GCTCGTCG (SEQ ID NO: 105) | modified α-helix lrep |
| EPYC rep1 H rev | ACGTACCGGTCTCTCTGATTTCTGAG | |
| | TGATTCAAGTTC (SEQ ID NO: 106) | |
| EPYC rep1 G for | ACGTACCGGTCTCCGTAGAAATGGT | pGBKT7_synthEPYC1 α- |
| | AACGGCAGC (SEQ ID NO: 107) | helix knockout 1 |
| EPYC rep1 G rev | ACGTACCGGTCTCCCTACGTGATTC | |
| | AAGTTCTTG (SEQ ID NO: 108) | |
| synthEPYC I for | ACGTACCGGTCTCATGGCTTGAATC | pGBKT7_synthEPYC1 α- |
| | ACTCAGAAATG (SEQ ID NO: 109 | helix knockout 2 |
| synthEPYC I rev | ACGTACCGGTCTCAGCCATTGCCTC | |
| | CAATTAGCTG (SEQ ID NO: 110) | |
| matLCIB Fw AD | ATACATATGCAAGCAGCATCAACAG | pGADT7_LCIB |
| | CGGTTGC (SEQ ID NO: 111) | |
| matLCIB Rev AD | ATACCCGGGGTTTTTTGGTGCTTCAA | |
| | ATGACGGGTG (SEQ ID NO: 112) | |
| matLCIC Fw AD | TATCCCGGGTAGTCAAGCTCTCACT | pGADT7_LCIC |
| | GTTAGCCAA (SEQ ID NO: 113) | |
| matLCIC Rev AD | TATGGATCCGTTCATATTAGCTAGCT | |
| | CGGGAGA (SEQ ID NO: 114) | |
| CAH3 BD&AD Fw | ATTTGAATTCCGAAGCGCAGTTCTT | pGADT7_CAH3 |
| | CAGAGAG (SEQ ID NO: 115) | |
| CAH3 BD&AD Rev | TTAGGATCCTCAGAGCTCATACTCC | |
| | ACAAGTCTA (SEQ ID NO: 116) | |
| CP12 Fw AD | TTTTGAATTCGGTCCGGTCCATTTGA | pGADT7_CP12 |
| | ACAATTCG (SEQ ID NO: 117) | |
| CP12 Rrev AD | TTTCCCGGGGCACTCGTTGGTCTCA | |
| | GGATTGTC (SEQ ID NO: 118) | |

Competent yeast cells (Y2H Gold, Clontech) were prepared from a 50 ml culture grown in YPDA medium supplemented with kanamycin (50 μg ml⁻¹). Cells were washed with ddH2O and a lithium acetate/TE solution (100 mM LiAc, 10 mM Tris-HCl [pH 7.5], 1 mM EDTA) before re-suspending in lithium acetate/TE solution. Cells were then co-transformed with binding and activation domain vectors by mixing 50 μl of competent cells with 1 μg of each plasmid vector and a PEG solution (100 mM LiAc, 10 mM Tris-HCl [pH 7.5], 1 mM EDTA, 40% [v/v] PEG 4000). Cells were incubated at 30° C. for 30 min, then subjected to a heat shock of 42° C. for 20 min. The cells were centrifuged, re-suspended in 500 μl YPDA and incubated at 30° C. for ca 90 min, then centrifuged and washed in TE (10 mM Tris-HCl [pH 7.5], 1 mM EDTA). The pellet was re-suspended in 200 μl TE, spread onto SD-L-W (standard dextrose medium (minimal yeast medium) lacking leucine and tryptophan, Anachem) and grown for 3 days at 30° C. Ten to fifteen of the resulting colonies were pooled per co-transformation and grown in a single culture for 24 hrs. The following day 1 ml of culture was harvested, cell density (OD₆₀₀) measured, centrifuged and then diluted in TE to give a final OD₆₀₀ of 0.5 or 0.1.

Yeast cultures were then plated onto SD-L-W (yeast synthetic minimal media lacking leucine (L) and tryptophan (W)) and SD-L-W-H (yeast synthetic minimal media lacking L, W, and histidine(H)) (Anachem). Yeast expressing both binding and activation domain constructs was grown on SD-L-W to confirm presence of both plasmids. To assess interaction strength, yeast was plated onto SD-L-W-H with differing concentrations of the HIS3 inhibitor 3-aminotriazole (3-AT). These plates were then incubated for 3 days before assessing for presence or absence of growth, to perform a semi-quantitative yeast two-hybrid assay as in van Nues and Beggs (van Nues and Beggs, Genetics (2000) 157: 1451-1467). The same yeast transformation was used for each interaction study. Different colonies on the same yeast transformation plate were considered independent biological replicates (as for E. coli). Two biological replicates (top and bottom row for each interaction) were spotted from different liquid culture concentrations (0.5 and 0.1 OD). Each interaction experiment was performed at least twice. Summary figures of the yeast interaction studies are shown in FIGS. 3C, 4J-4K, and 5E.

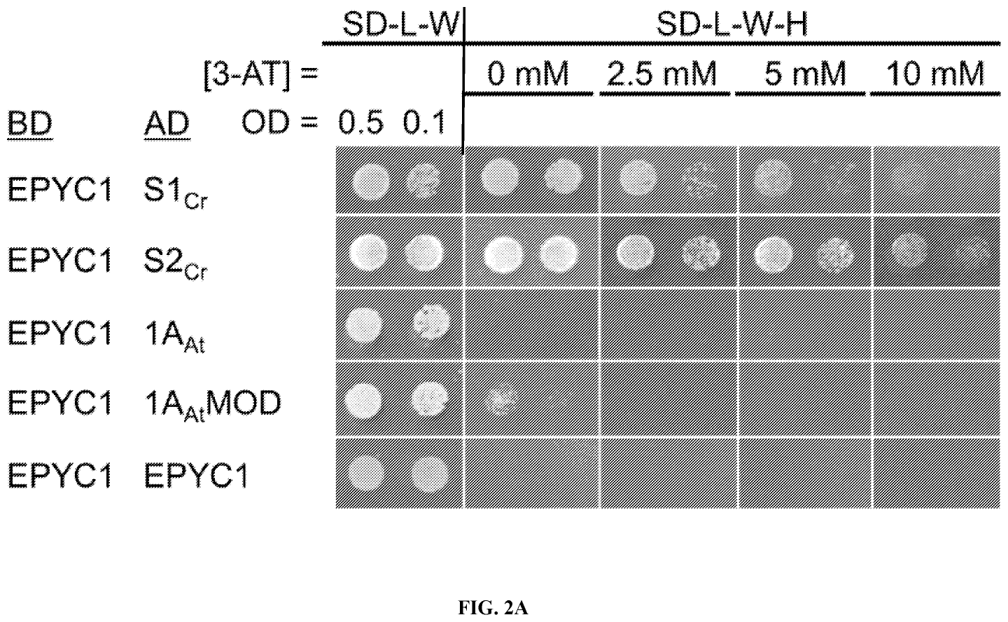

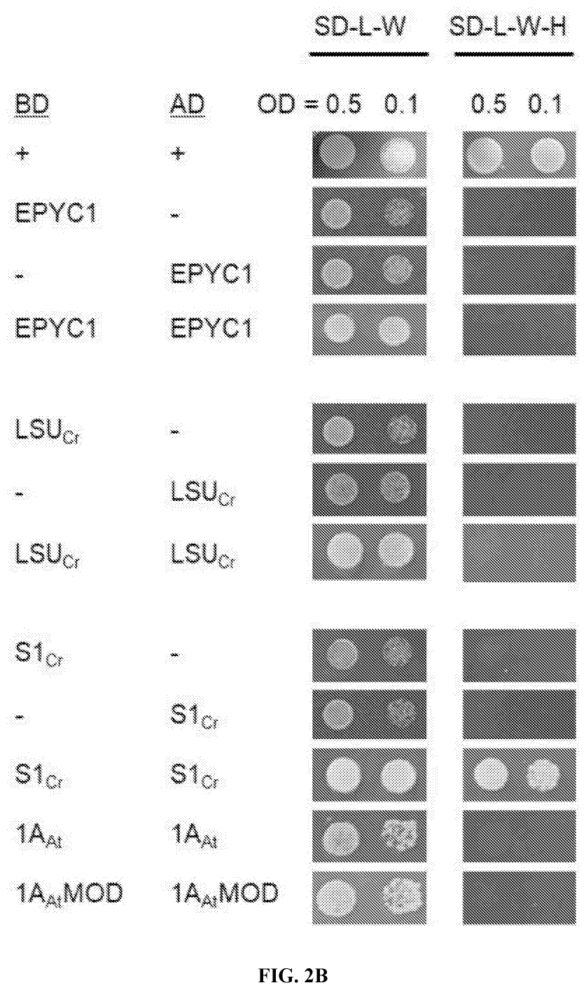

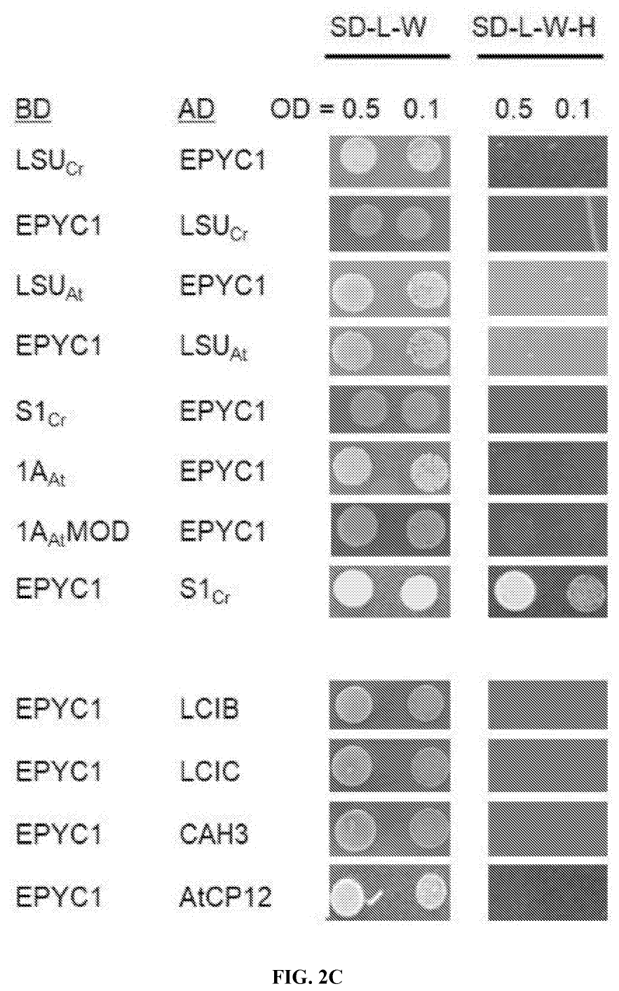

Table 2 provides descriptions of the vectors that were used in the yeast two-hybrid assays. FIGS. 2A-2B show exemplary results from assays using the first seven vectors listed in Table 2 (pGBKT7_EPYC1 to pGADT7_LSU_{Cr}); each interaction experiment had two biological replicates and was performed at least twice. FIGS. 3C, 4J-4K, and 5E show summary figures of results from assays using the middle thirty-one vectors (pGADT7 1A_{At}MOD(β-sheets) to pGBKT7_synthEPYC1 α-helix knockout 2). FIGS. 2B-2C show exemplary results from assays using the last ten vectors (pGBKT7_LSU_{Cr} to pGADT7_LSU_{At}); each interaction experiment had two biological replicates and was performed at least twice.

TABLE 2
Vectors used for yeast two-hybrid assays.

| Vector | Description |
|---|---|
| pGBKT7_EPYC1 | Full-length codon-optimized EPYC1 in yeast two-hybrid (Y2H) |
| | binding domain vector |
| pGADT7_EPYC1 | Full-length codon-optimized EPYC1 in Y2H Activation domain |
| | vector |
| pGADT7_S1_{Cr} | C. reinhardtii Rubisco small subunit (SSU) RbcS1 in Y2H |
| | activation domain vector |
| pGADT7_S2_{Cr} | C. reinhardtii SSU RbcS2 in Y2H activation domain vector |
| pGADT7_1A_{At} | A. thaliana SSU RbcS1A in Y2H activation domain vector |
| pGADT7_1A_{At}MOD(α-helices) | A. thaliana SSU RbcS1A with modified alpha-helices in Y2H |
| | activation domain vector |
| pGADT7_LSU_{Cr} | C. reinhardtii Rubisco large subunit in Y2H activation domain |
| | vector |
| pGADT7_1A_{At}MOD(β-sheets) | A. thaliana SSU RbcS1A with modified β-sheets in Y2H activation |
| | domain vector |
| pGADT7_1A_{At}MOD(loop) | A. thaliana SSU RbcS1A with modified loop in Y2H activation |
| | domain vector |
| pGADT7_1A_{At}MOD(β- | A. thaliana SSU RbcS1A with modified β-sheets and loop in Y2H |
| sheets + loop) | activation domain vector |
| pGADT7_1A_{At}MOD(α- | A. thaliana SSU RbcS1A with modified α-helices and β-sheets in |
| helices + β-sheets) | Y2H activation domain vector |
| pGADT7_1A_{At}MOD(α- | A. thaliana SSU RbcS1A with modified α-helices, β-sheets and |
| helices + β-sheets + loop) | loop in Y2H activation domain vector |
| pGBKT7_N-ter EPYC1 | N-terminus of EPYC1 in Y2H binding domain vector |
| pGBKT7_N-ter + 1rep EPYC1 | N-terminus and first repeat of EPYC1 in Y2H binding domain |
| | vector |
| pGBKT7_N-ter + 2reps EPYC1 | N-terminus and first two repeats of EPYC1 in Y2H binding domain |
| | vector |
| pGBKT7_N-ter + 3reps EPYC1 | N-terminus and first three repeats of EPYC1 in Y2H binding |
| | domain vector |
| pGBKT7_N-ter + 4reps EPYC1 | N-terminus and all four repeats of EPYC1 in Y2H binding domain |
| | vector |
| pGBKT7_4reps + C-ter EPYC1 | All four repeats plus C-terminus of EPYC1 in Y2H binding domain |
| | vector |
| pGBKT7_3reps + C-ter EPYC1 | First three repeats plus C-terminus of EPYC1 in Y2H binding |
| | domain vector |
| pGBKT7_2reps + C-ter EPYC1 | First two repeats plus C-terminus of EPYC1 in Y2H binding |
| | domain vector |
| pGBKT7_1rep1 + C-ter EPYC1 | First repeat plus C-terminus of EPYC1 in Y2H binding domain |
| | vector |
| pGBKT7_C-ter EPYC1 | C-terminus of EPYC1 in Y2H binding domain vector |
| pGBKT7_mEPYC1 | Mature EPYC (minus C-terminus) in Y2H binding domain vector |
| pGBKT7_mEPYC1-α1 | Mature EPYC with 1 α-helix mutation in Y2H binding domain |
| | vector |
| pGBKT7_mEPYC1-α1,2 | Mature EPYC with 1,2 α-helix mutations in Y2H binding domain |
| | vector |
| pGBKT7_mEPYC1-α1,2,3 | Mature EPYC with 1,2,3 α-helix mutations in Y2H binding domain |
| | vector |
| pGBKT7_mEPYC1-α1,2,3,4 | Mature EPYC with 1,2,3,4 α-helix mutations in Y2H binding |
| | domain vector |
| pGBKT7_mEPYC1-α3,4 | Mature EPYC with 3,4 α-helix mutations in Y2H binding domain |
| | vector |
| pGBKT7_mEPYC1-α4 | Mature EPYC with 4 α-helix mutation in Y2H binding domain |
| | vector |
| pGBKT7_synthEPYC1 1rep | Repeat 1 of EPYC1 in Y2H binding domain vector |
| pGBKT7_synthEPYC1 2reps | Two times repeat 1 of EPYC1 in Y2H binding domain vector |
| pGBKT7_synthEPYC1 4reps | Four times repeat 1 of EPYC1 in Y2H binding domain vector |
| pGBKT7_synthEPYC1 8reps | Eight times repeat 1 of EPYC1 in Y2H binding domain vector |
| pGBKT7_synthEPYC1 2α- | Four times repeat 1 of EPYC1 with double alpha helix in Y2H |
| helices 4reps | binding domain vector |
| pGBKT7_synthEPYC1 2α- | Repeat 1 of EPYC1 with double α-helix in Y2H binding domain |
| helices 1rep | vector |
| pGBKT7_synthEPYC1 | Repeat 1 of EPYC1 with modified α-helix in Y2H binding domain |
| modified α-helix 1rep | vector |
| pGBKT7_synthEPYC1 α-helix | Repeat 1 of EPYC1 with α-helix knockout version 1 in Y2H |
| knockout 1 | binding domain vector |
| pGBKT7_synthEPYC1 α-helix | Repeat 1 of EPYC1 with α-helix knockout version 2 in Y2H |
| knockout 2 | binding domain vector |
| pGBKT7_LSU_{Cr} | C. reinhardtii Rubisco large subunit in Y2H binding domain vector |
| pGBKT7_S1_{Cr} | C. reinhardtii SSU RbcS1 in Y2H binding domain vector |
| pGADT7_EPYC1 | Full-length EPYC1 in Y2H activation domain vector |
| pGADT7_LCIB | C. reinhardtii LCIB in Y2H activation domain vector |
| pGADT7_LCIC | C. reinhardtii LCIC in Y2H activation domain vector |
| pGADT7_CAH3 | C. reinhardtii CAH3 in Y2H activation domain vector |
| pGADT7_CP12 | A. thaliana CP12 in Y2H activation domain vector |
| pGBKT7_1A_{At}MOD(α-helices) | A. thaliana SSU RbcS1A with modified alpha-helices in Y2H |
| | binding domain vector |
| pGBKT7_LSU_{At} | A. thaliana Rubisco large subunit in Y2H binding domain vector |
| pGADT7_LSU_{At} | A. thaliana Rubisco large subunit in Y2H Activation domain vector |

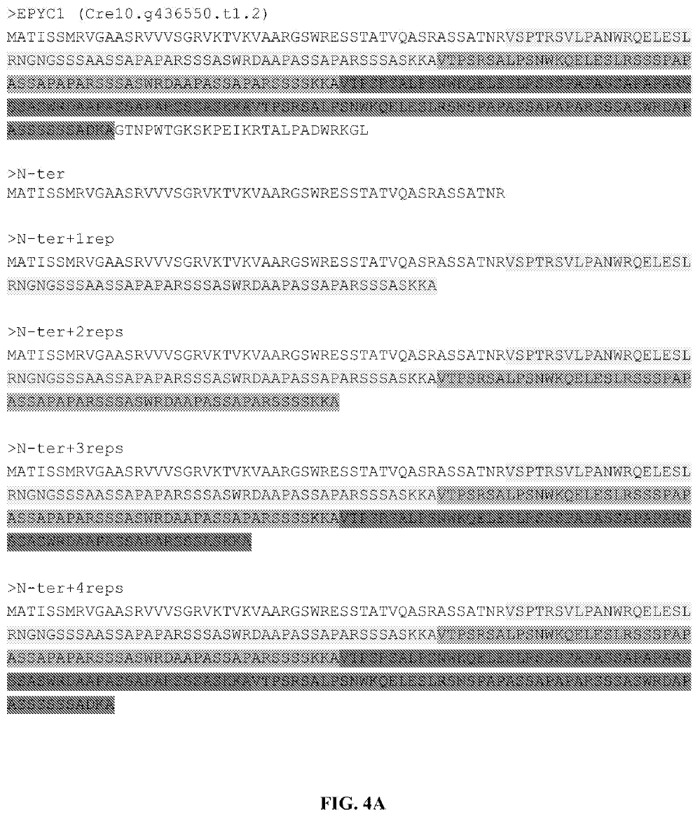

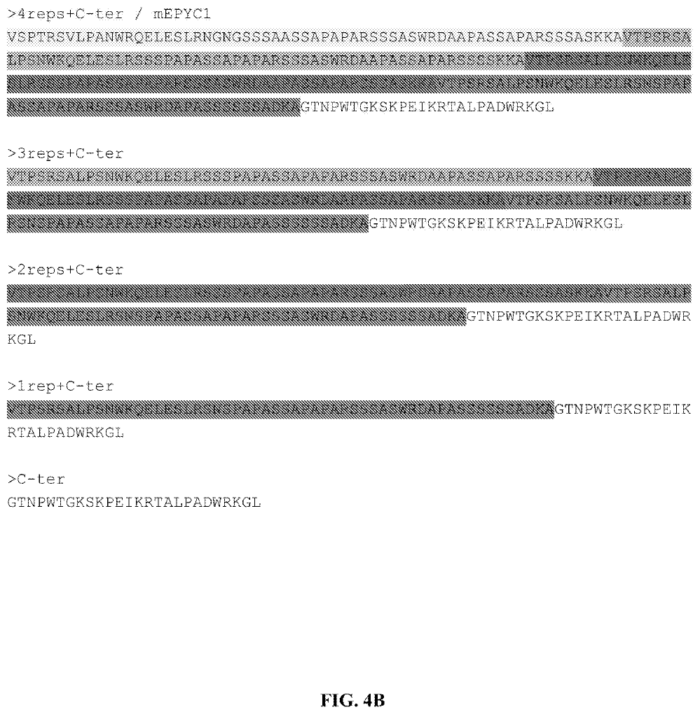

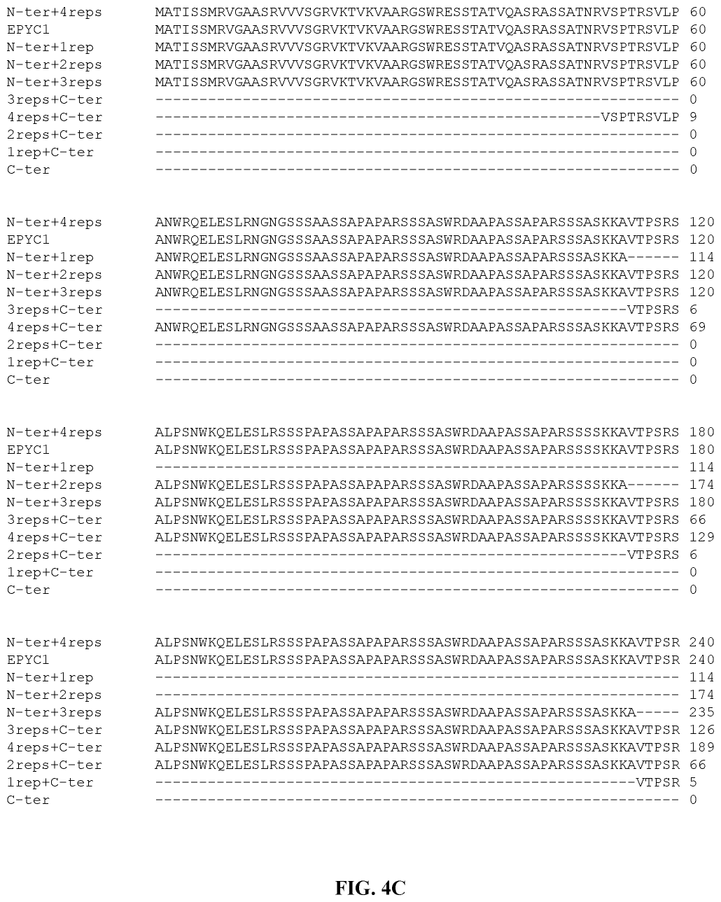

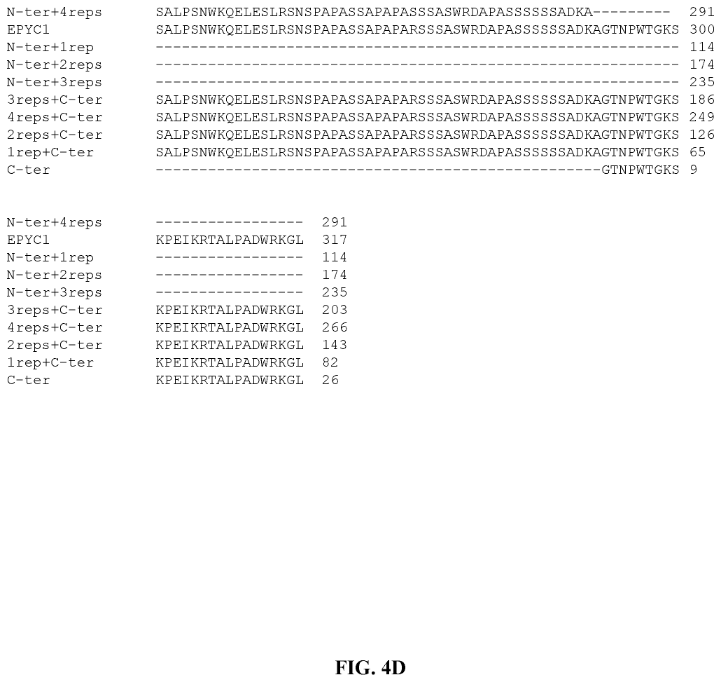

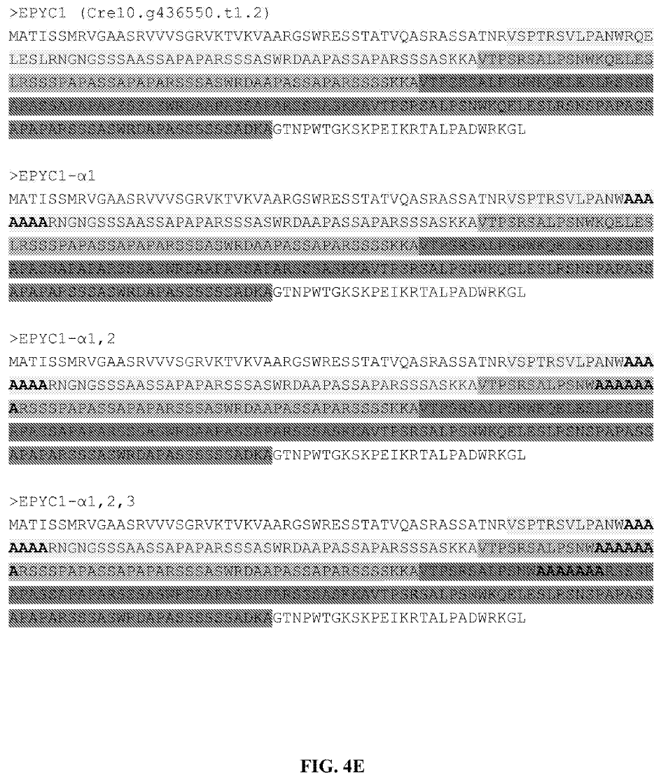

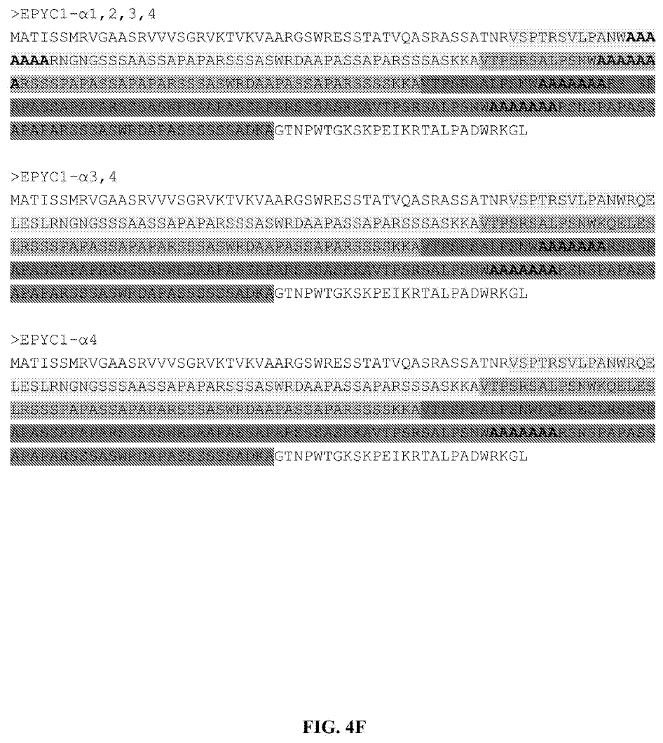

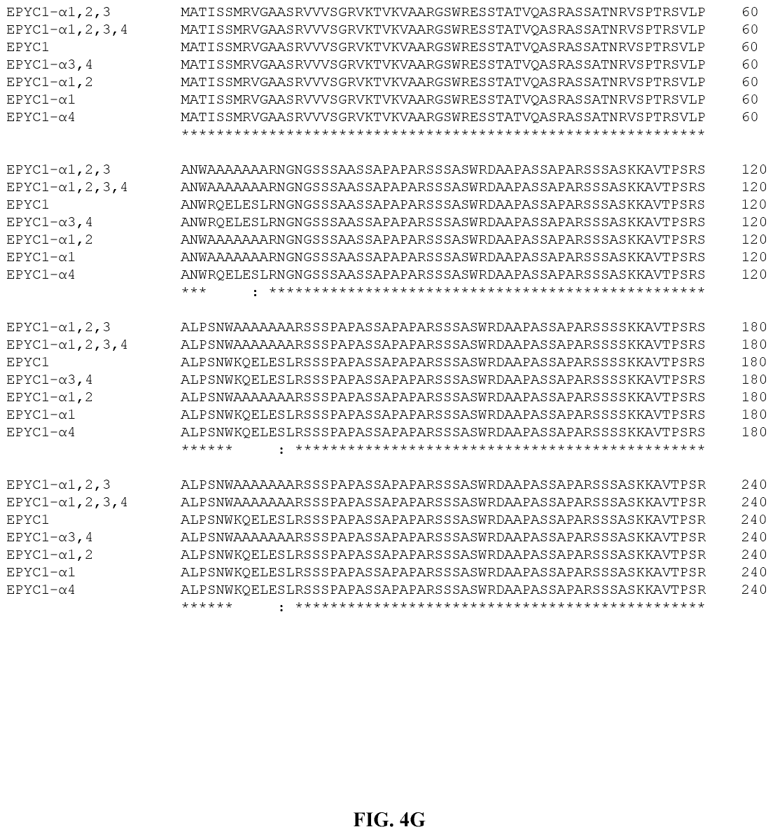

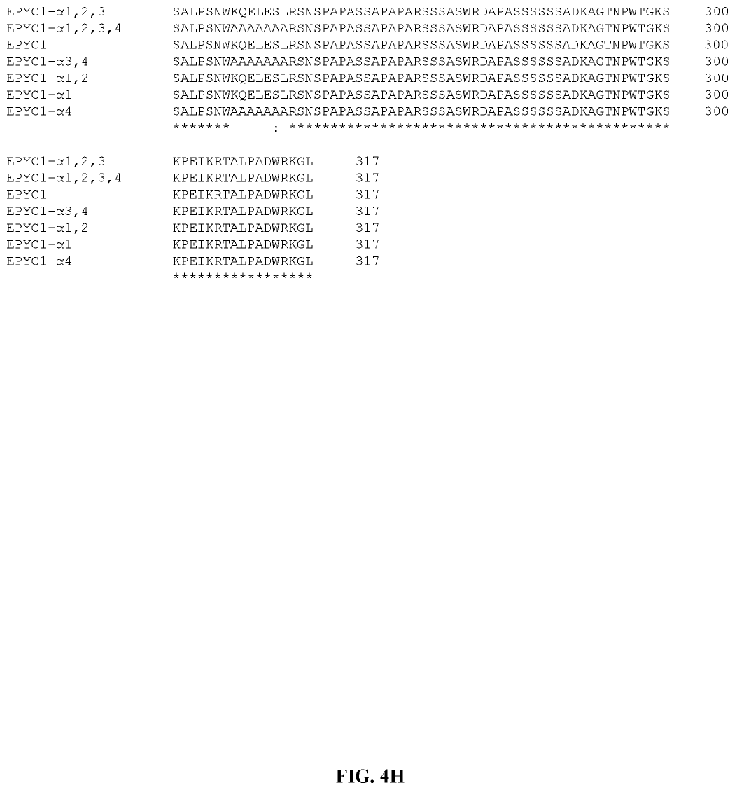

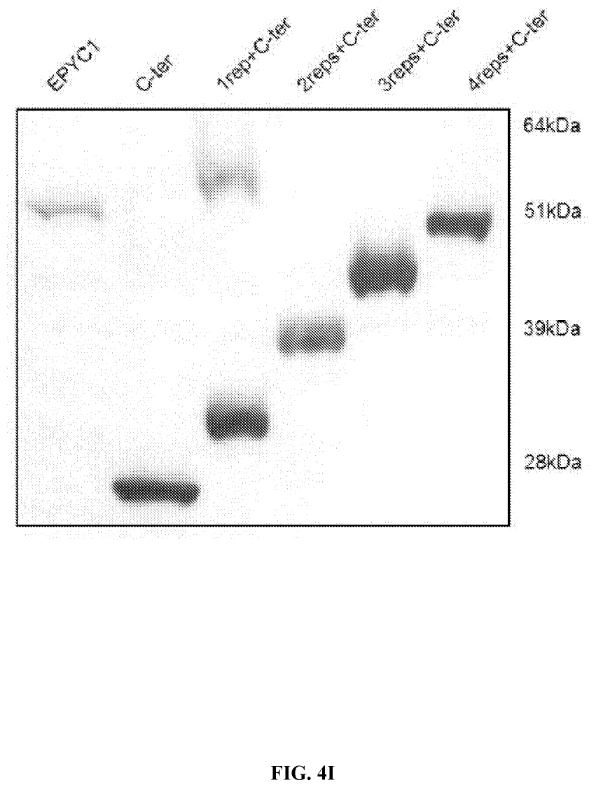

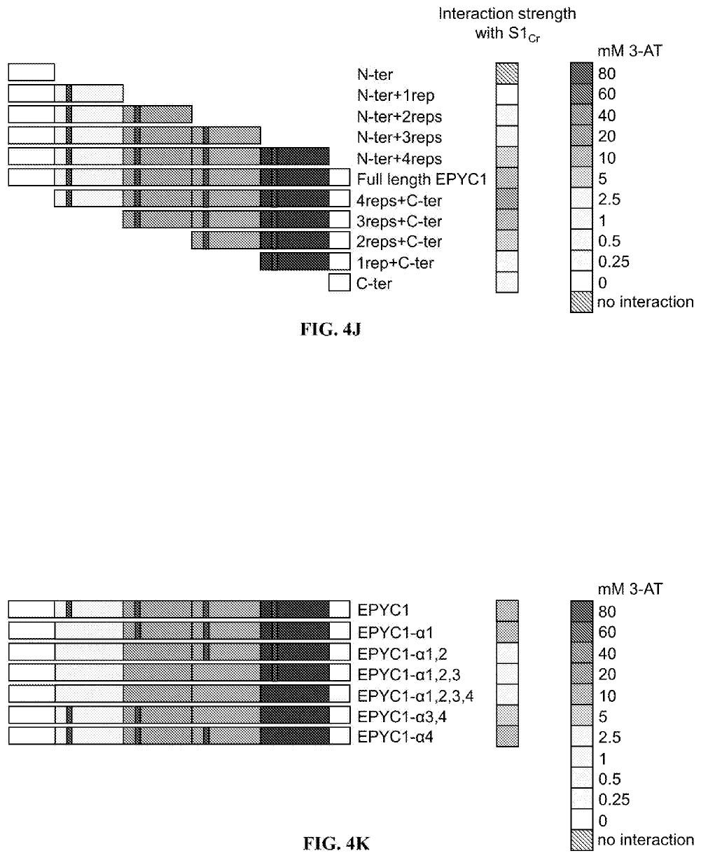

Protein extraction was carried out by re-suspending yeast cells to an OD₆₀₀ of 1 from an overnight liquid culture in a lysis buffer (50 mM Tris HCl [pH 233 6], 4% [v/v] SDS, 8 M urea, 30% [v/v] glycerol, 0.1 M DTT, 0.005% [w/v] Bromophenol blue), incubating 65° C. for 30 min, and loading directly onto a 10% (w/v) Bis-Tris protein gel (Expedeon). In the immunoblot shown in FIG. 4I, protein was extracted from yeast expressing N-terminus truncated versions of EPYC1::GAL4 binding domain and immunoblotted with anti-EPYC1.

### Liquid Chromatography-Mass Spectrometry (LC-MS)

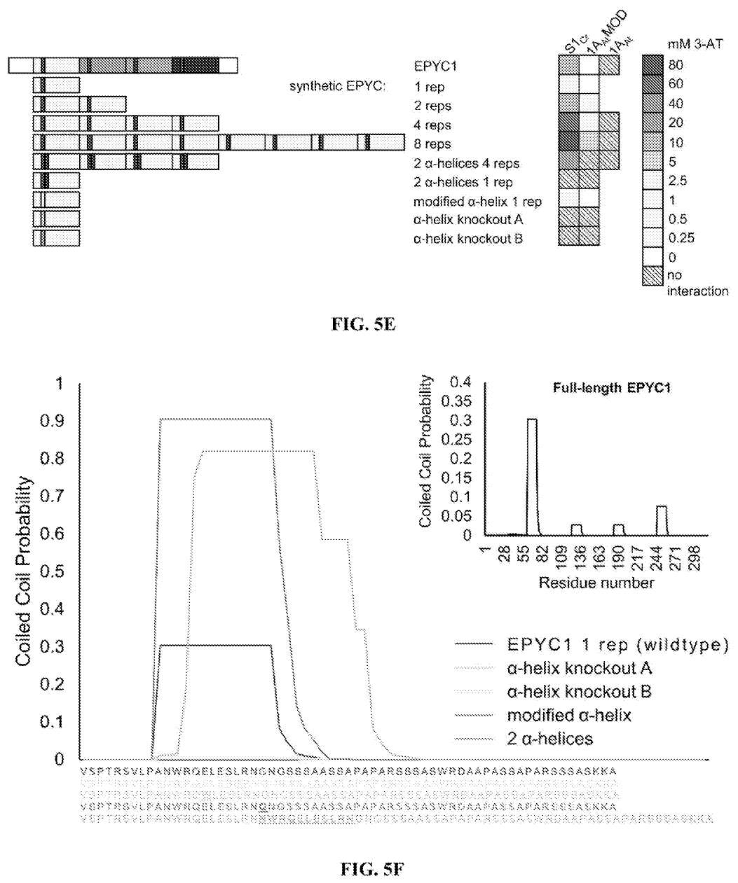

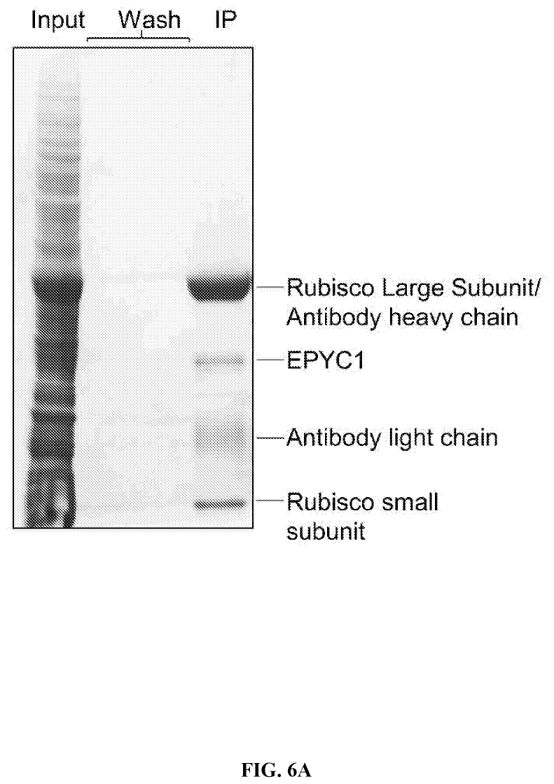

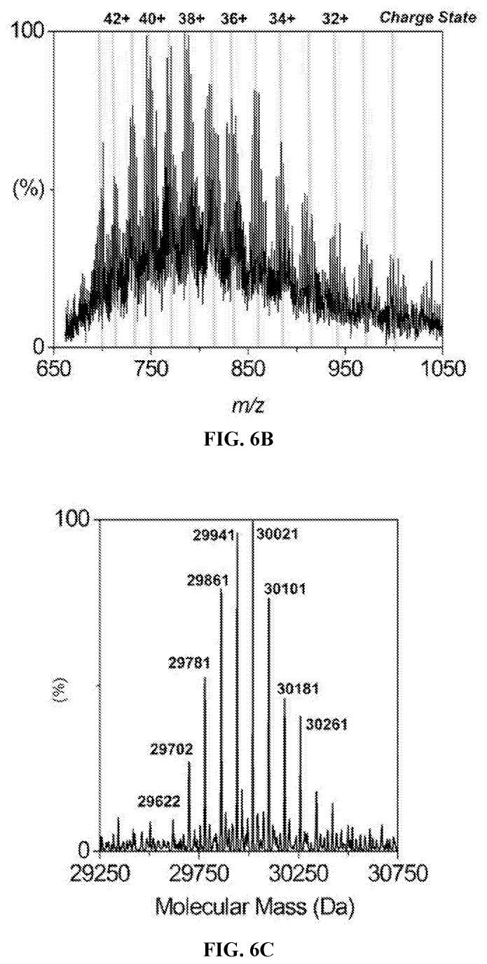

Cell lysate was prepared from C. reinhardtii cells according to Mackinder et al. (Mackinder, et al., PNAS (2016) 113: 5958-5963). Following membrane solubilization with 2% (w/v) digitonin, the clarified lysate was applied to 150 μl Protein A Dynabeads that had been incubated with 20 μg anti-EPYC1 antibody. The Dynabead-cell lysate was incubated for 1.5 hours with rotation at 4° C. The beads were then washed four times with IP buffer (50 mM HEPES, 50 mM KOAc, 2 mM Mg(OAc)₂.4H₂O, 1 mM CaCl₂), 200 mM sorbitol, 1 mM NaF, 0.3 mM NA₃VO₄, Roche cOmplete EDTA-free protease inhibitor) containing 0.1% (w/v) digitonin. EPYC1 was eluted from the beads by incubating for 10 minutes in elution buffer (50 mM Tris-HCl, 0.2 M glycine [pH 2.6]), and the eluate was immediately neutralized with 1:10 (v/v) Tris-HCl (pH 8.5). A small amount of the eluate was run on an SDS-PAGE gel and stained with coomassie (FIG. 6A), and the remaining sample was used for LC-MS.

Intact protein LC-MS experiments were performed on a Synapt G2 Q-ToF instrument equipped with electrospray ionization (i.e., electrospray ionization mass spectrometry (ESI-MS); Waters Corp., Manchester, UK). LC separation was achieved using an Acquity UPLC equipped with a reverse phase C4 Aeris Widepore 50×2.1 mm HPLC column (Phenomenex, Calif., USA) and a gradient of 5-95% acetonitrile (0.1% formic acid) over 10 minutes was employed. Data analysis was performed using MassLynx v4.1 and deconvolution was performed using MaxEnt.

### PCOILS Analysis of EPYC1

PCOILS is an online tool (https://toolkit.tuebingen.mpg.de/#/tools/pcoils) that predicts the probability (from 0-1) of the presence of coiled-coil domains in a submitted protein sequence. The direct output following submission is shown in FIG. 5F.

### Results

EPYC1 Interacts with C. reinhardtii SSUs and Modified A. thaliana SSUs in Y2H Assays

The two α-helices of the C. reinhardtii SSU (FIGS. 1C-1D) were previously proposed to be potential binding sites for EPYC1 (FIGS. 1A-1B) (Meyer, et al., PNAS (2012) 109: 19474-19479; Mackinder, et al., PNAS (2016) 113: 5958-5963). This hypothesis was tested using a semi-quantitative Y2H approach. In Y2H assays, EPYC1 showed a relatively strong protein-protein interaction (i.e., growth at 10 mM 3-AT) with both C. reinhardtii SSU homologs, S1_{Cr} and S2_{Cr} (FIG. 2A). In contrast, EPYC1 did not interact with the 1A SSU from A. thaliana (1A_{At}) but did interact weakly with a hybrid 1A SSU carrying the α-helices from C. reinhardtii (1A_{At}MOD; described in Atkinson, et al., New Phyt. (2017) 214: 655-667).

The Y2H assays further showed that EPYC1 did not interact with itself (FIGS. 2A-2B). As shown in FIGS. 2B-2C, EPYC1 also did not interact with other C. reinhardtii CCM components associated with the pyrenoid (i.e., LCIB, LCIC, and CAH3), or with another intrinsically disordered protein found in the chloroplast stroma (AtCP12, described in Lopez-Calcagno, et al., Front. Plant Sci. (2014) 5:9). These results indicated that EPYC1 was not prone to false positive protein-protein interactions in Y2H assays.

### Higher Plant Rubisco SSUs can be Engineered for Increased Affinity to EPYC1

Next, key domains on the C. reinhardtii SSU required for interaction with EPYC1 were identified. To isolate the structural components of the SSU, a total of six different chimeric versions of 1A_{At} bearing residues from S1_{Cr} associated with the three distinct β-sheets (βA, βC and βD), the βA-βB loop, and the two α-helices (αA and αB) (Spreitzer, Arch. Biochem. Biophys, (2003) 414: 141-149) were generated (FIG. 3B).

When tested in Y2H assays, as before, EPYC1 did not interact with 1A_{At} (FIG. 3C). The chimeric 1A_{At} with the β-sheets or the βA-βB loop from S1_{Cr}, or both together, also did not permit interaction. Interactions were only observed between EPYC1 and chimeric 1A_{At} with the two α-helices from the C. reinhardtii SSU (FIG. 3C). The S1_{Cr} 1A_{At} with the S1_{Cr} α-helices alone produced a minimal interaction (i.e., on 0 mM 3-AT), which was strengthened by the incorporation of the β-sheets and the βA-βB loop from S1_{Cr}. Notably, the modified 1A_{At} variant with the α-helices, β-sheets, and βA-βB loop from C. reinhardtii (i.e., with a 79% sequence identity to S1_{Cr}) showed a stronger interaction compared to S1_{Cr} (FIG. 3C). These results indicated that higher plant Rubisco SSUs could be engineered for increased affinity for EPYC1 by including structural components of the C. reinhardtii SSU.

EPYC1 can be Engineered for Increased Interaction Strength with the Rubisco SSU

A variety of truncated EPYC1 variants were generated to characterize the key regions of EPYC1 required for interaction with the Rubisco SSU. Because EPYC1 is a modular protein consisting of four highly similar repeat sequences flanked by shorter terminal regions at the N- and C-terminus, truncations were made to eliminate each region sequentially from either the N- or the C-terminus direction (FIGS. 4A-4B; alignment of these sequences with native EPYC1 protein shown in FIGS. 4C-4D). Truncated EPYC1 variants expressed well in yeast (FIG. 4I). The results of Y2H assays using the truncated EPYC1 variants are shown in FIG. 4J. The EPYC1 N-terminus alone (N-ter) did not interact with S1_{Cr}, but addition of the first EPYC1 repeat region was sufficient to detect interaction. Addition of each subsequent repeat region correlated with growth at increased concentrations of 3-AT, confirming both that EPYC1 was a modular protein and that each repeat had an additive effect on interaction with SSU. Addition of the C-terminal tail further increased the strength of the interaction. Interestingly, the C-terminus alone also interacted with S1_{Cr}, suggesting that SSU binding sites were not limited to the repeat regions.

It was hypothesized that the interaction between EPYC1 and the SSU could be mediated through the predicted conserved α-helix in each of the four repeats, which together would allow EPYC1 to bind at least four Rubisco complexes (Mackinder, et al., PNAS (2016) 113: 5958-5963; Freeman Rosenzweig, et al., Cell (2017) 171: 148-162). The relative contribution of each of the four domains was analyzed by eliminating the predicted α-helical structure through mutation of the residues “RQELESL” (SEQ ID NO: 119) in the first repeat and “KQELESL” (SEQ ID NO: 120) in the subsequent three repeats into seven alanines (FIGS. 4E-4F; alignment of these sequences with native EPYC1 protein shown in FIGS. 4G-4H). As shown in FIG. 4K, mutation of a single helix did not have an impact on interaction strength when tested in Y2H assays. However, sequentially weaker interactions with S1_{Cr} were observed with increasing (i.e., additional) mutations of the α-helical regions. If all four α-helices were mutated, the interaction was not eradicated completely. The latter finding supported the evidence for an additional SSU binding site(s) on the C-terminus, as in the absence of all four α-helices the interaction strength was reduced to the same as the interaction strength of the C-terminus alone (FIG. 4J). Overall, the data suggested that EPYC1 had at least five SSU interaction sites, located in each of its four repeat regions and the C-terminus, respectively.

Analysis of EPYC1 with PCOILS suggested that the putative α-helices of EPYC1 might behave like coiled-coil domains, with the first repeat showing the highest predicted value (FIG. 5C) (Gruber, et al., J. Struct. (2006) 155: 140-145; Zimmermann, et al., J. Mol. Bio. (2017) 430: 2237-2243). Thus, it was hypothesized that the first repeat region could be a useful target scaffold to engineer a synthetic EPYC1 with increased affinity for SSU interaction. Four synthetic EPYC1 variants containing 1, 2, 4 or 8 copies of the first repeat in tandem were constructed (FIG. 5A; alignment shown in FIGS. 5B-5D). As shown in FIG. 5E, four copies of the first repeat (synthetic EPYC1 4 reps) showed a stronger interaction strength with S1_{Cr} and 1A_{At}MOD compared to native mature EPYC1 when tested in Y2H assays. The strongest interaction was observed for the variant with 8 repeats (synthetic EPYC1 8 reps), which grew on the maximum 3-AT concentrations tested (80 mM).

Using the single copy variant (synthetic EPYC1 1 rep), modifications of the α-helix region based on predictions from the PCOILS tool (FIG. 5A) were compared for interaction strength (FIG. 5E). Duplication of the α-helix region (SVLPA [symbol] [symbol] NWRQELESLRNGNGSS (SEQ ID NO: 121)) or a G-Q substitution near the α-helix (WRQELESLRNQ (SEQ ID NO: 122)) predicted an increased probability of coiled-coil behavior (FIG. 5F). In contrast to the predictions by PCOILS, the former modification eradicated the interaction, while the latter did not change the interaction strength compared to the native 1 rep variant. Finally, a L-R substitution within the α-helix (WRQELESRRNG (SEQ ID NO: 123)) or an E-W R substitution within the α-helix (WRQWLESLRNG (SEQ ID NO: 124)) were each made to attempt to knock out the interaction. Both substitutions eradicated the interaction. These results suggested that EPYC1 α-helices did not behave like traditional coiled-coil domains, but that even single point mutations within the α-helix could affect interaction. These results supported those presented in FIG. 4K.

### The N-Terminus of EPYC1 Contains a Cleavage Site

Removal of the N-terminus also increased the interaction strength, which was consistent with the predicted role of the N-terminus as a chloroplastic transit peptide that would be cleaved during import into the chloroplast (Mackinder, et al., PNAS (2016) 113: 5958-5963). Prediction tools ChloroP and PredAlgo suggested cleavage at residues 78 and 170, respectively (Emanuelsson, et al., Nat. Protoc. (2007) 2: 953-971). However, both predictions were unconvincing as they would result in cleavage within the repeat regions required for EPYC1 function. To identify the potential cleavage site, EPYC1 from C. reinhardtii was immunoprecipitated and analyzed using electrospray ionization mass spectrometry (ESI-MS). Intact protein ESI-MS analysis revealed several proteoforms of mature EPYC1 ranging from 29622-30621 Da (FIG. 6C). The molecular mass difference between proteoforms was 80 Da, suggesting variable phosphorylation states. This observation was consistent with previous reports highlighting the highly phosphorylated nature of EPYC1 (Turkina, et al., Proteomics (2006) 6: 2693-2704; Wang, et al., MCP (2014) 13: 2337-2353). The highly post-translationally modified state of EPYC1 made determination of the precise molecular mass of the mature protein difficult. However, the smallest proteoform identified had a molecular mass of 29.6 kDa which, based on the theoretical mass of EPYC1, indicated a cleavage site between residues 26 (V) and 27 (A) (FIG. 1B).

### Example 2: EPYC1 can be Targeted to Chloroplasts in Higher Plants and EPYC1 Interacts with Rubisco in Planta

The following example describes the engineering of an EPYC1 construct that was able to successfully target EPYC1 expression to higher plant chloroplasts (e.g., N. benthamiana and A. thaliana). When expressed in higher plant chloroplasts, EPYC1 was shown to interact with Rubisco in planta.

### Materials and Methods

### Plant Material and Growth Conditions

Arabidopsis (Arabidopsis thaliana, Col-0) seeds were sown on compost, stratified for 3 days at 4° C. and grown at 20° C., ambient CO₂, 70% relative humidity and 150 μmol photons m⁻²s⁻¹ in 12 hours (h) light, 12 h dark conditions. For comparisons of different genotypes, plants were grown from seeds of the same age and storage history, and harvested from plants grown in the same environmental conditions. N. benthamiana was grown at 20° C. with 150 μmol photons m⁻²s⁻¹ in 12 h light, 12 h dark conditions.

### Construct Design and Transformation

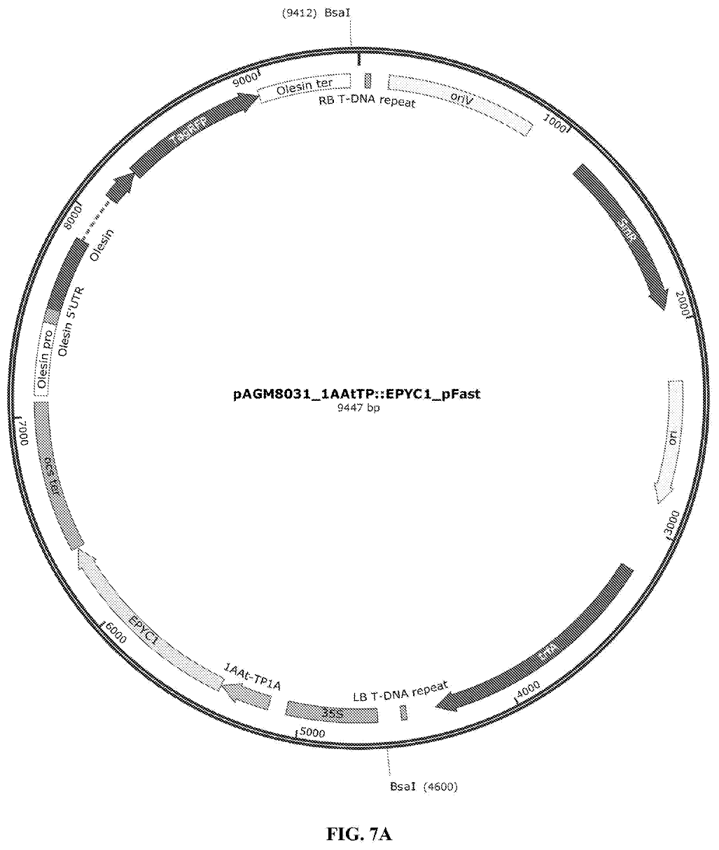

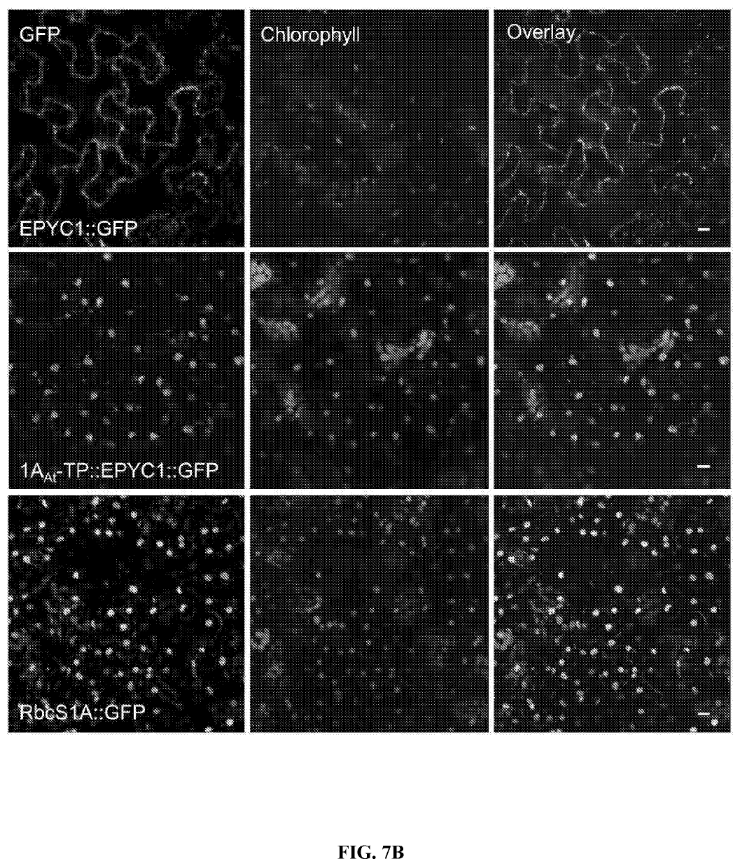

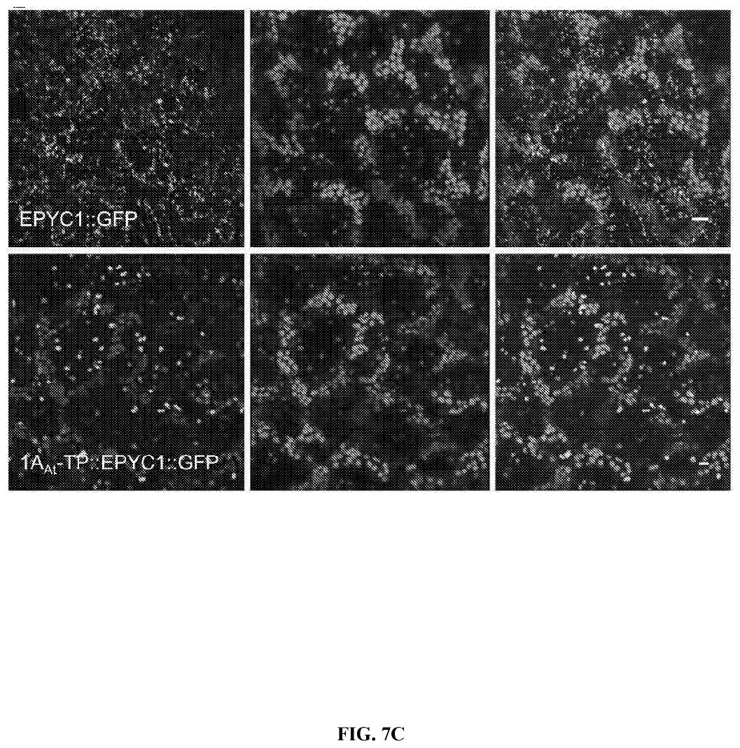

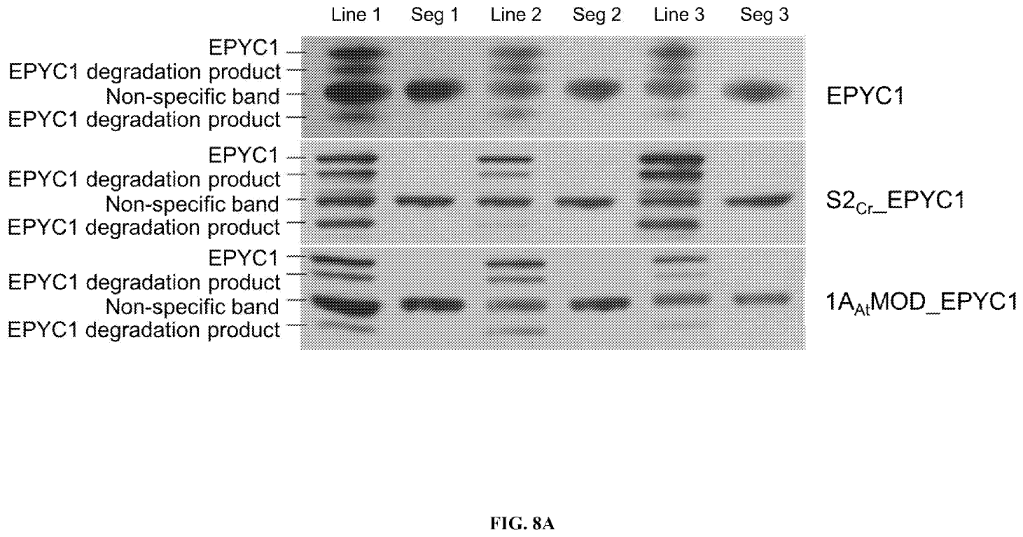

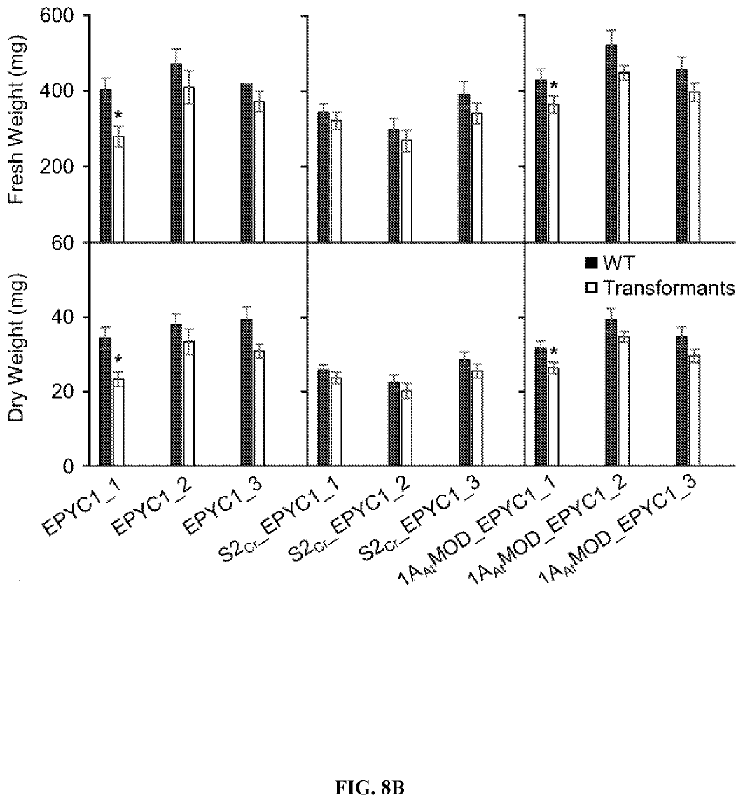

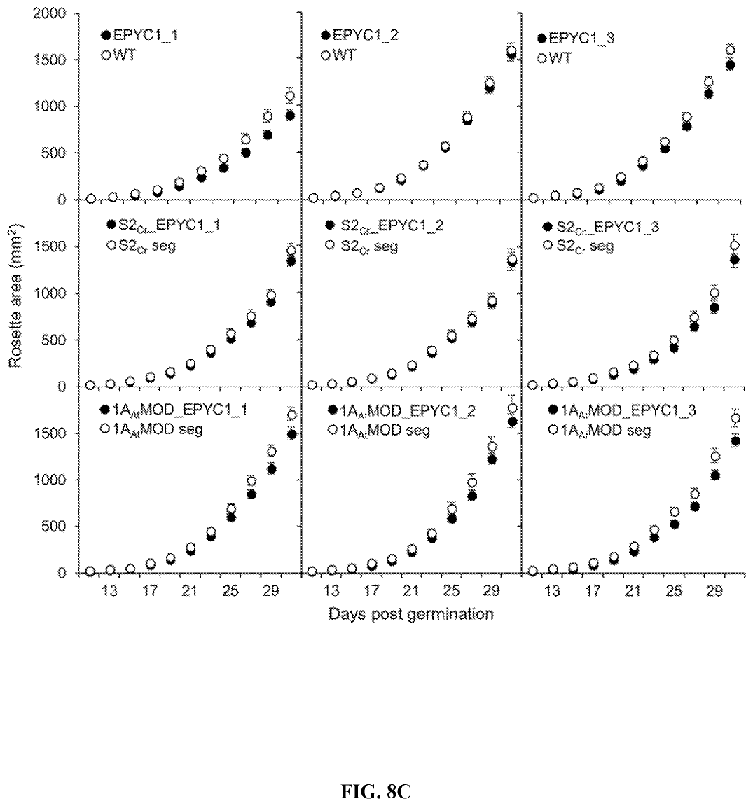

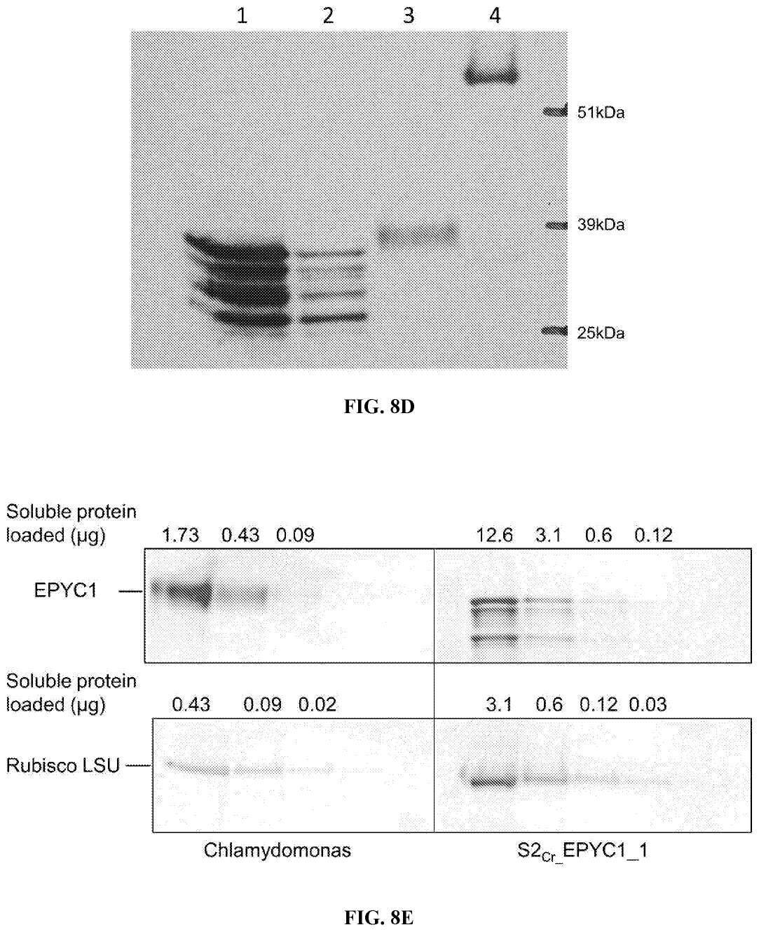

The coding sequence of EPYC1 was codon optimized for expression in higher plants using an online tool (www.idtdna.com/CodonOpt). All variants of EPYC1 were synthesized as Gblock fragments (IDT) and cloned directly into level 0 acceptor vectors pAGM1299 and pICH41264 of the Plant MoClo system (Engler, et al., ACS Synth. Bio. (2014) 3: 839-843) or pB7WG2,0 vectors containing C- or N-terminal YFP. Table 3 provides descriptions of the vectors that were used for plant transformation. FIGS. 7B-7C, 8A-8C, and 9A show exemplary results from assays using the first five vectors (pICH47742 EPYC1::GFP to pAGM8031_EPYC1::GFP_pFast). FIGS. 8D-8E show exemplary results from assays using the last eleven vectors (pB7_S2_{Cr}::YFP^{N} to pB7_S2_{Cr}::YFP^{N}).

TABLE 3
Vectors used for plant transformation.

| Vector | Description |
|---|---|
| pICH47742_EPYC1::GFP | Full-length codon-optimized EPYC1 with GFP in Golden |
| | Gate (GG) Level 1 expression vector |
| pICH47742_1A_{At}TP::EPYC1::GFP | Full-length codon-optimized EPYC1 with A. thaliana |
| | RbcS1A transit peptide and GFP in GG Level 1 |
| | expression vector |
| pAGM8031_1A_{At}TP::EPYC1_pFast | Full-length codon-optimized EPYC1 with A. thaliana |
| | RbcS1A transit peptide in GG Level M expression vector |
| | with pFast red selection marker |
| pAGM8031_1A_{At}TP::EPYC1::GFP_pFast | Full-length codon-optimized EPYC1 with A. thaliana |
| | RbcS1A transit peptide and GFP in GG Level M |
| | expression vector with pFast red selection marker |
| pAGM8031_EPYC1::GFP_pFast | Full-length codon-optimized EPYC1 with GFP in GG |
| | Level M expression vector with pFast red selection |
| | marker |
| pB7_S2_{Cr}::YFP^{N} | C. reinhardtii SSU RbcS2 fused to N terminus of YFP in |
| | pB7WG2,0 expression vector |
| pB7_S2_{Cr}::YFP^{C} | C. reinhardtii SSU RbcS2 fused to C terminus of YFP in |
| | pB7WG2,0 expression vector |
| pB7_1A_{At}TP::EPYC1::YFP^{N} | EPYC1 fused to N terminus of YFP in pB7WG2,0 |
| | expression vector |
| pB7_1A_{At}TP::EPYC1::YFP^{C} | EPYC1 fused to C terminus of YFP in pB7WG2,0 |
| | expression vector |
| pB7_1A_{At}MOD::YFP^{N} | A. thaliana SSU RbcS1A with modified alpha-helices |
| | fused to N terminus of YFP in pB7WG2,0 expression |
| | vector |
| pB7_1A_{At}MOD::YFP^{C} | A. thaliana SSU RbcS1A with modified alpha-helices |
| | fused to C terminus of YFP in pB7WG2,0 expression |
| | vector |
| pB7_1A_{At}::YFP^{N} | A. thaliana SSU RbcS1A fused to N terminus of YFP in |
| | pB7WG2,0 expression vector |
| pB7_1A_{At}::YFP^{C} | A. thaliana SSU RbcS1A fused to C terminus of YFP in |
| | pB7WG2,0 expression vector |
| pICH47732_CP12_{At}::YFP^{C} | A. thaliana CP12 fused to N terminus of YFP in Level 1 |
| | Golden Gate expression vector |
| pICH47732_CP12_{At}::YFP^{N} | A. thaliana CP12 fused to C terminus of YFP in Level 1 |
| | Golden Gate expression vector |
| pB7_S2_{Cr}::YFP^{N} | C. reinhardtii SSU RbcS2 fused to N terminus of YFP in |
| | pB7WG2,0 expression vector |

To generate fusion proteins, gene expression constructs were assembled into binary level M acceptor vectors. Level M vectors were transformed into Agrobacterium tumefaciens (AGL1) for transient gene expression in N. benthamiana (Schöb, et al., Mol. and Gen. Genetics (1997) 256: 581-585) or stable insertion in A. thaliana plants by floral dipping (Clough and Bent, Plant J. (1998) 16: 735-743). Homozygous insertion lines were identified in the T3 generation using the pFAST-R selection cassette (Shimada, et al., Plant J. (2010) 61: 519-528).

### DNA and Leaf Protein Analyses

PCR reactions were performed as in McCormick and Kruger (McCormick and Kruger, Plant J. (2015) 81: 570-683) using the gene-specific primers listed in Table 4.

TABLE 4
List of primers used for producing the vectors used for plant transformation.

| Primer name | Primer sequence | Vector |
|---|---|---|
| LCI5 full 1F | TACGGTCGAAGACGAAGGTATGGCTA | pICH47742_EPYC1::GFP |
| | CGATCAGTTCTATG (SEQ ID NO: 125) | pICH47742_1A_{At}TP::EPYC1::GFP |
| LCI5 full 1R | TACGGTCGAAGACGAGATGACTCTCTC | pAGM8031_1A_{At}TP::EPYC1_pFast |
| | CAAGATCCTCT (SEQ ID NO: 126) | pAGM8031_1A_{At}TP::EPYC1::GFP_pFast |
| LCI5 full 2F | ACGTACCGAAGACCACATCTACTGCTA | pAGM8031_EPYC1::GFP_pFast |
| | CAGTTCAAGC (SEQ ID NO: 127) | |
| L0 CDS1 | ACGTACCGAAGACCATGACCTAGCTGG | |
| LCI5+SP-1 R | TGCTGGCG (SEQ ID NO: 128) | |
| L0 CDS1 | ACGTACCGAAGACAGGTCATCCTCAGC | |
| LCI5+SP-2 F | TAGTTGGAG (SEQ ID NO: 129) | |
| L0 CDS1 | ACGTACCGAAGACAGAAGCTCAAAGG | |
| LCI5+SP-2 R | CCCTTTCTCCA (SEQ ID NO: 130) | |
| L0 SP SP1A_F | TGCACTCGAAGACAGAATGGCTTCCTC | pICH47742_1A_{At}TP::EPYC1::GFP |
| | TATGCTC (SEQ ID NO: 131) | pAGM8031_1A_{At}TP::EPYC1_pFast |
| L0 SP SP1A_R | TGCACTCGAAGACAGACCTTCGGAATC | pAGM8031_1A_{At}TP::EPYC1::GFP_pFast |
| | GGTAAG (SEQ ID NO: 132) | |
| L0 CDS1 | ACGTACCGAAGACAGAAGCTCAAAGG | pAGM8031_1A_{At}TP::EPYC1_pFast |
| LCI5+SP-2 R | CCCTTTCTCCA (SEQ ID NO: 130) | |
| AT1G67090_TP | CAACTTTGTACAAAAAAGCAGGCTCCG | pB7_S2_{Cr}::YFP^{C} |
| (+TOPO)_for | AATTCGCCCTTATGGCTTCCTCTATG | pB7_1A_{At}MOD::YFP^{C} |
| | (SEQ ID NO: 133) | pB7_1A_{At}::YFP^{C} |
| | | pB7_S2_{Cr}::YFP^{N} |
| | | pB7_1A_{At}MOD::YFP^{N} |
| | | pB7_1A_{At}::YFP^{N} |
| | | pB7_1A_{At}TP::EPYC1::YFP^{N} |
| | | pB7_1A_{At}TP::EPYC1::YFP^{C} |
| RbcS1A(+YFPc155) | AGCGTAATCTGGAACATCGTATGGGTA | pB7_1A_{At}::YFP^{C} |
| rev | CATACCGGTGAAGCTTGGTGGCTTG | pB7_1A_{At}MOD::YFP^{C} |
| | (SEQ ID NO: 134) | |
| RbcS1A(+YFPn173) | ATCCTCCTCAGAAATCAACTTTTGCTC | pB7_1A_{At}::YFP^{N} |
| rev | CATACCGGTGAAGCTTGGTGGCTTG | pB7_1A_{At}MOD::YFP^{N} |
| | (SEQ ID NO: 135) | |
| RbcS1(+YFPc155) | AGCGTAATCTGGAACATCGTATGGGTA | pB7_S2_{Cr}::YFP^{C} |
| rev | CATAACACTACGTTTGTTGGCTGG (SEQ | |
| | ID NO: 136) | |
| RbcS1(+YFPn173) | GATCCTCCTCAGAAATCAACTTTTGCT | pB7_S2_{Cr}::YFP^{N} |
| rev | CCATAACACTACGTTTGTTGGCTGG | |
| | (SEQ ID NO: 137) | |
| LCI5(+YFPc155) | AGCGTAATCTGGAACATCGTATGGGTA | pB7_1A_{At}TP::EPYC1::YFP^{C} |
| rev | CATAAGGCCCTTTCTCCAGTCTG (SEQ | |
| | ID NO: 138) | |
| LCI5(+YFPn173) | AAGATCCTCCTCAGAAATCAACTTTTG | pB7_1A_{At}TP::EPYC1::YFP^{N} |
| rev | CTCCATAAGGCCCTTTCTCCAGTCTG | |
| | (SEQ ID NO: 139) | |

Soluble protein was extracted from frozen leaf material of 21-d-old plants (sixth and seventh leaf) in 5× Bolt LDS sample buffer (ThermoFisher Scientific) with 200 mM DTT at 70° C. for 15 min. Extracts were centrifuged and the supernatants subjected to SDS-PAGE on a 4-12% (w/v) polyacrylamide gel and transferred to a nitrocellulose membrane. Membranes were probed with rabbit serum raised against wheat Rubisco at 1:10,000 dilution (Howe, et al., PNAS (1982) 79: 6903-6907) or against EPYC1 at 1:2,000 dilution (Mackinder, et al., PNAS (2016) 113: 5958-5963), followed by HRP-linked goat anti-rabbit IgG (Abcam) at 1:10,000 dilution, and visualized using Pierce ECL Western Blotting Substrate (Life Technologies).

### Growth Analysis and Photosynthetic Measurements

A. thaliana plant lines expressing EPYC1 fused with the 1A_{At}TP (1A_{At}-TP::EPYC1) in either WT, S2_{Cr} or the 1A_{At}MOD background were tested. Three independently transformed T3 lines (Line 1, Line 2, and Line 3) per background (WT, S2_{Cr} or the 1A_{At}MOD) were measured, and compared to their corresponding segregant lines (Line 1 Seg, Line 2 Seg, and Line 3 Seg) lacking EPYC1.

For growth analysis, plants were harvested at 31 days and the fresh (FW) and dry weights (DW) were measured. The values in FIGS. 8B-8C are the means±SE of measurements made on 12 rosettes (for FW and DW measurements) or 16 rosettes (for growth assays). Asterisks indicate significant difference in FW or DW between transformed lines and segregants (P<0.05) as determined by Student's paired sample t-tests. Rosette growth rates were quantified using an in-house imaging system (Dobrescu, et al., Plant Methods (2017) 13: 95).

For photosynthetic measurements, the same plants used in growth analysis were measured on day 31 (before harvest). Means±SE of measurements made on a single leaf from each of 12 plants are shown in Table 5, below. Maximum quantum yield of photosystem II (PSII) (dark-adapted leaf fluorescence; Fᵥ/Fₘ) was measured using a Hansatech Handy PEA continuous excitation chlorophyll fluorimeter (Hansatech Instruments Ltd.) (Maxwell and Johnson, J. of Exp. Bot. (2000) 51: 659-668).

### Co-Immunoprecipitation and Immunoblotting

Rosettes of 35-d-old A. thaliana plants expressing EPYC1 in a complemented Rubisco mutant background (S2_{Cr}, 1A_{At}MOD or 1A_{At}) were snap frozen and ground in liquid N₂. An equal volume of IP extraction buffer (100 mM HEPES [pH 7.5], 150 mM NaCl, 4 mM EDTA, 5 mM DTT, 0.4 mM PMSF, 10% [v/v] glycerol, 0.1% [v/v] Triton-X-100 and one Roche cOmplete EDTA-free protease inhibitor tablet per 10 ml) was added, samples were rotated at 4° C. for 15 min, centrifuged at 4° C. and filtered through two layers of Miracloth (Merck). Each extract (2 ml) was pre-cleared by incubating with 50 μl Protein A Dynabeads (ThermoFisher Scientific) pre-equilibrated in IP buffer for 1 hr at 4° C., before discarding the beads. Antibody-coated beads were generated by applying 3.5 μg anti-EPYC1 antibody to 50 μl Protein A Dynabeads, which were then rotated at 4° C. for 30 min. The antibody was crosslinked to the beads using Pierce BS3 cross-linking agent (Thermo Scientific). Each protein extract was incubated with the antibody-coated beads and rotated at 4° C. for 2 hrs. Unbound sample (flow-through) was discarded and the beads washed four times with washing buffer (20 mM Tris-HCl [pH 8], 150 147 mM NaCl, 0.1% [w/v] SDS, 1% [v/v] Triton-X-100, 2 mM EDTA). Immunocomplexes were eluted by adding 50 μl elution buffer (2× LDS sample buffer, 200 mM DTT) and heating for 15 min at 70° C., before discarding beads.

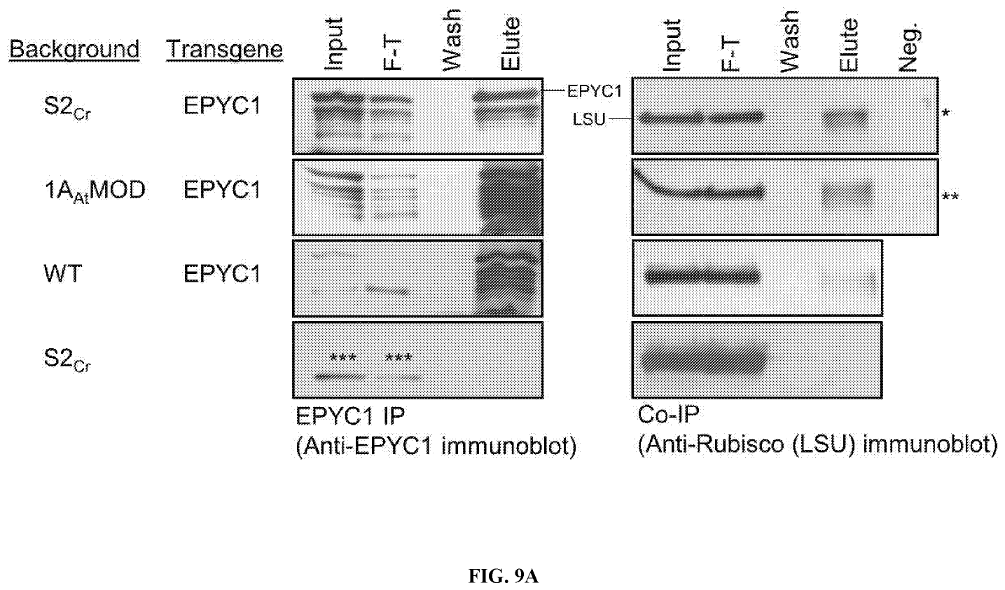

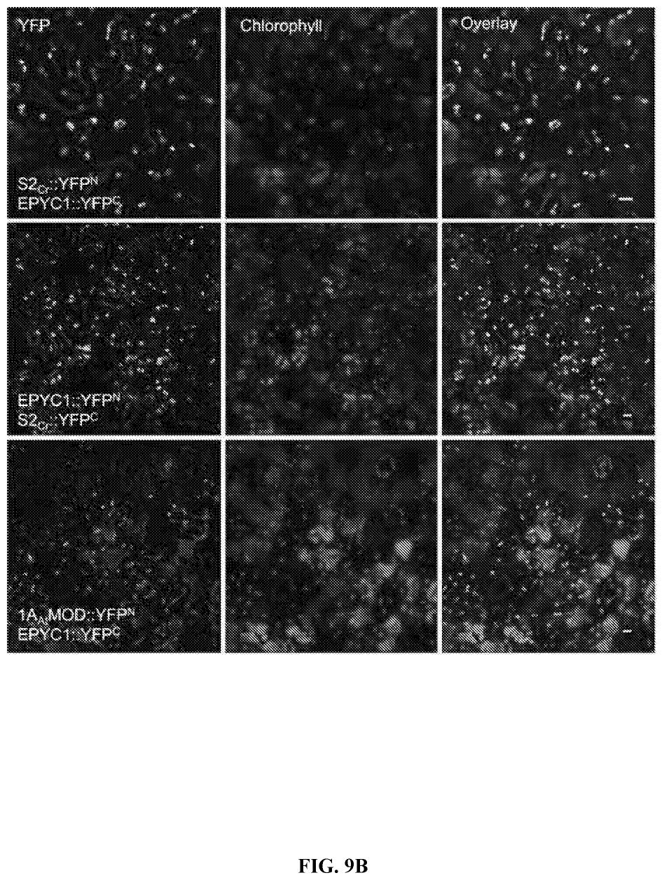

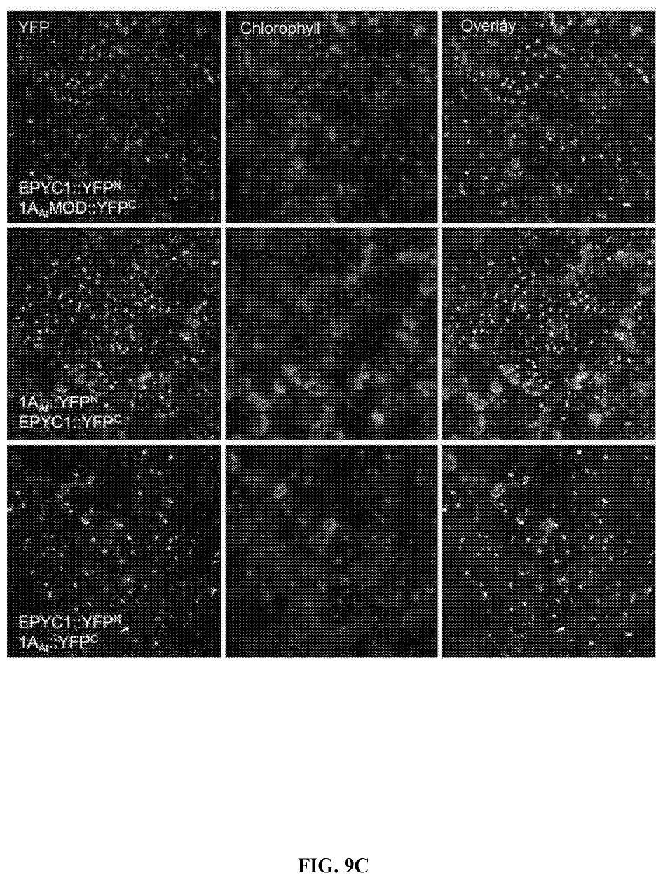

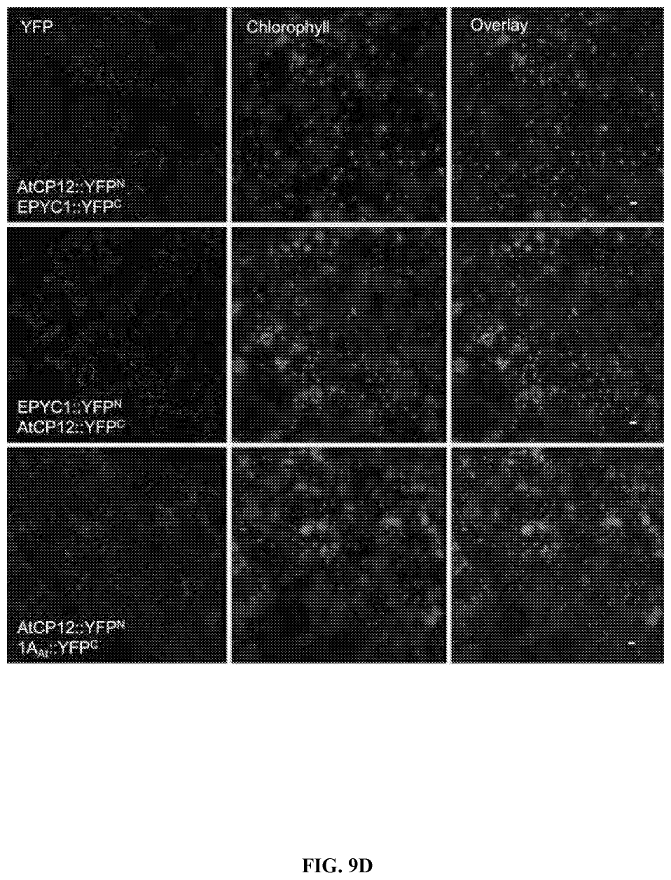

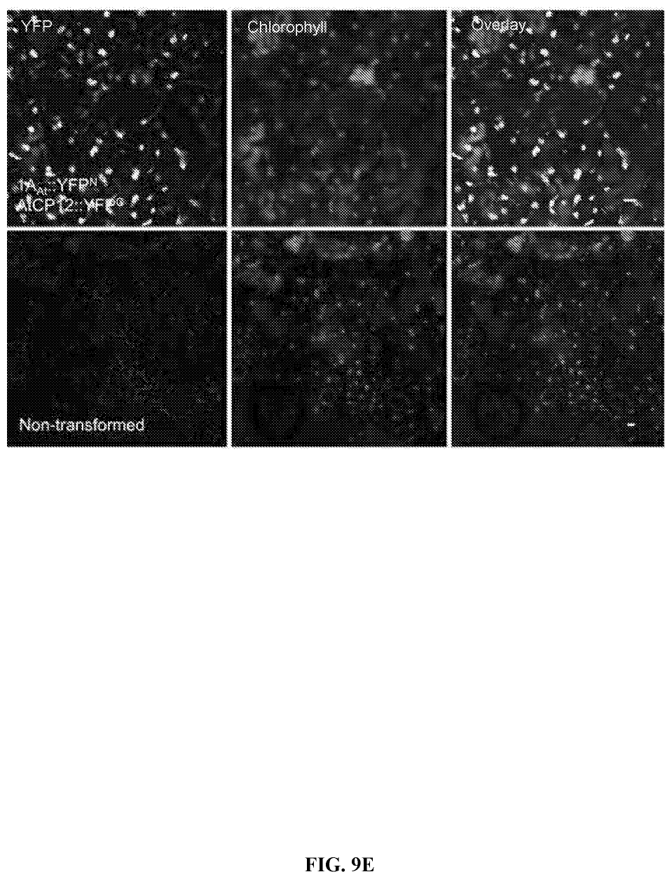

The eluted immunocomplexes were subjected to SDS-PAGE and immunoblotting. The 1A_{At}-TP::EPYC1 antibody serum targets the C-terminus of EPYC1 (Emanuelsson, et al., Nat. Protoc. (2007) 2: 953-971). For immunoblotting, two antibodies were used: anti-EPYC1 from Mackinder, et al., PNAS (2016) 171: 133-147, and anti-Rubisco (Rubisco antibody as used in Mackinder 2016 and first published in Howe, et al., PNAS (1982) 79: 6903-6907). In FIG. 8E, the ratio of EPYC1 in the A. thaliana protein extract was compared to that in the C. reinhardtii extract using densitometry. From this the stoichiometry of EPYC1 to Rubisco LSU was estimated. In FIG. 9A, the blots on the right (Co-IP) show the results when probed with an antibody against the Rubisco large subunit (LSU). Lanes from left to right display results from the input (Input), flow-through (F-T), 4th wash (Wash), and boiling elute (Elute), respectively, which were run on an SDS—page gel, transferred to a nitrocellulose membrane and probed with either anti-Rubisco or anti-EPYC1 antibody. Negative controls (Neg.) were carried out by replacing the anti-EPYC1 antibody on the Protein-A beads with either anti-HA antibody (*) or no antibody (**) and proceeding with IP as before (only the eluted sample is shown). Triple asterisks (***) indicate a non-specific band observed with the anti-EPYC1 antibody in all samples including the control line not expressing EPYC1 (S2_{Cr}).

### Bimolecular Fluorescence Complementation Analysis (BiFC)

Bimolecular fluorescence complementation analysis (BiFC) was carried out to provide additional information about the EPYC1-Rubisco interaction in vivo. Three Rubisco SSUs (1A_{At}, S2_{Cr} and 1A_{At}MOD) and EPYC1, each fused at the C-terminus to either YFP^{N} or YFP^{C} were transiently co-expressed in N. benthamiana (Walter, et al., Plant J. (2004) 40: 428-438).

### Confocal Laser Scanning Microscopy

Leaves were imaged with a Leica TCS SP2 laser scanning confocal microscope or a Leica TCS SP8 laser scanning confocal microscope as in Atkinson et al. (Atkinson, et al., Plant Biotech. J. (2016) 14: 1302-1315).

### Results

### EPYC1 can be Targeted to Higher Plant Chloroplasts

EPYC1 was codon-optimized for nuclear expression in higher plants (FIG. 7A), and binary expression vectors were constructed whereby EPYC1 was C-terminally fused to GFP and expressed under the control of the 35S constitutive promoter. The level M acceptor pAGM8031 was used for plasmid assembly. The vectors described in Table 3 above were used to agro-infiltrate the leaves of N. benthamiana plants and to stably transform A. thaliana plants. Localization of EPYC1::GFP was then visualized in N. benthamiana leaves (FIG. 7B) and in stably transformed A. thaliana plants (FIG. 7C). Unlike other chloroplast CCM components expressed in plants thus far (Atkinson, et al., Plant Biotech. J. (2016) 14: 1302-1315), EPYC1 was not able to localize to the chloroplast in either N. benthamiana or A. thaliana, with fluorescent signals absent from the chloroplast (see overlay images in FIGS. 5A-5B). The 1A_{At} chloroplastic transit peptide (1A_{At}-TP) was therefore added to the N-terminus of the full length EPYC1::GFP. Fusion to 1A_{At}-TP resulted in re-localization of EPYC1:: GFP to the chloroplast stroma in both N. benthamiana (row 1 vs. row 2 in FIG. 7B) and A. thaliana (row 1 vs. row 2 in FIG. 7C).

EPYC1 Expression in Plant Chloroplasts does not Hinder Plant Growth or Photosynthetic Efficiency

Wild-type A. thaliana plants and two Rubisco small subunit (1a3b) mutant lines complemented with S2_{Cr} or 1A_{At}MOD, previously made by Atkinson et al. (Atkinson, et al., New Phytol. (2017) 214: 655-667) (FIG. 3A), were transformed with 1A_{At}-TP::EPYC1 (lacking a GFP tag) (see FIG. 7A for the plasmid map). Three homozygous T3 lines from each background were selected for further analyses (EPYC1_1-3; S2_{Cr}_EPYC1_1-3 and 1A_{At}MOD_EPYC1_1-3).

Growth analyses showed a slightly reduced growth phenotype (i.e. area, FW and DW) for some plants expressing 1A_{At}-TP::EPYC1 compared to their corresponding segregants, but the observed decrease was not consistently significant (FIGS. 8B-8C).

Table 5 shows the maximum quantum yield of PSII (Fv/Fm) measurements for EPYC1 expressing A. thaliana plants. For each of the three genetic backgrounds (WT, S2_{Cr}, and 1A_{At}MOD), three independently transformed T3 lines (Line 1, Line 2, and Line 3) were measured, and compared to their corresponding segregants lacking EPYC1 (Line 1 Seg, Line 2 Seg, and Line 3 Seg). Regardless of genetic background, the addition of 1A_{At}-TP::EPYC1 did not affect photosynthetic efficiency as measured by dark-adapted leaf fluorescence; Fv/Fm).

TABLE 5
Maximum quantum yield of PSII (Fv/Fm) measurements for 1A_{At}-TP::EPYC1
expressing A. thaliana plants from three genetic backgrounds.

| Genetic | | | | | | |
|---|---|---|---|---|---|---|
| background | Line 1 | Line 1 Seg | Line 2 | Line 2 Seg | Line 3 | Line 3 Seg |
| WT | 0.856 ± | 0.856 ± | 0.856 ± | 0.856 ± | 0.856 ± | 0.856 ± |
| | 0.002 | 0.002 | 0.002 | 0.002 | 0.002 | 0.002 |
| S2_{Cr} | 0.856 ± | 0.856 ± | 0.856 ± | 0.856 ± | 0.856 ± | 0.856 ± |
| | 0.002 | 0.002 | 0.002 | 0.002 | 0.002 | 0.002 |
| 1A_{At}MOD | 0.859 ± | 0.859 ± | 0.859 ± | 0.859 ± | 0.859 ± | 0.859 ± |
| | 0.001 | 0.001 | 0.001 | 0.001 | 0.001 | 0.001 |

Immunoblots against 1A_{At}-TP::EPYC1 in A. thaliana produced a dominant band of approximately 34 kDa (slightly smaller than the mature native C. reinhardtii isoform [35 kDa]) which suggested cleavage of both 1A_{At}-TP and a portion of the N-terminal region of EPYC1 (the antibody serum targeted the C-terminus of EPYC1) (Emanuelsson, et al., Nat. Protoc. (2007) 2:953-971) (FIGS. 8D and 9A). Densitometry analysis showed that protein levels of EPYC1 in the highest expressing A. thaliana lines were roughly 14 times lower than protein levels of EPYC1 in C. reinhardtii in relation to the Rubisco LSU (FIG. 8E). Based on the reported ratio of ca. 1:6 for EPYC1 to Rubisco LSU in C. reinhardtii grown under low CO₂ conditions (Mackinder, et al., PNAS (2016) 171: 133-147), the stoichiometry of EPYC1 to the A. thaliana LSU in the transgenic line was therefore estimated as 1:84. This ratio was also lower than the observed occurrence of between 1 and 4 EPYC1 peptides per Rubisco (i.e., 8 LSUs) in phase-separated material in the in vitro reconstituted pyrenoidal system (Wunder, et al., Nat. Commun. (2018) 9: 5076). In addition to a non-specific band at 29 kDa, several smaller bands were also evident for EPYC1 in A. thaliana (FIG. 8A). Additional bands were not observed for EPYC1 extracted from C. reinhardtii or yeast (FIG. 8D), which suggested that EPYC1 may be targeted by plant proteases.

The above results showed that constitutive expression of EPYC1 in the chloroplast did not impact plant growth under the conditions tested. Further, the constitutive expression of EPYC1 in the chloroplast did not impact plant photosynthetic efficiency, as measured by Fv/Fm.

EPYC1 Interacts with Rubisco in Higher Plants

Having shown that specific SSUs can interact with EPYC1 in a yeast two-hybrid system, it was next investigated whether the interactions with Rubisco would occur in planta. Multiple A. thaliana plant lines were evaluated, specifically two complemented 1a3b mutant lines and one wild-type line expressing EPYC1 (S2_{Cr}_EPYC1_1, 1A_{At}MOD_EPYC1_1 and EPYC1_1, respectively). EPYC1 was immunoprecipitated from each of these lines using anti-EPYC1 antibody attached to Protein A coated beads, and the elutes were analyzed by immunoblot using antibodies against EPYC1 or Rubisco (FIG. 9A). Unexpectedly, the LSU was detected in the elutes of S2_{Cr}_EPYC1 and 1A_{At}MOD_EPYC1 lines, as well as the wild-type expressing EPYC1. To ensure that the observed co-immunoprecipitation (co-IP) was not a result of Rubisco promiscuity or non-specific binding onto the beads or antibodies, several negative controls were included. Rubisco was not detected in the elute of pull-downs with anti-HA coated beads or beads with no antibody, or in the elute from S2_{Cr} plants not transformed with EPYC1. Therefore, these results indicated that EPYC1 was able to interact with Rubisco in transformed plant lines in the absence of a C. reinhardtii or C. reinhardtii-like SSU. However, this interaction was not sufficient to facilitate visible aggregate akin to liquid-like phase separation as for a pyrenoid. It was not possible to fully quantify the relative strength of the interactions due to the inherent variation in EPYC1 expression levels between the three lines tested. Nevertheless, the levels of EPYC1 eluted in the EPYC1 IP assays were similar, while the greater amounts of Rubisco eluted in the 1A_{At}MOD_EPYC1 and S2_{Cr}_EPYC1 co-IP assays could suggest a stronger interaction with EPYC1 in those lines than in the wild-type background.

Consistent with the immunoprecipitation results shown in FIG. 9A, a BiFC signal for reconstituted YFP fluorescence was observed in plants co-expressing EPYC1 and each of the three SSUs, regardless of which protein was fused to YFP^{N} and which to YFP^{C} (FIGS. 9B-9E). The results described in Example 3, however, indicated that the apparent interaction observed between EPYC1 and the 1A_{At} SSU was not a true interaction. Instead, this interaction was likely observed as a result of the tendency for self-assembly of the split YFP halves (Waadt, et al., Plant J. (2008) 56: 506-516). Similarly, a negative control, AtCP12::YFP^{C}, unexpectedly produced a BiFC signal with 1A_{At}::YFP^{N}, but as no interaction was observed between 1A_{At}::YFP^{C} and AtCP12::YFP^{N}, this interaction was likely artifactual. The interpretation that the apparent interaction observed between EPYC1 and the 1A_{At} SSU was not a true interaction sufficient to facilitate phase separation was confirmed by the experimental results presented in Example 3, below.

### Example 3: EPYC1 can be Engineered to Exhibit Liquid-Like Aggregate in Heterologous Systems and Expression of TobiEPYC1 Constructs Results in Spherical Aggregates in Higher Plant Chloroplasts

The following example describes the detection of liquid-like aggregate of EPYC1, using an in vitro system. Further, the following example describes the detection of spherical aggregates of the TobiEPYC1::GFP construct in higher plant chloroplasts.

### Materials and Methods

### Protein Production, Droplet Sedimentation Assay and Microscopy

Rubisco was purified from 25- to 30-day-old A. thaliana rosettes (wild-type plants and S2_{Cr} lines) using a combination of ammonium sulfate precipitation, ion-exchange chromatography, and gel filtration (Shivhare and Mueller-Cajar, Plant Phys. (2017) 1505-1516). The hybrid Rubisco complexes in S2_{Cr} lines consisted of the A. thaliana LSU and a mixed population of A. thaliana SSUs and S2_{Cr} (roughly 1:1) (Atkinson, et al., New Phytol. (2017) 214: 655-667). Rubisco was also purified from C. reinhardtii cells (CC-2677). EPYC1 and EPYC1::GFP were produced in E. coli and purified as described in Wunder et al. (Wunder, et al., Nature Commun. (2018) 9: 5076).

EPYC1-Rubisco droplets were reconstituted at room temperature in 10 μl reactions for 5 min in buffer A (20 mM Tris-HCl [pH 8.0], and 50 mM NaCl), and were separated at 4° C. from the bulk solution by centrifugation for 4 min at 21,100×g. Liquid-liquid phase separation with EPYC1 was tested using an in vitro assay developed by Wunder et al. (Wunder, et al., Nature Commun. (2018) 9: 5076). Pellet (droplet) and supernatant (bulk solution) fractions were subjected to SDS-PAGE and Coomassie staining.

For light and fluorescence microscopy, reaction solutions (5 μl) were imaged after 3-5 min with a Nikon Eclipse Ti Inverted Microscope using the settings for differential interference contrast and epifluorescence microscopy (using fluorescein isothiocyanate filter settings) with a ×100 oil-immersion objective focusing on the coverslip surface. The coverslips used were 22×22 mm (Superior Marienfeld, Germany) and fixed in one-well Chamlide CMS chamber for 22×22 coverslip (Live Cell Instrument, South Korea). ImageJ was used to pseudocolor all images.

### Immunogold Labelling and Electron Microscopy

Leaf samples were taken from 21-d-old S2_{Cr} and S2_{Cr} EPYC1 plants and fixed with 4% (v/v) paraformaldehyde, 0.5% (v/v) glutaraldehyde and 0.05 M sodium cacodylate [pH 7.2]. Leaf strips (1 mm wide) were vacuum infiltrated with fixative three times for 15 min, then rotated overnight at 4° C. Samples were rinsed three times with PBS then dehydrated sequentially by vacuum infiltrating with 50%, 70%, 80% and 90% ethanol (v/v) for 1 hr each, then three times with 100% ethanol. Samples were infiltrated with increasing concentrations of LR White Resin (30%, 50%, 70% [w/v]) mixed with ethanol for 1 hr each, then 100% resin three times. The resin was polymerized in capsules at 50° C. overnight. Sections (1 μm thick) were cut on a Leica Ultracut ultramicrotome, stained with Toluidine Blue, and viewed in a light microscope to select suitable areas for investigation. Ultrathin sections (60 nm thick) were cut from selected areas and mounted onto plastic-coated copper grids. Grids were blocked with 1% (w/v) BSA in TBSTT (Tris-buffered saline with 0.05% [v/v] Triton X-100 and 0.05% [v/v] Tween 20), incubated overnight with anti-Rubisco antibody in TBSTT at 1:250 dilution, and washed twice each with TBSTT and water. Incubation with 15 nm gold particle-conjugated goat anti-rabbit secondary antibody (Abcam) in TBSTT was carried out for 1 hr at 1:200 dilution, before washing as before. Grids were stained in 2% (w/v) uranyl acetate then viewed in a JEOL JEM-1400 Plus TEM. Images were collected on a GATAN OneView camera.

### TobiEPYC1 Construct Design and Plant Transformation and Aggregate Data

TobiEPYC1 gene expression cassettes are shown in FIG. 12A. Cassette 1 (TobiEPYC1) contains a truncated version of native EPYC1, which contains a truncated N-terminal domain (SEQ ID NO: 40) full length first through fourth repeat regions (in lightest gray (SEQ ID NO: 36), gray (SEQ ID NO: 69), gray (SEQ ID NO: 70), and black (SEQ ID NO: 71)), and a full length C-terminal domain (SEQ ID NO: 41). Cassette 2 (TobiEPYC1::GFP) contains the same truncated version of native EPYC1 fused with GFP. Cassette 3 (4 reps TobiEPYC1) contains a synthetic version of EPYC1 with four copies of the first repeat region (SEQ ID NO: 38). Cassette 4 GFP (4 reps TobiEPYC1::GFP) contains the same synthetic version of EPYC1 with four copies of the first repeat region fused with GFP. Cassette 5 (8 reps TobiEPYC1) contains a synthetic version of EPYC1 with eight copies of the first repeat region (SEQ ID NO: 39). Cassette 6 (8 reps TobiEPYC1::GFP) contains the same synthetic version of EPYC1 with eight copies of the first repeat region fused with GFP.

Binary plasmid constructs were assembled by Golden Gate MoClo system (Engler, et al., ACS Synth. Bio. (2014) 3: 839-843). The plasmids contained two TobiEPYC1 expression cassettes, as shown in FIGS. 12B-12C. Table 6, below, provides descriptions of the vectors that were used for plant transformation with TobiEPYC1 gene cassettes

TABLE 6
TobiEPYC1 vectors used for plant transformation.

| Vector | Description |
|---|---|
| pAGM4723_TobiEPYC1 | Full-length codon-optimized TobiEYPC1 in |
| | Golden Gate (GG) Level 2 expression vector |
| pAGM4723_TobiEPYC1::GFP | Full-length codon-optimized TobiEYPC1 and |
| | GFP in GG Level 2 expression vector |
| pAGM4723_4_reps_TobiEPYC1 | Full-length codon-optimized 4 reps TobiEYPC1 in |
| | Golden Gate (GG) Level 2 expression vector |
| pAGM4723_4_reps_TobiEPYC1::GFP | Full-length codon-optimized 4 reps TobiEYPC1 |
| | and GFP in GG Level 2 expression vector |
| pAGM4723_8_reps_TobiEPYC1 | Full-length codon-optimized 8 reps TobiEYPC1 in |
| | Golden Gate (GG) Level 2 expression vector |
| pAGM4723_8_reps_TobiEPYC1::GFP | Full-length codon-optimized 8 reps TobiEYPC1 |
| | and GFP in GG Level 2 expression vector |

Transformation of the vectors into A. thaliana was done using the floral dipping method as described in Example 2. At least three separate plant lines were generated for each of the vectors in Table 6.

### Detection of Aggregate in TobiEPYC1::GFP Plant Lines

Tissue from TobiEPYC1::GFP transgenic plant lines was imaged using confocal microscopy, as described in Example 2. Confocal images were from intact leaf tissue (FIGS. 12D-F, 12L, 13A-B) or mesophyll protoplasts extracted from leaf tissue (FIGS. 12G-K). At least one replicate from at least two separate plant lines of each TobiEPYC1::GFP variant (shown in Table 6) was imaged.

Aggregate characteristics were analyzed by fluorescence recovery after photobleaching (FRAP). FRAP was carried out using a Leica SP8 confocal microscope and a 63× water immersion objective, with a PMT detector. GFP fluorescence was imaged by excitation at 488 nm and emission between 504-532 nm. For the pre- and post-bleach images, laser power was set to 2%, whilst the bleach itself was carried out at 56% laser power. Pre-bleach images were captured at 189 ms intervals (6 in total), and post-bleach images were captured at 400 ms intervals (150 in total). Photo-bleaching was carried out on leaf samples by directing the laser to a small area of one of the TobiEPYC1::GFP aggregates within one chloroplast. Recovery time after photo-bleaching was calculated by comparing GFP expression in the bleached versus an un-bleached region.

The presence of EPYC1 and the C. reinhardtii Rubisco SSU was confirmed by immunoblot, as described in Example 2.

### Results

Hybrid Rubisco Containing Higher Plant Large Subunits (LSUs) and Mixed Populations of Higher Plant and C. reinhardtii SSUs Phase Separates with EPYC1

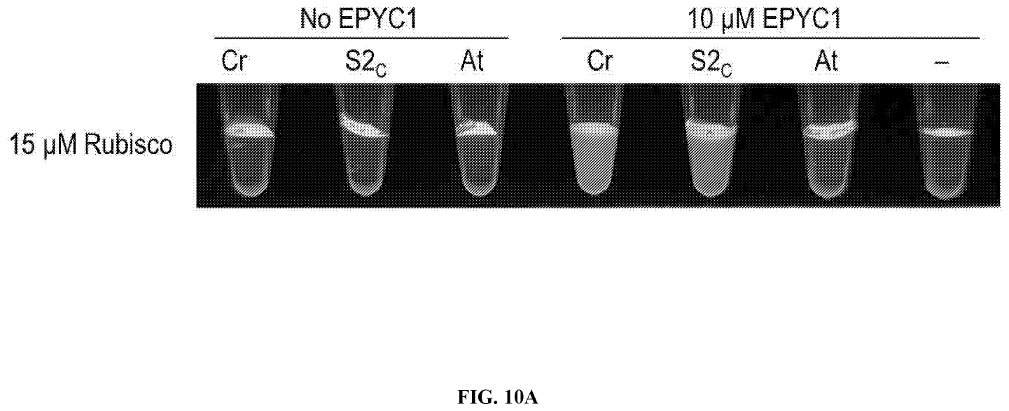

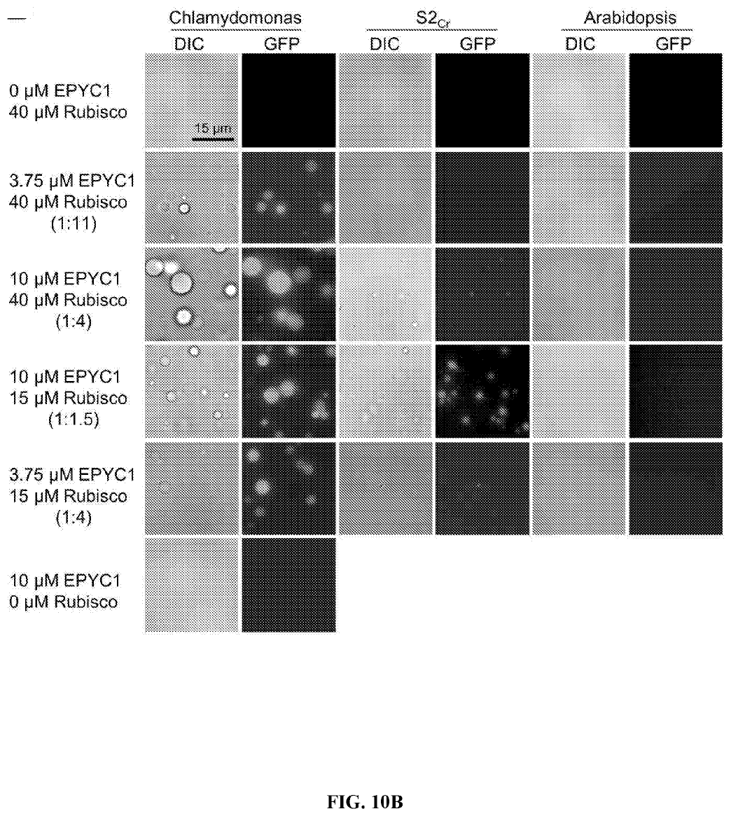

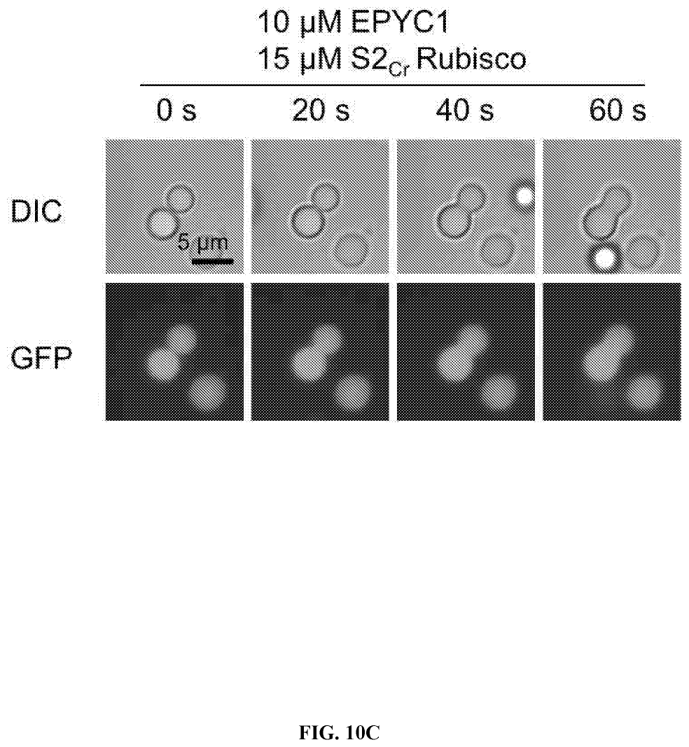

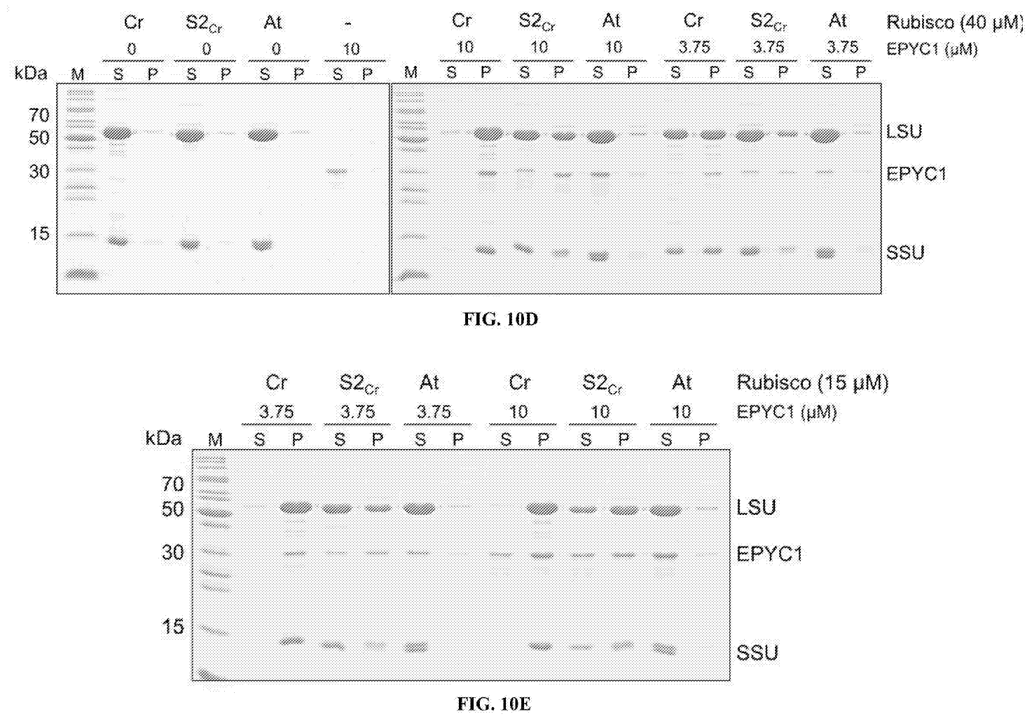

Current models of pyrenoid formation are based on specific weak multivalent interactions that promote liquid-like phase separation (Hyman, et al., Annu. Rev. Cell Biol. (2014) 30: 39-58; Freeman Rosenzweig, et al., Cell (2017) 171: 148-162). To observe if such interactions could occur with hybrid plant-derived Rubisco, it was examined whether Rubisco from A. thaliana 1a3b mutants complemented with S2_{Cr} was able to facilitate liquid-liquid phase separation with EPYC1 using an in vitro assay developed by Wunder et al. (Wunder, et al., Nature Commun. (2018) 9: 5076). Similarly to C. reinhardtii Rubisco, hybrid plant Rubisco (from the S2_{Cr} lines) was able to demix with EPYC1 and formed liquid-like droplets of comparable size, albeit at slightly higher ratios of EPYC1: Rubisco (FIGS. 10A-10B; time-course shown in FIG. 10C). In contrast, wild-type A. thaliana Rubisco did not phase separate under similar conditions, indicating that the presence of S2_{Cr} was critical for aggregate. In solutions containing C. reinhardtii or hybrid plant Rubisco, the droplets fused into a large homogeneous droplet (coalescence), supporting their liquid nature (FIG. 8C) (Hyman, et al., Annu. Rev. Cell Biol. (2014) 30: 39-58). Analysis by SDS-polyacrylamide gel electrophoresis (SDS-PAGE) analysis confirmed that both EPYC1 and Rubisco had entered the droplets (FIGS. 10D-10E).

### EPYC1 can be Engineered to Form Aggregates in Higher Plant Chloroplasts

To investigate the effect of EPYC1 on Rubisco aggregate in planta, the localization of Rubisco in the chloroplast of S2_{Cr} complemented A. thaliana 1a3b mutants expressing the highest levels of EPYC1 (S2_{Cr}_EPYC1_1) was examined. Immunogold labelling of Rubisco revealed an even distribution of gold particles throughout the chloroplast when visualized by TEM, which was similar to the S2_{Cr} control not expressing EPYC1 (FIGS. 11A-11B). This indicated that co-expression of EPYC1 and the C. reinhardtii SSU did not induce detectable rigid aggregates of Rubisco in these transformants.

Spherical Aggregate is Observed in Higher Plant Chloroplasts of Plants Transformed with TobiEPYC1

Initially, two versions of EPYC1 were tested for expression in plants. The first of these was EPYC1 truncated by 78 residues at the N-terminus (the predicted chloroplast transit peptide based on the ChloroP online tool) and fused to a long version of the chloroplast signal peptide for A. thaliana Rubisco SSU 1A (80 residues, MASSMLSSATMVASPAQATMVAPFNGLKSSAAFPATRKANNDITSITSNGGRVNCMQV WPPIGKKKFETLSYLPDLTDSE (SEQ ID NO: 62)). The second of these was the full length EPYC1 (317 residues; SEQ ID NO: 34) fused to a long version of the chloroplast signal peptide for A. thaliana Rubisco SSU 1A (80 residues; SEQ ID NO: 62). Neither of these two versions produced evidence of aggregate in either wild-type plants or in the stable transgenic A. thaliana line expressing C. reinhardtii SSU.

Compared to these two previous versions, the TobiEPYC1 constructs were optimized in three ways (TobiEPYC1 gene expression cassettes are shown in FIG. 12A). First, a new N-terminal truncation of EPYC1 (26 residues; SEQ ID NO: 40) was used. Second, the truncated EPYC1 was fused to a shorter chloroplast signal peptide for A. thaliana Rubisco SSU 1A (57 residues; SEQ ID NO: 63). The previous versions with the longer transit peptide were not successful, which indicated that the length of the transit peptide could be critical.

Third, two copies of the EPYC1 expression cassette were included on the binary plasmid with the aim to increase expression levels. Further, one copy had two terminators (see FIG. 12B), a strategy that reportedly increased expression circa 25 fold (Diamos and Mason, Plant Biotech. J. (2018) 16: 1971-1982). Although aggregates were still observed in lines with lower levels of GFP expression, the aggregates in those lines were smaller, indicating that two copies of the EPYC1 expression cassette may be necessary. These results indicated that the amounts of Rubisco SSU and EPYC1 may be important for observing aggregate. The A. thaliana 1a3b mutant used to express the C. reinhardtii SSU had reduced amounts of native SSU (Izumi, et al., J. Exp Bot. (2012) 63(5): 2159-2170). Therefore, it was previously estimated that the transgenic line expressed 50% native SSU and 40% C. reinhardtii SSU (Atkinson, et al., New Phytol. (2017) 214, 655-667). It was estimated that 60 mg m⁻² C. reinhardtii SSU was present the transgenic line based on Rubisco content measurement and immunoblot analysis (Supp. Table S3 in Atkinson, et al., New Phytol. (2017) 214, 655-667). Based on a 16 kD weight, 60 mg m⁻² C. reinhardtii SSU was equivalent to 3.75 μmol m⁻² C. reinhardtii SSU. The ratios of EPYC1 to Rubisco reported in C. reinhardtii ranged from 1:6 for the large subunit of Rubisco and 1:1 for the small subunit (Mackinder, et al., PNAS (2016) 113: 5958-5963) to 1:8 for the small subunit (Hammel, et al., Front. Plant Sci. (2018) 9: 1265). Wunder, et al. (Wunder, et a., Nat. Commun. (2018) 9: 5076) found that 7.5 μM EPYC1 was able to completely demix 30 μM Rubisco active sites, corresponding to a ratio of two EPYC1 molecules per Rubisco. The, the precise ratio of EPYC1 to Rubisco that would be optimal in planta is as yet unresolved. However, the above results indicated that 40% C. reinhardtii SSU in the total SSU pool was sufficient for aggregate when two copies of EPYC1 were expressed under constitutive promoters with single and double terminators, respectively.

FIG. 12D shows transient expression of EPYC1::GFP in N. benthamiana imaged at gain 25 and laser 2%, while FIG. 12E shows transient expression of TobiEPYC1::GFP in N. benthamiana imaged at gain 10 and laser 1%. These images show that transient expression levels of TobiEPYC1::GFP in N. benthamiana are very high. FIG. 12F shows fluorescence microscopy images of stable expression of TobiEPYC1::GFP in A. thaliana S2_{Cr} lines. The overlay images clearly indicate that TobiEPYC1::GFP aggregated in the chloroplast. These aggregates appeared to be highly spherical, which was indicative of phase separation bodies. FIGS. 12G-12I show fluorescence microscopy images of stable expression of TobiEPYC1::GFP in A. thaliana protoplasts. FIG. 12I shows that lower chlorophyll was observed at the location of the TobiEPYC1 aggregate (indicated by arrows). This was also observed in the images of FIG. 12J (note that the middle row is the same image as in FIG. 12I), where the overlay of the GFP, chlorophyll, and bright field images did not contain regions of overlapping fluorescence. These results suggested that the chloroplast thylakoids were being excluded from the EPYC1 aggregate. The images shown in FIG. 12K were of EPYC1 aggregates leaving the chloroplasts (indicated by arrows). These chloroplast-external EPYC1 aggregates remained aggregated within the media during the observation time period. The images shown in FIG. 12L are fluorescence microscopy images of protoplasts from wild type A. thaliana stably expressing TobiEPYC1::GFP. The overlay of the GFP and chlorophyll autofluorescence channel showed regions of overlapping fluorescence in white. This indicated that, unlike in the A. thaliana S2_{Cr} lines, EPYC1 was unable to form aggregates in the wild type A. thaliana lines, but instead only diffuse expression throughout the chloroplast was observed. These results indicated that the structural features of the C. reinhardtii SSU are required to observe the EPYC1 aggregate.

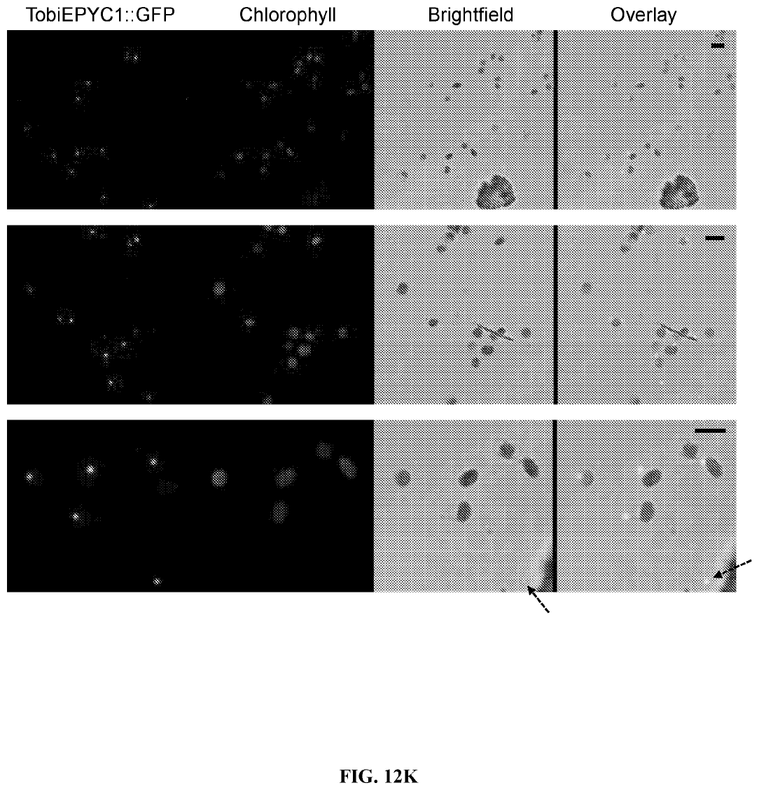

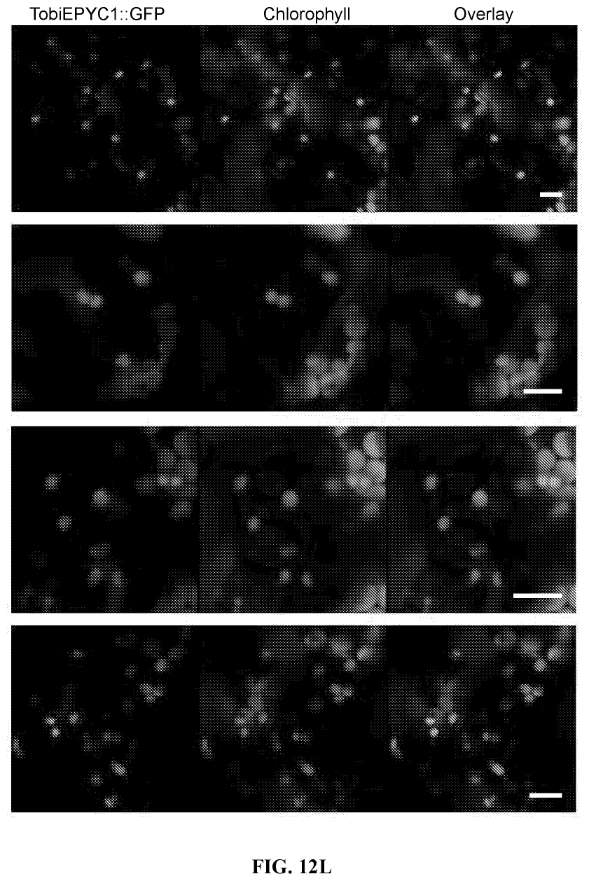

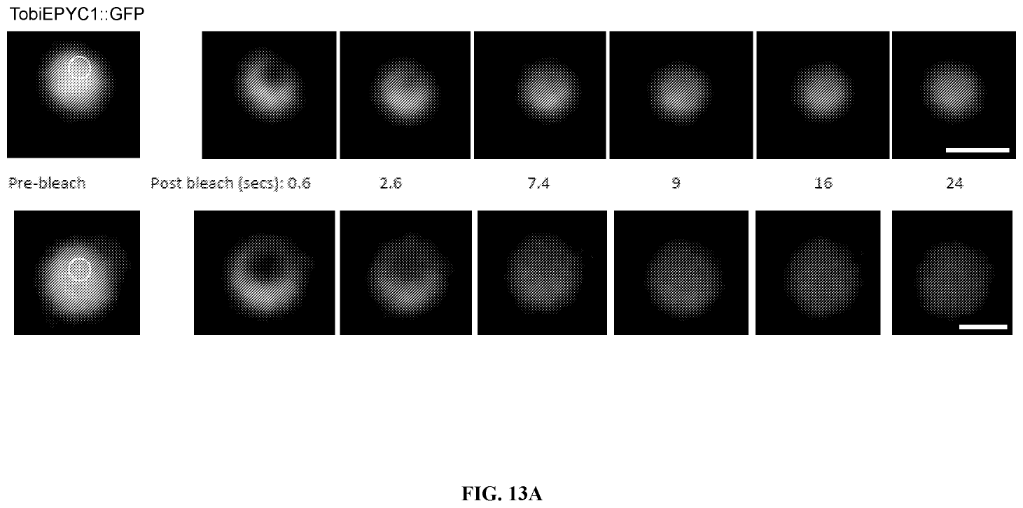

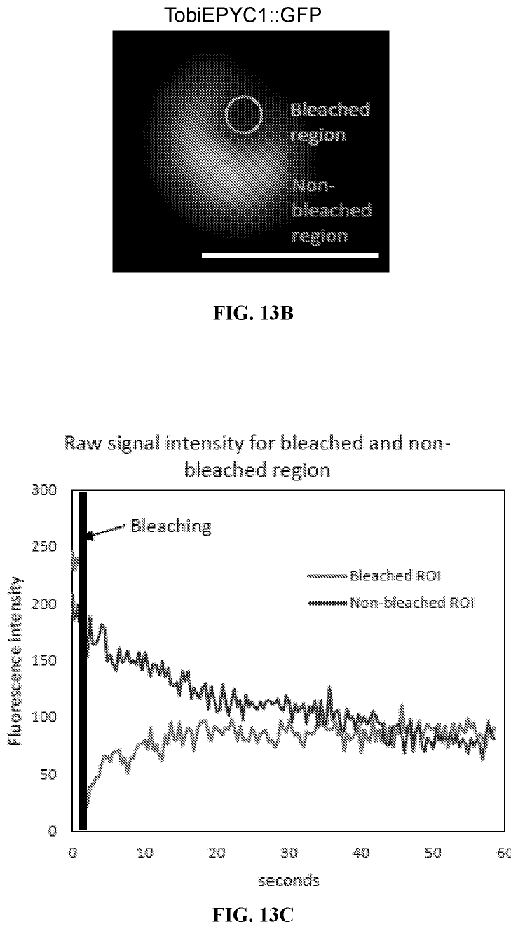

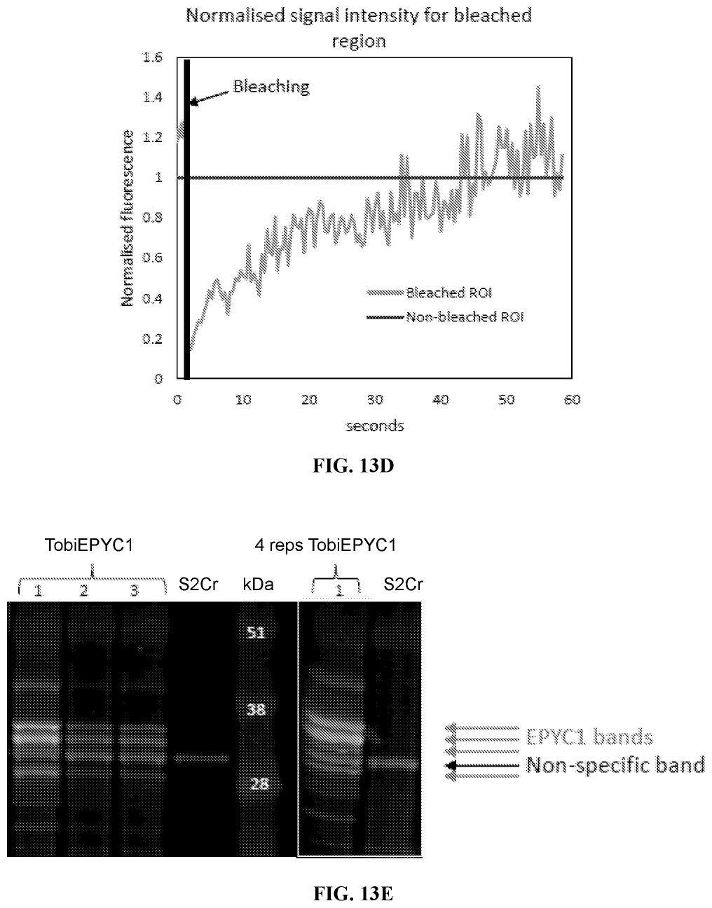

FIGS. 13A-13D show the results of FRAP imaging time courses to characterize EPYC1:: GFP aggregates in A. thaliana tissue. The recovery time after photobleaching was similar to that observed for demixed droplets in vitro in Wunder et al. (Wunder, et al., Nat. Commun. (2018) 9: 5076). The Western blot results shown in FIG. 13E indicated that the TobiEPYC1 gene expression cassettes still produced several bands in planta, which was indicative of degradation, despite the N-terminal truncation and the higher levels of expression. Overall, these results indicated that expression of TobiEPYC1 gene expression constructs in higher plants (e.g., A. thaliana) expressing the structural features of the C. reinhardtii SSU resulted in the formation of spherical aggregates in higher plant chloroplasts.

### Example 4: Increased Expression of a Truncated, Mature Form of EPYC1 Stably Aggregates Rubisco into Phase-Separated, Liquid-Like Condensate Structures in Higher Plant Chloroplasts

The following example describes molecular and cellular characterization of EPYC1-Rubisco chloroplastic condensates in Arabidopsis thaliana plant lines expressing high levels of a truncated, mature form of EPYC1 from a binary expression vector, alongside a plant-algal hybrid Rubisco. Further, it describes the impact of the condensates on plant metabolism, when plants are grown under different light levels.

This Example uses the same construct shown in FIG. 12C and in the second line of FIG. 12B, referred to above in Example 3 as “TobiEPYC1::GFP”. However, this Example and corresponding Figures refer to the construct to as “EPYC1-dGFP” rather than “TobiEPYC1::GFP”.

### Materials and Methods

### Plant Material and Growth Conditions

Arabidopsis (Arabidopsis thaliana, Col-0 background) seeds were sown on compost, stratified for 3 d at 4° C. and grown at 20° C., ambient CO₂ and 70% relative humidity under either 200 or 900 μmol photons m⁻² s⁻¹ supplied by cool white LED lights (Percival SE-41AR3cLED, CLF PlantClimatics GmbH, Wertingen, Germany) in 12 h light, 12 h dark. For comparisons of different genotypes, plants were grown from seeds of the same age and storage history, harvested from plants grown in the same environmental conditions.

The S2_{Cr} A. thaliana background line (1a3b Rubisco mutant complemented with an SSU from C. reinhardtii) is described in Atkinson et al. (New Phytol 214, 655-667, doi:10.1111/nph.14414 (2017)). The 1A_{At}MOD A. thaliana background line is described in Meyer et al. (PNAS, 109, 19474-19479, doi:10.1073/pnas.1210993109 (2012)) and Atkinson et al. (New Phytol 214, 655-667, doi:10.1111/nph.14414 (2017)).

### Construct Design and Transformation

The coding sequence of EPYC1 was codon-optimized for expression in higher plants as in Atkinson et al. (J. Exp. Bot. 70, 5271-5285, doi:10.1093/jxb/erz275 (2019)). Truncated mature EPYC1 was cloned directly into the level 0 acceptor vector pAGM1299 of the Plant MoClo system (Engler, C. et al. A Golden Gate Modular Cloning Toolbox for Plants. Acs Synth Biol 3, 839-843, doi:10.1021/sb4001504 (2014)). To generate fusion proteins, gene expression constructs were assembled into binary level 2 acceptor vectors. Level 2 vectors were transformed into Agrobacterium tumefaciens (AGL1) for stable insertion in A. thaliana plants by floral dipping as described in Example 2. Homozygous transgenic and azygous lines were identified in the T2 generation using the pFAST-R selection cassette (Shimada, et al., Plant J. (2010) 61: 519-528).

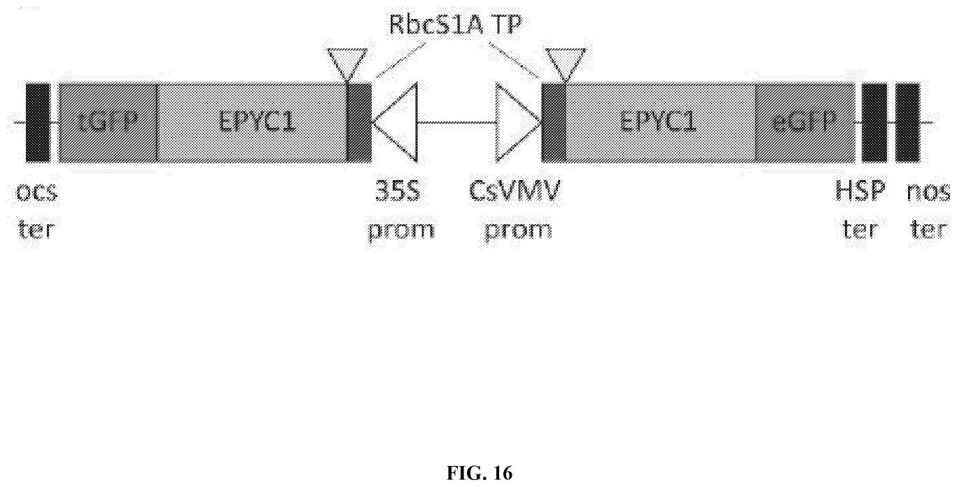

A schematic representation of the binary vector for dual GFP expression (EPYC1-dGFP) is shown in FIG. 16. The annotated full sequence of the EPYC1 expression cassettes is provided in SEQ ID NO: 171.

### Protein Analyses

Soluble protein was extracted from frozen leaf material of 21-d-old plants (sixth and seventh leaf) in protein extraction buffer (50 mM HEPES-KOH pH 7.5 with 17.4% glycerol, 2% Triton X-100 and cOmplete Mini EDTA-free Protease Inhibitor Cocktail (Roche, Basel, Switzerland). Samples were heated at 70° C. for 15 min with 1× Bolt LDS sample buffer (ThermoFisher Scientific, UK) and 200 mM DTT. Extracts were centrifuged and the supernatants subjected to SDS-PAGE on a 12% (w/v) polyacrylamide gel and transferred to a nitrocellulose membrane.

Membranes were probed with: rabbit serum raised against wheat Rubisco at 1:10,000 dilution (Howe, et al., PNAS (1982) 79: 6903-6907), rabbit serum raised against the SSU RbcS2 from C. reinhardtii (CrRbcS2) (raised to the C-terminal region of the SSU (KSARDWQPANKRSV (SEQ ID NO: 172)) by Eurogentec, 205 Southampton, UK) at 1:1,000 dilution, anti-Actin antibody (beta Actin Antibody 60008-1-Ig from Proteintech, UK) at 1:1000 dilution, and/or an anti-EPYC1 antibody at 1:2,000 dilution (Mackinder, et al., PNAS (2016) 113: 5958-5963 doi:10.1073/pnas.1522866113), followed by IRDye 800CW goat anti-rabbit IgG (LI-COR Biotechnology, Cambridge, UK) at 1:10,000 dilution, and visualized using the Odyssey CLx imaging system (LI-COR Biotechnology).

### Condensate Extraction

Soluble protein was extracted as described above in the “Protein analyses” section, then filtered through Miracloth (Merck Millipore, Burlington, Mass., USA), and centrifuged at 500 g for 3 min at 4° C., as in Mackinder et al. (PNAS 113: 5958-5963 (2016)). The pellet was discarded, and the extract centrifuged again for 12 min. The resulting pellet was washed once in protein extraction buffer, then re-suspended in a small volume of buffer and centrifuged again for 5 min. Finally, the pellet was re-suspended in 25 μl of extraction buffer and used in confocal analysis or SDS-PAGE electrophoresis as described below.

### Growth Analysis and Photosynthetic Measurements

Rosette growth rates were quantified using the imaging system described in Dobrescu et al. (Plant methods 13, 95 (2017)). Maximum quantum yield of photosystem II (PSII) (Fᵥ/Fₘ) was measured on 32-day-old plants using a Hansatech Handy PEA continuous excitation chlorophyll fluorimeter (Hansatech Instruments Ltd, King's 222 Lynn, UK) (Maxwell and Johnson, J Exp Bot 412 51, 659-668 (2000)).Gas exchange and chlorophyll fluorescence were determined using a LI-COR LI-6400 (LI-COR, Lincoln, Nebr., USA) portable infra-red gas analyzer with a 6400-40 leaf chamber on either the sixth or seventh leaf of 35- to 45-day-old non-flowering rosettes grown in large pots under 200 μmol photons m⁻² s⁻¹ to generate leaf area sufficient for gas exchange measurements as in Flexas et al. (New Phytologist 175, 501-511, doi:10.1111/j.1469-8137.2007.02111.x (2007)). The response of net CO₂ assimilation (A) to the intercellular CO₂ concentration (Cᵢ) was measured at 50, 100, 150, 200, 250, 300, 350, 400, 600, 800, 1000, and 1200 μmol mol⁻¹ CO₂ under saturating light (1,500 μmol photons m⁻² s⁻¹). For all gas exchange experiments, the flow rate was kept at 200 μmol mol⁻¹, leaf temperature was controlled at 25° C. and approximately 70% relative humidity was maintained inside the chamber. Measurements were performed after net assimilation and stomatal conductance had reached steady state. Gas exchange data were corrected for CO₂ diffusion from the measuring chamber as in Bellasio et al (Plant Cell Environ 39, 1180-1197, doi:10.1111/pce.12560 (2015)). The means±standard error of the mean (SEM) shown in Table 7, below, are from measurements made on seven 35- to 45-day-old rosettes for gas exchange variables, or on twelve 32-day-old rosettes for Fᵥ/Fₘ. The Fᵥ/Fₘ values shown in Table 7, below, are for attached leaves that had been dark-adapted for 45 minutes prior to fluorescence measurements.

To estimate the maximum rate of Rubisco carboxylation (Vₘₐₓ), the maximum electron transport rate (Jₘₐₓ), the net CO₂ assimilation rate at ambient concentrations of CO₂ normalized to Rubisco (A_{Rubisco}), the CO₂ compensation point (F), and the mesophyll conductance to CO₂ (conductance of CO₂ across the pathway from intercellular airspace to chloroplast stroma; gₘ), the A/Cᵢ data were fitted to the C₃ photosynthesis model as in Ethier and Livingston (Plant Cell Environ 27, 137-153, doi:10.1111/j.1365-3040.2004.01140.x (2004)) using the catalytic parameters K_{c}^{air} and affinity for O₂ (KO values for wild-type A. thaliana Rubisco at 25° C. and the Rubisco content of WT and S2_{Cr} lines (Atkinson, N. et al. New Phytol 214, 655-667, doi:10.1111/nph.14414 (2017)). gₘ was measured as in Ethier and Livingston (Plant Cell Environ 27, 137-153, doi:10.1111/j.1365-3040.2004.01140.x (2004)) and Diamos, et al. (Plant Biotech J 16, 1971-1982, doi:10.1111/pbi.12931 (2018)).

### Confocal Laser Scanning and Super-Resolution Image Microscopy

Leaves were imaged with a Leica TCS SP8 laser scanning confocal microscope (Leica Microsystems, Milton Keynes, UK) as in Atkinson et al. (Plant Biotech J 14, 1302-1315, doi:10.1111/pbi.12497 (2016)). Image processing was done with Leica LAS AF Lite software. Condensate and chloroplast dimensions were measured from confocal images using Fiji (ImageJ, v1.52n) (Schindelin et al., Nature Methods 9, 676-682, doi:10.1038/nmeth.2019 (2012)). Condensate volume was calculated as a sphere. Chloroplast volume was calculated as an ellipsoid in which depth was estimated as 25% of the measured width. Chloroplast volumes varied between 24-102 μm³, which was within the expected size range and distribution for A. thaliana chloroplasts (Crumpton-Taylor et al., Plant Phys 158, 905-916, doi:10.1104/pp. 111.186957 (2012)). Comparative pyrenoid area measurements were performed using Fiji on TEM cross-section images of WT C. reinhardtii cells (cMJ030) as described in Itakura et al. (PNAS 116, 18445-18454, doi:10.1073/pnas.1904587116 (2019)).

Super-resolution images were acquired using structured illumination microscopy. Samples were prepared on high precision cover-glass (Zeiss, Jena, Germany). 3D SIM images were acquired on an N-SIM (Nikon Instruments, UK) using a 100×1.49NA lens and refractive index matched immersion oil (Nikon Instruments). Samples were imaged using a Nikon Plan Apo TIRF objective (NA 1.49, oil immersion) and an Andor DU-897X-5254 camera using a 488 nm laser line. Z-step size for z stacks was set to 0.120 μm as required by manufacturer's software. For each focal plane, 15 images (5 phases, 3 angles) were captured with the NIS-Elements software. SIM image processing, reconstruction, and analyses were carried out using the N-SIM module of the NIS-Element Advanced Research software. Images were checked for artefacts using the SIMcheck software (http://www.micron.ox.ac.uk/software/SIMCheck.php). Images were reconstructed using NiS Elements software v4.6 (Nikon Instruments) from a z stack comprising of no less than 1 μm of optical sections. In all SIM image reconstructions, the Wiener and Apodization filter parameters were kept constant.

### Immunogold Labelling and Electron Microscopy

Leaf samples were taken from 21-day-old S2_{Cr} plants and S2_{Cr} transgenic lines expressing EPYC1-dGFP, and fixed, prepared, and sectioned as described in Example 3 above. Blocked grids were incubated overnight with anti-Rubisco antibody in TBSTT at 1:250 dilution or anti-CrRbcS2 antibody at 1:50 dilution, and washed twice each with TBSTT and water. Incubation with 15 nm gold particle-conjugated goat anti-rabbit secondary antibody (Abcam, Cambridge, UK) in TBSTT was carried out for 1 hr at 1:200 dilution for Rubisco labelling or 1:10 for CrRbcS2 labelling, before washing as described above in Example 3. Staining, viewing, and image collection were performed as described above in Example 3.

### Statistical Analyses

Results were subjected to analysis of variance (ANOVA) to determine the significance of the difference between sample groups. When ANOVA was performed, Tukey's honestly significant difference (HSD) post-hoc tests were conducted to determine the differences between the individual treatments (IBM SPSS Statistics Ver. 26.0, Chicago, Ill., USA).

### Results

Dual-GFP-Tagged Truncated EPYC1 Expressed in S2_{Cr} Transgenic A. thaliana Plants Underwent Less Proteolytic Degradation

EPYC1 was truncated according to the predicted transit peptide cleavage site between residues 26 (V) and 27 (A) (Atkinson et al., J Exp Bot 70, 5271-5285, doi:10.1093/jxb/erz275 (2019)). A dual GFP expression system (FIG. 16) was developed to achieve high levels of EPYC1 expression and a favorable stoichiometry with Rubisco. This consisted of a binary vector containing two gene expression cassettes, each encoding truncated EPYC1 with an A. thaliana chloroplastic signal peptide and fused to a different version of GFP (turboGFP (tGFP) or enhanced GFP (eGFP)) to reduce the changes of recombination events. The annotated full sequence of the EPYC1 expression cassettes is provided in SEQ ID NO: 171.

The dual GFP construct (EPYC1-dGFP) was transformed into WT plants or into the A. thaliana 1a3b Rubisco mutant complemented with a Rubisco SSU from C. reinhardtii (S2_{Cr}). The resulting transgenic plants (three lines, termed Ep1, Ep2, and Ep3, respectively) expressed both EPYC1::eGFP and EPYC1::tGFP, of which the latter was generally more highly expressed (FIG. 17).

In Example 2 above and in Atkinson et al. (J Exp Bot 70, 5271-5285, doi:10.1093/jxb/erz275 (2019)), immunoblots against full length EPYC1 expressed using other constructs in S2_{Cr} or WT plants showed additional lower molecular weight bands indicative of proteolytic degradation (FIG. 8A). In contrast, expression of mature EPYC1 resulted in reduced levels of degradation products (as indicated by lower-weight bands) when the EPYC1-dGFP construct was expressed in S2_{Cr} compared to WT plants (FIG. 17).

EPYC1-dGFP Expression in S2_{Cr} and 1A_{At}/MOD A. thaliana Backgrounds Caused Condensate Formation in the Chloroplast Stroma

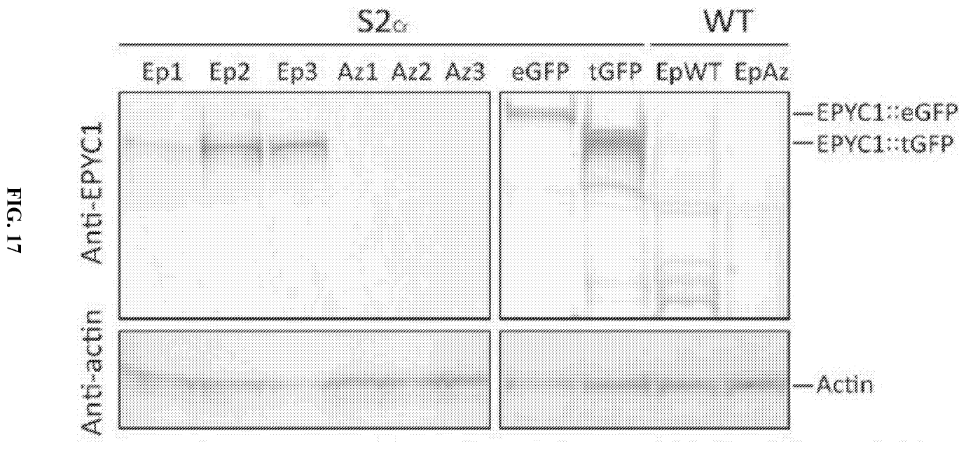

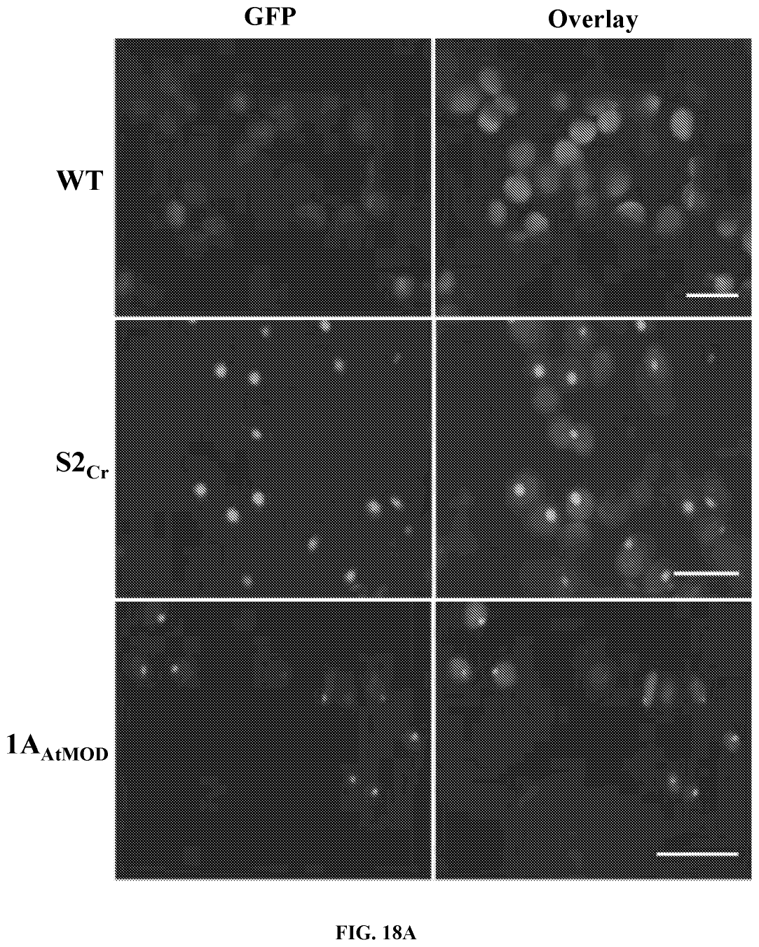

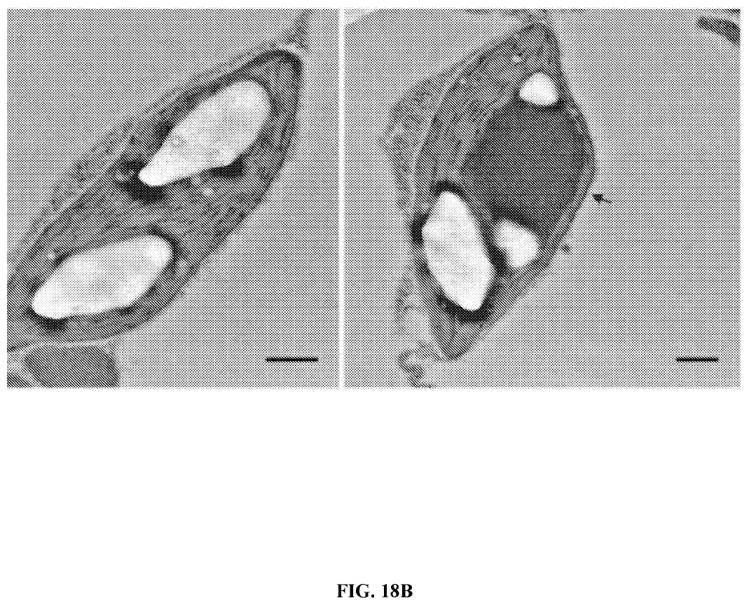

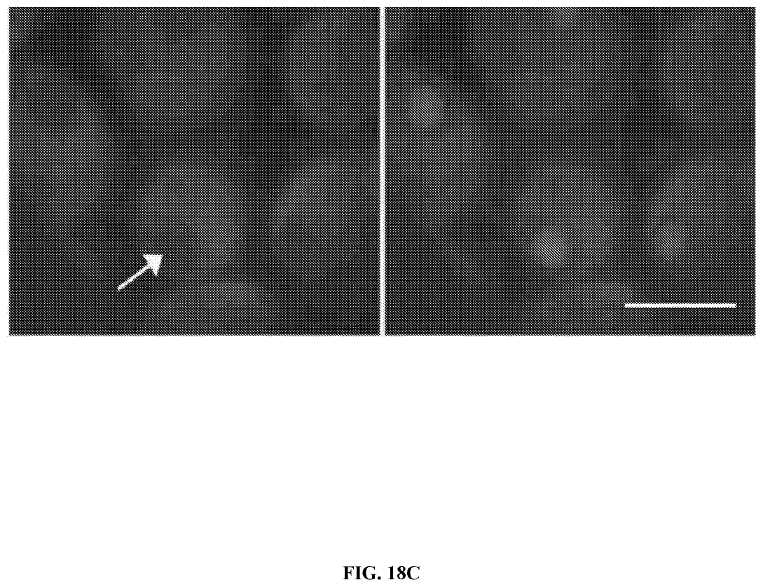

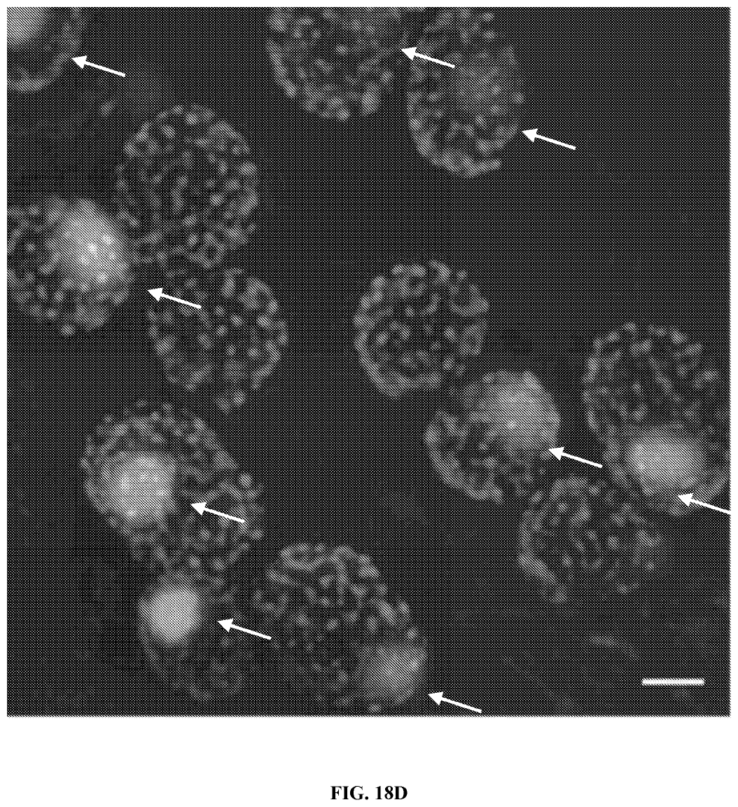

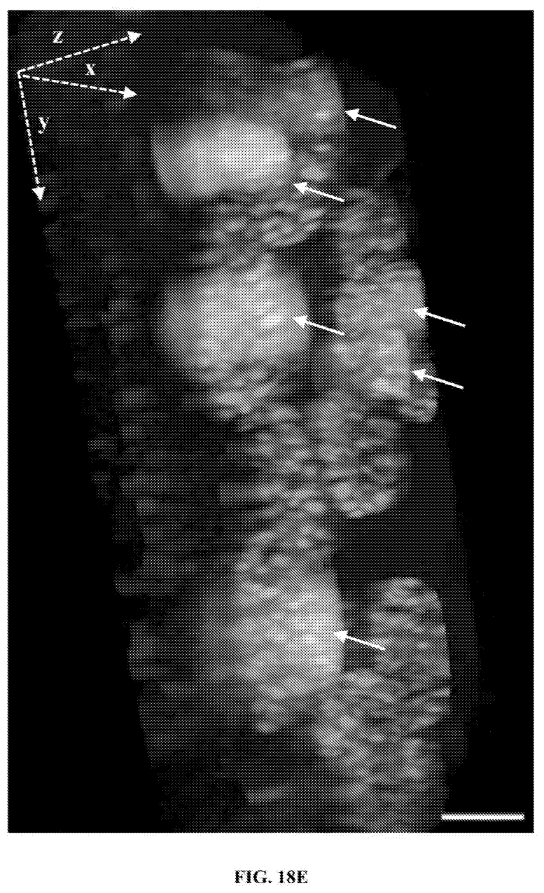

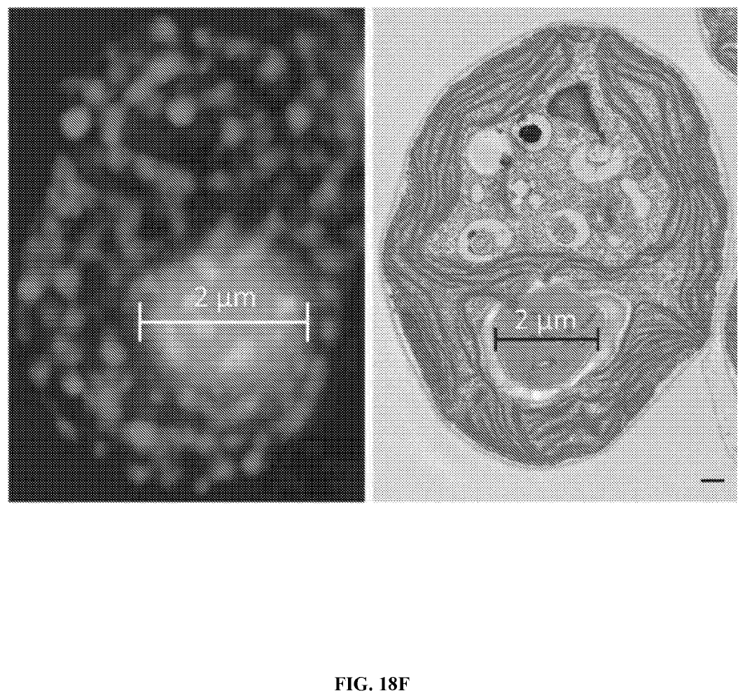

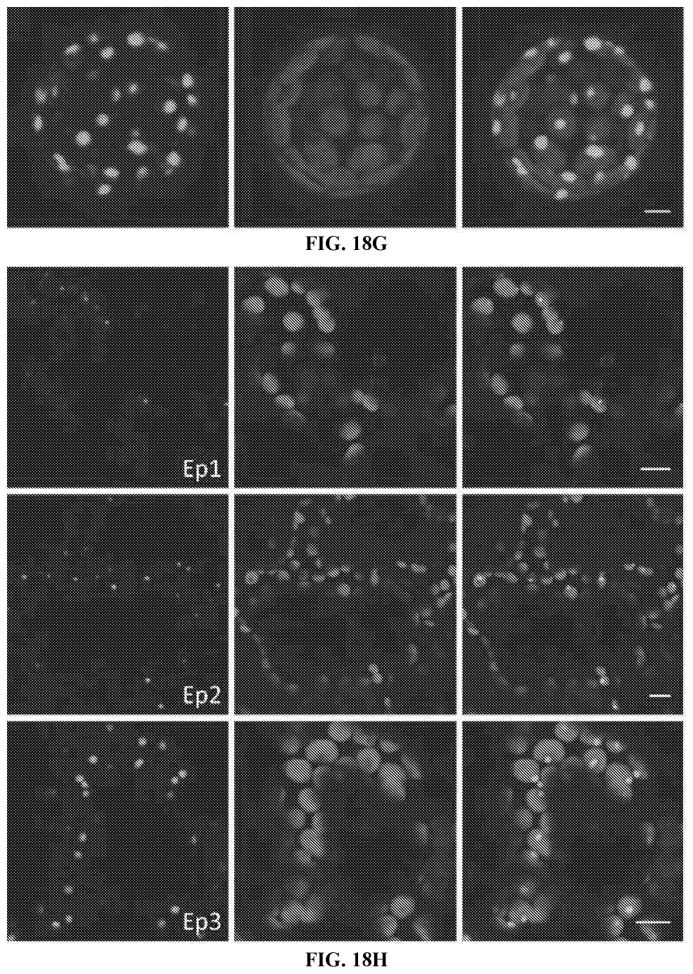

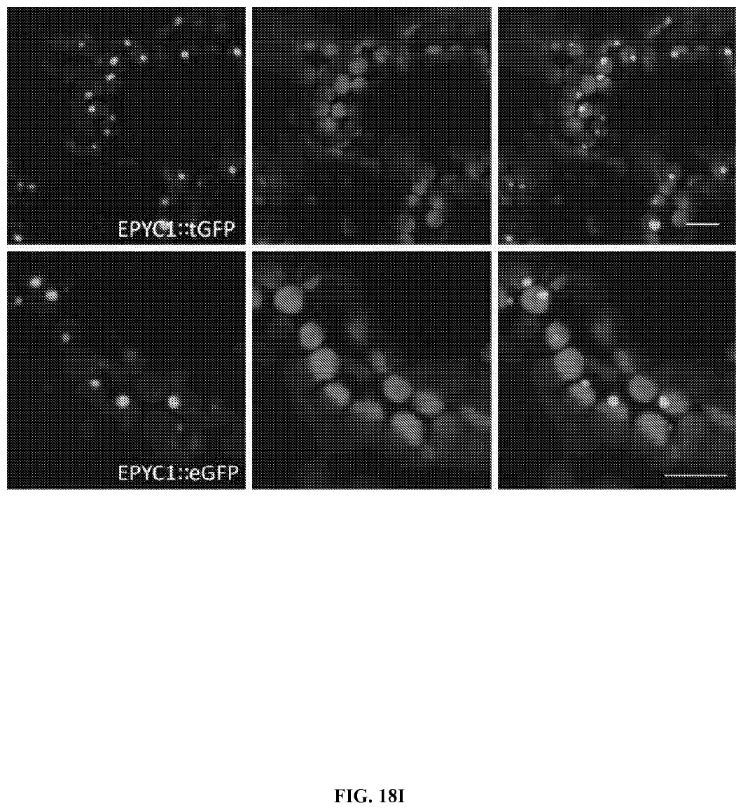

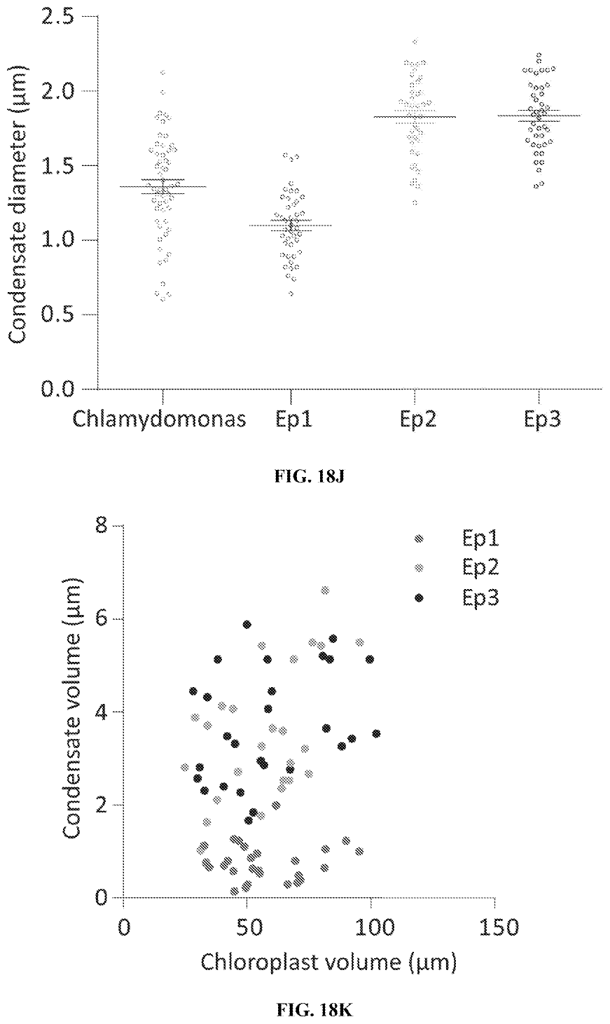

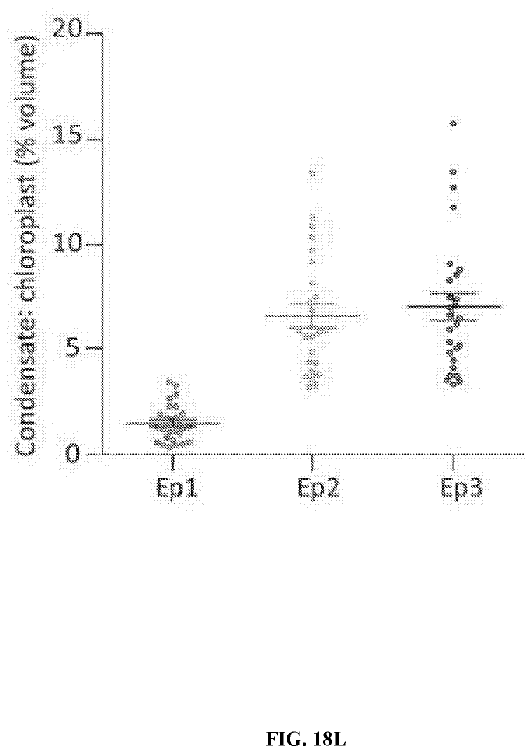

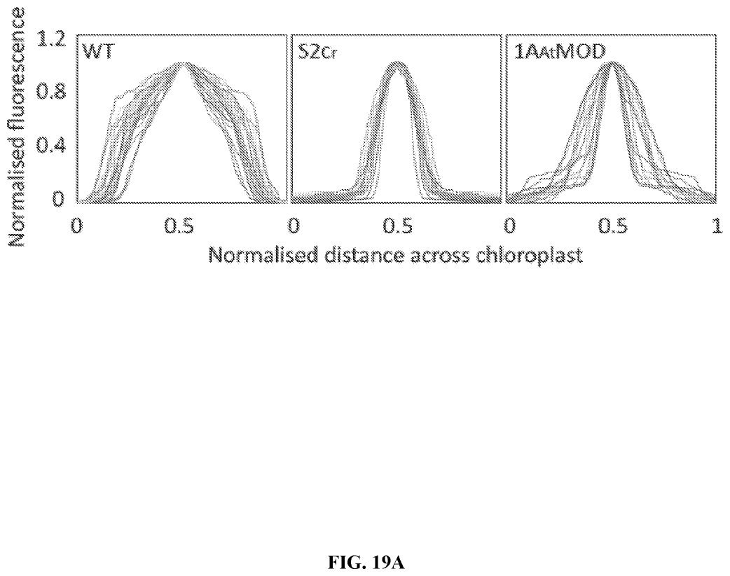

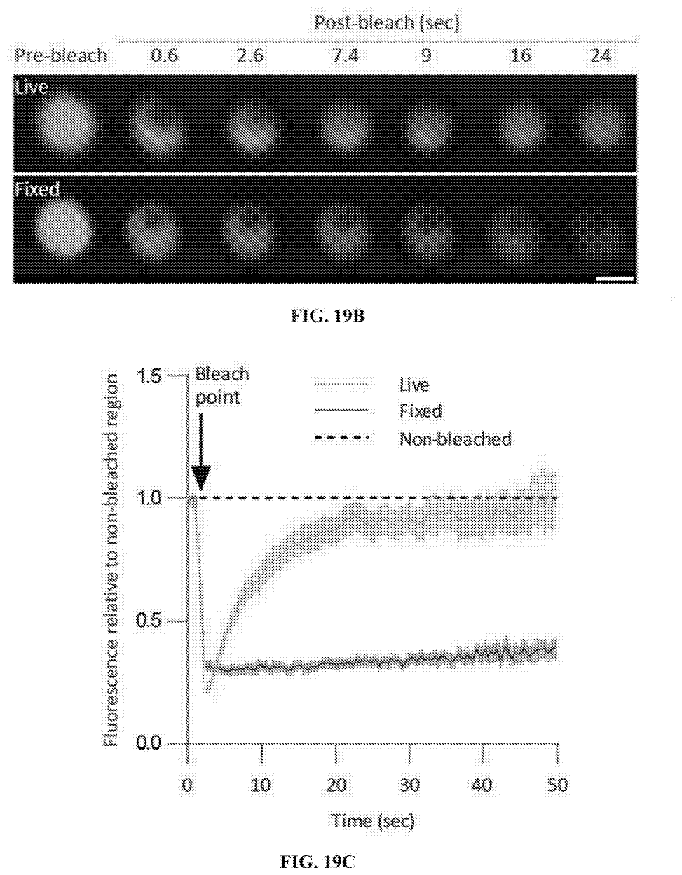

The fluorescence signal for EPYC1-dGFP in WT plants was distributed evenly throughout the chloroplast (FIG. 18A, top row; FIG. 19A, left panel). In contrast, EPYC1-dGFP in the hybrid S2_{Cr} plants showed only a single dense chloroplastic signal (FIG. 18A, middle row; FIG. 19A, middle panel). Transmission electron microscopy confirmed the presence of a single prominent condensed complex in the chloroplast stroma (FIG. 18B). The condensates were spherical in shape and displaced native chlorophyll autofluorescence (FIGS. 18C-18E), indicating that the thylakoid membrane matrix was excluded from the condensate. In protoplasts of leaf mesophyll cells, a condensate was visible in each chloroplast (FIG. 18G), and the average size of the condensates was related to the expression level of EPYC1-dGFP (FIGS. 17, 18H, 18J-18L).

The average diameter of the condensates was 1.6±0.1 μm (n=126; 42 each from three individual S2_{Cr} transgenic lines) (FIGS. 18F, 18J), which was comparable to the measured size range of the C. reinhardtii pyrenoid (1.4±0.1 μm; n=55) (Itakura et al., PNAS 116, 18445-18454, doi:10.1073/pnas.1904587116 (2019)). The estimated volume of the condensates was 2.7±0.2 μm³ (approximately 5% of the chloroplast volume) (FIGS. 18K-18L). Variations in condensate volume within individual S2_{Cr} transgenic Ep lines were not correlated with chloroplast volume (FIGS. 18K-18L), suggesting that regulation of condensate formation and size was largely independent of chloroplast morphology.

Condensates were also observed when EPYC1-dGFP was expressed in the A. thaliana 1a3b Rubisco mutant complemented with a native A. thaliana SSU modified to contain the two α-helices necessary for pyrenoid formation from the Rubisco small subunit from C. reinhardtii (1A_{At}MOD) (FIG. 18A, bottom row). However, condensates in the 1A_{At}MOD background were less punctate (FIG. 19A, right panel), which was consistent with the lower affinity of the modified native Rubisco SSU for EPYC1 observed in yeast two-hybrid experiments (FIGS. 2A-2C, 3C, 5E) (Atkinson et al., J Exp Bot 70, 5271-5285, doi:10.1093/jxb/erz275 (2019)). Condensate formation in the 1A_{At}MOD background (FIGS. 18A, 19A), in which catalytic characteristics of the hybrid Rubisco were indistinguishable from that of WT Rubisco (Atkinson et al., New Phytol 214, 655-667, doi:10.1111/nph.14414 (2017)), indicated that the SSU can be further engineered to optimize phase separation, Rubisco content and performance.

Furthermore, visible condensates formed when either EPYC1::tGFP or EPYC1::eGFP expression cassettes were individually transformed into the S2n-A. thaliana background (FIG. 18I).

In Example 2 above, expression of a full length (i.e., non-truncated) variant of EPYC1-dGFP in A. thaliana chloroplasts did not result in phase separation (FIG. 7C; Atkinson et al., J Exp Bot 70, 5271-5285, doi:10.1093/jxb/erz275 (2019)), which was attributed to low levels of expression and an incompatible stoichiometry between EPYC1 and Rubisco, and possible proteolytic degradation. In contrast, the results of this Example indicate that condensate formation may depend more on expression of a mature EPYC1 variant than on the level of EPYC1 expression per se. This Example also showed that the stoichiometry between EPYC1 and Rubisco required for condensate formation was achievable in higher plants. Furthermore, the apparent reduction in proteolytic degradation of EPYC1 observed in the results of this Example (FIG. 17) may be caused by sequestration of EPYC1 within a phase-separated compartment, as these compartments are hypothesized to be less accessible to large protease complexes (van der Hoorn and Rivas, New Phytol 218, 879-881, doi:10.1111/nph.15156 (2018)).

### The Condensates Exhibit Liquid-Like Characteristics

Fluorescence recovery after photobleaching (FRAP) assays were conducted on condensates in live S2_{Cr}-A. thaliana leaf cells expressing EPYC1-dGFP to test for the presence of internal mixing characteristics consistent with the liquid-like behavior of pyrenoids. Condensates recovered full fluorescence 20-40 seconds after photobleaching (FIGS. 19B-19C). This indicated that the EPYC1-dGFP molecules in A. thaliana condensates mix at similar or increased rates compared to previous in vitro (Wunder et al., Nat Commun 9, 5076, doi:10.1038/s41467-018-07624-w (2018)) and in alga (Freeman Rosenzweig et al., Cell 171, 148-162, doi:10.1016/j.cell.2017.08.008 (2017)) reports. It is thought that the more rapid interchange in transgenic A. thaliana condensates compared to C. reinhardtii pyrenoids may be due to a relatively reduced availability of EPYC1 binding sites on Rubisco in the S2_{Cr} plant-algal hybrid Rubisco background compared to that in C. reinhardtii (Mackinder, et al., PNAS (2016) 113: 5958-5963; Freeman Rosenzweig et al., Cell 171, 148-162, doi:10.1016/j.cell.2017.08.008 (2017)). In contrast, condensates in leaf tissue chemically cross-linked with formaldehyde showed no recovery after photobleaching (FIGS. 19B-19C), which was consistent with that observed in C. reinhardtii pyrenoids (Freeman Rosenzweig et al., Cell 171, 148-162, doi:10.1016/j.cell.2017.08.008 (2017)).

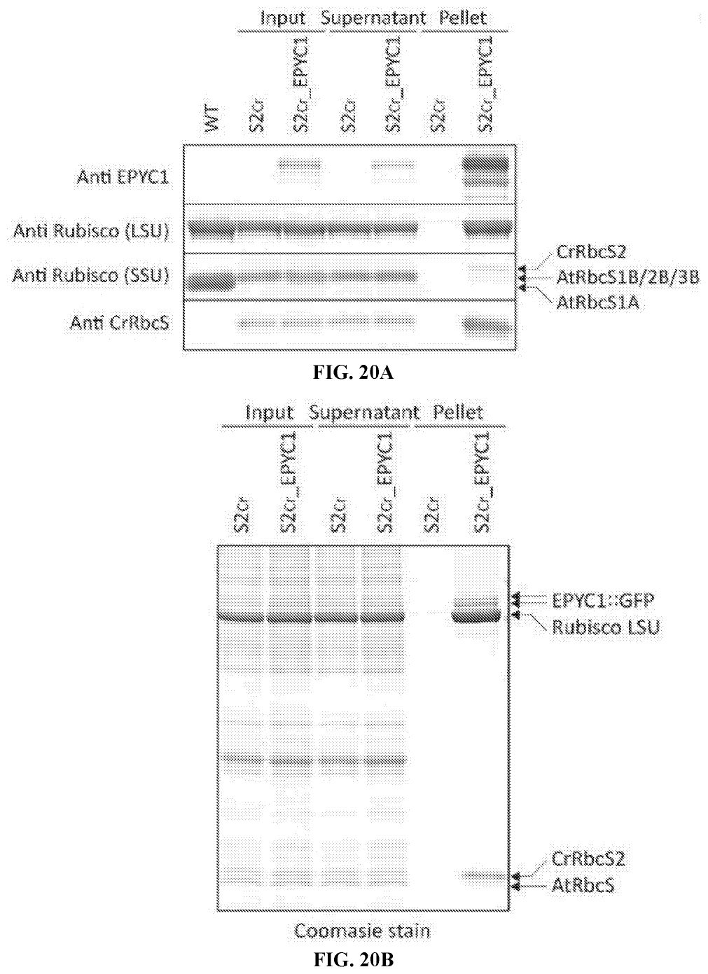

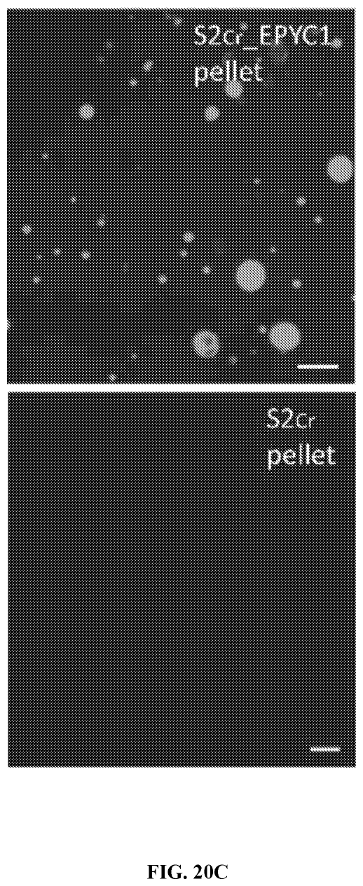

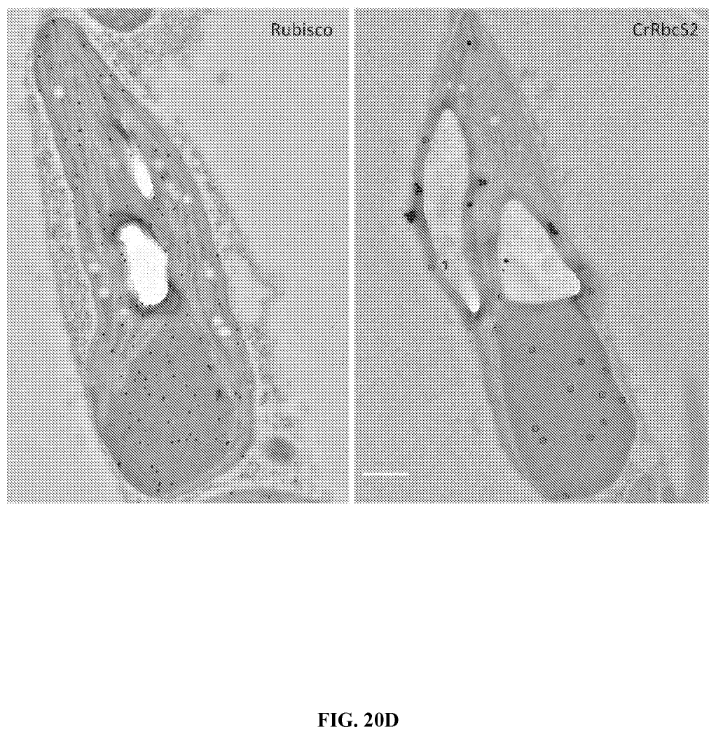

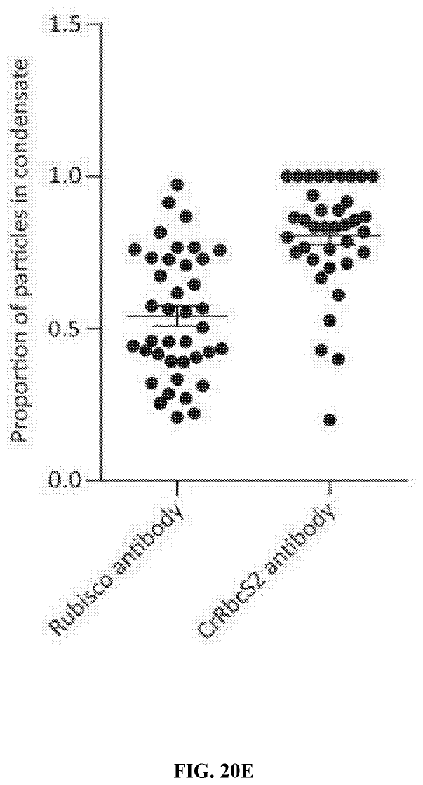

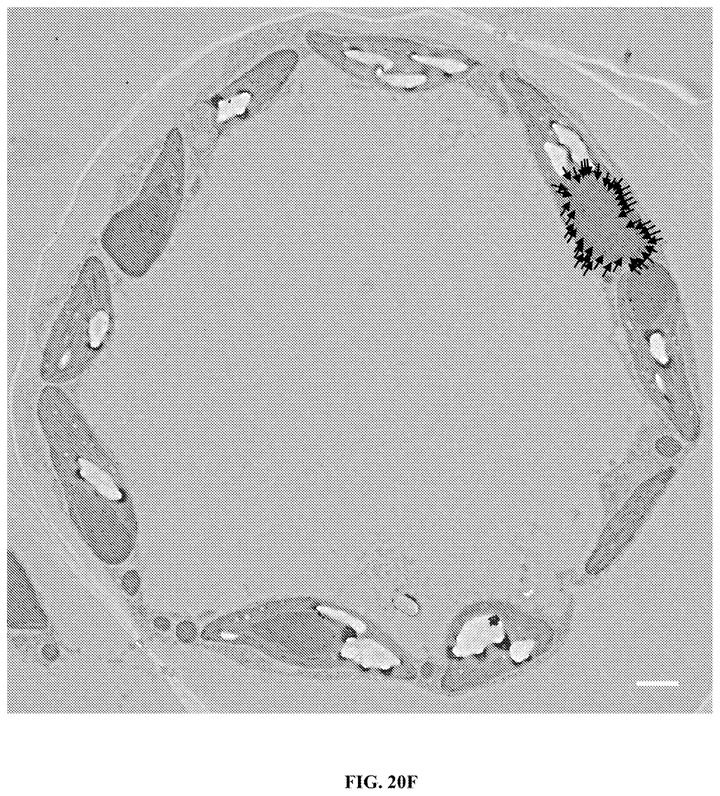

Further, condensates that were extracted from S2_{Cr} A. thaliana plants expressing EPYC1-dGFP and then resuspended in vitro coalesced into larger droplets (FIG. 20C). Droplet formation is a liquid-like behavior known to be associated with EPYC1-Rubisco interactions in vitro (Wunder et al., Nat Commun 9, 5076, doi:10.1038/s41467-018-07624-w (2018)).

Condensates in A. thaliana Chloroplasts Expressing EPYC1-dGFP are Enriched in EPYC1-dGFP and Rubisco

To test for the presence of Rubisco, condensates were extracted from A. thaliana leaf tissue by gentle centrifugation and examined by immunoblot. Isolated condensates (pellet fraction) from S2_{Cr} A. thaliana plants expressing EPYC1-dGFP were shown to be enriched in EPYC1-dGFP and both the large and small subunits of Rubisco (FIG. 20A).

Regarding the Rubisco SSU, the Western shown in FIG. 20A provided qualitative evidence that isolated condensates were enriched in the C. reinhardtii SSU compared to native A. thaliana SSUs (i.e., increase in C. reinhardtii SSU (CrRbcS) vs. decrease in native A. thaliana SSU (AtRbcS)). Subsequent Coomasie staining of denatured, gel-separated extracts was used to generate quantitative differences (in percentage) between total S2_{Cr} soluble protein extract and the condensate enriched pellet. This revealed that nearly half (49%) of Rubisco in the initial extract contained C. reinhardtii SSU, while 82% of Rubisco in the pelleted condensate contained C. reinhardtii SSU (FIG. 20B).

Consistent with the Coomasie staining, immunogold analysis of TEM images of chloroplasts from S2_{Cr} expressing EPYC1-dGFP (FIGS. 20D, 20F) showed that approximately half (54%) of Rubisco localized to the condensate (when assessed with a polyclonal Rubisco antibody with a greater specificity for higher plant LSU and SSUs than for C. reinhardtii LSU and SSUs), while 81% of the C. reinhardtii SSU localized to the condensate (FIG. 20E). Thus, condensation of Rubisco was strongly associated with Rubisco complexes bearing the C. reinhardtii SSU, which constituted approximately 50% of the Rubisco SSU pool in the A. thaliana S2_{Cr} background (FIGS. 20A-20B). The latter is consistent with the expected expression levels of plant-algal hybrid Rubisco in S2Cr (Atkinson et al., New Phytol 214, 655-667, doi:10.1111/nph.14414 (2017)).

EPYC1-dGFP Expression in A. thaliana does not Impair Growth

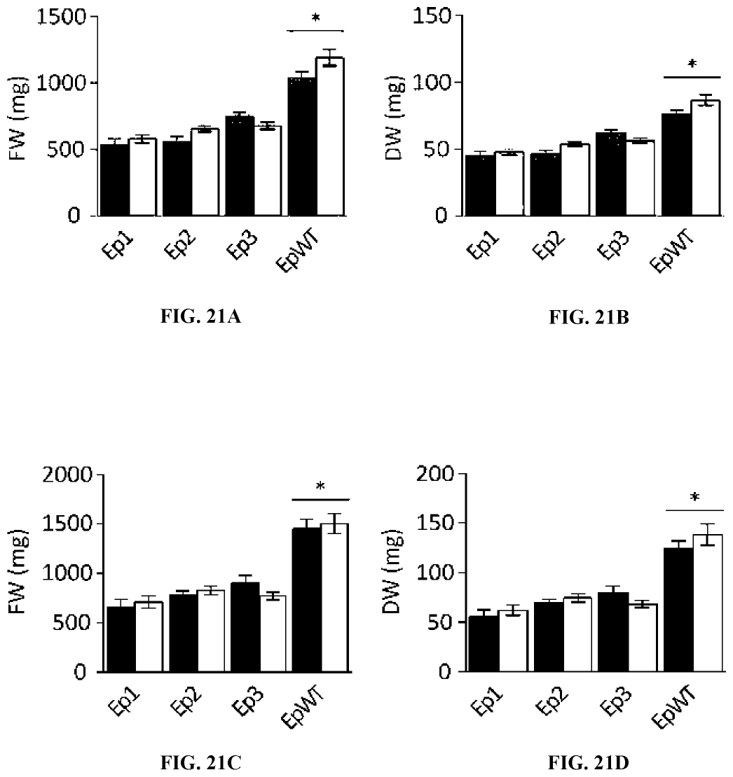

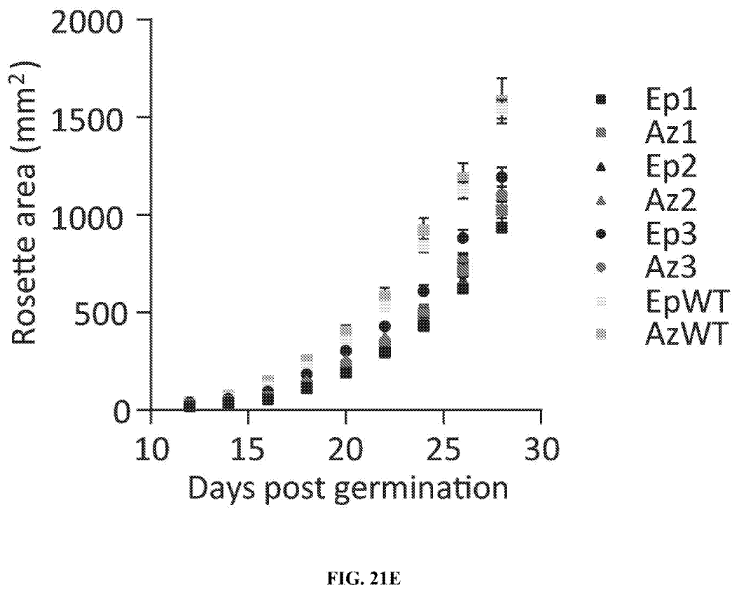

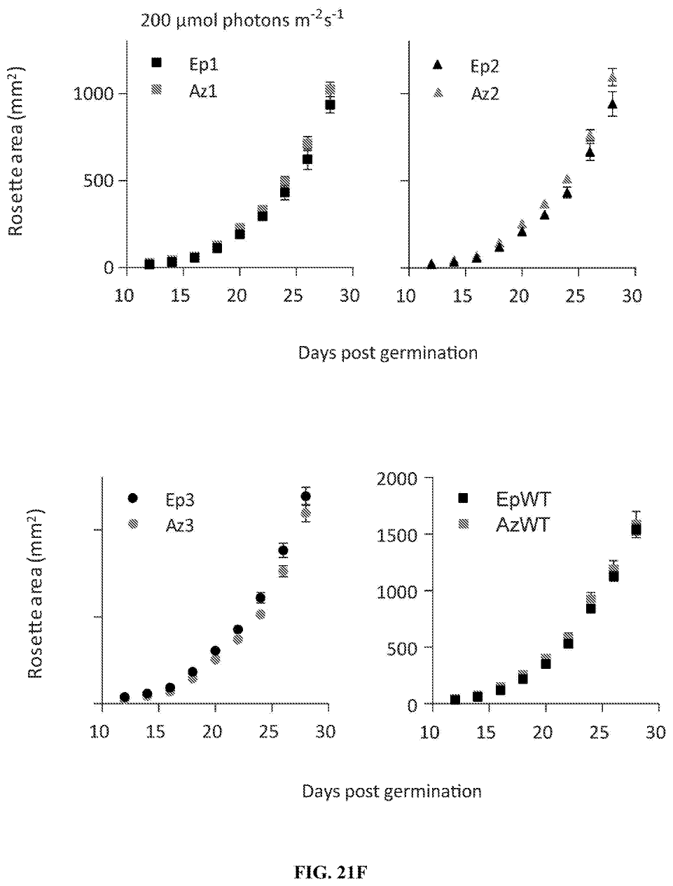

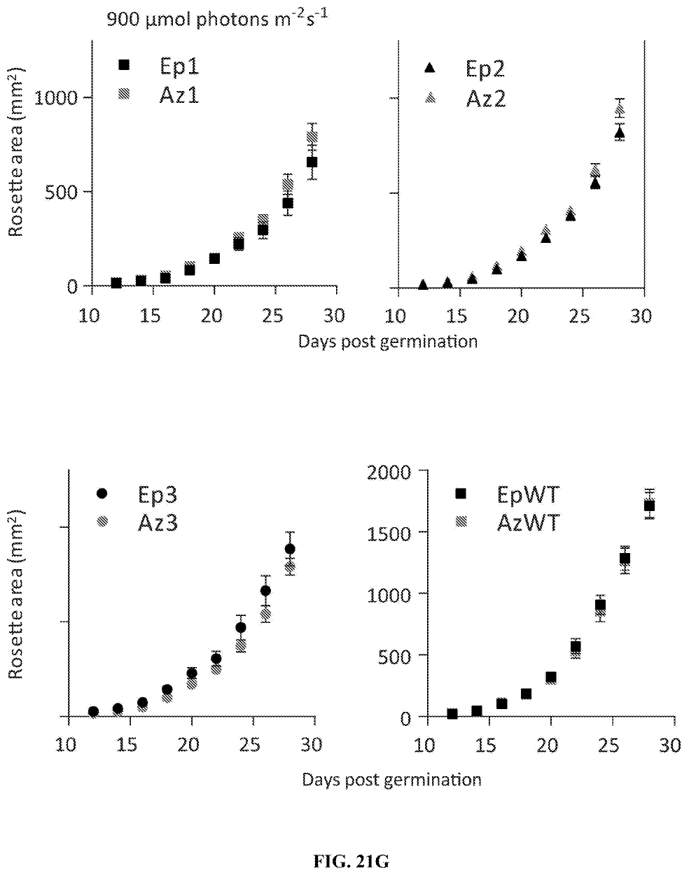

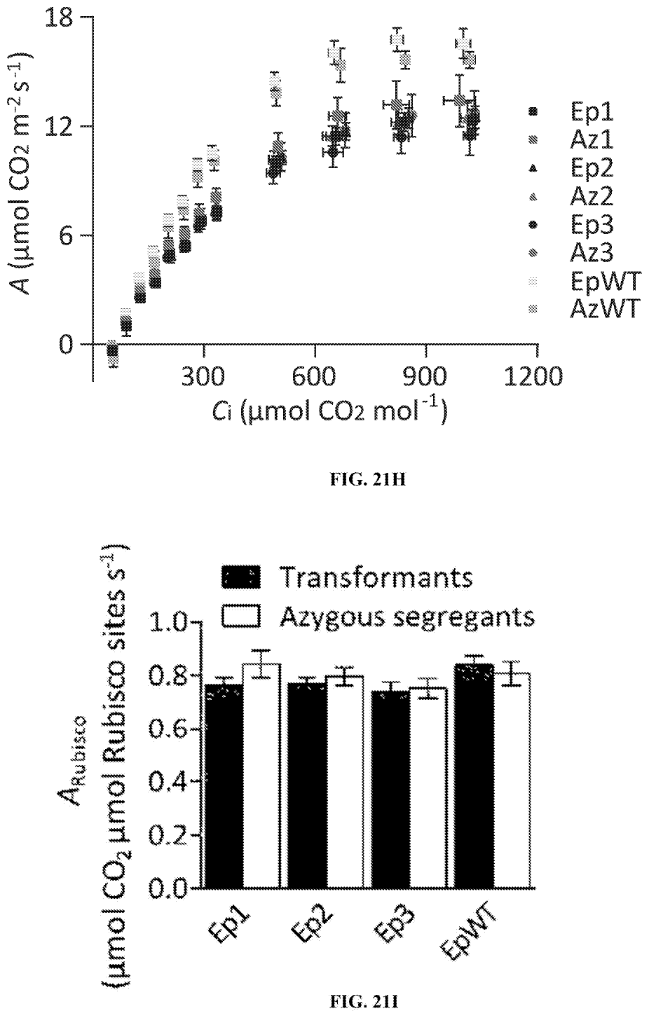

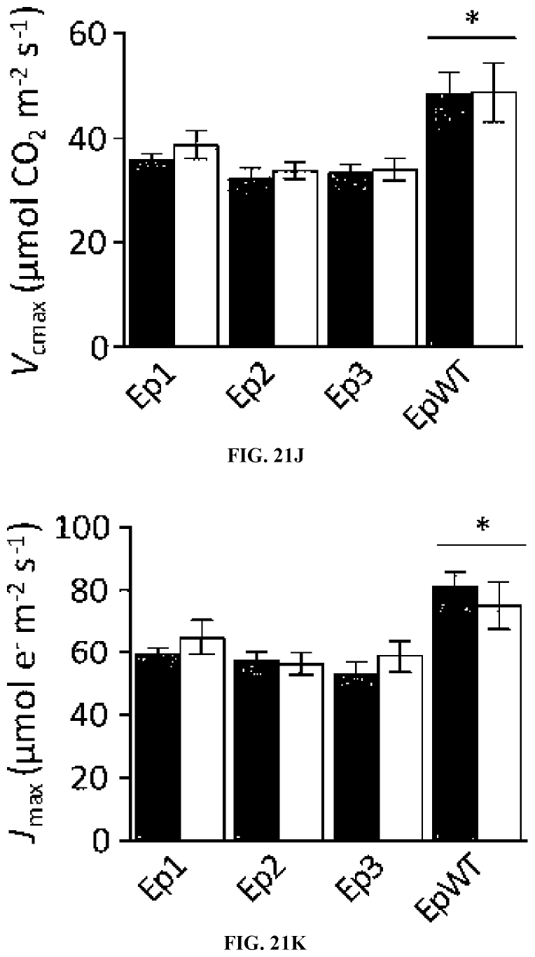

Growth comparisons were conducted on three separate T2 EPYC1-dGFP S2Cr transgenic lines (Ep1-3), which had been screened for the presence of condensates, and their respective T2 azygous segregant S2Cr lines (Az1-3). Growth was assessed after cultivation under two different light levels: those typical for A. thaliana growth (200 μmol photons m⁻² s⁻¹) (FIGS. 21A-21B, 21E-21F), and higher than typical light levels (900 μmol photons m⁻² s⁻¹) (FIGS. 21C-21D, 21G). Previous studies have shown that plant growth is more limited by Rubisco activity under 900 μmol photons m⁻² s⁻¹ than under 200 μmol photons m⁻² s⁻¹ (Lauerer et al., Planta 190, 332-345, doi:10.1007/bf00196962 (1993)).

Regardless of the growth conditions, rosette expansion rates or biomass accumulation were not distinguishable between S2_{Cr} transformants and their segregant controls (FIGS. 21A-21G). Similarly, T2 EPYC1-dGFP WT plants (EpWT) showed no significant differences compared to T2 segregant lines (AzWT) (FIGS. 21A-21G). The performance of the S2_{Cr} lines was slightly decreased compared to WT plants (FIGS. 21A-21E), which was thought to be due to the reduced Rubisco content in the S2_{Cr} background (Atkinson et al., New Phytol 214, 655-667, doi:10.1111/nph.14414 (2017)). The observed differences in growth between the S2_{Cr} and WT lines were in line with those reported previously for S2_{Cr} and WT plants in the absence of EPYC1 (Atkinson et al., New Phytol 214, 655-667, doi:10.1111/nph.14414 (2017)).

EPYC1-dGFP Expression in A. thaliana does not Impair Photosynthesis

Photosynthetic parameters derived from response curves of CO₂ assimilation rate to the intercellular CO₂ concentration under saturating light were similar between respective EPYC1-dGFP-expressing and azygous segregant lines (FIGS. 21H-21K; Table 7, below). The presence of condensates did not influence the maximum achievable rates of Rubisco carboxylation (V_{cmax}; FIG. 21J; Table 7, below).

Table 7 shows photosynthetic parameters derived from gas exchange and fluorescence measurements for S2_{Cr} and WT transgenic lines of A. thaliana. The mean and standard error of the mean (SEM) are shown for seven 35- to 45-day-old rosettes for gas exchange variables, and for twelve 32-day-old rosettes for the maximum potential quantum efficiency of photosystem II (Fᵥ/Fₘ). Fᵥ/Fₘ is shown for attached leaves dark-adapted for 45 minutes prior to fluorescence measurements. Letters after the SEM indicate significant difference within the data in the same row (P<0.05) as determined by ANOVA followed by Tukey's HSD tests. Values followed by the same letter within a row are not statistically significantly different from each other. Terms are abbreviated as follows: V_{cmax} is the maximum rate of Rubisco carboxylation, measured in μmol CO₂ m⁻²s⁻¹; Jₘₐₓ is the maximum electron transport rate, measured in μmol e⁻m⁻² s⁻¹); F is the CO₂ compensation point, measured in μmol CO₂ m-2 s-1 and calculated as Cᵢ−A; gₛ is stomatal conductance to water vapor, measured in mol H₂O m⁻²s⁻¹; gₘ is mesophyll conductance to CO₂ (i.e., the conductance of CO₂ across the pathway from intercellular airspace to the chloroplast stroma), measured in mol CO₂ m⁻²s⁻¹; Fᵥ/Fₘ is the maximum potential quantum efficiency of photosystem II; ML denotes measurements taken under medium light (200 μmol photons m⁻²s⁻¹); HL denotes measurements taken under high light (900 μmol photons m⁻²s⁻¹); Ep1, Ep2, and Ep3 are the same three T2 EPYC1-dGFP S2_{Cr} transgenic lines shown in the other Figures in this Example; Az1, Az2, Az3 are the respective azygous segregants of Ep1-3; EpWT is an EPYC1-dGFP WT transformant; AzWT is an azygous segregant of EpWT.

TABLE 7
Photosynthetic parameters for S2_{Cr} and WT A. thaliana lines
expressing EPYC1-dGFP and azygous segregants thereof.

| Parameter | Ep1 | Az1 | Ep2 | Az2 | Ep3 | Az3 | EpWt | AzWt |
|---|---|---|---|---|---|---|---|---|
| V_{cmax} | 35.6 ± | 36.4 ± | 32.2 ± | 33.6 ± | 33.1 ± | 33.8 ± | 44.9 ± | 43.3 ± |
| | 1.5 | 2.0 | 1.9 | 1.6 | 1.9 | 2.2 | 1.6 | 1.7 |
| | a | a | a | a | a | a | b | b |
| Jₘₐₓ | 59.2 ± | 61.9 ± | 57.2 ± | 56.1 ± | 52.9 ± | 58.6 ± | 76.4 ± | 74.9 ± |
| | 2.3 | 6.3 | 2.6 | 3.5 | 4.4 | 5.2 | 2.4 | 7.5 |
| | a | a | a | a | a | a | b | b |
| Γ | 63 ± | 53 ± | 52 ± | 54 ± | 53 ± | 56 ± | 51 ± | 64 ± |
| | 8 | 5 | 6 | 7 | 7 | 8 | 7 | 12 |
| | a | a | a | a | a | a | a | a |
| gₛ | 0.249 ± | 0.279 ± | 0.233 ± | 0.251 ± | 0.233 ± | 0.236 ± | 0.287 ± | 0.306 ± |
| | 0.031 | 0.051 | 0.017 | 0.015 | 0.021 | 0.016 | 0.018 | 0.011 |
| | a | a | a | a | a | a | a | a |
| gₘ | 0.034 ± | 0.035 ± | 0.032 ± | 0.033 ± | 0.034 ± | 0.032 ± | 0.045 ± | 0.046 ± |
| | 0.001 | 0.003 | 0.002 | 0.002 | 0.003 | 0.002 | 0.002 | 0.003 |
| | b | b | b | b | b | b | a | a |
| Fᵥ/Fₘ | 0.848 ± | 0.849 ± | 0.848 ± | 0.847 ± | 0.847 ± | 0.845 ± | 0.851 ± | 0.850 ± |
| (ML) | 0.002 | 0.002 | 0.001 | 0.001 | 0.002 | 0.002 | 0.002 | 0.001 |
| | a | a | a | a | a | a | a | a |
| Fᵥ/Fₘ | 0.852 ± | 0.845 ± | 0.850 ± | 0.855 ± | 0.846 ± | 0.849 ± | 0.850 ± | 0.852 ± |
| (HL) | 0.002 | 0.002 | 0.001 | 0.004 | 0.002 | 0.001 | 0.003 | 0.002 |
| | a | a | a | a | a | a | a | a |

Notably, the CO₂ assimilation rates at ambient concentrations of CO₂ for EPYC1-dGFP-expressing and azygous segregant lines were comparable to WT lines when normalized for Rubisco content (A_{Rubisco}; FIG. 21I). This suggested that the known modest reductions in Rubisco turnover rate (k_{cat}^{c}) and specificity (Scio) for the plant-algal hybrid Rubisco in S2_{Cr} compared to WT plants had only a mild impact on the efficiency of photosynthetic CO₂ assimilation, and that the observed differences in growth rates were more associated with the reduced levels of Rubisco in S2_{Cr} plants (Atkinson et al., New Phytol 214, 655-667, doi:10.1111/nph.14414 (2017)).

Mesophyll conductance (gₘ) levels were also reduced in all S2_{Cr} lines compared to WT plants (Table 7), which was consistent with the impact of reduced Rubisco content on gₘ observed in transplastomic tobacco (Galmes et al., Photosynth Res 115, 153-166, doi:10.1007/s11120-013-9848-8 (2013)).

Measurements of the maximum electron transport rate (Jₘₐₓ) and the maximum potential quantum efficiency of photosystem II (Fᵥ/Fₘ) were also indistinguishable between transformant and segregant lines (Table 7). Thus, the apparent displacement of the thylakoid membrane matrix by the condensates (FIG. 18C) had no apparent impact on the efficiency of the light reactions of photosynthesis.

The results described in this Example show that EPYC1 and specific residues on the SSU were sufficient to aggregate Rubisco into a single proto-pyrenoid condensate, and that this condensate had no apparent negative impact on plant growth. The overall photosynthetic performances of S2_{Cr} transgenic lines appeared unaffected by the condensate, which suggested that conditions inside higher plant chloroplasts were highly compatible with the presence of pyrenoid-type bodies. This data provides a platform for adding additional components of the algal biophysical carbon concentrating mechanism (CCM) to higher plants in order to create a “fully assembled” biophysical CCM. The data presented here is arguably the key step for the assembly of a pyrenoid-based CCM into plants that could increase crop yield potentials by >60% (McGrath and Long, Plant Phys 164, 2247-2261, doi:10.1104/pp. 113.232611 (2014); Long et al. in Sustaining Global Food Security: The Nexus of Science and Policy. (ed R. S. Zeigler) Ch. 9, (CSIRO Publishing, 2019); Price et al., Plant Phys 155, 20-26, doi:10.1104/pp. 110.164681 (2011)). Previously described approaches for engineering the cyanobacterial carboxysome-based CCM required engineering of the chloroplast-encoded Rubisco large subunit, an approach that is not currently feasible in major grain crops such as wheat and rice (Long et al., Nat Commun 9, doi:Artn 3570 10.1038/S41467-018-06044-0 (2018)). The results of this Example demonstrated that condensation of Rubisco was achievable through modification of the nuclear-encoded SSU, which is significantly more amenable to genetic modification.

### Example 5: TobiEPYC1 Will Stably Aggregate Rubisco into Pyrenoid-Like Structures in N. benthamiana Chloroplasts

The following example describes characterization of the molecular properties of the chloroplastic EPYC1 aggregates in TobiEPYC1 N. benthamiana lines. Further, it describes the impact of the EPYC1 aggregates on plant metabolism, when plants are grown under different light levels.

### Materials and Methods

Materials and Methods for Characterizing TobiEPYC1 N. benthamiana Lines

The materials and methods described in Examples 2, 3, and 4 are used to characterize TobiEPYC1 N. benthamiana lines.

The EPYC1 aggregates in the TobiEPYC1 N. benthamiana lines are characterized. In particular, the type of Rubisco present in the aggregate (i.e., the ratio of C. reinhardtii SSUs to native SSUs) is characterized. Further, the liquid-liquid like behavior of the aggregate is characterized (e.g., using FRAP analysis). In addition, the physical properties of the aggregate (e.g., shape/architecture/density) are characterized (e.g., by TEM/CryoEM). Moreover, the aggregates are isolated, and in the isolated aggregates, EPYC1 is characterized for cleavage/degradation and Rubisco content and activity are measured. The BiFC experiments described in Example 2 are also used to characterize the TobiEPYC1 lines. Instead of the BiFC system used in Example 2, a more stringent system based on tri-partite GFP (Liu et al., 2018 Plant Journal) is used.

The impact of the EPYC1 aggregates is characterized in plants of the TobiEPYC1 N. benthamiana lines grown under medium light levels and high (i.e., Rubisco-limiting) light levels. In particular, the leaf area, fresh weight, and dry weight is measured. Further, chlorophyll content, protein content, and total Rubisco content are measured. In addition, photosynthetic parameters are measured using fluorescence (e.g., Fv/Fm) and gas exchange analyses (e.g., A:Ci curves). Gas exchange and fluorescence are done with a LICOR 6400.

### Results

Immunogold and/or fluorescence co-localization data will show the presence of Rubisco in the EPYC1 chloroplast aggregates.

Immunogold and/or fluorescence co-localization data will estimating the relative distribution of Rubisco aggregates in chloroplasts vs. Rubisco aggregates throughout the stroma, and will show that there are more Rubisco aggregates in chloroplasts.

Fluorescence localization data will show that aggregates form when TobiEPYC1 is expressed in higher plants carrying different permutations of the Rubisco SSU (e.g., an A. thaliana SSU mutant background complemented with: the whole C. reinhardtii RbcS2; modified A. thaliana SSUs carrying the C. reinhardtii α-helices; modified A. thaliana SSUs carrying the C. reinhardtii α-helices and β-sheets; modified A. thaliana SSUs carrying the C. reinhardtii α-helices, β-sheets, and βA-βB loop; etc.).

Immunoblot data will show that TobiEPYC1 and TobiEPYC1::GFP are stable when expressed in higher plants.

Fluorescence recovery after photobleaching (FRAP) data will show that fluorescently-tagged EPYC1 and Rubisco exhibit liquid-like mixing in the aggregates in higher plant chloroplasts.

Plant growth data (e.g., fresh weight, dry weight, rosette area, etc.) will show that growth of plants with aggregated Rubisco will be comparable to untransformed plants. Chlorophyll content, protein content, and total Rubisco content will also be comparable to untransformed plants.

Photosynthetic measurements (e.g., Fᵥ/Fₘ, A: Ci curves, etc.) will show that plants with aggregated Rubisco perform photosynthesis at similar efficiencies compared to untransformed plants.

Biochemical data (e.g., from isolated aggregates) will show that aggregated Rubisco is catalytically active. In addition, biochemical data will demonstrate that EPYC1 is present in the aggregate, and will characterize the EPYC1 in the aggregate for cleavage/degradation.

TEM/cryo-EM data will demonstrate the presence of the EPYC1 aggregate, and will characterize the physical properties of the EPYC1 aggregate.

### Example 6: A Variety of Other Higher Plants Will be Engineered to Express Pyrenoid-Like EPYC1-Rubisco Aggregates in the Chloroplast Stroma

The following example describes characterization of the molecular properties of the chloroplastic EPYC1 aggregates in TobiEPYC1 cowpea, soybean, cassava, rice, wheat, and tobacco lines. In addition, the following example describes characterization of the molecular properties of the chloroplastic EPYC1 aggregates in TobiEPYC1 cowpea, soybean, cassava, rice, wheat, and tobacco lines.

### Materials and Methods

Materials and Methods Relevant for Engineering Crop Plants with EPYC1-Rubisco Aggregates

The most promising constructs from Examples 3, 4, and 5 are used to design constructs for expression of EPYC1 in cowpea, soybean, cassava, rice, wheat, and tobacco (N. tabacum, Petite Havana). Species-specific optimization of the chloroplast signal peptide is done as needed. In addition, endogenous SSUs in cowpea, soybean, cassava, rice, wheat, and tobacco are reduced (e.g., using a CRISPR knockout approach). A C. reinhardtii SSU or a modified endogenous SSU having C. reinhardtii SSU motifs is introduced. Plants are transformed using nuclear transformation approaches.

The transformed plant lines are characterized as described in Examples 3-4.

### Results

Transformation of TobiEPYC1 into cowpea, soybean, cassava, rice, wheat, and tobacco and subsequent immunoblot data will show that the generated lines can stably express EPYC1.

Immunogold microscopy/other aggregate detection method of the above lines will show that they form EPYC1 and Rubisco aggregates in the chloroplast stroma.

Plant growth data (e.g., fresh weight, dry weight, yield, etc.) will show that growth of plants with aggregated Rubisco will be comparable to untransformed plants. Chlorophyll content, protein content, and total Rubisco content will also be comparable to untransformed plants.

Photosynthetic measurements (e.g., Fᵥ/Fₘ, A:Ci curves, etc.) will show that plants with aggregated Rubisco perform photosynthesis at similar efficiencies compared to untransformed plants.

What is claimed is:

1. A genetically altered higher plant or part thereof, comprising a modified Rubisco for formation of an aggregate of Essential Pyrenoid Component 1 (EPYC1) polypeptides and modified Rubiscos, wherein the modified Rubisco comprises an algal Rubisco small subunit (SSU) polypeptide or a modified higher plant Rubisco SSU polypeptide wherein at least part of the higher plant Rubisco SSU polypeptide is replaced with at least part of an algal Rubisco SSU polypeptide.

2. The plant or part thereof of claim 1, further comprising the EPYC1 polypeptides and the aggregate.

3. The plant or part thereof of claim 1, wherein the modified Rubisco comprising the algal Rubisco SSU polypeptide has increased affinity for the EPYC1 polypeptides as compared to unmodified Rubisco.

4. The plant or part thereof of claim 1, wherein the modified higher plant Rubisco SSU polypeptide was modified by substituting one or more higher plant Rubisco SSU α-helices with one or more algal Rubisco SSU α-helices; substituting one or more higher plant Rubisco SSU β-strands with one or more algal Rubisco SSU β-strands; and/or substituting a higher plant Rubisco SSU βA-βB loop with an algal Rubisco SSU βA-βB loop.

5. The plant or part thereof of claim 1, wherein the modified higher plant Rubisco SSU polypeptide has increased affinity for the EPYC1 polypeptides as compared to the higher plant Rubisco SSU polypeptide without the modification.

6. A genetically altered higher plant or part thereof, comprising EPYC1 polypeptides for formation of an aggregate of the EPYC1 polypeptides and modified Rubiscos.

7. The plant or part thereof of claim 6, wherein the EPYC1 polypeptides are algal EPYC1 polypeptides or modified EPYC1 polypeptides comprising one or more, two or more, four or more, or eight tandem copies of a first algal EPYC1 repeat region.

8. The plant or part thereof of claim 7, wherein the algal EPYC1 polypeptides are truncated mature EPYC1 polypeptides.

9. The plant or part thereof of claim 8, wherein the truncated mature EPYC1 polypeptides have increased affinity for the modified Rubiscos as compared to the non-truncated EPYC1 polypeptides.

10. The plant or part thereof of claim 7, wherein the modified EPYC1 polypeptides are expressed without the native EPYC1 leader sequence and/or comprise a C-terminal cap.

11. The plant or part thereof of claim 10, wherein the modified EPYC1 polypeptides have increased affinity for the modified Rubiscos as compared to the corresponding unmodified EPYC1 polypeptide.

12. The plant or part thereof of claim 6, wherein the aggregate is localized to a chloroplast stroma of at least one chloroplast of a plant cell, and wherein the plant cell is a leaf mesophyll cell.

13. A genetically altered higher plant or part thereof, comprising a first nucleic acid sequence encoding an EPYC1 polypeptide and a second nucleic acid sequence encoding a modified Rubisco polypeptide.

14. The plant or part thereof of claim 13, wherein the first nucleic acid sequence is operably linked to a third nucleic acid sequence encoding a chloroplastic transit peptide functional in the higher plant cell, and wherein the first nucleic acid sequence does not comprise the native EPYC1 leader sequence and is not operably linked to the native EPYC1 leader sequence, and wherein the second nucleic acid sequence is operably linked to a fourth nucleic acid sequence encoding a chloroplastic transit peptide functional in the higher plant cell and wherein the second nucleic acid sequence does not encode the native algal SSU leader sequence and is not operably linked to a nucleic acid sequence encoding the native algal SSU leader sequence.

15. The plant or part thereof of claim 13, wherein the EPYC1 polypeptide is a truncated mature EPYC1 polypeptide or a modified EPYC1 polypeptide comprising one or more, two or more, four or more, or eight tandem copies of a first algal EPYC1 repeat region.

16. The plant or part thereof of claim 13, wherein the modified Rubisco polypeptide comprises an algal Rubisco small subunit (SSU) polypeptide or a modified higher plant Rubisco SSU polypeptide wherein at least part of the higher plant Rubisco SSU polypeptide is replaced with at least part of an algal Rubisco SSU polypeptide.

17. The plant or part thereof of claim 13, wherein the plant or part thereof further comprises an aggregate of the modified Rubisco polypeptides and the EPYC1 polypeptides.

18. A method of producing the genetically altered higher plant of claim 1, comprising:
a) introducing a first nucleic acid sequence encoding an EPYC1 polypeptide into a plant cell, tissue, or other explant;
b) regenerating the plant cell, tissue, or other explant into a genetically altered plantlet; and
c) growing the genetically altered plantlet into a genetically altered plant with the first nucleic acid encoding the EPYC1 polypeptide.

19. The method of claim 18, further comprising introducing a second nucleic acid sequence encoding a modified Rubisco SSU polypeptide into a plant cell, tissue, or other explant prior to step (a) or concurrently with step (a), wherein the genetically altered plant of step (c) further comprises the second nucleic acid encoding the modified Rubisco SSU polypeptide.

20. The method of claim 18, wherein the first nucleic acid sequence is introduced with a first vector, and wherein the first vector comprises a first copy of the first nucleic acid sequence wherein the first nucleic acid sequence does not comprise the native EPYC1 leader sequence and is not operably linked to the native EPYC1 leader sequence, wherein the first nucleic acid sequence is operably linked to the third nucleic acid sequence encoding a chloroplastic transit peptide functional in the higher plant cell, wherein the first nucleic acid sequence is operably linked to the first promoter, and wherein the first nucleic acid sequence is operably linked to one terminator; and wherein the first vector further comprises a second copy of the first nucleic acid sequence wherein the first nucleic acid sequence does not comprise the native EPYC1 leader sequence and is not operably linked to the native EPYC1 leader sequence, wherein the first nucleic acid sequence is operably linked to the third nucleic acid sequence encoding a chloroplastic transit peptide functional in the higher plant cell, wherein the first nucleic acid sequence is operably linked to a third promoter, and wherein the first nucleic acid sequence is operably linked to two terminators.


SEQUENCE LISTING (172 sequences)

SEQ ID NO: 1 - protein, 125 aa, Arabidopsis thaliana
MQVWPPIGKKKFETLSYLPDLTDSELAKEVDYLIRNKWIPCVEFELEHGFVYREHGNSPGYYDGRYWTMWKLPLFGCTDSAQVLKEVEECKKEYPNAFIRIIGFDNTRQVQCISFIAYKPPSFTG

SEQ ID NO: 2 - protein, 140 aa, Chlamydomonas reinhardtii
MMVWTPVNNKMFETFSYLPPLSDEQIAAQVDYIVANGWIPCLEFAESDKAYVSNESAIRFGSVSCLYYDNRYWTMWKLPMFGCRDPMQVLREIVACTKAFPDAYVRLVAFDNQKQVQIMGFLVQRPKSARDWQPANKRSV

SEQ ID NO: 3 - protein, 13 aa, Arabidopsis thaliana
DSELAKEVDYLIR

SEQ ID NO: 4 - protein, 14 aa, Arabidopsis thaliana
SAQVLKEVEECKKE

SEQ ID NO: 5 - protein, 7 aa, Arabidopsis thaliana
IPCVEFE

SEQ ID NO: 6 - protein, 3 aa, Arabidopsis thaliana
TMW

SEQ ID NO: 7 - protein, 8 aa, Arabidopsis thaliana
FIRIIGFD

SEQ ID NO: 8 - protein, 9 aa, Arabidopsis thaliana
VQCISFIAY

SEQ ID NO: 9 - protein, 22 aa, Arabidopsis thaliana
LEHGFVYREHGNSPGYYDGRYW

SEQ ID NO: 10 - protein, 13 aa, Chlamydomonas reinhardtii
DEQIAAQVDYIVA

SEQ ID NO: 11 - protein, 7 aa, Chlamydomonas reinhardtii
IPCLEFA

SEQ ID NO: 12 - protein, 14 aa, Chlamydomonas reinhardtii
PMQVLREIVACTKA

SEQ ID NO: 13 - protein, 8 aa, Chlamydomonas reinhardtii
YVRLVAFD

SEQ ID NO: 14 - protein, 9 aa, Chlamydomonas reinhardtii
VQIMGFLVQ

SEQ ID NO: 15 - protein, 28 aa, Chlamydomonas reinhardtii
ESDKAYVSNESAIRFGSVSCLYYDNRYW

SEQ ID NO: 16 - protein, 28 aa, Chlamydomonas reinhardtii
EADKAYVSNESAIRFGSVSCLYYDNRYW

SEQ ID NO: 17 - protein, 55 aa, Arabidopsis thaliana
MASSMLSSAAVVTSPAQATMVAPFTGLKSSASFPVTRKANNDITSITSNGGRVSC

SEQ ID NO: 18 - protein, 126 aa, Arabidopsis thaliana
MKVWPPIGKKKFETLSYLPDLTDVELAKEVDYLLRNKWIPCVEFELEHGFVYREHGNTPGYYDGRYWTMWKLPLFGCTDSAQVLKEVEECKKEYPGAFIRIIGFDNTRQVQCISFIAYKPPSFTDA

SEQ ID NO: 19 - protein, 55 aa, Arabidopsis thaliana
MASSMFSSTAVVTSPAQATMVAPFTGLKSSASFPVTRKANNDITSITSNGGRVSC

SEQ ID NO: 20 - protein, 55 aa, Arabidopsis thaliana
MASSMLSSAAVVTSPAQATMVAPFTGLKSSAAFPVTRKTNKDITSIASNGGRVSC

SEQ ID NO: 21 - protein, 126 aa, Arabidopsis thaliana
MKVWPPIGKKKFETLSYLPDLSDVELAKEVDYLLRNKWIPCVEFELEHGFVYREHGNTPGYYDGRYWTMWKLPLFGCTDSAQVLKEVEECKKEYPGAFIRIIGFDNTRQVQCISFIAYKPPSFTEA

SEQ ID NO: 22 - protein, 195 aa, Arabidopsis thaliana
MASSMLSSATMVASPAQATMVAPFNGLKSSAAFPATRKANNDITSITSNGGRVNCMMVWTPVNNKMFETFSYLPPLSDEQIAAQVDYIVANGWIPCLEFAESDKAYVSNESAIRFGSVSCLYYDNRYWTMWKLPMFGCRDPMQVLREIVACTKAFPDAYVRLVAFDNQKQVQIMGFLVQRPKSARDWQPANKRSV

SEQ ID NO: 23 - protein, 125 aa, Arabidopsis thaliana
MQVWPPIGKKKFETLSYLPDLTDSELAKEVDYLIRNKWIPCLEFALEHGFVYREHGNSPGYYDGRYWTMWKLPLFGCTDSAQVLKEVEECKKEYPNAYVRLVAFDNTRQVQIMGFLVQKPPSFTG

SEQ ID NO: 24 - protein, 131 aa, Arabidopsis thaliana
MQVWPPIGKKKFETLSYLPDLTDSELAKEVDYLIRNKWIPCVEFEEADKAYVSNESAIRFGSVSCLYYDNRYWTMWKLPLFGCTDSAQVLKEVEECKKEYPNAFIRIIGFDNTRQVQCISFIAYKPPSFTG

SEQ ID NO: 25 - protein, 131 aa, Arabidopsis thaliana
MQVWPPIGKKKFETLSYLPDLTDSELAKEVDYLIRNKWIPCLEFAEADKAYVSNESAIRFGSVSCLYYDNRYWTMWKLPLFGCTDSAQVLKEVEECKKEYPNAYVRLVAFDNTRQVQIMGFLVQKPPSFTG

SEQ ID NO: 26 - protein, 125 aa, Arabidopsis thaliana
MQVWPPIGKKKFETLSYLPDLTDEQIAAQVDYIVANKWIPCVEFELEHGFVYREHGNSPGYYDGRYWTMWKLPLFGCTDPMQVLREIVACTKAYPNAFIRIIGFDNTRQVQCISFIAYKPPSFTG

SEQ ID NO: 27 - protein, 125 aa, Arabidopsis thaliana
MQVWPPIGKKKFETLSYLPDLTDEQIAAQVDYIVANKWIPCLEFALEHGFVYREHGNSPGYYDGRYWTMWKLPLFGCTDPMQVLREIVACTKAYPNAYVRLVAFDNTRQVQIMGFLVQKPPSFTG

SEQ ID NO: 28 - protein, 131 aa, Arabidopsis thaliana
MQVWPPIGKKKFETLSYLPDLTDEQIAAQVDYIVANKWIPCLEFAEADKAYVSNESAIRFGSVSCLYYDNRYWTMWKLPLFGCTDPMQVLREIVACTKAYPNAYVRLVAFDNTRQVQIMGFLVQKPPSFTG

SEQ ID NO: 29 - protein, 45 aa, Chlamydomonas reinhardtii
MAAVIAKSSVSAAVARPARSSVRPMAALKPAVKAAPVAAPAQANQ

SEQ ID NO: 30 - protein, 140 aa, Chlamydomonas reinhardtii
MMVWTPVNNKMFETFSYLPPLTDEQIAAQVDYIVANGWIPCLEFAEADKAYVSNESAIRFGSVSCLYYDNRYWTMWKLPMFGCRDPMQVLREIVACTKAFPDAYVRLVAFDNQKQVQIMGFLVQRPKTARDFQPANKRSV

SEQ ID NO: 31 - protein, 184 aa, Chlamydomonas reinhardtii
MAAVIAKSSVSAAVARPARSSVRPMAALKPAVKAAPVAAPAQANQMMVWTPVNNKMFETFSYLPPLTDEQIAAQVDYIVANGWIPCLEFAEADKAYVSNESAIRFGSVSCLYYDNRYWTMWKLPMFGCRDPMQVLREIVACTKAFPDAYVRLVAFDNQKQVQIMGFLVQRPKTARDFQPANKRS

SEQ ID NO: 32 - protein, 185 aa, Chlamydomonas reinhardtii
MAAVIAKSSVSAAVARPARSSVRPMAALKPAVKAAPVAAPAQANQMMVWTPVNNKMFETFSYLPPLSDEQIAAQVDYIVANGWIPCLEFAESDKAYVSNESAIRFGSVSCLYYDNRYWTMWKLPMFGCRDPMQVLREIVACTKAFPDAYVRLVAFDNQKQVQIMGFLVQRPKSARDWQPANKRSV

SEQ ID NO: 33 - protein, 180 aa, Arabidopsis thaliana
MASSMLSSATMVASPAQATMVAPFNGLKSSAAFPATRKANNDITSITSNGGRVNCMQVWPPIGKKKFETLSYLPDLTDEQIAAQVDYIVANKWIPCVEFELEHGFVYREHGNSPGYYDGRYWTMWKLPLFGCTDPMQVLREIVACTKAYPNAFIRIIGFDNTRQVQCISFIAYKPPSFTG

SEQ ID NO: 34 - protein, 317 aa, Chlamydomonas reinhardtii
MATISSMRVGAASRVVVSGRVKTVKVAARGSWRESSTATVQASRASSATNRVSPTRSVLPANWRQELESLRNGNGSSSAASSAPAPARSSSASWRDAAPASSAPARSSSASKKAVTPSRSALPSNWKQELESLRSSSPAPASSAPAPARSSSASWRDAAPASSAPARSSSSKKAVTPSRSALPSNWKQELESLRSSSPAPASSAPAPARSSSASWRDAAPASSAPARSSSASKKAVTPSRSALPSNWKQELESLRSNSPAPASSAPAPARSSSASWRDAPASSSSSSADKAGTNPWTGKSKPEIKRTALPADWRKGL

SEQ ID NO: 35 - protein, 291 aa, Chlamydomonas reinhardtii
AARGSWRESSTATVQASRASSATNRVSPTRSVLPANWRQELESLRNGNGSSSAASSAPAPARSSSASWRDAAPASSAPARSSSASKKAVTPSRSALPSNWKQELESLRSSSPAPASSAPAPARSSSASWRDAAPASSAPARSSSSKKAVTPSRSALPSNWKQELESLRSSSPAPASSAPAPARSSSASWRDAAPASSAPARSSSASKKAVTPSRSALPSNWKQELESLRSNSPAPASSAPAPARSSSASWRDAPASSSSSSADKAGTNPWTGKSKPEIKRTALPADWRKGL

SEQ ID NO: 36 - protein, 63 aa, Artificial Sequence
  Synthetic Construct
VSPTRSVLPANWRQELESLRNGNGSSSAASSAPAPARSSSASWRDAAPASSAPARSSSASKKA

SEQ ID NO: 37 - protein, 126 aa, Artificial Sequence
  Synthetic Construct
VSPTRSVLPANWRQELESLRNGNGSSSAASSAPAPARSSSASWRDAAPASSAPARSSSASKKAVSPTRSVLPANWRQELESLRNGNGSSSAASSAPAPARSSSASWRDAAPASSAPARSSSASKKA

SEQ ID NO: 38 - protein, 252 aa, Artificial Sequence
  Synthetic Construct
VSPTRSVLPANWRQELESLRNGNGSSSAASSAPAPARSSSASWRDAAPASSAPARSSSASKKAVSPTRSVLPANWRQELESLRNGNGSSSAASSAPAPARSSSASWRDAAPASSAPARSSSASKKAVSPTRSVLPANWRQELESLRNGNGSSSAASSAPAPARSSSASWRDAAPASSAPARSSSASKKAVSPTRSVLPANWRQELESLRNGNGSSSAASSAPAPARSSSASWRDAAPASSAPARSSSASKKA

SEQ ID NO: 39 - protein, 504 aa, Artificial Sequence
  Synthetic Construct
VSPTRSVLPANWRQELESLRNGNGSSSAASSAPAPARSSSASWRDAAPASSAPARSSSASKKAVSPTRSVLPANWRQELESLRNGNGSSSAASSAPAPARSSSASWRDAAPASSAPARSSSASKKAVSPTRSVLPANWRQELESLRNGNGSSSAASSAPAPARSSSASWRDAAPASSAPARSSSASKKAVSPTRSVLPANWRQELESLRNGNGSSSAASSAPAPARSSSASWRDAAPASSAPARSSSASKKAVSPTRSVLPANWRQELESLRNGNGSSSAASSAPAPARSSSASWRDAAPASSAPARSSSASKKAVSPTRSVLPANWRQELESLRNGNGSSSAASSAPAPARSSSASWRDAAPASSAPARSSSASKKAVSPTRSVLPANWRQELESLRNGNGSSSAASSAPAPARSSSASWRDAAPASSAPARSSSASKKAVSPTRSVLPANWRQELESLRNGNGSSSAASSAPAPARSSSASWRDAAPASSAPARSSSASKKA

SEQ ID NO: 40 - protein, 25 aa, Artificial Sequence
  Synthetic Construct
AARGSWRESSTATVQASRASSATNR

SEQ ID NO: 41 - protein, 26 aa, Artificial Sequence
  Synthetic Construct
GTNPWTGKSKPEIKRTALPADWRKGL

SEQ ID NO: 42 - protein, 26 aa, Artificial Sequence
  Synthetic Construct
MATISSMRVGAASRVVVSGRVKTVKV

SEQ ID NO: 43 - protein, 114 aa, Artificial Sequence
  Synthetic Construct
MATISSMRVGAASRVVVSGRVKTVKVAARGSWRESSTATVQASRASSATNRVSPTRSVLPANWRQELESLRNGNGSSSAASSAPAPARSSSASWRDAAPASSAPARSSSASKKA

SEQ ID NO: 44 - protein, 174 aa, Artificial Sequence
  Synthetic Construct
MATISSMRVGAASRVVVSGRVKTVKVAARGSWRESSTATVQASRASSATNRVSPTRSVLPANWRQELESLRNGNGSSSAASSAPAPARSSSASWRDAAPASSAPARSSSASKKAVTPSRSALPSNWKQELESLRSSSPAPASSAPAPARSSSASWRDAAPASSAPARSSSSKKA

SEQ ID NO: 45 - protein, 235 aa, Artificial Sequence
  Synthetic Construct
MATISSMRVGAASRVVVSGRVKTVKVAARGSWRESSTATVQASRASSATNRVSPTRSVLPANWRQELESLRNGNGSSSAASSAPAPARSSSASWRDAAPASSAPARSSSASKKAVTPSRSALPSNWKQELESLRSSSPAPASSAPAPARSSSASWRDAAPASSAPARSSSSKKAVTPSRSALPSNWKQELESLRSSSPAPASSAPAPARSSSASWRDAAPASSAPARSSSASKKA

SEQ ID NO: 46 - protein, 291 aa, Artificial Sequence
  Synthetic Construct
MATISSMRVGAASRVVVSGRVKTVKVAARGSWRESSTATVQASRASSATNRVSPTRSVLPANWRQELESLRNGNGSSSAASSAPAPARSSSASWRDAAPASSAPARSSSASKKAVTPSRSALPSNWKQELESLRSSSPAPASSAPAPARSSSASWRDAAPASSAPARSSSSKKAVTPSRSALPSNWKQELESLRSSSPAPASSAPAPARSSSASWRDAAPASSAPARSSSASKKAVTPSRSALPSNWKQELESLRSNSPAPASSAPAPARSSSASWRDAPASSSSSSADKA

SEQ ID NO: 47 - protein, 266 aa, Artificial Sequence
  Synthetic Construct
VSPTRSVLPANWRQELESLRNGNGSSSAASSAPAPARSSSASWRDAAPASSAPARSSSASKKAVTPSRSALPSNWKQELESLRSSSPAPASSAPAPARSSSASWRDAAPASSAPARSSSSKKAVTPSRSALPSNWKQELESLRSSSPAPASSAPAPARSSSASWRDAAPASSAPARSSSASKKAVTPSRSALPSNWKQELESLRSNSPAPASSAPAPARSSSASWRDAPASSSSSSADKAGTNPWTGKSKPEIKRTALPADWRKGL

SEQ ID NO: 48 - protein, 203 aa, Artificial Sequence
  Synthetic Construct
VTPSRSALPSNWKQELESLRSSSPAPASSAPAPARSSSASWRDAAPASSAPARSSSSKKAVTPSRSALPSNWKQELESLRSSSPAPASSAPAPARSSSASWRDAAPASSAPARSSSASKKAVTPSRSALPSNWKQELESLRSNSPAPASSAPAPARSSSASWRDAPASSSSSSADKAGTNPWTGKSKPEIKRTALPADWRKGL

SEQ ID NO: 49 - protein, 143 aa, Artificial Sequence
  Synthetic Construct
VTPSRSALPSNWKQELESLRSSSPAPASSAPAPARSSSASWRDAAPASSAPARSSSASKKAVTPSRSALPSNWKQELESLRSNSPAPASSAPAPARSSSASWRDAPASSSSSSADKAGTNPWTGKSKPEIKRTALPADWRKGL

SEQ ID NO: 50 - protein, 82 aa, Artificial Sequence
  Synthetic Construct
VTPSRSALPSNWKQELESLRSNSPAPASSAPAPARSSSASWRDAPASSSSSSADKAGTNPWTGKSKPEIKRTALPADWRKGL

SEQ ID NO: 51 - protein, 317 aa, Artificial Sequence
  Synthetic Construct
MATISSMRVGAASRVVVSGRVKTVKVAARGSWRESSTATVQASRASSATNRVSPTRSVLPANWAAAAAAARNGNGSSSAASSAPAPARSSSASWRDAAPASSAPARSSSASKKAVTPSRSALPSNWKQELESLRSSSPAPASSAPAPARSSSASWRDAAPASSAPARSSSSKKAVTPSRSALPSNWKQELESLRSSSPAPASSAPAPARSSSASWRDAAPASSAPARSSSASKKAVTPSRSALPSNWKQELESLRSNSPAPASSAPAPARSSSASWRDAPASSSSSSADKAGTNPWTGKSKPEIKRTALPADWRKGL

SEQ ID NO: 52 - protein, 317 aa, Artificial Sequence
  Synthetic Construct
MATISSMRVGAASRVVVSGRVKTVKVAARGSWRESSTATVQASRASSATNRVSPTRSVLPANWAAAAAAARNGNGSSSAASSAPAPARSSSASWRDAAPASSAPARSSSASKKAVTPSRSALPSNWAAAAAAARSSSPAPASSAPAPARSSSASWRDAAPASSAPARSSSSKKAVTPSRSALPSNWKQELESLRSSSPAPASSAPAPARSSSASWRDAAPASSAPARSSSASKKAVTPSRSALPSNWKQELESLRSNSPAPASSAPAPARSSSASWRDAPASSSSSSADKAGTNPWTGKSKPEIKRTALPADWRKGL

SEQ ID NO: 53 - protein, 317 aa, Artificial Sequence
  Synthetic Construct
MATISSMRVGAASRVVVSGRVKTVKVAARGSWRESSTATVQASRASSATNRVSPTRSVLPANWAAAAAAARNGNGSSSAASSAPAPARSSSASWRDAAPASSAPARSSSASKKAVTPSRSALPSNWAAAAAAARSSSPAPASSAPAPARSSSASWRDAAPASSAPARSSSSKKAVTPSRSALPSNWAAAAAAARSSSPAPASSAPAPARSSSASWRDAAPASSAPARSSSASKKAVTPSRSALPSNWKQELESLRSNSPAPASSAPAPARSSSASWRDAPASSSSSSADKAGTNPWTGKSKPEIKRTALPADWRKGL

SEQ ID NO: 54 - protein, 317 aa, Artificial Sequence
  Synthetic Construct
MATISSMRVGAASRVVVSGRVKTVKVAARGSWRESSTATVQASRASSATNRVSPTRSVLPANWAAAAAAARNGNGSSSAASSAPAPARSSSASWRDAAPASSAPARSSSASKKAVTPSRSALPSNWAAAAAAARSSSPAPASSAPAPARSSSASWRDAAPASSAPARSSSSKKAVTPSRSALPSNWAAAAAAARSSSPAPASSAPAPARSSSASWRDAAPASSAPARSSSASKKAVTPSRSALPSNWAAAAAAARSNSPAPASSAPAPARSSSASWRDAPASSSSSSADKAGTNPWTGKSKPEIKRTALPADWRKGL

SEQ ID NO: 55 - protein, 317 aa, Artificial Sequence
  Synthetic Construct
MATISSMRVGAASRVVVSGRVKTVKVAARGSWRESSTATVQASRASSATNRVSPTRSVLPANWRQELESLRNGNGSSSAASSAPAPARSSSASWRDAAPASSAPARSSSASKKAVTPSRSALPSNWKQELESLRSSSPAPASSAPAPARSSSASWRDAAPASSAPARSSSSKKAVTPSRSALPSNWAAAAAAARSSSPAPASSAPAPARSSSASWRDAAPASSAPARSSSASKKAVTPSRSALPSNWAAAAAAARSNSPAPASSAPAPARSSSASWRDAPASSSSSSADKAGTNPWTGKSKPEIKRTALPADWRKGL

SEQ ID NO: 56 - protein, 317 aa, Artificial Sequence
  Synthetic Construct
MATISSMRVGAASRVVVSGRVKTVKVAARGSWRESSTATVQASRASSATNRVSPTRSVLPANWRQELESLRNGNGSSSAASSAPAPARSSSASWRDAAPASSAPARSSSASKKAVTPSRSALPSNWKQELESLRSSSPAPASSAPAPARSSSASWRDAAPASSAPARSSSSKKAVTPSRSALPSNWKQELESLRSSSPAPASSAPAPARSSSASWRDAAPASSAPARSSSASKKAVTPSRSALPSNWAAAAAAARSNSPAPASSAPAPARSSSASWRDAPASSSSSSADKAGTNPWTGKSKPEIKRTALPADWRKGL

SEQ ID NO: 57 - protein, 74 aa, Artificial Sequence
  Synthetic Construct
VSPTRSVLPANWRQELESLRNNWRQELESLRNGNGSSSAASSAPAPARSSSASWRDAAPASSAPARSSSASKKA

SEQ ID NO: 58 - protein, 296 aa, Artificial Sequence
  Synthetic Construct
VSPTRSVLPANWRQELESLRNNWRQELESLRNGNGSSSAASSAPAPARSSSASWRDAAPASSAPARSSSASKKAVSPTRSVLPANWRQELESLRNNWRQELESLRNGNGSSSAASSAPAPARSSSASWRDAAPASSAPARSSSASKKAVSPTRSVLPANWRQELESLRNNWRQELESLRNGNGSSSAASSAPAPARSSSASWRDAAPASSAPARSSSASKKAVSPTRSVLPANWRQELESLRNNWRQELESLRNGNGSSSAASSAPAPARSSSASWRDAAPASSAPARSSSASKKA

SEQ ID NO: 59 - protein, 63 aa, Artificial Sequence
  Synthetic Construct
VSPTRSVLPANWRQELESLRNQNGSSSAASSAPAPARSSSASWRDAAPASSAPARSSSASKKA

SEQ ID NO: 60 - protein, 63 aa, Artificial Sequence
  Synthetic Construct
VSPTRSVLPANWRQELESRRNGNGSSSAASSAPAPARSSSASWRDAAPASSAPARSSSASKKA

SEQ ID NO: 61 - protein, 63 aa, Artificial Sequence
  Synthetic Construct
VSPTRSVLPANWRQWLESLRNGNGSSSAASSAPAPARSSSASWRDAAPASSAPARSSSASKKA

SEQ ID NO: 62 - protein, 80 aa, Arabidopsis thaliana
MASSMLSSATMVASPAQATMVAPFNGLKSSAAFPATRKANNDITSITSNGGRVNCMQVWPPIGKKKFETLSYLPDLTDSE

SEQ ID NO: 63 - protein, 57 aa, Arabidopsis thaliana
MASSMLSSATMVASPAQATMVAPFNGLKSSAAFPATRKANNDITSITSNGGRVNCGG

SEQ ID NO: 64 - protein, 55 aa, Arabidopsis thaliana
MASSMLSSATMVASPAQATMVAPFNGLKSSAAFPATRKANNDITSITSNGGRVNC

SEQ ID NO: 65 - DNA, 1745 bp, Chlamydomonas reinhardtii
ggcaagcactcgcagccgctccatctgtagcgtcgacctttcagaaccactccaaaacaatggccactatctcgtcgatgcgcgttggcgctgcttcccgcgtggtcgtctctggccgcgtgaagaccgtgaaggtcgccgcccgcggcagctggcgcgagtcttccactgccaccgtgcaggccagccgcgcctcgtcggccaccaaccgcgtgagccccacccgctccgtcctgcccgccaactggcgccaggagctggagagcctgcgcaacggcaacggctcctcctcggctgcctcgtcggcccccgccccggcccgctcctcgtcggccagctggcgcgacgccgccccggcctcgtcggcccctgcccgctccagctctgcctccaagaaggccgtgaccccgtcgcgcagcgccctgccctccaactggaagcaggagctggagagcctgcgcagcagctcccccgcccccgcctcgtcggcccccgccccggcccgctcctcgtcggccagctggcgtgatgccgccccggcctcgtcggcccccgcccgctccagctcctccaagaaggctgtgaccccgtcgcgcagcgccctgccctccaactggaagcaggagctggagagcctgcgcagcagctcccccgcccccgcctcgtcggcccctgccccggcccgctcctcgtcggccagctggcgtgacgccgccccggcctcgtcggcccctgcccgctccagctctgcctccaagaaggccgtgaccccgtcgcgcagcgccctgccctccaactggaagcaggagctggagagcctgcgcagcaactcccctgcccccgcctcgtcggcccctgccccggcccgctcctcgtcggccagctggcgtgacgcccccgcctcgagctccagctcgagcgccgacaaggccggcaccaacccctggactggcaagtccaagcccgagatcaagcgcaccgccctgcccgctgactggcgcaagggcctgtaagcagcttgcctaaccagcagctggcttaaagcatgatgccttggtacgcgtgtgttatgtacaacactaatgtacatcactagagcgcgttaactagcggcgtgggttcatgttggagagagaagagactgtgcagcaggagggtcgggggaaccgcgggtacctgtgcggcatgttagcgccgggtcttcagtttttgttgcgttccgtgggtgtttgcgtggtgcatgccacacagatgtgagttcgtgagttctgatatgtctggttgcaaacctgaggtgcggaggaacacatgcgttccatactcgccctcaaattttggttgagacgtgagagggtgcctgcagagcgtgcgggtgcctaatccggagcggcgcagcagagccctgccgccgcgcacttcattcagtggagtgactaacttgcacgactgcttttgggagcgcgggcaaggagggcttagagaggagagaacaagtagcactgccgataggtgatggatgtacacaagcattagaggatggatcaatcgaacgatgcaaaaagaggtgctgcggctgtcaaccggcacagcgctcgaaaaccctcgtgtgtaagcaacgagcgagactttctgtggctctggcagtgtctcatgcccatgatgcactgcgtctgtatggcttgtcagcaggtgttggctcgatcttggtctcgatcaggttatcagtccactataaatttctgcat

SEQ ID NO: 66 - DNA, 2258 bp, Chlamydomonas reinhardtii
ggcaagcactcgcagccgctccatctgtagcgtcgacctttcagaaccactccaaaacaatggccactatctcgtcgatgcgcgttggcgctgcttcccgcgtggtcgtctctggccgcgtgaaggtgaggcacttgatcgcacttcttcttcagtagtgtcaaggcggcaggttcatggcgcggcttgataggcgaccagagcgacttgcaagcttggcctcggcgcaaatgccgttacacgaccgcgcttgcttggcttcgcgcgccgagcagcgcagctgcatggcaatggttgttatgatcatagtctccaccacgccttgctcttcagcctttctgatgcgactgttactgtccttcgcgcgcccttgcagaccgtgaaggtcgccgcccgcggcagctggcgcgagtcttccactgccaccgtgcaggccaggtgagcacacttctgcagctatgagatgcatctgggtccagcttaaagcggctcgcgttgtgtggcgcgccgcgatcccttatccgctcgcctgccagccgggccttttcgcacttgtttcctaagtcaagttcgaacctgcagctggctgtgcatatcttgctaagtgatagcgcggttgtacgcggtttgagtacgctgctcaactggtgtactgacacgtttgcttgccgtttcccctggtgccccttcgcccctgcagccgcgcctcgtcggccaccaaccgcgtgagccccacccgctccgtcctgcccgccaactggcgccaggagctggagagcctgcgcaacggcaacggctcctcctcggctgcctcgtcggcccccgccccggcccgctcctcgtcggccagctggcgcgacgccgccccggcctcgtcggcccctgcccgctccagctctgcctccaagaaggccgtgaccccgtcgcgcagcgccctgccctccaactggaagcaggagctggagagcctgcgcagcagctcccccgcccccgcctcgtcggcccccgccccggcccgctcctcgtcggccagctggcgtgatgccgccccggcctcgtcggcccccgcccgctccagctcctccaagaaggctgtgaccccgtcgcgcagcgccctgccctccaactggaagcaggagctggagagcctgcgcagcagctcccccgcccccgcctcgtcggcccctgccccggcccgctcctcgtcggccagctggcgtgacgccgccccggcctcgtcggcccctgcccgctccagctctgcctccaagaaggccgtgaccccgtcgcgcagcgccctgccctccaactggaagcaggagctggagagcctgcgcagcaactcccctgcccccgcctcgtcggcccctgccccggcccgctcctcgtcggccagctggcgtgacgcccccgcctcgagctccagctcgagcgccgacaaggccggcaccaacccctggactggcaagtccaagcccgagatcaagcgcaccgccctgcccgctgactggcgcaagggcctgtaagcagcttgcctaaccagcagctggcttaaagcatgatgccttggtacgcgtgtgttatgtacaacactaatgtacatcactagagcgcgttaactagcggcgtgggttcatgttggagagagaagagactgtgcagcaggagggtcgggggaaccgcgggtacctgtgcggcatgttagcgccgggtcttcagtttttgttgcgttccgtgggtgtttgcgtggtgcatgccacacagatgtgagttcgtgagttctgatatgtctggttgcaaacctgaggtgcggaggaacacatgcgttccatactcgccctcaaattttggttgagacgtgagagggtgcctgcagagcgtgcgggtgcctaatccggagcggcgcagcagagccctgccgccgcgcacttcattcagtggagtgactaacttgcacgactgcttttgggagcgcgggcaaggagggcttagagaggagagaacaagtagcactgccgataggtgatggatgtacacaagcattagaggatggatcaatcgaacgatgcaaaaagaggtgctgcggctgtcaaccggcacagcgctcgaaaaccctcgtgtgtaagcaacgagcgagactttctgtggctctggcagtgtctcatgcccatgatgcactgcgtctgtatggcttgtcagcaggtgttggctcgatcttggtctcgatcaggttatcagtccactataaatttctgcat

SEQ ID NO: 67 - DNA, 9447 bp, Artificial Sequence
  Synthetic Construct
cacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagcgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccattacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactctgtgggtctcatgccgatgtatcacctctcgcaagaattcaagcttggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatggcttcctctatgctctcttccgctactatggttgcctctccggctcaggccactatggtcgctcctttcaacggacttaagtcctccgctgccttcccagccacccgcaaggctaacaacgacattacttccatcacaagcaacggcggaagagttaactgcatgcaggtgtggcctccgattggaaagaagaagtttgagactctctcttaccttcctgaccttaccgattccgaaggtatggctacgatcagttctatgagagttggagctgcgtctagggtcgttgtttcaggaagagttaaaaccgtgaaggtagcagcaagaggatcttggagagagtcatctactgctacagttcaagcctcaagagctagtagtgcaacgaacagagtgagcccaacaagaagcgttctcccagctaattggaggcaagaacttgaatcactcagaaatggtaacggcagctcgtcggcggcgtcctctgctcctgctcctgcgagatcgagcagcgcatcgtggcgagacgccgctcctgcatcatcagcgcctgccagaagtagctcagcttcaaagaaggccgttactccttcaagaagtgccttgccttcaaattggaagcaggaattggagtcattgagaagctccagccctgcaccggcttcatcagcgccagctccggccagaagtagttctgcctcatggagggacgcagcgcctgcatctagcgctccagctaggagttcttccagcaagaaggccgtcactccgtctcgttcagctctgccttctaactggaaacaggaacttgaatcccttcgatcaagcagtccagcaccagcttctagcgcgccagcaccagctaggtcatcctcagctagttggagggatgcagctcctgcttcttccgccccagctcgtagctcatcggcgagcaaaaaagcagtcacccctagtagatcggcccttccaagtaattggaaacaggagttggagtcactccgatccaacagtcccgcacctgcaagcagtgcccctgcacctgctagatcatcatccgcttcctggagagacgctcccgcttcgtcatctagttcctccgctgacaaagccggaactaatccttggacaggtaaaagcaagccagaaattaagagaacggctctccctgcagactggagaaagggcctttgagcttgtcctgctttaatgagatatgcgagaagcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggaccgctactagaattcgagctcggagttctagaatgtcgcggaacaaattttaaaactaaatcctaaatttttctaattttgttgccaatagtggatatgtgggccgtatagaaggaatctattgaaggcccaaacccatactgacgagcccaaaggttcgttttgcgttttatgtttcggttcgatgccaacgccacattctgagctaggcaaaaaacaaacgtgtctttgaatagactcctctcgttaacacatgcagcggctgcatggtgacgccattaacacgtggcctacaattgcatgatgtctccattgacacgtgacttctcgtctcctttcttaatatatctaacaaacactcctacctcttccaaaatatatacacatctttttgatcaatctctcattcaaaatctcattctctctagtaaacaagaacaaaaaaatggcggatacagctagaggaacccatcacgatatcatcggcagagatcagtacccgatgatgggccgagatcgtgaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtaagcacacatttatcatcttacttcataattttgtgcaatatgtgcatgcatgtgttgagccagtagctttggatcaatttttttggtcgaataacaaatgtaacaataagaaattgcaaattctagggaacatttggttaactaaatacgaaatttgacctagctagcttgaatgtgtctgtgtatatcatctatataggtaaaatgcttggtatgatacctattgattgtgaataggtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggccagcacactactatgagcgagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaagcgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagaaacctacgtcgagcagcacgaggtggctgtggccagatactgcgacctccctagcaaactggggcacaagtgagcttaccccactgatgtcatcgtcatagtccaataactccaatgtcggggagttagtttatgaggaataaagtgtttagaatttgatcagggggagataataaaagccgagtttgaatctttttgttataagtaatgtttatgtgtgtttctatatgttgtcaaatggtaccatgttttttttcctctctttttgtaacttgcaagtgttgtgttgtactttatttggcttctttgtaagttggtaacggtggtctatatatggaaaaggtcttgttttgttaaacttatgttagttaactggattcgtctttaaccacaaaaagttttcaataagctacaaatttagacacgcaagccgatgcagtcattagtacatatatttattgcaagtgattacatggcaacccaaacttcaaaaacagtaggttgctccatttagtcgctttactgagaccgaggatgcacatgtgaccgaggga

SEQ ID NO: 68 - protein, 51 aa, Artificial Sequence
  Synthetic Construct
MATISSMRVGAASRVVVSGRVKTVKVAARGSWRESSTATVQASRASSATNR

SEQ ID NO: 69 - protein, 60 aa, Artificial Sequence
  Synthetic Construct
VTPSRSALPSNWKQELESLRSSSPAPASSAPAPARSSSASWRDAAPASSAPARSSSSKKA

SEQ ID NO: 70 - protein, 61 aa, Artificial Sequence
  Synthetic Construct
VTPSRSALPSNWKQELESLRSSSPAPASSAPAPARSSSASWRDAAPASSAPARSSSASKKA

SEQ ID NO: 71 - protein, 56 aa, Artificial Sequence
  Synthetic Construct
VTPSRSALPSNWKQELESLRSNSPAPASSAPAPARSSSASWRDAPASSSSSSADKA

SEQ ID NO: 72 - DNA, 36 bp, Artificial Sequence
  Synthetic Construct
ttttgaattcatggctacgatcagttctatgagagt

SEQ ID NO: 73 - DNA, 32 bp, Artificial Sequence
  Synthetic Construct
ataggatcctcaaaggccctttctccagtctg

SEQ ID NO: 74 - DNA, 34 bp, Artificial Sequence
  Synthetic Construct
aaaagaattcgtgtggacaccggtgaacaacaag

SEQ ID NO: 75 - DNA, 33 bp, Artificial Sequence
  Synthetic Construct
atacccgggacgtttgttggctggttggaaatc

SEQ ID NO: 76 - DNA, 28 bp, Artificial Sequence
  Synthetic Construct
tatcccgggacgtttgttggctggttgc

SEQ ID NO: 77 - DNA, 28 bp, Artificial Sequence
  Synthetic Construct
aaacccgggcatgcaggtgtggcctccg

SEQ ID NO: 78 - DNA, 31 bp, Artificial Sequence
  Synthetic Construct
aaaggatccttaaccggtgaagcttggtggc

SEQ ID NO: 79 - DNA, 37 bp, Artificial Sequence
  Synthetic Construct
atatgaattcatggttccacaaacagaaactaaagca

SEQ ID NO: 80 - DNA, 38 bp, Artificial Sequence
  Synthetic Construct
cccggatccttaaagtttgtcaatagtatcaaattcga

SEQ ID NO: 81 - DNA, 34 bp, Artificial Sequence
  Synthetic Construct
tttggatcctctgttcgttgcactactagctctt

SEQ ID NO: 82 - DNA, 34 bp, Artificial Sequence
  Synthetic Construct
tttggatccggccttctttgaagctgagctactt

SEQ ID NO: 83 - DNA, 34 bp, Artificial Sequence
  Synthetic Construct
aatggatccggccttcttgctggaagaactccta

SEQ ID NO: 84 - DNA, 36 bp, Artificial Sequence
  Synthetic Construct
tttggatcctgcttttttgctcgccgatgagctacg

SEQ ID NO: 85 - DNA, 37 bp, Artificial Sequence
  Synthetic Construct
ataggatccggctttgtcagcggaggaactagatgac

SEQ ID NO: 86 - DNA, 34 bp, Artificial Sequence
  Synthetic Construct
ttttgaattcgtgagcccaacaagaagcgttctc

SEQ ID NO: 87 - DNA, 35 bp, Artificial Sequence
  Synthetic Construct
ttttgaattcgttactccttcaagaagtgccttgc

SEQ ID NO: 88 - DNA, 32 bp, Artificial Sequence
  Synthetic Construct
ttttgaattcgtcactccgtctcgttcagctc

SEQ ID NO: 89 - DNA, 31 bp, Artificial Sequence
  Synthetic Construct
ttttgaattcgtcacccctagtagatcggcc

SEQ ID NO: 90 - DNA, 37 bp, Artificial Sequence
  Synthetic Construct
aaaagaattcggaactaatccttggacaggtaaaagc

SEQ ID NO: 91 - DNA, 44 bp, Artificial Sequence
  Synthetic Construct
acgtaccggtctccacatcccgggggtgagcccaacaagaagcg

SEQ ID NO: 92 - DNA, 43 bp, Artificial Sequence
  Synthetic Construct
acgtaccggtctccacaaggatccggccttctttgaagctgag

SEQ ID NO: 93 - DNA, 37 bp, Artificial Sequence
  Synthetic Construct
acgtaccggtctcctgtaagcccaacaagaagcgttc

SEQ ID NO: 94 - DNA, 36 bp, Artificial Sequence
  Synthetic Construct
acgtaccggtctcctacagccttctttgaagctgag

SEQ ID NO: 95 - DNA, 37 bp, Artificial Sequence
  Synthetic Construct
acgtaccggtctccggttagcccaacaagaagcgttc

SEQ ID NO: 96 - DNA, 36 bp, Artificial Sequence
  Synthetic Construct
acgtaccggtctccaaccgccttctttgaagctgag

SEQ ID NO: 97 - DNA, 37 bp, Artificial Sequence
  Synthetic Construct
acgtaccggtctcccgtcagcccaacaagaagcgttc

SEQ ID NO: 98 - DNA, 36 bp, Artificial Sequence
  Synthetic Construct
acgtaccggtctccgacggccttctttgaagctgag

SEQ ID NO: 99 - DNA, 30 bp, Artificial Sequence
  Synthetic Construct
acgtaccggtctccacatcccgggggtgag

SEQ ID NO: 100 - DNA, 31 bp, Artificial Sequence
  Synthetic Construct
gccacttggtctcgacaaggatccggccttc

SEQ ID NO: 101 - DNA, 32 bp, Artificial Sequence
  Synthetic Construct
ctctgtgaagacaggtctcgagtgagcccaac

SEQ ID NO: 102 - DNA, 31 bp, Artificial Sequence
  Synthetic Construct
cttcgtgaagggtctcacactgccttctttg

SEQ ID NO: 103 - DNA, 36 bp, Artificial Sequence
  Synthetic Construct
ttgaatcactcagaaataattggaggcaagaacttg

SEQ ID NO: 104 - DNA, 36 bp, Artificial Sequence
  Synthetic Construct
caagttcttgcctccaattatttctgagtgattcaa

SEQ ID NO: 105 - DNA, 33 bp, Artificial Sequence
  Synthetic Construct
acgtaccggtctcatcagaacggcagctcgtcg

SEQ ID NO: 106 - DNA, 38 bp, Artificial Sequence
  Synthetic Construct
acgtaccggtctctctgatttctgagtgattcaagttc

SEQ ID NO: 107 - DNA, 34 bp, Artificial Sequence
  Synthetic Construct
acgtaccggtctccgtagaaatggtaacggcagc

SEQ ID NO: 108 - DNA, 34 bp, Artificial Sequence
  Synthetic Construct
acgtaccggtctccctacgtgattcaagttcttg

SEQ ID NO: 109 - DNA, 36 bp, Artificial Sequence
  Synthetic Construct
acgtaccggtctcatggcttgaatcactcagaaatg

SEQ ID NO: 110 - DNA, 35 bp, Artificial Sequence
  Synthetic Construct
acgtaccggtctcagccattgcctccaattagctg

SEQ ID NO: 111 - DNA, 32 bp, Artificial Sequence
  Synthetic Construct
atacatatgcaagcagcatcaacagcggttgc

SEQ ID NO: 112 - DNA, 36 bp, Artificial Sequence
  Synthetic Construct
atacccggggttttttggtgcttcaaatgacgggtg

SEQ ID NO: 113 - DNA, 34 bp, Artificial Sequence
  Synthetic Construct
tatcccgggtagtcaagctctcactgttagccaa

SEQ ID NO: 114 - DNA, 33 bp, Artificial Sequence
  Synthetic Construct
tatggatccgttcatattagctagctcgggaga

SEQ ID NO: 115 - DNA, 32 bp, Artificial Sequence
  Synthetic Construct
atttgaattccgaagcgcagttcttcagagag

SEQ ID NO: 116 - DNA, 34 bp, Artificial Sequence
  Synthetic Construct
ttaggatcctcagagctcatactccacaagtcta

SEQ ID NO: 117 - DNA, 34 bp, Artificial Sequence
  Synthetic Construct
ttttgaattcggtccggtccatttgaacaattcg

SEQ ID NO: 118 - DNA, 33 bp, Artificial Sequence
  Synthetic Construct
tttcccggggcactcgttggtctcaggattgtc

SEQ ID NO: 119 - protein, 7 aa, Artificial Sequence
  Synthetic Construct
RQELESL

SEQ ID NO: 120 - protein, 7 aa, Artificial Sequence
  Synthetic Construct
KQELESL

SEQ ID NO: 121 - protein, 32 aa, Artificial Sequence
  Synthetic Construct
SVLPANWRQELESLRNNWRQELESLRNGNGSS

SEQ ID NO: 122 - protein, 11 aa, Artificial Sequence
  Synthetic Construct
WRQELESLRNQ

SEQ ID NO: 123 - protein, 11 aa, Artificial Sequence
  Synthetic Construct
WRQELESRRNG

SEQ ID NO: 124 - protein, 11 aa, Artificial Sequence
  Synthetic Construct
WRQWLESLRNG

SEQ ID NO: 125 - DNA, 40 bp, Artificial Sequence
  Synthetic Construct
tacggtcgaagacgaaggtatggctacgatcagttctatg

SEQ ID NO: 126 - DNA, 38 bp, Artificial Sequence
  Synthetic Construct
tacggtcgaagacgagatgactctctccaagatcctct

SEQ ID NO: 127 - DNA, 37 bp, Artificial Sequence
  Synthetic Construct
acgtaccgaagaccacatctactgctacagttcaagc

SEQ ID NO: 128 - DNA, 35 bp, Artificial Sequence
  Synthetic Construct
acgtaccgaagaccatgacctagctggtgctggcg

SEQ ID NO: 129 - DNA, 36 bp, Artificial Sequence
  Synthetic Construct
acgtaccgaagacaggtcatcctcagctagttggag

SEQ ID NO: 130 - DNA, 37 bp, Artificial Sequence
  Synthetic Construct
acgtaccgaagacagaagctcaaaggccctttctcca

SEQ ID NO: 131 - DNA, 34 bp, Artificial Sequence
  Synthetic Construct
tgcactcgaagacagaatggcttcctctatgctc

SEQ ID NO: 132 - DNA, 33 bp, Artificial Sequence
  Synthetic Construct
tgcactcgaagacagaccttcggaatcggtaag

SEQ ID NO: 133 - DNA, 53 bp, Artificial Sequence
  Synthetic Construct
caactttgtacaaaaaagcaggctccgaattcgcccttatggcttcctctatg

SEQ ID NO: 134 - DNA, 52 bp, Artificial Sequence
  Synthetic Construct
agcgtaatctggaacatcgtatgggtacataccggtgaagcttggtggcttg

SEQ ID NO: 135 - DNA, 52 bp, Artificial Sequence
  Synthetic Construct
atcctcctcagaaatcaacttttgctccataccggtgaagcttggtggcttg

SEQ ID NO: 136 - DNA, 51 bp, Artificial Sequence
  Synthetic Construct
agcgtaatctggaacatcgtatgggtacataacactacgtttgttggctgg

SEQ ID NO: 137 - DNA, 52 bp, Artificial Sequence
  Synthetic Construct
gatcctcctcagaaatcaacttttgctccataacactacgtttgttggctgg

SEQ ID NO: 138 - DNA, 50 bp, Artificial Sequence
  Synthetic Construct
agcgtaatctggaacatcgtatgggtacataaggccctttctccagtctg

SEQ ID NO: 139 - DNA, 53 bp, Artificial Sequence
  Synthetic Construct
aagatcctcctcagaaatcaacttttgctccataaggccctttctccagtctg

SEQ ID NO: 140 - protein, 180 aa, Nicotiana benthamiana
MASSVLSSAAVATRSNVAQANMVAPFTGLKSAASFPVSRKQNLDITSIASNGGRVQCMQVWPPINKKKYETLSYLPDLSVEQLLSEIEYLLKNGWVPCLEFETEHGFVYREHHKSPGYYDGRYWTMWKLPMFGCTDATQVLAEVEEAKKAYPQAWIRIIGFDNVRQVQCISFIAYKPEGY

SEQ ID NO: 141 - protein, 180 aa, Nicotiana benthamiana
MASSVLSSAAVATRSNVAQANMVAPFTGLKSAASFPVSRKQNLDITSIASNGGRVQCMQVWPPINKKKYETLSYLPDLSVEQLLSEIEYLLKNGWVPCLEFETERGFVYREHHKSPGYYDGRYWTMWKLPMFGCTDATQVLAEVEEAKKAYPQAWIRIIGFDNVRQVQCISFIAYKPEGY

SEQ ID NO: 142 - protein, 180 aa, Vigna unguiculata
MASSMISSPAVTTVNRAGAGAGMVAPFTGLKSLGGFPTRKTNNDITSVANNGGRVQCMQVWPTTGKKKFETLSYLPDLTEEQLLKEIDYLLRNGWIPCLEFTLQDPFPYREQNRSPGYYDGRYWTMWKLPMFGCTDATQVLQEVVEARTAHPNGFVRIIGFDNVRQVQCISFIAYKAPGF

SEQ ID NO: 143 - protein, 169 aa, Vigna unguiculata
MSAATFSAQVAGAGFVGLKSNSSSLCQISGSITWKRKIASNSSKTYCMKTWNPINNKKFETLSYLPPLSDESIAKEIDYMIKKGWIPCLEFDELGCIRRENSHMPGYYDGRYWTLWKLPMFGCSESSQVLNEIHECRKAYPNAYIRCLAFDNKRHMQSMAFIIHTPSTT

SEQ ID NO: 144 - protein, 173 aa, Vigna unguiculata
MSAATFSAQVAGAGFVGLKSNSSSLCQISGSITWKRKIASNSSKTYCMKNLFQTWNPINNKKFETLSYLPPLSDESIAKEIDYMIKKGWIPCLEFDELGCIRRENSHMPGYYDGRYWTLWKLPMFGCSESSQVLNEIHECRKAYPNAYIRCLAFDNKRHMQSMAFIIHTPSTT

SEQ ID NO: 145 - protein, 178 aa, Glycine max
MASSMISSPAVTTVNRAGAGMVAPFTGLKSMAGFPTRKTNNDITSIASNGGRVQCMQVWPPVGKKKFETLSYLPDLDDAQLAKEVEYLLRKGWIPCLEFELEHGFVYREHNRSPGYYDGRYWTMWKLPMFGCTDASQVLKELQEAKTAYPNGFIRIIGFDNVRQVQCISFIAYKPPGF

SEQ ID NO: 146 - protein, 178 aa, Glycine max
MASSMISSPAVTTVNRAGAGMVAPFTGLKSMAGLPTRKTNNDITSIASNGGRVQCMQVWPPVGKKKFETLSYLPDLDDAQLAKEVEYLLRKGWIPCLEFELEHGFVYREHNRSLGYYDGRYWTMWKLPMFGCTDASQVLKELQEAKTAYPNGFIRIIGFDNVRQVQCISFIAYKPPSF

SEQ ID NO: 147 - protein, 172 aa, Glycine max
MSAATFAAHIAGAGFVGLKSNSSNLCPSTGSIGWKRKIVSNGSKTYCMKTWNPINNKKFETLSYLPPLSDESIAKEIDYMLKKGWIPCLEFDELGCVRRENSHMPGYYDGRYWTLWKLPMFACSDSSQVLKEIHECRRVYPNAYIRCLAFDNQRHMQSMAFIVHKPDITTTT

SEQ ID NO: 148 - protein, 171 aa, Manihot esculenta
MSTAGIFTAPIIGSGYQGLKAKSTNELFPAKDSIAWSRKTITNGSRIHCMKTWNPINNKKFETLSYLPPLSDESIAKEIDYMMQKGWIPCLEFDQVGHVRRENSQTPGYYDGRYWTMWKLPMFGCNDSSQVLNEIHECKQAYPNAYIRCLAFDNKHQGQCMAFIIQKPNTP

SEQ ID NO: 149 - protein, 182 aa, Manihot esculenta
MASSMLSTATVASINRVSPAQATMVAPFTGLKSTPVFPTTRKTNSDITSITSNGGKVQCMKVWPTLGMKKFETLSYLPPLTREQLASEVEYLLRSGWIPCLEFELEHGLVYREHARVPGYYDGRYWTMWKLPMFGCTDAAQVLKELDELIKHHPDGYARIIGFDNVRQVQCISFLAYKPPGA

SEQ ID NO: 150 - protein, 184 aa, Manihot esculenta
MATSMLSTATVASINRASPAQASMVAPFTGLKSTSAFPATTKTSADITSLASNGGRVQCMQVWPTRGKKKFETLSYLPPLSREQLASEIDYLLRSGWIPCLEFELEHGFVYRAHGSLPGYYDGRYWTMWKLPMFGCTDSSQVLKELDELIKAHPDGFARIIGFDNVRQVQCISFIAYKPPGTDY

SEQ ID NO: 151 - protein, 175 aa, Oryza sativa
MAPTVMASSATSVAPFQGLKSTAGLPVSRRSTNSGFGNVSNGGRIKCMQVWPIEGIKKFETLSYLPPLTVEDLLKQIEYLLRSKWVPCLEFSKVGFVYRENHRSPGYYDGRYWTMWKLPMFGCTDATQVLKELEEAKKAYPDAFVRIIGFDNVRQVQLISFIAYKPPGCEESGGN

SEQ ID NO: 152 - protein, 175 aa, Oryza sativa
MAPSVMASSATSVAPFQGLKSTAGLPVNRRSSSSSFGNVSNGGRIRCMQVWPIEGIKKFETLSYLPPLTVEDLLKQIEYLLRSKWVPCLEFSKVGFVYRENHRSPGYYDGRYWTMWKLPMFGCTDATQVLKELEEAKKAYPDAFVRIIGFDNVRQVQLISFIAYKPPGCEESGGN

SEQ ID NO: 153 - protein, 234 aa, Oryza sativa
MPMPLPSQVHRCHLLPAPPHSPSLVTLLYSPLLNLFTPPPATTTTLYSGDIMFINTASFVAGAVVASPEQPAKLVRDQRRVVPGSCRARRGAASNGFRTYCMQTWNPFTNRRYEAMSYLPPLSAKSISKEIEFIMSKGWVPCLEFDKEGEIHRSNSRMPGYYDGRYWTLWKLPMFGCSDAAAVLREVEECRREYPDAFIRLIAFDSSRQCQCMSFVVHKPPSAAASPATVAGAE

SEQ ID NO: 154 - protein, 175 aa, Triticum aestivum
MAPAVMASSATSVAPFQGLKSTAGLPVSRRSNGASLGSVSNGGRIRRMQVWPIEGIKKFETLSYLPPLSTEALLKQVDYLIRSKWVPCLEFSKVGFIFREHNASPGYYDGRYWTMWKLPMFGCTDATQVINEVEEVKKEYPDAYVRIIGFDNMRQVQCVSFIAFKPPGCEESGKA

SEQ ID NO: 155 - protein, 175 aa, Triticum aestivum
MAPAVMASSATSVAPFQGLKSTAGLPVSRRSSSAGLSSVSNGGRIRCMQVWPIEGIKKFETLSYLPPLSTEALLKQVDYLIRSKWVPCLEFSKVGFVFREHNSSPGYYDGRYWTMWKLPMFGCTDATQVLNEVEEVKKEYPDAYVRVIGFDNLRQVQCVSFIAFRPPGCEESGKA

SEQ ID NO: 156 - protein, 174 aa, Triticum aestivum
MAPAVMASSATTVAPFQGLKSTAGLPVSRRSSGSLGRVSNGGRIRCMQVWPIEGIKKFETLSYLPPLSTEALLKQVDYLIRSKWVPCLEFSKVGFVFREHNSSSGYYDGRYWTMWKLPMFGCTDATQVLNEVEEVKKEYPDAYVRVIGFDNLRQVQCVSFIAFRPPGCEESGKA

SEQ ID NO: 157 - protein, 211 aa, Volvox carteri f. nagariensis
MAAMVMKSSVATAVVRPARSSVRPCAVLKPAVKAATVTAPAQANKMMVWTPVNNKASMYHTDLLHLPCYNTKNPCFFQSGRGFRNPHGIRFLTARWLRWFAACKRPPGWIPCLEFAEADKAYVSNESTVRFGPVSCLYYDNRYWTMWKLPMFGCRDPMQVLREIVACTKAFPDAYVRLVAFDNVKQVQIMGFLVQRPKSARDWQPANKRSV

SEQ ID NO: 158 - protein, 185 aa, Volvox carteri f. nagariensis
MAAVIAKSSVATAVARPARSGVRPVAVLKPSVRATPVATPTQANKMMVWTPVNNKMFETFSYLPPLSDEQIAAQVDYIVANGWIPCLEFAEADKAYVSNESTVRFGPVSCLYYDNRYWTMWKLPMFGCRDPMQVLREIVACTKAFPDAYVRLVAFDNVKQVQIMGFLVQRPKSARDWQPANKRSV

SEQ ID NO: 159 - protein, 284 aa, Volvox carteri f. nagariensis
MQLGGWGEFRRVLDGASLRVPVSLILHGPLRCRFDLEQEGFRVRDETLAKALEKLGRAPYHGQETPPYVDAAVWRWSCLWIAVKVITLDSLVRISYFLRVPGVYVVLGLTTQTANGSSRSVRPCAVLKPAVKAATVAAPAQANKMMVWTPVNNKMFETFSYLPPLTDEQIAAQVDYIVANGWIPCLEFAEADKAYVSNESTVRFGPVSCLYYDNRYWTMWKLPMFGCRDPMQVLREIVACTKAFPDAYVRLVAFDNVKQVQIMGFLVQRPKSARDWQPANKRSV

SEQ ID NO: 160 - protein, 186 aa, Volvox carteri f. nagariensis
MAAIVAKSSVASAVARPSRNSVQRSVAALKPAVKAAPVTAPAQANKMMVWTPVNNKMFETFSYLPPLTDEQIAAQVDYIVANGWIPCLEFAEADKAYVSNESTVRFGPVSCLYYDNRYWTMWKLPMFGCRDPMQVLREIVACTKAFPDAYVRLVAFDNVKQVQIMGFLVQRPKSARDWQPANKRSV

SEQ ID NO: 161 - protein, 185 aa, Volvox carteri f. nagariensis
MAALLAKSSVAAAVARPQRSSVRPCAALKPAVKAAPVATPAQANKMMVWTPVNNKMFETFSYLPPLTDEQIAAQVDYIVANGWIPCLEFAEADKAYVSNESTVRFGPVSCLYYDNRYWTMWKLPMFGCRDPMQVLREIVACTKAFPDAYVRLVAFDNVKQVQIMGFLVQRPKSARDWQPANKRSV

SEQ ID NO: 162 - protein, 185 aa, Volvox carteri f. nagariensis
MAAIVAKSSVAAVVARPARSSVRPVAGLKPAVKAAPVAAPAQANKMMVWTPVNNKMFETFSYLPPLTDEQIAAQVDYIVANGWIPCLEFAEADKAYVSNESTVRFGPVSCLYYDNRYWTMWKLPMFGCRDPMQVLREIVACTKAFPDAYVRLVAFDNVKQVQIMGFLVQRPKSARDWQPANKRSV

SEQ ID NO: 163 - protein, 185 aa, Volvox carteri f. nagariensis
MAAIVAKSSVATAVVRPARSSVRPVAVLKPAIKAAPVASPAQANKMMVWTPVNNKMFETFSYLPPLTDEQIAAQVDYIVANGWIPCLEFAEADKAYVSNESTVRFGPVSCLYYDNRYWTMWKLPMFGCRDPMQVLREIVACTKAFPDAYVRLVAFDNVKQVQIMGFLVQRPKSARDWQPANKRSV

SEQ ID NO: 164 - protein, 185 aa, Gonium pectorale
MAAMIAKSSVSAAVARPARSSARVSAVLKPAVKAAPVAAPSSANKMMVWTPVNNKMFETFSYLPPLSDEQIAAQVDYIVANGWIPCLEFAEADKAYVSNESTVRFGPVSVLYYDNRYWTMWKLPMFGCRDPMQVLREIVACTKAFPDAYVRLVAFDNVKQVQIMGFLVQRPKSARDWQPANKRSV

SEQ ID NO: 165 - protein, 200 aa, Tetrabaena socialis
MATLSSMRIGAAPRVAVARTQRASTVKVVAKGSWRDAPTVTAQPGRAASSAKPTSPTRSVLPANWRQELESLRGGNGNGAAAAPAAAAPRAQSAGWRDAPASAPAASAPMKKTATPARTALPANWKQELESLRSSSTGGASAAPAAAPARASSASWRDAPAAAPASKSSSPAPAGTNPWTGKSKIEIKRTALPADWRKGL

SEQ ID NO: 166 - protein, 298 aa, Volvox carteri f. nagariensis
MAMSTMRVGAAPRVAVARSQSVKVVARGSWRESATVTAQPAGRASSSNRVSPTRSVLPANWRQELESLRNGNGNGAAAAPAPAPAPARSSSASWRSESSAAPAAASTPSRSTKKPVTPTRTSLPANWKQELESLRGSSSSSPAAAAPAPARSSSSPKKAVTPTRSSLPANWKQELESLRGGSSSAASAPAAAAAPAAASAPSRSPKKAVTPTRSSLPANWKQELESLRGGSSSSSSAPAPAAAPAPARSSSASWRTESPAPANESSSAAAKAGTNPWTGKAKIEIKRTTLPADWRRQL

SEQ ID NO: 167 - protein, 245 aa, Gonium pectorale
MALSAMRVGAAPRAAVSRPQTVQVVARGSWRESSTVTATPAGRSSSAANRVSPTRSVLPANWRQELESLRNGNGNGSSAAAAPAPAPARSASASWRDAPAAAAPARPSSSPKKAVTPSRSSLPANWKQELEALRGGSSSSSASWRTESAPAAAPARSGSKKAVTPSRSSLPANWKQELESMRSASPAPSSAPAAPARSSSASWRSESGSSSSSAAADKAGTNPWTGKAKVEIKRTALPADWRKGL

SEQ ID NO: 168 - DNA, 13441 bp, Artificial Sequence
  Synthetic Construct
gaacactctgtgccgaattcggatccagcggtcctgctgagcctcgacatgttgtcgcaaaattcgccctggacccgcccaacgatttgtcgtcactgtcaaggtttgacctgcacttcatttggggcccacatacaccaaaaaaatgctgcataattctcggggcagcaagtcggttacccggccgccgtgctggaccgggttgaatggtgcccgtaactttcggtagagcggacggccaatactcaacttcaaggaatctcacccatgcgcgccggcggggaaccggagttcccttcagtgagcgttattagttcgccgctcggtgtgtcgtagatactagcccctggggcacttttgaaatttgaataagatttatgtaatcagtcttttaggtttgaccggttctgccgctttttttaaaattggatttgtaataataaaacgcaattgtttgttattgtggcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcttctcgcatatctcattaaagcaggacaagcttatcattcctcaccagcatcagcatcaggggtcttgaaagcatgttggtactcaacgattccaagctcggtgttagagtgatcttcctcaactcttctgaaagcgaacataggtccaccgttttgaaggatagaagggtggatagcagacttgaagtgcatgtgagaatccaccacagaagagtagtaaccaccatctctaagtgagaaggttctggtgaaagatccatcgagatcgttatctcccataggatgaagatgctcaacagtagcgttagacctgatgatcttgtcggtgaagataacagaatcctcagggaatccagttcccataacctgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatatcaaaggtagaagcagaaacttaccttgaaatctccgatcactcttccagcctcgtatctgtaagagaagctaacgtgaagaacaccaccatcctcgtacttctcgatcctagtgttggtgtatccaccgttgttgatagcatgaaggaaagggttctcgtatccagatgggtaagttccgaagtggtagaatccgtatcccataacgtgagaaagaaggtatggagagaaggtaagagcacccttggtagacttcatcttgttagtcattcttccctgctcaggagttccctcaccacctccaacaagctcgaactcaacaccgttaagggttccagtgattctacactcgatttccatagcaggaagtccagactcatcagactcagatccagatcctctcgaaaggccctttctccagtctgcagggagagccgttctcttaatttctggcttgcttttacctgtccaaggattagttccggctttgtcagcggaggaactagatgacgaagcgggagcgtctctccaggaagcggatgatgatctagcaggtgcaggggcactgcttgcaggtgcgggactgttggatcggagtgactccaactcctgtttccaattacttggaagggccgatctactaggggtgactgcttttttgctcgccgatgagctacgagctggggcggaagaagcaggagctgcatccctccaactagctgaggatgacctagctggtgctggcgcgctagaagctggtgctggactgcttgatcgaagggattcaagttcctgtttccagttagaaggcagagctgaacgagacggagtgacggccttcttgctggaagaactcctagctggagcgctagatgcaggcgctgcgtccctccatgaggcagaactacttctggccggagctggcgctgatgaagccggtgcagggctggagcttctcaatgactccaattcctgcttccaatttgaaggcaaggcacttcttgaaggagtaacggccttctttgaagctgagctacttctggcaggcgctgatgatgcaggagcggcgtctcgccacgatgcgctgctcgatctcgcaggagcaggagcagaggacgccgccgacgagctgccgttaccatttctgagtgattcaagttcttgcctccaattagctgggagaacgcttcttgttgggctcactctgttcgttgcactactagctcttgaggcttgaactgtagcagtagatgactctctccaagatcctcttgctgcacctccgcagttaactcttccgccgttgcttgtgatggaagtaatgtcgttgttagccttgcgggtggctgggaaggcagcggaggacttaagtccgttgaaaggagcgaccatagtggcctgagccggagaggcaaccatagtagcggaagagagcatagaggaagccattgtatcgataattgtaaatgtaattgtaatgttgtttgttgtttgttgttgttggtaattgttgtaaaaatgagctcttatactcgagcgtgtcctctccaaatgaaatgaacttccttatatagaggaagggtcttgcgaaggatagtgggattgtgcgtcatcccttacgtcagtggagatgtcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttgacctccgcaagaattcaagcttggagccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgatgacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtaatggcttcctctatgctctcttccgctactatggttgcctctccggctcaggccactatggtcgctcctttcaacggacttaagtcctccgctgccttcccagccacccgcaaggctaacaacgacattacttccatcacaagcaacggcggaagagttaactgcggaggtgcagcaagaggatcttggagagagtcatctactgctacagttcaagcctcaagagctagtagtgcaacgaacagagtgagcccaacaagaagcgttctcccagctaattggaggcaagaacttgaatcactcagaaatggtaacggcagctcgtcggcggcgtcctctgctcctgctcctgcgagatcgagcagcgcatcgtggcgagacgccgctcctgcatcatcagcgcctgccagaagtagctcagcttcaaagaaggccgttactccttcaagaagtgccttgccttcaaattggaagcaggaattggagtcattgagaagctccagccctgcaccggcttcatcagcgccagctccggccagaagtagttctgcctcatggagggacgcagcgcctgcatctagcgctccagctaggagttcttccagcaagaaggccgtcactccgtctcgttcagctctgccttctaactggaaacaggaacttgaatcccttcgatcaagcagtccagcaccagcttctagcgcgccagcaccagctaggtcatcctcagctagttggagggatgcagctcctgcttcttccgccccagctcgtagctcatcggcgagcaaaaaagcagtcacccctagtagatcggcccttccaagtaattggaaacaggagttggagtcactccgatccaacagtcccgcacctgcaagcagtgcccctgcacctgctagatcatcatccgcttcctggagagacgctcccgcttcgtcatctagttcctccgctgacaaagccggaactaatccttggacaggtaaaagcaagccagaaattaagagaacggctctccctgcagactggagaaagggcctttcgtacccatacgatgttcctgactatgcgggctatccctatgacgtcccggactatgcaggattgtatccatatgacgttccagattacgccactagagctgcttacccatacgatgttcctgactatgcgggctatccctatgacgtcccggactatgcaggattgtatccatatgacgttccagattacgccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcttatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataagagatgtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgctactagaattcgagctcggagttctagaatgtcgcggaacaaattttaaaactaaatcctaaatttttctaattttgttgccaatagtggatatgtgggccgtatagaaggaatctattgaaggcccaaacccatactgacgagcccaaaggttcgttttgcgttttatgtttcggttcgatgccaacgccacattctgagctaggcaaaaaacaaacgtgtctttgaatagactcctctcgttaacacatgcagcggctgcatggtgacgccattaacacgtggcctacaattgcatgatgtctccattgacacgtgacttctcgtctcctttcttaatatatctaacaaacactcctacctcttccaaaatatatacacatctttttgatcaatctctcattcaaaatctcattctctctagtaaacaagaacaaaaaaatggcggatacagctagaggaacccatcacgatatcatcggcagagatcagtacccgatgatgggccgagatcgtgaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtaagcacacatttatcatcttacttcataattttgtgcaatatgtgcatgcatgtgttgagccagtagctttggatcaatttttttggtcgaataacaaatgtaacaataagaaattgcaaattctagggaacatttggttaactaaatacgaaatttgacctagctagcttgaatgtgtctgtgtatatcatctatataggtaaaatgcttggtatgatacctattgattgtgaataggtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggccagcacactactatgagcgagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaagcgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagaaacctacgtcgagcagcacgaggtggctgtggccagatactgcgacctccctagcaaactggggcacaagtgagcttaccccactgatgtcatcgtcatagtccaataactccaatgtcggggagttagtttatgaggaataaagtgtttagaatttgatcagggggagataataaaagccgagtttgaatctttttgttataagtaatgtttatgtgtgtttctatatgttgtcaaatggtaccatgttttttttcctctctttttgtaacttgcaagtgttgtgttgtactttatttggcttctttgtaagttggtaacggtggtctatatatggaaaaggtcttgttttgttaaacttatgttagttaactggattcgtctttaaccacaaaaagttttcaataagctacaaatttagacacgcaagccgatgcagtcattagtacatatatttattgcaagtgattacatggcaacccaaacttcaaaaacagtaggttgctccatttagtcgctttacgaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggagtcagccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaagcttctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaacagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggctccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtt

SEQ ID NO: 169 - protein, 10 aa, Artificial Sequence
  Synthetic Construct
WRQELESLRN

SEQ ID NO: 170 - protein, 10 aa, Artificial Sequence
  Synthetic Construct
WKQELESLRS

SEQ ID NO: 171 - DNA, 6199 bp, Artificial Sequence
  Synthetic Construct
  misc_feature (1)..(718): ocs terminator
  misc_feature (715)..(718): MoClo overhang in ocs terminator
  misc_feature (719)..(1624): turbo GFP (Pontellina pluma)
  misc_feature (1622)..(1624): MoClo overhang in turbo GFP (Pontellina pluma)
  misc_feature (1625)..(2497): mature EPYC1
  misc_feature 1625: MoClo overhang in mature EPYC1
  misc_feature (2498)..(2669): Rbcs1A transit peptide
  misc_feature (2498)..(2501), (2666)..(2669): MoClo overhang in Rbcs1A transit peptide
  misc_feature (2670)..(3181): 35S promoter
  misc_feature (3182)..(3205): linking region
  misc_feature (3182)..(3185), (3202)..(3205): MoClo overhang in linking region
  misc_feature (3206)..(3722): CSVMV promoter
  misc_feature 3722: MoClo overhang in CSVMV promoter
  misc_feature (3723)..(3893): Rbcs1A transit peptide
  misc_feature (3723)..(3725), (3890)..(3893): MoClo overhang in Rbcs1A transit peptide
  misc_feature (3894)..(4766): mature EPYC1
  misc_feature 4766: MoClo overhang in mature EPYC1
  misc_feature (4767)..(5678): eGFP (with N-terminal HA-tag)
  misc_feature (4767)..(4769): MoClo overhang in eGFP (with N-terminal HA-tag)
  misc_feature (5679)..(5932): HSP terminator
  misc_feature (5679)..(5682): MoClo overhang in HSP terminator
  misc_feature (5933)..(6199): nos terminator
  misc_feature (5933)..(5936): MoClo overhang in nos terminator
gtcctgctgagcctcgacatgttgtcgcaaaattcgccctggacccgcccaacgatttgtcgtcactgtcaaggtttgacctgcacttcatttggggcccacatacaccaaaaaaatgctgcataattctcggggcagcaagtcggttacccggccgccgtgctggaccgggttgaatggtgcccgtaactttcggtagagcggacggccaatactcaacttcaaggaatctcacccatgcgcgccggcggggaaccggagttcccttcagtgagcgttattagttcgccgctcggtgtgtcgtagatactagcccctggggcacttttgaaatttgaataagatttatgtaatcagtcttttaggtttgaccggttctgccgctttttttaaaattggatttgtaataataaaacgcaattgtttgttattgtggcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcttctcgcatatctcattaaagcaggacaagcttatcattcctcaccagcatcagcatcaggggtcttgaaagcatgttggtactcaacgattccaagctcggtgttagagtgatcttcctcaactcttctgaaagcgaacataggtccaccgttttgaaggatagaagggtggatagcagacttgaagtgcatgtgagaatccaccacagaagagtagtaaccaccatctctaagtgagaaggttctggtgaaagatccatcgagatcgttatctcccataggatgaagatgctcaacagtagcgttagacctgatgatcttgtcggtgaagataacagaatcctcagggaatccagttcccataacctgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatatcaaaggtagaagcagaaacttaccttgaaatctccgatcactcttccagcctcgtatctgtaagagaagctaacgtgaagaacaccaccatcctcgtacttctcgatcctagtgttggtgtatccaccgttgttgatagcatgaaggaaagggttctcgtatccagatgggtaagttccgaagtggtagaatccgtatcccataacgtgagaaagaaggtatggagagaaggtaagagcacccttggtagacttcatcttgttagtcattcttccctgctcaggagttccctcaccacctccaacaagctcgaactcaacaccgttaagggttccagtgattctacactcgatttccatagcaggaagtccagactcatcagactcagatccagatcctctcgaaaggccctttctccagtctgcagggagagccgttctcttaatttctggcttgcttttacctgtccaaggattagttccggctttgtcagcggaggaactagatgacgaagcgggagcgtctctccaggaagcggatgatgatctagcaggtgcaggggcactgcttgcaggtgcgggactgttggatcggagtgactccaactcctgtttccaattacttggaagggccgatctactaggggtgactgcttttttgctcgccgatgagctacgagctggggcggaagaagcaggagctgcatccctccaactagctgaggatgacctagctggtgctggcgcgctagaagctggtgctggactgcttgatcgaagggattcaagttcctgtttccagttagaaggcagagctgaacgagacggagtgacggccttcttgctggaagaactcctagctggagcgctagatgcaggcgctgcgtccctccatgaggcagaactacttctggccggagctggcgctgatgaagccggtgcagggctggagcttctcaatgactccaattcctgcttccaatttgaaggcaaggcacttcttgaaggagtaacggccttctttgaagctgagctacttctggcaggcgctgatgatgcaggagcggcgtctcgccacgatgcgctgctcgatctcgcaggagcaggagcagaggacgccgccgacgagctgccgttaccatttctgagtgattcaagttcttgcctccaattagctgggagaacgcttcttgttgggctcactctgttcgttgcactactagctcttgaggcttgaactgtagcagtagatgactctctccaagatcctcttgctgcacctccgcagttaactcttccgccgttgcttgtgatggaagtaatgtcgttgttagccttgcgggtggctgggaaggcagcggaggacttaagtccgttgaaaggagcgaccatagtggcctgagccggagaggcaaccatagtagcggaagagagcatagaggaagccattgtatcgataattgtaaatgtaattgtaatgttgtttgttgtttgttgttgttggtaattgttgtaaaaatgagctcttatactcgagcgtgtcctctccaaatgaaatgaacttccttatatagaggaagggtcttgcgaaggatagtgggattgtgcgtcatcccttacgtcagtggagatgtcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttgaccctcgcaagaattcaagcttggagccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgatgacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtaatggcttcctctatgctctcttccgctactatggttgcctctccggctcaggccactatggtcgctcctttcaacggacttaagtcctccgctgccttcccagccacccgcaaggctaacaacgacattacttccatcacaagcaacggcggaagagttaactgcggaggtgcagcaagaggatcttggagagagtcatctactgctacagttcaagcctcaagagctagtagtgcaacgaacagagtgagcccaacaagaagcgttctcccagctaattggaggcaagaacttgaatcactcagaaatggtaacggcagctcgtcggcggcgtcctctgctcctgctcctgcgagatcgagcagcgcatcgtggcgagacgccgctcctgcatcatcagcgcctgccagaagtagctcagcttcaaagaaggccgttactccttcaagaagtgccttgccttcaaattggaagcaggaattggagtcattgagaagctccagccctgcaccggcttcatcagcgccagctccggccagaagtagttctgcctcatggagggacgcagcgcctgcatctagcgctccagctaggagttcttccagcaagaaggccgtcactccgtctcgttcagctctgccttctaactggaaacaggaacttgaatcccttcgatcaagcagtccagcaccagcttctagcgcgccagcaccagctaggtcatcctcagctagttggagggatgcagctcctgcttcttccgccccagctcgtagctcatcggcgagcaaaaaagcagtcacccctagtagatcggcccttccaagtaattggaaacaggagttggagtcactccgatccaacagtcccgcacctgcaagcagtgcccctgcacctgctagatcatcatccgcttcctggagagacgctcccgcttcgtcatctagttcctccgctgacaaagccggaactaatccttggacaggtaaaagcaagccagaaattaagagaacggctctccctgcagactggagaaagggcctttcgtacccatacgatgttcctgactatgcgggctatccctatgacgtcccggactatgcaggattgtatccatatgacgttccagattacgccactagagctgcttacccatacgatgttcctgactatgcgggctatccctatgacgtcccggactatgcaggattgtatccatatgacgttccagattacgccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcttatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataagagatgtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcga

SEQ ID NO: 172 - protein, 14 aa, Artificial Sequence
  Synthetic Construct
KSARDWQPANKRSV